(19) United States
(12) Patent Application Publication
(10) Pub. No.: US 2023/0346934 A1
(43) Pub. Date: Nov. 2, 2023

(54) CHIMERIC SWITCH RECEPTORS FOR THE CONVERSION OF IMMUNESUPPRESSIVE SIGNALS TO COSTIMULATORY SIGNALS

(71) Applicant: Allogene Therapeutics, Inc., South San Francisco, CA (US)

(72) Inventors: Yi ZHANG, Foster City, CA (US); Silvia K. TACHEVA-GRIGOROVA, Foster City, CA (US); Barbra Johnson SASU, San Francisco, CA (US); Siler PANOWSKI, Berkeley, CA (US); Regina Junhui LIN, Santa Mateo, CA (US); Zhe LI, Burlingame, CA (US)

(21) Appl. No.: 18/191,955

(22) Filed: Mar. 29, 2023

Related U.S. Application Data
(60) Provisional application No. 63/453,936, filed on Mar. 22, 2023.
(60) Provisional application No. 63/325,069, filed on Mar. 29, 2022.

(51) Int. Cl. A61K 39/00; C07K 14/705; C07K 14/71; C07K 16/28; C07K 16/22; C07K 14/725; A61P 35/00

(52) U.S. Cl. CPC A61K 39/4611; C07K 14/70521; C07K 14/71; C07K 16/2827; C07K 16/22; C07K 14/70507; C07K 14/70517; C07K 14/7051; A61K 39/4631; A61K 39/4643; A61P 35/00; C07K 2319/03; C07K 2317/622; C07K 2319/02; A61K 2239/13; A61K 2239/21; A61K 2239/22; A61K 2239/23; A61K 2239/28

(57) ABSTRACT

Provided herein are chimeric switch receptors (CSRs) comprising an ectodomain and/or transmembrane domain derived from an inhibitory receptor (e.g. PD1 or TGFβR2) fused to the transmembrane domain and/or intracellular signaling domain derived from one or more costimulatory proteins (e.g. CD2, CD28, MyD88, DAP10 or ICOS), or variants thereof. The chimeric switch receptors are designed to convert a signal e.g. an inhibitory signal such as an immunosuppressive signal in the form of PD-L1 or TGFβ into a costimulatory signal. Also provided are engineered immune cells engineered to functionally express a chimeric switch receptor and/or a CAR and optionally also a chimeric cytokine receptor (CCR), and populations thereof, methods of making and using the engineered cells, compositions and kits comprising them, and methods of treating e.g. cancer (e.g. solid or hematologic tumors) by administering the cells and the compositions.

### CROSS REFERENCE TO RELATED APPLICATIONS

The present application claims the benefit of priority to U.S. Provisional Application No. 63/325,069, filed on Mar. 29, 2022; and U.S. Provisional Application No. 63/453,936, filed on Mar. 22, 2023, the contents of both of which are hereby incorporated by reference in their entireties.

### SEQUENCE LISTING

This application contains a Sequence Listing which has been submitted electronically in XML format and is hereby incorporated by reference in its entirety. Said XML copy, created on Mar. 24, 2023, is named AT-050_03_SL.xml and is 294,461 bytes in size.

### FIELD

The present disclosure relates generally to chimeric polypeptides, engineered immune cells (e.g., T cells) comprising the same, polynucleotide encoding the same, and use of the engineered immune cells in therapeutic applications.

### BACKGROUND

Adoptive transfer of immune cells genetically modified to recognize malignancy-associated antigens is showing promise as a new approach to treating cancer (see, e.g., Brenner et al., Current Opinion in Immunology, 22(2): 251-257 (2010); Rosenberg et al., Nature Reviews Cancer, 8(4): 299-308 (2008)). Immune cells can be genetically modified to express chimeric antigen receptors (CARs), fusion proteins comprised of an antigen recognition moiety and T cell activation domains (see, e.g., Eshhar et al., Proc. Natl. Acad. Sci. USA, 90(2): 720-724 (1993)). Immune cells that contain CARs, e.g., CAR-T cells (CAR-Ts), are engineered to endow them with antigen specificity while retaining or enhancing their ability to recognize and kill a target cell.

Improvements in CAR-T cell therapy, e.g. to improve efficiency and/or accuracy of treatment, will benefit the patient community.

### SUMMARY

Provided herein are, inter alia, chimeric polypeptides, specifically chimeric switch receptors, which are fusion proteins comprising an ectodomain and/or transmembrane domain derived from an inhibitory receptor (e.g. PD1 or TGFbR2 (TGF beta receptor II)) fused to the transmembrane domain and/or intracellular signaling domain derived from one or more costimulatory proteins (e.g. CD2, CD28, MyD88, DAP10 or ICOS).

Chimeric switch receptors may be leveraged in cell-based immunotherapies (e.g. CAR T therapy) by subverting immunesuppression and enhancing potency. The benefit of chimeric switch receptors is multi-fold. First, the ectodomain can serve as a decoy/dominant negative receptor that protects CAR T cells from immunosuppression (e.g. PD1 ligands PDL 1 or TGFb). Second, the intracellular signaling domain provides additional costimulatory signals that may potentiate CAR T cell activity and potency. Third, signaling by chimeric switch receptors is activated in the presence of the ectodomain's ligand. Consequently, it can be preferentially turned on in suppressive tumor microenvironments (e.g. where the ectodomain ligand is abundant) where potency enhancement is most needed, and be turned down or off elsewhere (e.g. in environments with little or no ectodomain ligand) to maintain an adequate safety profile. In certain embodiments, engineered immune cells comprising or expressing the chimeric polypeptides disclosed herein overcome the immunosuppressive tumor microenvironment, demonstrate potentiated activity, while secret acceptable levels of cytokine and achieve adequate or improved safety profile.

Also provided herein are polynucleotides and vectors that encode the disclosed chimeric switch receptors, cells e.g. engineered immune cells such as engineered T cells that comprise and/or express the disclosed chimeric switch receptors and/or the polynucleotides and vectors that encode them, populations of such cells, compositions comprising such cells and populations of cells, methods of treating conditions e.g. cancers comprising administering the cells and populations of cells disclosed herein, methods of engineering such cells, and cells prepared by such methods.

In one aspect, the present disclosure provides a polynucleotide encoding a chimeric polypeptide comprising an extracellular domain, a transmembrane domain, and one or more intracellular domains, wherein the extracellular domain comprises an extracellular domain of an inhibitory protein. In some embodiments, the inhibitory protein is an inhibitory receptor. In some embodiments, the encoded chimeric polypeptide's extracellular domain comprises a PD-1 (programmed cell death protein-1) extracellular domain. In some embodiments, the PD-1 extracellular domain comprises the amino acid sequence of a wild-type hPD-1 (human PD-1) extracellular domain (e.g. the amino acid sequence of SEQ ID NO:9). In some embodiments, the PD-1 extracellular domain comprises the amino acid sequence of a variant of a wild-type hPD-1 extracellular domain (e.g. the amino acid sequence of a variant of SEQ ID NO:9 such as an amino acid sequence that is at least 90% identical to SEQ ID NO:9). In some embodiments, the PD-1 extracellular domain variant comprises one or more amino acid insertions, deletions and/or substitutions relative to the wild-type PD-1 extracellular domain e.g. relative to SEQ ID NO:9. In some embodiments, the encoded chimeric polypeptide's PD-1 extracellular domain variant comprises the amino acid sequence of a high affinity (“HA”) hPD-1 extracellular domain (e.g. an exemplary HA hPD-1 extracellular domain comprises or has the amino acid sequence of SEQ ID NO:10; see, e.g., R. L. Maute et al., Proc Natl Acad Sci USA. 2015; 112(47):E6506-E6514. doi:10.1073/pnas.1519623112 for HA hPD-1 proteins).

In some embodiments of the polypeptides and encoded polypeptides disclosed herein in which a second amino acid sequence is at least 90% identical to a first amino acid sequence, one or more (e.g. all or 1, 2, 3, 4, 5, 6, 7, 8, 9 or 10) of the amino acids in the second amino acid sequence that are not identical to the corresponding amino acid in the first amino acid sequence, e.g., amino acid substitutions. In some embodiments, the amino acid substitutions are conservative amino acid substitutions.

In some embodiments, the encoded chimeric polypeptide's extracellular domain comprises a TGFβ receptor (e.g. TGFBR2) extracellular domain. In some embodiments, the TGFβ receptor extracellular domain comprises the amino acid sequence of a wild-type hTGFβ receptor extracellular domain (e.g. the amino acid sequence of SEQ ID NO: 12). In some embodiments, the encoded chimeric polypeptide's extracellular domain comprises a variant of a TGFβ receptor (e.g. TGFBR2) extracellular domain. In some embodiments, the encoded chimeric polypeptide's extracellular domain comprises a variant of the amino acid sequence of SEQ ID NO:12 e.g. an amino acid sequence that is at least 90% identical to the amino acid sequence of SEQ ID NO:12). In some embodiments, the extracellular domain comprises a hTGFb receptor extracellular domain variant comprising one or more amino acid insertions, deletions and/or substitutions relative to the wild-type hTGFβ receptor extracellular domain e.g. relative to SEQ ID NO:12. In some embodiments, the variant comprises a 25-amino acid deletion from the N-terminus of the mature wild-type hTGFβ receptor and comprises the amino acid sequence of SEQ ID NO:13.

In some embodiments, the encoded chimeric polypeptide's extracellular domain comprises an antibody or an antigen-binding portion of an antibody that specifically recognizes and binds to an inhibitory ligand, e.g. an inhibitory ligand such as PDL1 or TGFβ or one or more than one specific TGFβ isoform. In some embodiments, the encoded chimeric polypeptide's extracellular domain comprises a single-chain variable fragment (scFv) that specifically recognizes and binds to an inhibitory ligand, such as PDL1 or TGFβ or one or more TGFβ isoforms, e.g. a TGFβ isoform selected from TGFβ1, TGFβ2 or TGFβ3. In some embodiments, the encoded chimeric polypeptide's extracellular domain comprises an anti-PDL1 scFv. In some embodiments, the anti-PDL1 scFv may comprise the antigen binding domains of, for example, avelumab, durvalumab or atezolizumab. In some embodiments, the encoded chimeric polypeptide's extracellular domain comprises an anti-TGFβ scFv. In some embodiments, the encoded chimeric polypeptide's extracellular domain comprises an anti-TGFβ scFv that binds to and recognizes only one TGFβ isoform, such as a TGFβ isoform selected from TGFβ1, TGFβ2 or TGFβ3, e.g. an anti-TGFβ scFv that binds to and recognizes only TGFβ1. In some embodiments, the anti-TGFβ scFv may comprise the antigen binding domain of fresolimumab or SAR438459. Anti-TGFβ isoform antibodies e.g. anti-TGFβ isoform scFvs and methods of making them are disclosed in, for example, US 20190071493, which is incorporated herein by reference in its entirety.

In some embodiments, the encoded polypeptide comprises one or only one intracellular signalling domain. In some embodiments, the encoded polypeptide comprises more than one intracellular signalling domains. In some embodiments, the encoded polypeptide comprises more than one intracellular signalling domains, all of which are the same as each other. In some embodiments, the encoded polypeptide comprises more than one intracellular signalling domains, all of which are different from each other. In some embodiments, the encoded polypeptide comprises more than one intracellular signalling domains, some of which are the same as each other and some of which are different from the others. In some embodiments, the encoded polypeptide comprises two, three, four, five or six intracellular signalling domains, of which all are the same as each other, none of which are the same as each other, or some of which are the same as each other.

In some embodiments, the one or more intracellular domains of the encoded chimeric polypeptide comprise one or more intracellular signalling domains. In some embodiments, the one or more intracellular signalling domains are selected from the group consisting of a CD28 intracellular signalling domain, CD2 intracellular signalling domain, MyD88 intracellular signalling domain, ICOS intracellular signalling domain, DAP10 intracellular signalling domain, OX40 intracellular signalling domain, BAFFR intracellular signalling domain, and CD40 intracellular signalling domain, any variants thereof, and any combinations thereof. In some embodiments, the one or more intracellular signalling domains comprises a CD28 intracellular signalling domain. In some embodiments, the one or more intracellular signalling domains comprises a CD2 intracellular signalling domain. In some embodiments, the one or more intracellular signalling domains comprises a MyD88 intracellular signalling domain. In some embodiments, the one or more intracellular signalling domains comprises an ICOS intracellular signalling domain. In some embodiments, the one or more intracellular signalling domains comprises a DAP10 intracellular signalling domain. In some embodiments, the one or more intracellular signalling domains comprises an OX40 intracellular signalling domain. In some embodiments, the one or more intracellular signalling domains comprises a BAFFR intracellular signalling domain. In some embodiments, the one or more intracellular signalling domains comprises a CD40 intracellular signalling domain. In some embodiments, the one or more intracellular signalling domains comprises any combination of CD28, CD2, MyD88, ICOS, DAP10, OX40, BAFFR, and CD40 intracellular signalling domains.

In some embodiments, the encoded chimeric polypeptide's one or more intracellular domains comprise the amino acid sequence of, or two or more copies of the amino acid sequence of, any one or more than one of SEQ ID NO:18, SEQ ID NO:19, SEQ ID NO:20, SEQ ID NO:23, SEQ ID NO:24, SEQ ID NO:15, SEQ ID NO:29, SEQ ID NO:27, SEQ ID NO:33, SEQ ID NO:31, and SEQ ID NO:35, set forth in Table 1. The corresponding polynucleotide sequences are set forth in Table 2. In some embodiments, the encoded chimeric polypeptide's one or more intracellular domains comprise any combination of the amino acid sequences of (e.g. any two of, any three of, any four of, or any five of, or multiple copies of any two of, any three of, any four of, or any five of) SEQ ID NO:15, SEQ ID NO:18, SEQ ID NO:19, SEQ ID NO:20, SEQ ID NO:23, SEQ ID NO:24, SEQ ID NO:27, SEQ ID NO:29, SEQ ID NO:31, SEQ ID NO:33, and SEQ ID NO:35 as set forth in Table 1. The corresponding polynucleotide sequences are set forth in Table 2.

In some embodiments, the encoded chimeric polypeptide's transmembrane domain comprises a CD28 transmembrane domain, CD2 transmembrane domain, PD-1 transmembrane domain, ICOS transmembrane domain, DAP10 transmembrane domain, OX40 transmembrane domain, BAFFR transmembrane domain or CD40 transmembrane domain. In some embodiments, the encoded chimeric polypeptide's transmembrane domain comprises the amino acid sequence of SEQ ID NO:14, SEQ ID NO:17, SEQ ID NO:22, SEQ ID NO:26, SEQ ID NO:28, SEQ ID NO:30, SEQ ID NO:32, or SEQ ID NO:34, set forth in Table 1. The corresponding polynucleotide sequences are set forth in Table 2.

In some embodiments, the intracellular domain of the encoded chimeric polypeptide or chimeric switch receptor comprises a CD28 or CD2 intracellular domain, or a variant thereof. In some embodiments, the intracellular domain comprises the amino acid sequence of SEQ ID NO:19, SEQ ID NO:20 or SEQ ID NO: 24. In some embodiments, the chimeric polypeptide comprises a variant CD28 or a variant CD2 intracellular domain and the engineered immune cells comprising or expressing the chimeric polypeptide express or secret reduced levels of cytokine upon binding of the extracellular domain of the chimeric polypeptide to its ligand. In some embodiments, the intracellular domain comprises a variant CD28 or a variant CD2 intracellular domain and the reduced level of cytokine is about 1.5 fold, 2 fold, 2.5 fold, 3 fold, 3.5 fold, 4 fold, 4.5 fold, and 5 fold less than the levels of cytokine secreted by the engineered immune cells comprising or expressing a chimeric polypeptide that comprises a corresponding wildtype intracellular domain. In certain embodiments, the extracellular domain comprises a PD1 or TGFβ receptor extracellular domain. In some embodiments, the cytokine is GM-CSF, INFγ, TNFα or IL2. In some embodiments, the engineered immune cells comprising or expressing the chimeric polypeptide of the disclosure secret reduced levels of cytokine and exhibit improved safety profile when used in adoptive cell therapy. In some embodiments, the engineered immune cells are CAR T cells.

In some embodiments, the encoded chimeric polypeptide's intracellular domain comprises a CD2 intracellular domain or a variant thereof. In some embodiments, the encoded chimeric polypeptide's intracellular domain comprises a CD2 intracellular domain or a variant thereof and the encoded chimeric polypeptide's transmembrane domain comprises a CD2 transmembrane domain. In some embodiments, the encoded chimeric polypeptide's intracellular domain comprises a CD2 intracellular domain having the amino acid sequence of SEQ ID NO:23. In some embodiments, the encoded chimeric polypeptide's intracellular domain comprises an amino acid sequence that is at least about 90% identical to the amino acid sequence of SEQ ID NO:23.

In some embodiments of the polynucleotide disclosed herein encoding a chimeric polypeptide comprising an extracellular domain, a transmembrane domain, and one or more intracellular domains, the intracellular domain comprises a truncated CD2 intracellular domain. In some embodiments, the truncated CD2 intracellular domain comprises the amino acid sequence of SEQ ID NO:24. In some embodiments, the truncated CD2 intracellular domain comprises an amino acid sequence that is at least about 90% identical to the amino acid sequence of SEQ ID NO:24.

In some embodiments of the polynucleotide disclosed herein, the encoded transmembrane domain comprises a CD2 transmembrane domain. In some embodiments, the CD2 transmembrane domain comprises the amino acid sequence of SEQ ID NO:22. In some embodiments, the CD2 transmembrane domain comprises an amino acid sequence that is at least about 90% identical to the amino acid sequence of SEQ ID NO:22.

In some embodiments of the polynucleotide disclosed herein, the encoded polypeptide comprises a signal peptide. In some embodiments, the encoded polypeptide comprises a CD8a, signal peptide. In some embodiments, the signal peptide comprises the amino acid sequence of SEQ ID NO:1. In some embodiments, the signal peptide comprises the amino acid sequence of SEQ ID NO:2.

In some embodiments of the polynucleotide disclosed herein encoding a chimeric polypeptide comprising an extracellular domain, a transmembrane domain, and one or more intracellular domains, the polypeptide further comprises a hinge domain located between the extracellular domain and the transmembrane domain. In some embodiments, the hinge domain comprises the amino sequence of SEQ ID NO:36. In some embodiments, the hinge domain comprises an amino acid sequence that is at least about 90% identical to the amino acid sequence of SEQ ID NO:36.

In some embodiments of the polynucleotide disclosed herein encoding a chimeric polypeptide comprising an extracellular domain, a transmembrane domain, and one or more intracellular domains e.g. chimeric switch receptor, the polynucleotide comprises the nucleic acid sequence of any one of SEQ ID NOs:118-158 (set forth in Table 4) and the polypeptide comprises the amino acid sequence of any one of SEQ ID NOS:75-115 (set forth in Table 3). In some embodiments, the polypeptide comprises an amino acid sequence that is at least 90% identical to the amino acid sequence of any one of SEQ ID NOS:75-115. In some embodiments, the encoded polypeptide is a PD1 chimeric switch receptor and comprises the amino acid sequence of any one of SEQ ID NOS:75-94. In some embodiments, the encoded polypeptide comprises an amino acid sequence that is at least 90% identical to the amino acid sequence of any one of SEQ ID NOS:75-94. In some embodiments, the encoded polypeptide is a TGF-beta R2 (TGFBR2 or BR2) chimeric switch receptor and comprises the amino acid sequence of any one of SEQ ID NOS:95-115. In some embodiments, the encoded polypeptide comprises an amino acid sequence that is at least 90% identical to the amino acid sequence of any one of SEQ ID NOS:95-115. In some embodiments, the encoded polypeptide further comprises a signal peptide. In some embodiments, the signal peptide comprises the amino acid sequence of SEQ ID NO: 1. In some embodiments, the signal peptide comprises the amino acid sequence of SEQ ID NO:2.

In some embodiments of the polynucleotide disclosed herein encoding a chimeric polypeptide comprising an extracellular domain, a transmembrane domain, and one or more intracellular domains, the chimeric polypeptide is a first polypeptide, and the polynucleotide further encodes a second polypeptide. In some embodiments, the second polypeptide comprises or is a chimeric cytokine receptor (CCR). In some embodiments, the CCR is constitutively active. In some embodiments, the CCR is inducible. In some embodiments, the second polypeptide comprises or is a chimeric antigen receptor (CAR).

In a further aspect, the present disclosure provides a vector comprising the polynucleotide disclosed herein e.g. a polynucleotide encoding a chimeric polypeptide comprising an extracellular domain, a transmembrane domain, and one or more intracellular domains. In some embodiments, the vector is a viral vector. In some embodiments, the viral vector is a lentiviral vector or an adenoviral vector.

In a further aspect, the present disclosure provides a chimeric polypeptide encoded by the polynucleotide disclosed herein. In another aspect, the present disclosure provides a chimeric polypeptide encoded by the vector disclosed herein.

In a further aspect, the present disclosure provides a chimeric polypeptide comprising an extracellular domain, a transmembrane domain, and one or more intracellular domains, wherein the extracellular domain comprises the extracellular domain of an inhibitory protein such as an inhibitory receptor e.g. the extracellular domain of an inhibitory transmembrane receptor. In some embodiments, the chimeric polypeptide's extracellular domain comprises a PD-1 extracellular domain. In some embodiments, the PD-1 extracellular domain comprises the amino acid sequence of a wild-type hPD-1 extracellular domain (e.g. the amino acid sequence of SEQ ID NO:9). In some embodiments, the PD-1 extracellular domain comprises the amino acid sequence of a variant of a wild-type hPD-1 extracellular domain (e.g. the amino acid sequence of a variant of SEQ ID NO:9 such as an amino acid sequence that is at least 90% identical to SEQ ID NO:9). In some embodiments, the PD-1 extracellular domain variant comprises one or more amino acid insertions, deletions and/or substitutions relative to the wild-type PD-1 extracellular domain e.g. relative to SEQ ID NO:9. In some embodiments, the chimeric polypeptide's PD-1 extracellular domain variant comprises the amino acid sequence of a high affinity (“HA”) hPD-1 extracellular domain (e.g. comprises or has the amino acid sequence of SEQ ID NO:10).

In some embodiments, the chimeric polypeptide's extracellular domain comprises a TGFβ receptor (e.g. TGFBR2) extracellular domain. In some embodiments, the TGFβ receptor extracellular domain comprises the amino acid sequence of a wild-type hTGFb receptor extracellular domain (e.g. the amino acid sequence of SEQ ID NO: 12). In some embodiments, the chimeric polypeptide's extracellular domain comprises a variant of a TGFβ receptor (e.g. TGFBR2) extracellular domain. In some embodiments, the chimeric polypeptide's extracellular domain comprises a variant of the amino acid sequence of SEQ ID NO:12, e.g. an amino acid sequence that is at least 90% identical to the amino acid sequence of SEQ ID NO:12). In some embodiments, the extracellular domain comprises a hTGFb receptor extracellular domain variant comprising one or more amino acid insertions, deletions and/or substitutions relative to the wild-type hTGFb receptor extracellular domain e.g. relative to SEQ ID NO:12. In some embodiments, the variant comprises a 25-amino acid deletion and comprises the amino acid sequence of SEQ ID NO:13 (TGFβR2 ECD ΔN25, also referred to herein as TGFβR2 ECD dN25).

In some embodiments, the chimeric polypeptide's extracellular domain comprises an antibody or an antigen-binding portion of an antibody that specifically recognizes and binds to an inhibitory ligand, such as PDL1 or TGFβ or one or more than one specific TGFβ isoform. In some embodiments, the chimeric polypeptide's extracellular domain comprises a single-chain variable fragment (scFv) that specifically recognizes and binds to an inhibitory ligand, such as PDL1 or TGFβ or one or more TGFβ isoforms, e.g. a TGFβ isoform selected from TGFβ1, TGFβ2 or TGFβ3. In some embodiments, the chimeric polypeptide's extracellular domain comprises an anti-PDL1 scFv. In some embodiments, the chimeric polypeptide's extracellular domain comprises an anti-TGFβ scFv. In some embodiments, the chimeric polypeptide's extracellular domain comprises an anti-TGFβ scFv that binds to and recognizes only one TGFβ isoform, such as a TGFβ isoform selected from TGFβ1, TGFβ2 or TGFβ3, e.g. an anti-TGFβ scFv that binds to and recognizes only TGFβ1. Anti-TGFβ isoform antibodies e.g. anti-TGFβ isoform scFvs and methods of making them are disclosed in, for example, US 20190071493, which is incorporated herein by reference in its entirety.

In some embodiments, the chimeric polypeptide provided herein comprises one or only one intracellular signalling domain. In some embodiments, the chimeric polypeptide comprises more than one intracellular signalling domains. In some embodiments, the chimeric polypeptide comprises more than one intracellular signalling domains, all of which are the same as each other. In some embodiments, the chimeric polypeptide comprises more than one intracellular signalling domains, all of which are different from each other. In some embodiments, the chimeric polypeptide comprises more than one intracellular signalling domains, some of which are the same as each other and some of which are different from the others. In some embodiments, the chimeric polypeptide comprises two, three, four, five or six intracellular signalling domains, of which all are the same as each other, none of which are the same as each other, or some of which are the same as each other.

In some embodiments, the one or more intracellular domains of the chimeric polypeptide provided herein comprise one or more intracellular signalling domains. In some embodiments, the one or more intracellular signalling domains are selected from the group consisting of a CD28 intracellular signalling domain, CD2 intracellular signalling domain, MyD88 intracellular signalling domain, ICOS intracellular signalling domain, DAP10 intracellular signalling domain, OX40 intracellular signalling domain, BAFFR intracellular signalling domain, and CD40 intracellular signalling domain, and any combination thereof. In some embodiments, the one or more intracellular signalling domains comprises a CD28 intracellular signalling domain. In some embodiments, the one or more intracellular signalling domains comprises a CD2 intracellular signalling domain. In some embodiments, the one or more intracellular signalling domains comprises a MyD88 intracellular signalling domain. In some embodiments, the one or more intracellular signalling domains comprises an ICOS intracellular signalling domain. In some embodiments, the one or more intracellular signalling domains comprises a DAP10 intracellular signalling domain. In some embodiments, the one or more intracellular signalling domains comprises an OX40 intracellular signalling domain. In some embodiments, the one or more intracellular signalling domains comprises a BAFFR intracellular signalling domain. In some embodiments, the one or more intracellular signalling domains comprises a CD40 intracellular signalling domain. In some embodiments, the one or more intracellular signalling domains comprises any combination of CD28, CD2, MyD88, ICOS, DAP10, OX40, BAFFR, and CD40 intracellular signalling domains.

In some embodiments, the presently disclosed chimeric polypeptide's one or more intracellular domains comprise the amino acid sequence of, or two or more copies of the amino acid sequence of, any one or more than one of SEQ ID NO:18, SEQ ID NO: 19, SEQ ID NO:20, SEQ ID NO:23, SEQ ID NO:24, SEQ ID NO:15, SEQ ID NO:29, SEQ ID NO:27, SEQ ID NO:33, SEQ ID NO:31, and SEQ ID NO:35. In some embodiments, the presently disclosed chimeric polypeptide's one or more intracellular domains comprise any combination of the amino acid sequences of (e.g. any two of, any three of, any four of, or any five of, or multiple copies of any two of, any three of, any four of, or any five of) SEQ ID NO:18, SEQ ID NO:19, SEQ ID NO:20, SEQ ID NO:23, SEQ ID NO:24, SEQ ID NO:15, SEQ ID NO:29, SEQ ID NO:27, SEQ ID NO:33, SEQ ID NO:31, and SEQ ID NO:35.

In some embodiments, the intracellular domain comprises a CD2 intracellular domain or a variant thereof. In some embodiments, the intracellular domain comprises a variant CD2 intracellular domain, wherein the intracellular domain comprises at least one SH3 domain and/or at least one GYF binding domain, and optionally the variant CD2 intracellular domain is a truncated CD2 intracellular domain. In some embodiments, the SH3 domain comprises the amino acid sequence of SEQ ID NO: 168 or 169 and the GYF binding domain comprises the amino acid sequence of SEQ ID NO: 176, In some embodiments, the intracellular domain comprises the amino acid sequence of at least one, at least two, at least three, at least four, at least five, at least six, or all, of SEQ ID NOs: 170, 171, 172, 173, 174, 175 and 176, and optionally the variant CD2 intracellular domain is a truncated CD2 intracellular domain. In some embodiments, the intracellular domain comprises a variant CD2 intracellular domain that comprises SEQ ID NO: 170 and 176, and optionally the variant CD2 intracellular domain is a truncated CD2 intracellular domain. In some embodiments, the intracellular domain comprises a variant CD2 intracellular domain that comprises SEQ ID NOs: 174 and 175 and optionally the variant CD2 intracellular domain is a truncated CD2 intracellular domain. In some embodiments, the intracellular domain comprises a variant CD2 intracellular domain that comprises SEQ ID NOs: 170, 171, 173, 174 and 175, and optionally the variant CD2 intracellular domain is a truncated CD2 intracellular domain. In some embodiments, the intracellular domain comprises a variant of a CD2 intracellular domain that comprises SEQ ID NOs: 170-176, and optionally the variant CD2 intracellular domain is a truncated CD2 intracellular domain. In some embodiments, the truncated CD2 intracellular domain comprises at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 amino acid deletion from the full-length CD2 intracellular domain, and optionally the truncated CD2 intracellular domain comprises one or more GYF binding domains and/or one or more SH3 domains. In some embodiments, the full-length CD2 intracellular domain comprises the amino acid sequence of SEQ ID NO:23.

In some embodiments, the presently disclosed chimeric polypeptide's intracellular domain comprises a CD2 intracellular domain or a variant thereof. In some embodiments, the presently disclosed chimeric polypeptide's intracellular domain comprises a CD2 intracellular domain or a variant thereof and the chimeric polypeptide's transmembrane domain comprises a CD2 transmembrane domain. In some embodiments, the presently disclosed chimeric polypeptide's intracellular domain comprises a CD2 intracellular domain having the amino acid sequence of SEQ ID NO:23. In some embodiments, the encoded chimeric polypeptide's intracellular domain comprises an amino acid sequence that is at least about 90% identical to the amino acid sequence of SEQ ID NO:23.

In some embodiments of the chimeric polypeptide disclosed herein, the intracellular domain comprises a truncated CD2 intracellular domain. In some embodiments, the truncated CD2 intracellular domain comprises the amino acid sequence of SEQ ID NO:24. In some embodiments, the truncated CD2 intracellular domain comprises an amino acid sequence that is at least about 90% identical to the amino acid sequence of SEQ ID NO:24.

In some embodiments, the presently disclosed chimeric polypeptide's transmembrane domain comprises a CD28 transmembrane domain, CD2 transmembrane domain, PD-1 transmembrane domain, ICOS transmembrane domain, DAP10 transmembrane domain, OX40 transmembrane domain, BAFFR transmembrane domain or CD40 transmembrane domain. In some embodiments, the presently disclosed chimeric polypeptide's transmembrane domain comprises the amino acid sequence of SEQ ID NO:17, SEQ ID NO:22, SEQ ID NO:26, SEQ ID NO:28, SEQ ID NO:30, SEQ ID NO:32, or SEQ ID NO:34.

In some embodiments, the transmembrane domain optionally comprises amino acid residues from the adjacent extracellular domains. In some embodiments, the transmembrane domain optionally comprises 1-20, 1-19, 1-18, 1-17, 1-16, 1-15, 1-14, 1-13, 1-12, 1-11, 1-10, 1-9, 1-8, 1-7, 1-6, 1-5, 1-4, 1-3, or 1-2 amino acid residues from the adjacent extracellular domains.

In some embodiments of the chimeric polypeptide disclosed herein, the transmembrane domain comprises a CD2 transmembrane domain. In some embodiments, the CD2 transmembrane domain comprises the amino acid sequence of SEQ ID NO:22. In some embodiments, the CD2 transmembrane domain comprises an amino acid sequence that is at least about 90% identical to the amino acid sequence of SEQ ID NO:22.

In some embodiments, the chimeric polypeptide comprises a PD1 extracellular domain comprising the amino acid sequence that is at least 85%, 90%, 95%, 96%, 97%, 98%, 99% or 100% identical to SEQ ID NO: 9 or SEQ ID NO: 10. In some embodiments, the chimeric polypeptide comprises a CD28 transmembrane domain comprising the amino acid sequence that is at least about 85%, 90%, 95%, 96%, 97%, 98%, 99% or 100% identical to SEQ ID NO: 17. In some embodiments, the chimeric polypeptide comprises the CD28 intracellular domain that is at least about 85%, 90%, 95%, 96%, 97%, 98%, 99% or 100% identical to SEQ ID NO: 19. In some embodiments, the chimeric polypeptide comprises a CD2 transmembrane domain comprising the amino acid sequence that is at least about 85%, 90%, 95%, 96%, 97%, 98%, 99% or 100% identical to SEQ ID NO; 22. In some embodiments, the chimeric polypeptide comprises a CD2 intracellular domain comprising the amino acid sequence that is at least about 85%, 90%, 95%, 96%, 97%, 98%, 99% or 100% identical to SEQ ID NO: 24. In some embodiments, the chimeric polypeptide comprises the amino acid sequence that is at least about 85%, 90%, 95%, 96%, 97%, 98%, 99% or 100% identical to SEQ ID NO: 76, 79, 85 or 88.

In some embodiments of the chimeric polypeptide disclosed herein, the chimeric polypeptide comprises a signal peptide. In some embodiments, the signal peptide comprises a CD8a signal peptide. In some embodiments, the signal peptide comprises the amino acid sequence of SEQ ID NO:1. In some embodiments, the signal peptide comprises the amino acid sequence of SEQ ID NO:2.

In some embodiments of the chimeric polypeptide disclosed herein, the polypeptide further comprises a hinge domain located between the extracellular domain and the transmembrane domain. In some embodiments, the hinge domain comprises the amino sequence of SEQ ID NO:36. In some embodiments, the hinge domain comprises an amino acid sequence that is at least about 90% identical to the amino acid sequence of SEQ ID NO:36.

In some embodiments of the chimeric polypeptide disclosed herein, the chimeric polypeptide comprises the amino acid sequence of any one of SEQ ID NOS:75-115. In some embodiments, the polypeptide comprises an amino acid sequence that is at least 90% identical to any one of SEQ ID NOS: 75-115.

In a further aspect, the present disclosure provides an engineered cell e.g. an engineered immune cell comprising the polynucleotide encoding a chimeric polypeptide disclosed herein. In some embodiments, the engineered cell e.g. the engineered immune cell expresses the encoded chimeric polypeptide e.g. the engineered immune cell functionally expresses the encoded chimeric polypeptide e.g. on the cell's surface. In some embodiments, the engineered immune cell functionally expresses a chimeric polypeptide comprising a signal peptide, the signal peptide of the chimeric polypeptide is cleaved and the chimeric polypeptide lacking the signal peptide is expressed on the engineered immune cell's surface. In some embodiments, the engineered immune cell comprises and/or expresses the chimeric polypeptide without its signal peptide e.g. on the engineered immune cell surface. In some embodiments, the engineered cell e.g. the engineered immune cell comprising the polynucleotide encoding a chimeric polypeptide disclosed herein does not express the chimeric polypeptide.

In a further aspect, the present disclosure provides an engineered immune cell comprising the vector disclosed herein comprising the polynucleotide disclosed herein. In some embodiments, the engineered cell e.g. the engineered immune cell expresses the encoded chimeric polypeptide e.g. the engineered immune cell functionally expresses the encoded chimeric polypeptide e.g. on the cell's surface. In some embodiments, the engineered immune cell functionally expresses a chimeric polypeptide comprising a signal peptide and the signal peptide of the chimeric polypeptide is cleaved and the chimeric polypeptide lacking the signal peptide is expressed on the engineered immune cell's surface. In some embodiments, the engineered immune cell comprises and/or expresses a chimeric polypeptide without a signal peptide e.g. on the engineered immune cell surface. In some embodiments, the engineered cell e.g. the engineered immune cell comprising the polynucleotide encoding a chimeric polypeptide disclosed herein does not express the chimeric polypeptide.

In a further aspect, the present disclosure provides an engineered immune cell comprising the chimeric polypeptide disclosed herein. In some embodiments, the engineered immune cell comprises the chimeric polypeptide. In some embodiments, the engineered immune cell comprises the chimeric polypeptide lacking a signal peptide.

In a further aspect, the present disclosure provides an engineered immune cell that expresses the chimeric polypeptide disclosed herein. In some embodiments, the engineered immune cell comprises the chimeric polypeptide. In some embodiments, the engineered immune cell comprises the chimeric polypeptide lacking a signal peptide.

In some embodiments, the engineered immune cells comprising or expressing the chimeric polypeptide of the disclosure comprising a CD28 or CD2 intracellular domain, or a variant thereof. In some embodiments, the intracellular domain comprises the amino acid sequence of SEQ ID NO:19, SEQ ID NO:20 or SEQ ID NO: 24. In some embodiments, the chimeric polypeptide comprises a variant CD28 or a variant CD2 intracellular domain and the engineered immune cells express or secret reduced levels of cytokine upon binding of the extracellular domain of the chimeric polypeptide to its ligand. In some embodiments, the reduced level of cytokine is about 1.5 fold, 2 fold, 2.5 fold, 3 fold, 3.5 fold, 4 fold, 4.5 fold, and 5 fold less than the level of cytokine secreted by the engineered immune cells comprising or expressing a chimeric polypeptide that comprises a corresponding wildtype intracellular domain. In certain embodiments, the extracellular domain comprises a PD1 or TGFβ receptor extracellular domain. In some embodiments, the cytokine is GM-CSF, INFγ, TNFα or IL2. In some embodiments, the engineered immune cells comprising or expressing the chimeric polypeptide of the disclosure secret reduced levels of cytokine and exhibit acceptable or improved safety profile when used in adoptive cell therapy. In some embodiments, the engineered immune cells are CAR T cells.

In a further aspect, an engineered immune cell disclosed herein further comprises and/or expresses a chimeric antigen receptor (CAR), wherein the CAR comprises an extracellular ligand-binding domain, a transmembrane domain, and an intracellular signaling domain. In some embodiments, the engineered immune cell comprises a polynucleotide or a vector that encodes the CAR. In some embodiments, the CAR intracellular signaling domain comprises one copy or more than one copy of any one or more of a CD3ζ signaling domain, a CD28 signaling domain, and a 4-1 BB signaling domain. In some embodiments, the CAR comprises an extracellular ligand-binding domain that specifically recognizes and/or binds to DLL3 e.g. DLL3 CARs described and disclosed in WO 2020/180591. In some embodiments, the CAR comprises the amino acid sequence of SEQ ID NO:73 set forth in Table 3, comprising the components rituximab mimotope, 2G1 scFv (specifically recognizes and binds to DLL3), rituximab mimotope, CD8α hinge, CD8α transmembrane, CD8α cytoplasmic domain (truncated), 4-1BB (TNFRSF9, CD137) cytoplasmic domain, and CD3ζ cytoplasmic domain. In some embodiments, the CAR comprises the following components: rituximab mimotope, 2G1 scFv (specifically recognizes and binds to DLL3), rituximab mimotope, CD8α hinge, CD8α transmembrane, CD8α cytoplasmic domain (truncated), 4-1BB (TNFRSF9, CD137) cytoplasmic domain, and CD3ζ cytoplasmic domain. In some embodiments, the CAR comprises the amino acid sequence encoded by the nucleic acid sequence of SEQ ID NO:116. In some embodiments, the CAR amino acid sequence is the amino acid sequence encoded by the nucleic acid sequence of SEQ ID NO:116. In some embodiments, the CAR is encoded by the nucleic acid sequence of SEQ ID NO:116 set forth in Table 4.

In a further aspect, an engineered immune cell disclosed herein further comprises and/or expresses a chimeric cytokine receptor (CCR). In some embodiments, the CCR is an inducible CCR. In some embodiments, the CCR is a constitutively active CCR (CACCR). CCRs are disclosed and described in, for example, WO2019169290, WO2020180694, WO2020180664, and WO2021041806, each of which is hereby incorporated herein by reference in its entirety. In some embodiments, the CCR is one that is disclosed and/or described in any of WO2019169290, WO2020180694, WO2020180664, and WO2021041806. In some embodiments, the engineered immune cell comprises a polynucleotide or a vector that encodes the CCR e.g. the inducible CCR or the CACCR. In some embodiments, the engineered immune cell comprises a polynucleotide or a vector that encodes a CCR comprising the amino acid sequence of SEQ ID NO:162. In some embodiments, the CCR further comprises a signal peptide. In some embodiments, the signal peptide comprises the amino acid sequence of SEQ ID NO:1. In some embodiments of the engineered cell e.g. engineered immune cell disclosed herein, the cell comprises: a first polynucleotide or vector encoding a chimeric polypeptide disclosed herein e.g. a chimeric switch receptor, a second polynucleotide or vector encoding a CAR e.g. a DLL3 CAR, and a third polynucleotide or vector encoding a CCR. In some embodiments of the engineered cell e.g. engineered immune cell disclosed herein, the cell comprises: a first polynucleotide or vector encoding any two of a chimeric polypeptide disclosed herein e.g. a chimeric switch receptor, a CAR e.g. a DLL3 CAR, and a CCR, and a second polynucleotide or vector encoding whichever of a chimeric polypeptide e.g. a chimeric switch receptor, a CAR e.g. a DLL3 CAR, and a CCR that the first polynucleotide or vector does not encode. In some embodiments, one polynucleotide or vector encodes a chimeric polypeptide disclosed herein e.g. a chimeric switch receptor, a CAR e.g. a DLL3 CAR, and a CCR. In some embodiments, the engineered immune cell comprises one or more polynucleotides encoding a DLL3 CAR that comprises the amino acid sequence of SEQ ID NO:73 or 165, with or without a signal sequence, and a CCR that comprises the amino acid sequence of SEQ ID NO:162. In some embodiments, the polynucleotide encodes a CCR and DLL3 CAR that comprise the amino acid sequence of SEQ ID NO:74, with or without a signal sequence. In some embodiments, the DLL3 CAR and/or the CCR further comprises a CD8 signal sequence comprising the amino acid sequence of SEQ ID NO:1. In some embodiments, a first polynucleotide or vector encodes a chimeric switch receptor as disclosed herein and a second polynucleotide or vector encodes both a CAR e.g. a DLL3 CAR and a CCR and further optionally encodes a self-cleaving peptide e.g. a 2A peptide between the CAR and the CCR. For example, in some embodiments, the second polynucleotide or vector encodes a CCR-P2A-CAR polypeptide, e.g. a CCR-P2A-DLL3 CAR polypeptide comprising the components CD8 signal sequence, TpoR (thrombopoietin receptor) (S505N W515K), IL2Rb-YY, P2A, CD8 signal sequence, rituximab mimotope, 2G1 scFv, rituximab mimotope, CD8α hinge, CD8α transmembrane, CD8α cytoplasmic domain (truncated), 4-1BB (TNFRSF9, CD137) cytoplasmic domain, and CD3ζ cytoplasmic domain, e.g. a CCR-P2A-DLL3 CAR polypeptide comprising the amino acid sequence of SEQ ID NO:74, encoded by a polynucleotide comprising a nucleic acid sequence of SEQ ID NO:117 or a polypeptide e.g. a DLL3 CAR-P2A-CCR polypeptide that comprises an amino acid sequence that is at least 90% identical to the amino acid sequence of SEQ ID NO:74.

In a further aspect, the present disclosure provides an engineered immune cell that comprises one or more polynucleotides encoding a DLL3 CAR that comprises the amino acid sequence of SEQ ID NO:73 or 165, and a CCR that comprises the amino acid sequence of SEQ ID NO:162. In a related aspect, the present disclosure provides an engineered immune cell that comprises and/or expresses a DLL3 CAR that comprises the amino acid sequence of SEQ ID NO:73 or 165, and a CCR that comprises the amino acid sequence of SEQ ID NO:162. In a further related aspect, the present disclosure provides a method of making an engineered immune cell wherein the engineered immune cell comprises and/or expresses a DLL3 CAR that comprises the amino acid sequence of SEQ ID NO:73 or 165, and a CCR that comprises the amino acid sequence of SEQ ID NO:162. In an embodiment, the method comprises introducing into a cell e.g. an immune cell, either sequentially or simultaneously, a nucleic acid that encodes a polypeptide comprising the amino acid sequence of SEQ ID NO:73 or 165 and a nucleic acid that encodes a polypeptide comprising the amino acid sequence of SEQ ID NO:162. In some embodiments, a single nucleic acid (e.g. a vector, expression vector, retroviral vector) comprises the nucleic acid that encodes the amino acid sequence of SEQ ID NO:73 or 165 and the nucleic acid that encodes the amino acid sequence of SEQ ID NO:162. In yet another related aspect, the present disclosure provides a method of treatment using an engineered immune cell wherein the engineered immune cell comprises and/or expresses a DLL3 CAR that comprises the amino acid sequence of SEQ ID NO: 73 or 165, and a CCR that comprises the amino acid sequence of SEQ ID NO:162.

In some embodiments of the engineered immune cell disclosed herein, the engineered immune cell is a T cell, tumor infiltrating lymphocyte (TIL), NK cell, TCR-expressing cell, dendritic cell, or NK-T cell. In some embodiments of the engineered immune cell disclosed herein, the cell is an autologous T cell. In some embodiments of the engineered immune cell disclosed herein, the cell is an allogeneic T cell.

In a further aspect, the present disclosure provides a population of cells comprising one or more than one of the engineered immune cells disclosed herein. In some embodiments, the population of cells comprises at least about 1×10⁴, 1×10⁵, 1×10⁶, 1×10⁷, 1×10⁸, 1×10⁹, or 1×10¹⁰ of the engineered cells e.g. engineered immune cells disclosed herein. In an embodiment, the population of engineered immune cells comprises between about 1×10⁴ and about 1×10¹⁰ engineered immune cells provided herein.

In a further aspect, the present disclosure provides a composition comprising a cell e.g. an engineered immune cell disclosed herein. In a further aspect, the present disclosure provides a composition comprising a population of cells disclosed herein and a pharmaceutically acceptable carrier.

In a further aspect, the present disclosure provides a method of treating a disease or condition in a patient comprising administering to the patient a cell e.g. an engineered immune cell disclosed herein. In a further aspect, the present disclosure provides a method of treating a disease or condition in a patient comprising administering to the patient a population of cells disclosed herein. In a further aspect, the present disclosure provides a method of treating a disease or condition in a patient comprising administering to the patient a composition disclosed herein. In some embodiments, the patient is a human. In some embodiments, the patient is a non-human mammal.

In some embodiments of a method of treating disclosed herein, the patient is a human and the condition is a cancer. In some embodiments, the cancer is a hematological malignancy or a solid cancer. In some embodiments, the cancer is a hematological malignancy optionally selected from acute lymphoblastic leukemia (ALL), acute myeloid leukemia (AML), chronic myelogenous leukemia (CML), chronic eosinophilic leukemia (CEL), myelodysplasia syndrome (MDS), non-Hodgkin's lymphoma (NHL), and multiple myeloma (MM). In some embodiments, the cancer is a solid cancer. In some embodiments, the cancer is a solid cancer optionally selected from biliary cancer, bladder cancer, bone and soft tissue carcinoma, brain tumor, breast cancer, cervical cancer, colon cancer, colorectal adenocarcinoma, colorectal cancer, desmoid tumor, embryonal cancer, endometrial cancer, esophageal cancer, gastric cancer, gastric adenocarcinoma, glioblastoma multiforme, gynecological tumor, head and neck squamous cell carcinoma, hepatic cancer, lung cancer, malignant melanoma, osteosarcoma, ovarian cancer, pancreatic cancer, pancreatic ductal adenocarcinoma, primary astrocytic tumor, primary thyroid cancer, prostate cancer, renal cancer, renal cell carcinoma, rhabdomyosarcoma, skin cancer, soft tissue sarcoma, testicular germ-cell tumor, urothelial cancer, uterine sarcoma, and uterine cancer.

In a further aspect, the present disclosure provides a method of making an engineered immune cell disclosed herein. In some embodiments, the method comprises the step of introducing one or more polynucleotides and/or vectors disclosed herein into a cell e.g. an immune cell. In some embodiments, the cell is a T cell, tumor infiltrating lymphocyte (TIL), NK cell, TCR-expressing cell, dendritic cell, or NK-T cell. In various embodiments, the method comprises the use of any gene editing technology, such as TALEN, zinc fingers, Cas-CLOVER, and a CRISPR/Cas system, and/or the use of any known gene knockdown methods e.g. those that employ any of various RNA-based techniques (e.g. shRNA, antisense RNA, miRNA, siRNA; see, e.g., Lam et al., Mol. Ther.-Nucleic Acids 4:e252 (2015), doi:10.1038/mtna.2015.23; Sridharan and Gogtay, Brit. J. Clin. Pharmacol. 82: 659-72 (2016)) to reduce functional expression of specific genes. In some embodiments, the specific genes are, for example, PD1, TRAC, TGFβR, CD70, CD52, TIM3, LAG3, CISH, cbl-b, TIGIT, or A2AR.

In some embodiments of the method of making an engineered immune cell disclosed herein, the cell that is engineered is an autologous T cell. In some embodiments, the cell that is engineered is an allogeneic T cell. In some embodiments, the method comprises or further comprises introducing into the genome of the cell e.g. immune cell one or more genomic modifications of one or more of an endogenous CD70, CD52, PD1, TIM3, CISH, cbl-b, TIGIT, and TCRa gene. In some embodiments, the one or more genomic modifications disrupts and/or prevents, wholly or partly, the functional expression of one or more of an endogenous CD70, CD52, PD1, TIM3, CISH, cbl-b, TIGIT, and TCRa gene. In some embodiments of the method of making an engineered immune cell disclosed herein, the polynucleotide or vector is integrated into the immune cell genome. In some embodiments, the vector is a viral vector, for example, a lentiviral vector. In some embodiments, the polynucleotide or vector is integrated into the genome by random integration. In some embodiments, the polynucleotide or vector is integrated into the genome by site specific integration mediated by homologous recombination. In some embodiments, the one or more polynucleotides and/or vectors is integrated into a CD70, CD52, PD1, TIM3, CISH, cbl-b, TIGIT, and/or TCRa locus. In some embodiments, the method comprises integrating a first polynucleotide or vector as disclosed herein into a first genetic locus and integrating a second polynucleotide or vector as disclosed herein into a second genetic locus.

In some embodiments of the method of making an engineered immune cell disclosed herein, the method comprises site-specifically integrating a first polynucleotide or vector into a first genetic locus such as a CD70 locus, CD52 locus, PD1 locus, TIM3 locus, CISH locus, TIGIT locus, or cbl-b locus and site-specifically integrating a second polynucleotide or vector into a second genetic locus such as a TRAC locus. In some embodiments, the first polynucleotide or vector encodes a polypeptide as disclosed herein comprising an extracellular domain, a transmembrane domain, and one or more intracellular domains, wherein the extracellular domain comprises an extracellular domain of an inhibitory protein, e.g. the vector encodes a chimeric switch receptor (CSR), and the second polynucleotide or vector encodes a CAR as described herein e.g. a DLL3 CAR as described herein, and optionally the second polynucleotide or vector further encodes a chimeric cytokine receptor (CCR) as described herein e.g. an inducible CCR or a constitutively active CCR (CACCR). In some embodiments, the CAR, CSR, and/or CCR further comprises a signal peptide. In some embodiments, the first polynucleotide or vector encodes a chimeric polypeptide comprising the amino acid sequence of any one of SEQ ID NOS: 75-115. In some embodiments, the first polynucleotide or vector encodes a chimeric polypeptide comprising an amino acid sequence that is at least 90% identical to any one of SEQ ID NOS: 75-115.

In various embodiments, the method of making an engineered immune cell provided herein can be applied to a cell or cells from any of various sources. The engineered immune cell can be prepared or derived from cells e.g. stem cells or immune cells from a person other than the person to whom the engineered immune cells will be administered, e.g. a donor (e.g. a healthy volunteer) other than the recipient, or can be prepared or derived from cells e.g. stem cells or immune cells from the person to whom the engineered immune cells will be administered (the recipient), or can be derived from one or more induced pluripotent stem cells (iPSCs). In various embodiments, the immune cell is an immune cell obtained from a healthy volunteer, is obtained from a patient, or is derived from an iPSC.

In a further aspect, the present disclosure provides an engineered immune cell made by any of the methods of making an engineered immune cell disclosed herein. In a further aspect, the present disclosure provides a method of treating a condition e.g. a cancer as described herein comprising e.g. administering to a patient in need of such treatment an engineered immune cell made by any of the methods disclosed herein or a population of cells comprising one or more engineered immune cells (e.g. about 10⁵, 10⁶, 10⁷, 10⁸, 10⁹, 10¹⁰, 10¹¹ engineered immune cells) made by any of the methods disclosed herein.

### BRIEF DESCRIPTION OF THE DRAWINGS

FIGS. 1A-1B. Design of exemplary chimeric switch receptors. FIG. 1A and FIG. 1B show schematic diagrams of the domain structure of representative PD1 chimeric switch receptors (FIG. 1A) and TGFbR2 chimeric switch receptors (FIG. 1). ECD: ectodomain; TM: transmembrane domain; ICD: intracellular domain.

FIGS. 2A-2B show in vivo data from an SHP-77 subcutaneous model of anti-tumor response and persistence of CAR T cells expressing the DLL3 CAR T clone 2G1 with a CCR version 15.1 or CCR version 15.3. FIGS. 2C-2E show the anti-tumor response and persistence data and cytokine secretion data from an NCI-H82 subcutaneous model. FIG. 2F. Experimental data showing wild-type (WT) PD1 or high-affinity (HA) PD1 chimeric switch receptors, comprising CD28, CD2 or CD2-short intracellular domains, expressed alone (top row) or together with the DLL3 CAR and CCR (bottom row) on the surface of primary T-cells. FIG. 2G. Bar graph summarizing the percentage of T cells in which expression of CAR was (CAR+) or was not (CAR−) detected and in which expression of a PD1 chimeric switch receptor was (iPD1+) or was not (iPD1−) detected in cells that were transduced with one vector or both vectors (see example 2 for methods). FIG. 2H. Experimental data showing expansion of transduced T-cells.

FIGS. 3A-3B. Cytotoxic activity of T-cells expressing CAR and CCR (2G1.15.1 clone encodes both CAR and CCR) without or with chimeric switch receptors comprising wild-type (WT) or high affinity (HA) PD1 domain fused with the CD28, CD2, or CD2-short (also referred to herein as “truncated”) intracellular domains. Serial killing assays were used to determine the cytotoxicity of the T-cells produced according to the methods in Example 3. CD28 or CD2short ICD, paired with WT (FIG. 3A) or HA PD1 (FIG. 3B) ectodomain, enhanced cytotoxic activity of 2G1.15.1. Target cells were DMS273 (PDL1-low) (top row) or DMS273-PDL1 (PDL1-high) (bottom row). FIG. 3B. Exemplary sequences tested were: WT PD1-CD28, SEQ ID NO: 75; HA PD1-CD28, SEQ ID NO: 84; WT PD1-CD2, SEQ ID NO: 78; HA PD1-CD2, SEQ ID NO: 87; WT PD1-CD2short, SEQ ID NO: 79; and HA PD1-CD2short, SEQ ID NO: 88. A CD8α signal peptide (SEQ ID NO:1) was used for these constructs.

FIGS. 4A-4B. Cytokine (as exemplified by IL-2 or IFNγ) secretion by various CAR T cells in the presence of target cells expressing low or high level of PDL1. NTD: non-transduced.

FIGS. 5A-5D. Production of T-cells expressing DLL3 CAR and CCR (2G1.15.1) alone or together with one of various PD1 chimeric switch receptors or a dominant negative PD-1 construct (dnWT-PD1) using multiplexed site-specific integration (SSI) from one human T-cell donor. FIG. 5A. Results showing TALEN mediated gene knockout efficiency at TRAC and CD52 loci and AAV mediated transduction efficiency of CAR and various PD1 constructs on Day 6 of production. FIG. 5B. Flow cytometry plots depicting percentage of T-cells expressing no transduced polypeptides, DLL3 CAR and CCR (2G1.15.1), with or without dnWT-PD1, PD1 chimeric switch receptor on Day 16 of production. FIG. 5C. Bar graphs demonstrating mean fluorescence intensity of DLL3 CAR (left panel) and PD1 chimeric switch receptors (right panel). FIG. 5D. Expansion, during production, of T-cells expressing DLL3 CAR and CCR (2G1.15.1) alone or together with one of various PD1 constructs. NTD: non transduced; dnWT-PD1: dominant negative wild-type PD1.

FIGS. 6A-6C. Production of T-cells expressing DLL3 CAR and CCR (2G1.15.1) alone or together with dominant negative-HA PD1 or one of various HA PD1 chimeric switch receptors using SSI from a human T-cell donor different from the donor shown in FIGS. 5A-5C. The named constructs are listed with their corresponding amino acid and nucleic acid sequences in Tables 3 and 4. FIG. 6A. Flow cytometry results showing TALEN mediated gene knockout efficiency and AAV mediated transduction efficiency on day 14 of production of cells expressing the indicated polypeptides. More than 75% of T-cells lost TCRa/b expression and most TCRa/b negative T-cells express DLL3 CAR and CCR. FIG. 6B. A majority of T-cells lost CD52 expression, and a subset of CD52 negative T-cells express PD1 chimeric switch receptor (bottom panel). FIG. 6C. Flow cytometry plots depicting percentages of T-cells expressing DLL3 CAR and/or chimeric switch receptor on day 14 of production. FIG. 6D. T-cell expansion during production of T-cells expressing the indicated polypeptides (and see Tables 3 and 4 for corresponding sequences). HA: high affinity; dnHACiPD1: dominant negative high affinity PD1 (Maute R L, et al. Proc Natl Acad Sci USA. 2015).

FIG. 7. CD2 or CD2short enhances cytotoxic activity in CAR T cells generated by SSI, similar to CAR T cells generated by lentivirus transduction. Cytotoxicity of T cells co-expressing DLL3 CAR and CCR (2G1.15.1) alone or together with one of various PD1 chimeric switch receptors (either with wildtype (WT) PD1 extracellular domain or high affinity (HA) PD1 extracellular domain) against target cells expressing a low or high level of DLL3 and PDL1. Dominant negative PD1 (dnWT PD1) alone did not enhance cytotoxic activity in the assay, while switch receptors with intracellular signaling domains from CD2 or CD2short enhanced DLL3 CAR cytotoxic activity.

FIGS. 8A-8B. FIG. 8A. Data showing serial killing assay of CAR T cells expressing additional PD1 chimeric switch receptors as compared to parental CAR T cells all produced by site-specific integration. FIG. 8B. Results of a single stimulation long-term killing assay show enhanced cytotoxicity when co-cultured with NCI-H82-PDL1 cells and concomitant expansion of CAR-T cells co-expressing different PD1 chimeric switch receptor.

FIGS. 9A-9B present data showing cytokine secretion from T cells expressing chimeric switch receptors produced by site-specific integration.

FIGS. 10A-10C present data showing cytokine secretion from CAR T cells expressing chimeric switch receptors produced by site specific integration. When the CAR T cells were co-cultured with PDL1-high cells (condition D), many chimeric switch receptors induced higher levels of cytokine secretion than when CAR T cells were co-cultured with PDL1-low DMS 273 cells (condition C). The data values are shown in FIG. 10C.

FIG. 11 presents data showing the expansion of DLL3 CAR T (2G1.15.1) cells co-expressing one of various PD1 chimeric switch receptors in the absence of target cells or growth factors. Total live cell count was quantified using flow cytometry.

FIGS. 12A-12B show results of long-term killing assay at different E: T ratios of target cells expressing either low (FIG. 12A) or high (FIG. 12B) levels of PDL1 and increasing levels of DLL3 and CAR T cells expressing a DLL3 CAR without a CCR (2G1) and co-expressing either a PD1-dominant negative receptor (DN PD1) or one of various PD1 chimeric switch receptors. The CAR T cells were generated by LVV transduction.

FIGS. 12C-12D show results of long-term killing assay of PDL1-low target cells (FIG. 12C) or PDL1-high target cells (FIG. 12D) and DLL3 CAR (2G1) T cells, in which DLL3 CAR T cells were generated by multiplexed SSI co-expressing one of various PD1 chimeric switch receptors (iPD-1CD2sh, iPD1-CD28.YMFM) or an inducible chimeric cytokine receptor (iPD1-15.1, iPD1-15.3). Results of CAR T cells from two different donors were shown. FIG. 12E show results of the expansion of either total live cells or CAR+DLL3 CAR T cells co-expressing one of various PD1 chimeric switch receptors or an inducible PD1 chimeric cytokine receptor in the absence of target cells or growth factors. Results of CAR T cells from two different donors were shown. The PD1 chimeric switch receptors tested in FIGS. 12A-12E comprise a high affinity (HA) PD1 ECD, and a CD2 transmembrane domain and a truncated CD2 signaling domain (iPD1-CD2sh), or a CD28 transmembrane domain and a variant CD28 signaling domain YMFM (iPD1-CD28 YMFM) or AYAA (iPD1-CD28 AYAA). The PD1 inducible chimeric cytokine receptor tested comprise an HA PD1 ECD, a variant TPOR transmembrane domain (TPOR N+4), and an IL2Rb signaling domain comprising the amino acid sequences of SEQ ID NO: 4 (iPD1-15.1) or SEQ ID NO: 180 (iPD1-15.3). DLL3 CAR (2G1) T cells generated by lentivirus transduction were used for comparison. NTD: non-transduced.

FIGS. 13A-13B depict results of long-term killing assay at different E:T ratios of an exemplary CD70 CAR T cells co-expressing a high affinity PD1 chimeric switch receptor comprising either the wild-type or variant CD28 signaling domain and CD70-high target cells (FIG. 13A) or CD70-low target cells (FIG. 13B), expressing either low (top panel) or high (bottom panel) levels of PDL1.

### DETAILED DESCRIPTION

The present disclosure provides chimeric polypeptides, specifically chimeric switch receptors, and related polynucleotides, vectors, engineered cells e.g. engineered immune cells, compositions, methods of making engineered cells e.g. engineered immune cells, and methods of treating. The disclosure provides numerous advantages including improvements CAR T cell therapy.

As detailed herein, in chimeric switch receptors disclosed herein containing an ectodomain (extracellular domain) derived from PD1, the ectodomain may either be the wildtype sequence, or be modified to bind PD1 ligands with higher affinity. The high affinity PD1 ectodomain may enable the chimeric switch receptor to more effectively, (i) out-compete endogenous PD1 for binding to its ligands, and (ii) transmit a costimulatory signal in PDL1/2-low (or TGFb-low) environments.

In solid tumors that express little or no costimulatory ligands, the coupling of an inhibitory signal to a costimulatory signal can overcome the need for cognate costimulation.

Chimeric switch receptors can be fused to various intracellular costimulatory signaling domains, either singly or in tandem. Tandem fusions will enable two or more costimulatory signaling domains to be simultaneously activated upon ligand binding.

In the case where an intracellular signaling domain does not have a transmembrane domain (e.g. MyD88), an orthogonal transmembrane domain is used to localize MyD88 to the cell membrane. For example, the PD1 transmembrane domain may be used to minimize tonic signaling of the chimeric switch receptor.

Individual intracellular costimulatory domains may be optimized to reduce vector cargo and enhance functional activity by the removal of non-signaling intervening sequences or negative regulatory sequences (e.g. CD2short).

### General Techniques

The practice of the instant disclosure will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995). Gene editing techniques using TALENs, CRISPR/Cas9, and megaTAL nucleases, for example, are within the skill of the art and explained fully in the literature, such as T. Gaj et al., Genome-Editing Technologies: Principles and Applications, Cold Spring Harb Perspect Biol 2016; 8:a023754 and citations therein.

### Definitions

Unless otherwise noted, the terms “a” or “an” are to be construed as meaning “at least one or more of.”

As used herein “autologous” means that cells, a cell line, or population of cells used for treating subjects that are obtained from said subject.

As used herein “allogeneic” means that cells or population of cells used for treating subjects that are not obtained from said subject, but instead from a donor.

As used herein, the term “endogenous” refers to any material from or produced inside an organism, cell, tissue or system.

As used herein, the term “exogenous” refers to any material introduced from or produced outside an organism, cell, tissue or system.

As used herein, “immune cell” refers to a cell of hematopoietic origin functionally involved in the initiation and/or execution of innate and/or adaptative immune response. Examples of immune cells include T cells, e.g., alpha/beta T cells and gamma/delta T cells, Regulatory T (Treg) cells, B cells, natural killer (NK) cells, natural killer T (NKT) cells, mast cells, and myeloid-derived phagocytes.

As used herein, the term “expression” refers to the transcription and/or translation of a particular nucleotide sequence driven by a promoter.

As used herein, “expression vector” refers to a vector comprising a recombinant polynucleotide comprising expression control sequences operatively linked to a nucleotide sequence to be expressed. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

Engineered immune cells of the present disclosure express e.g. functionally express a chimeric polypeptide e.g. chimeric switch receptor as disclosed herein. For example, engineered immune cells of the present disclosure can functionally express a chimeric polypeptide e.g. chimeric switch receptor protein from an exogenous nucleic acid encoding the antigen binding protein introduced into the cell by techniques described herein, and/or they can comprise genomic modifications e.g. mutations at endogenous genes such as e.g. CD70, CD52, PD1, TIM3, CISH, cbl-b, TIGIT and/or TCRa that decrease or eliminate functional expression of the gene at the site of the genomic modification, and/or they can express one or more additional proteins (e.g. a CAR and/or a CCR) from an exogenous nucleic acid introduced into the cell by techniques described herein. As described herein, engineered immune cells of the present disclosure can derive, e.g., be prepared from cells, e.g., immune cells obtained from various sources.

As used herein, to “functionally express” a gene means that a gene is expressed and that expression yields a functioning gene end product. For example, if a gene encodes a protein, then a cell functionally expresses the gene if expression of the gene ultimately produces a properly functioning protein. Thus, if a gene is not transcribed, or expression of the gene ultimately produces an RNA that is not translated or translation yields only a non-functioning protein e.g. the protein does not fold correctly or is not transported to its site of action (e.g. membrane, for membrane-bound proteins), for example, then the gene is not functionally expressed. Functional expression can be measured directly (e.g. by assaying for the gene product itself) or indirectly (e.g. by assaying for the effects of the gene product).

As used herein, “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter).

As used herein, “expression control sequence” means a nucleic acid sequence that directs transcription of a nucleic acid. An expression control sequence can be a promoter, such as a constitutive or an inducible promoter, or an enhancer. The expression control sequence is operably linked to the nucleic acid sequence to be transcribed.

“Promoter” and “promoter sequence” are used interchangeably and refer to a DNA sequence capable of controlling the expression of a coding sequence or functional RNA. In general, a coding sequence is located 3′ to a promoter sequence. It is understood by those skilled in the art that different promoters can direct the expression of a gene in different tissues or cell types, or at different stages of development, or in response to different environmental or physiological conditions.

In any of the vectors of the present disclosure, the vector optionally comprises a promoter disclosed herein.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of the instant disclosure.

The term “extracellular ligand-binding domain” as used herein refers to an oligo- or polypeptide that is capable of binding a ligand. Preferably, the domain will be capable of interacting with a cell surface molecule. For example, the extracellular ligand-binding domain can be chosen to recognize a ligand that acts as a cell surface marker on target cells associated with a particular disease state. The term “stalk domain” is used herein to refer to any oligo- or polypeptide that functions to link the transmembrane domain to the extracellular ligand-binding domain. In particular, stalk domains are used to provide more flexibility and accessibility for the extracellular ligand-binding domain.

The term “intracellular signaling domain” refers to the portion of a protein which transduces the effector signal function signal and directs the cell to perform a specialized function.

A “co-stimulatory molecule” as used herein refers to the cognate binding partner on a T cell that specifically binds with a co-stimulatory ligand, thereby mediating a co-stimulatory response by the cell, such as, but not limited to proliferation. Co-stimulatory molecules include, but are not limited to, an MHC class I molecule, BTLA and Toll ligand receptor. Examples of costimulatory molecules include CD27, CD28, CD8, 4-1BB (CD137), OX40, CD30, CD40, PD-1, ICOS, lymphocyte function-associated antigen-1 (LFA-1), CD2, CD7, LIGHT, NKG2C, B7-H3 and a ligand that specifically binds with CD83 and the like.

A “co-stimulatory ligand” refers to a molecule on an antigen presenting cell that specifically binds a cognate co-stimulatory signal molecule on a T cell, thereby providing a signal which, in addition to the primary signal provided by, for instance, binding of a TCR/CD3 complex with an MHC molecule loaded with peptide, mediates a T cell response, including, but not limited to, proliferation activation, differentiation and the like. A co-stimulatory ligand can include but is not limited to CD7, B7-1 (CD80), B7-2 (CD86), PD-L2, 4-1 BBL, OX40L, inducible costimulatory ligand (ICOS-L), intercellular adhesion molecule (ICAM, CD30L, CD40, CD70, CD83, HLA-G, MICA, M1 CB, HVEM, lymphotoxin β receptor, 3/TR6, ILT3, ILT4, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as but not limited to, CD27, CD28, 4-1 BB, OX40, CD30, CD40, ICOS, lymphocyte function-associated antigen-1 (LFA-1), CD2, CD7, LTGHT, NKG2C, B7-H3, a ligand that specifically binds with CD83.

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact (or full-length) polyclonal or monoclonal antibodies, but also antigen-binding fragments thereof (such as Fab, Fab′, F(ab′)2, and Fv), and any other modified configuration of the immunoglobulin molecule that comprises an antigen recognition site including, for example without limitation, single chain (scFv) and domain antibodies (including, for example, shark and camelid antibodies), and fusion proteins comprising an antibody. An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these can be further divided into subclasses (isotypes), e.g., lgG1, lgG2, lgG3, lgG4, lgA1 and lgA2. The heavy-chain constant regions that correspond to the different classes of immunoglobulins are called alpha, delta, epsilon, gamma, and mu, respectively. The subunit structures and three-dimensional configurations of different classes of immunoglobulins are well known.

The term “antigen-binding fragment” or “antigen binding portion” of an antibody, as used herein, refers to one or more fragments of an intact antibody that retain the ability to specifically bind to a given antigen. Antigen binding functions of an antibody can be performed by fragments of an intact antibody. Examples of binding fragments encompassed within the term “antigen binding fragment” of an antibody include Fab; Fab′; F(ab′)2; an Fd fragment consisting of the VH and CH1 domains; an Fv fragment consisting of the VL and VH domains of a single arm of an antibody; a single domain antibody (dAb) fragment (see, e.g., Ward et al., Nature 341:544-546, 1989), and an isolated complementarity determining region (CDR).

An antibody, an antibody conjugate, or a polypeptide that “specifically binds” to a target is a term well understood in the art, and methods to determine such specific binding are also well known in the art. A molecule is said to exhibit “specific binding” if it reacts or associates more frequently, more rapidly, with greater duration and/or with greater affinity with a particular cell or substance than it does with alternative cells or substances. An antibody “specifically binds” to a target if it binds with greater affinity, avidity, more readily, and/or with greater duration than it binds to other substances. It is also understood that by reading this definition, for example, an antibody (or moiety or epitope) that specifically binds to a first target may or may not specifically bind to a second target. As such, “specific binding” does not necessarily require (although it can include) exclusive binding.

A “variable region” of an antibody refers to the variable region of the antibody light chain or the variable region of the antibody heavy chain, either alone or in combination. As known in the art, the variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen binding site of antibodies. There are several techniques for determining CDRs, e.g., an approach based on cross-species sequence variability (i.e., Kabat et al. Sequences of Proteins of Immunological Interest, (5th ed., 1991, National Institutes of Health, Bethesda MD)); an approach based on crystallographic studies of antigen-antibody complexes (Al-lazikani et al., 1997, J. Molec. Biol. 273:927-948), the Chothia system (i.e., Chothia and Lesk, J. Mol. Biol. (1987) 196(4):901-917. As used herein, a CDR can refer to CDRs defined by either approach or by a combination of both approaches.

A “CDR” of a variable domain are amino acid residues within the variable region that are identified in accordance with the definitions of the Kabat, Chothia, the accumulation of both Kabat and Chothia, AbM, contact, and/or conformational definitions or any method of CDR determination well known in the art. Antibody CDRs can be identified as the hypervariable regions originally defined by Kabat et al. See, e.g., Kabat et al., 1992, Sequences of Proteins of Immunological Interest, 5th ed., Public Health Service, NIH, Washington D.C. The positions of the CDRs can also be identified as the structural loop structures originally described by Chothia and others. See, e.g., Chothia et al., Nature 342:877-883, 1989. Other approaches to CDR identification include the “AbM definition,” which is a compromise between Kabat and Chothia and is derived using Oxford Molecular's AbM antibody modeling software (now Accelrys®), or the “contact definition” of CDRs based on observed antigen contacts, set forth in MacCallum et al., J. Mol. Biol., 262:732-745, 1996. In another approach, referred to herein as the “conformational definition” of CDRs, the positions of the CDRs can be identified as the residues that make enthalpic contributions to antigen binding. See, e.g., Makabe et al., Journal of Biological Chemistry, 283:1 156-1 166, 2008. Still other CDR boundary definitions may not strictly follow one of the above approaches, but will nonetheless overlap with at least a portion of the Kabat CDRs, although they can be shortened or lengthened in light of prediction or experimental findings that particular residues or groups of residues or even entire CDRs do not significantly impact antigen binding. As used herein, a CDR can refer to CDRs defined by any approach known in the art, including combinations of approaches. The methods used herein can utilize CDRs defined according to any of these approaches. For any given embodiment containing more than one CDR, the CDRs can be defined in accordance with any of Kabat, Chothia, extended, AbM, contact, AHo and/or conformational definitions.

Antibodies of the instant disclosure can be produced using techniques well known in the art, e.g., recombinant technologies, phage display technologies, synthetic technologies or combinations of such technologies or other technologies readily known in the art (see, for example, Jayasena, S. D., Clin. Chem., 45: 1628-50, 1999 and Fellouse, F. A., et al, J. Mol. Biol., 373(4):924-40, 2007).

As known in the art, “polynucleotide,” or “nucleic acid,” as used interchangeably herein, refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide can comprise modified nucleotides, such as methylated nucleotides and their analogs. If present, modification to the nucleotide structure can be imparted before or after assembly of the chain. The sequence of nucleotides can be interrupted by non-nucleotide components. A polynucleotide can be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars can be replaced, for example, by phosphonate groups, phosphate groups, protected by standard protecting groups, or activated to prepare additional linkages to additional nucleotides, or can be conjugated to solid supports. The 5′ and 3′ terminal OH can be phosphorylated or substituted with amines or organic capping group moieties of from 1 to 20 carbon atoms. Other hydroxyls can also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha- or beta-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages can be replaced by alternative linking groups. These alternative linking groups include, but are not limited to, embodiments wherein phosphate is replaced by P(O)S(“thioate”), P(S)S (“dithioate”), (O)NR2 (“amidate”), P(O)R, P(O)OR′, CO or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

As used herein, “transfection” refers to the uptake of exogenous or heterologous RNA or DNA by a cell. A cell has been “transfected” by exogenous or heterologous RNA or DNA when such RNA or DNA has been introduced inside the cell. A cell has been “transformed” by exogenous or heterologous RNA or DNA when the transfected RNA or DNA effects a phenotypic change. The transforming RNA or DNA can be integrated (covalently linked) into chromosomal DNA making up the genome of the cell.

As used herein, “transformation” refers to the transfer of a nucleic acid fragment into the genome of a host organism, resulting in genetically stable inheritance. Host organisms containing the transformed nucleic acid fragments are referred to as “transgenic” or “recombinant” or “transformed” organisms.

As used herein, “substantially pure” refers to material which is at least 50% pure (i.e., free from contaminants), more preferably, at least 90% pure, more preferably, at least 95% pure, yet more preferably, at least 98% pure, and most preferably, at least 99% pure. The term “compete”, as used herein with regard to an antibody, means that a first antibody, or an antigen binding fragment (or portion) thereof, binds to an epitope in a manner sufficiently similar to the binding of a second antibody, or an antigen binding portion thereof, such that the result of binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). Both competing and cross-competing antibodies are encompassed by the instant disclosure. Regardless of the mechanism by which such competition or cross-competition occurs (e.g., steric hindrance, conformational change, or binding to a common epitope, or portion thereof), the skilled artisan would appreciate, based upon the teachings provided herein, that such competing and/or cross-competing antibodies are encompassed and can be useful for the methods disclosed herein.

As used herein, “treatment” is an approach for obtaining a beneficial or desired clinical result. For purposes of the instant disclosure, beneficial or desired clinical results include, but are not limited to, one or more of the following: reducing the proliferation of (or destroying) neoplastic or cancerous cells, inhibiting metastasis of neoplastic cells, shrinking or decreasing the size of tumor, remission of a disease (e.g., cancer), decreasing symptoms resulting from a disease (e.g., cancer), increasing the quality of life of those suffering from a disease (e.g., cancer), decreasing the dose of other medications required to treat a disease (e.g., cancer), delaying the progression of a disease (e.g., cancer), curing a disease (e.g., cancer), and/or prolong survival of subjects having a disease (e.g., cancer).

“Ameliorating” means a lessening or improvement of one or more symptoms as compared with not administering a treatment. “Ameliorating” also includes shortening or reduction in duration of a symptom. As used herein, an “effective dosage” or “effective amount” of drug, compound, or pharmaceutical composition is an amount sufficient to effect any one or more beneficial or desired results. For prophylactic use, beneficial or desired results include eliminating or reducing the risk, lessening the severity, or delaying the outset of the disease, including biochemical, histological and/or behavioral symptoms of the disease, its complications and intermediate pathological phenotypes presenting during development of the disease. For therapeutic use, beneficial or desired results include clinical results such as reducing incidence or amelioration of one or more symptoms of various diseases or conditions (such as for example cancer), decreasing the dose of other medications required to treat the disease, enhancing the effect of another medication, and/or delaying the progression of the disease. An effective dosage can be administered in one or more administrations. For purposes of the instant disclosure, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” can be considered in the context of administering one or more therapeutic agents, and a single agent can be considered to be given in an effective amount if, in conjunction with one or more other agents, a desirable result can be or is achieved.

As used herein, a “subject” is any mammal, e.g a human, or a monkey. Mammals include, but are not limited to, farm animals, sport animals, pets, primates, horses, dogs, cats, mice and rats. In an exemplary embodiment, the subject is a human. In an exemplary embodiment, the subject is a monkey, e.g. a cynomolgus monkey.

As used herein, “vector” means a construct, which is capable of delivering, and, preferably, expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

As used herein, “pharmaceutically acceptable carrier” or “pharmaceutically acceptable excipient” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline (PBS) or normal (0.9%) saline. Compositions of the instant disclosure comprising such carriers are formulated by well-known conventional methods (see, for example, Remington's Pharmaceutical Sciences, 18th edition, A. Gennaro, ed., Mack Publishing Co., Easton, P A, 1990; and Remington, The Science and Practice of Pharmacy 21 st Ed. Mack Publishing, 2005).

As used herein, “alloreactivity” refers to the ability of T cells to recognize MHC complexes that were not encountered during thymic development. Alloreactivity manifests itself clinically as host-versus-graft rejection and graft-versus-host disease.

Reference to “about” a value or parameter herein includes (and describes) embodiments that are directed to plus or minus 1, 2, 3, 4, 5, 6, 7, 8, 9 or 10% that value or parameter per se. For example, description referring to “about X” includes description of “X.” Numeric ranges are inclusive of the numbers defining the range.

It is understood that wherever embodiments are described herein with the language “comprising,” otherwise analogous embodiments described in terms of “consisting of and/or “consisting essentially of” are also provided.

Where aspects or embodiments of the instant disclosure are described in terms of a Markush group or other grouping of alternatives, the instant disclosure encompasses not only the entire group listed as a whole, but also each member of the group individually and all possible subgroups of the main group, and also the main group absent one or more of the group members. The instant disclosure also envisages the explicit exclusion of one or more of any of the group members in the disclosed and/or claimed embodiments.

Unless otherwise defined, all technical and scientific terms used herein have the same meaning as commonly understood by one of ordinary skill in the art to which the present disclosure belongs. In case of conflict, the present specification, including definitions, will control. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular.

Exemplary methods and materials are described herein, although methods and materials similar or equivalent to those described herein can also be used in the practice or testing of the instant disclosure. The materials, methods, and examples are illustrative only and not intended to be limiting.

An “antigen binding protein” comprises one or more antigen binding domains. An “antigen binding domain” as used herein means any polypeptide that binds a specified target antigen. In some embodiments, the antigen binding domain binds to an antigen on a tumor cell. In some embodiments, the antigen binding domain binds to an antigen on a cell involved in a hyperproliferative disease or to a viral or bacterial antigen.

Antigen binding domains include, but are not limited to, antibody binding regions that are immunologically functional fragments. The term “immunologically functional fragment” (or “fragment”) of an antigen binding domain is a species of antigen binding domain comprising a portion (regardless of how that portion is obtained or synthesized) of an antibody that lacks at least some of the amino acids present in a full-length chain, but which is still capable of specifically binding to a target antigen. Such fragments are biologically active in that they bind to the target antigen and can compete with other antigen binding domains, including intact antibodies, for binding to a given epitope.

Immunologically functional immunoglobulin fragments include, but are not limited to, scFv fragments, Fab fragments (Fab′, F(ab′)2, and the like), one or more complementarity determining regions (“CDRs”), a diabody (heavy chain variable domain on the same polypeptide as a light chain variable domain, connected via a short peptide linker that is too short to permit pairing between the two domains on the same chain), domain antibodies, bivalent antigen binding domains (comprises two antigen binding sites), multispecific antigen binding domains, and single-chain antibodies. These fragments can be derived from any mammalian source, including but not limited to human, mouse, rat, camelid or rabbit. As will be appreciated by one of skill in the art, an antigen binding domain can include non-protein components.

The variable regions typically exhibit the same general structure of relatively conserved framework regions (FR) joined by the 3 hypervariable regions (CDRs). The CDRs from the two chains of each pair typically are aligned by the framework regions, which can enable binding to a specific epitope. From N-terminal to C-terminal, both light and heavy chain variable regions typically comprise the domains FR1, CDR1, FR2, CDR2, FR3, CDR3 and FR4. By convention, CDR regions in the heavy chain are typically referred to as HC CDR1, CDR2, and CDR3. The CDR regions in the light chain are typically referred to as LC CDR1, CDR2, and CDR3.

In some embodiments, antigen binding domains comprise one or more complementarity binding regions (CDRs) present in the full-length light or heavy chain of an antibody, and in some embodiments comprise a single heavy chain and/or light chain or portion thereof. These fragments can be produced by recombinant DNA techniques or can be produced by enzymatic or chemical cleavage of antigen binding domains, including intact antibodies.

In some embodiments, the antigen binding domain is an antibody or fragment thereof, including one or more of the complementarity determining regions (CDRs) thereof. In some embodiments, the antigen binding domain is a single chain variable fragment (scFv), comprising light chain CDRs: CDR1, CDR2 and CDR3, and heavy chain CDRs: CDR1, CDR2 and CDR3.

The assignment of amino acids to each of the framework, CDR, and variable domains is typically in accordance with numbering schemes of Kabat numbering (see, e.g., Kabat et al. in Sequences of Proteins of Immunological Interest, 5th Ed., NIH Publication 91-3242, Bethesda Md. 1991), Chothia numbering (see, e.g., Chothia & Lesk, (1987), J Mol Biol 196: 901-917; Al-Lazikani et al., (1997) J Mol Biol 273: 927-948; Chothia et al., (1992) J Mol Biol 227: 799-817; Tramontano et al., (1990) J Mol Biol 215(1): 175-82; and U.S. Pat. No. 7,709,226), contact numbering, the AbM scheme (Antibody Modeling program, Oxford Molecular) or the AHo system (Honneger and Pluckthun, J Mol Biol (2001) 309(3):657-70).

In some embodiments, the antigen binding domain is a recombinant antigen receptor. The term “recombinant antigen receptor” as used herein refers broadly to a non-naturally occurring surface receptor that comprises an extracellular antigen-binding domain or an extracellular ligand-binding domain, a transmembrane domain and an intracellular domain. In some embodiments, the recombinant antigen receptor is a chimeric antigen receptor (CAR). Chimeric antigen receptors (CARs) are well-known in the art. A CAR is a fusion protein that comprises an antigen recognition moiety, a transmembrane domain and T cell activation domains (see, e.g., Eshhar et al., Proc. Natl. Acad. Sci. USA, 90(2): 720-724 (1993)).

In some embodiments, the intracellular domain of a recombinant antigen receptor comprises a co-stimulatory domain and an ITAM-containing domain. In some embodiments, the intracellular domain of a recombinant antigen receptor comprises an intracellular protein or a functional variant thereof (e.g., truncation(s), insertion(s), deletion(s) or substitution(s)).

The term “extracellular ligand-binding domain” or “extracellular antigen-binding domain” as used herein refers to a polypeptide that is capable of binding a ligand or an antigen or capable of interacting with a cell surface molecule, such as a ligand or a surface antigen. For example, the extracellular ligand-binding or antigen-binding domain can be chosen to recognize a ligand that acts as a cell surface marker on target cells associated with a particular disease state, e.g., a tumor-specific antigen. In some embodiments, the antigen-binding domain comprises an antibody, or an antigen binding fragment or an antigen binding portion of an antibody. In some embodiments, the antigen binding domain comprises an Fv or scFv, an Fab or scFab, an F(ab′)2 or a scF(ab′)2, an Fd, a monobody, a affibody, a camelid antibody, a VHH antibody, a single domain antibody, or a darpin. In some embodiments, the ligand-binding domain comprises a partner of a binding pair, such as a ligand that binds to a surface receptor, or an ectodomain of a surface receptor that binds to a ligand.

The term “stalk domain” or “hinge domain” are used interchangeably herein to refer to any polypeptide that functions to link the transmembrane domain to the extracellular ligand-binding domain. In particular, stalk domains are often used to provide more flexibility and accessibility for the extracellular ligand-binding domain.

The term “intracellular signaling domain” refers to the portion of a protein which transduces the effector signal function signal and directs the cell to perform a specialized function.

### Vectors

Expression vectors and methods for the administration of polynucleotide compositions are known in the art and further described herein.

In another aspect, the instant disclosure provides a method of making any of the polynucleotides described herein.

Polynucleotides complementary to any such sequences are also encompassed by the instant disclosure. Polynucleotides can be single-stranded (coding or antisense) or double-stranded, and can be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences can, but need not, be present within a polynucleotide of the instant disclosure, and a polynucleotide can, but need not, be linked to other molecules and/or support materials.

Polynucleotides can comprise a native sequence (i.e., an endogenous sequence that encodes an antibody or a portion thereof) or can comprise a variant of such a sequence. Polynucleotide variants contain one or more substitutions, additions, deletions and/or insertions such that the immunoreactivity of the encoded polypeptide is not diminished, relative to a native immunoreactive molecule. The effect on the immunoreactivity of the encoded polypeptide can generally be assessed as described herein. Variants preferably exhibit at least about 70% identity, more preferably, at least about 80% identity, yet more preferably, at least about 90% identity, and most preferably, at least about 95% identity to a polynucleotide sequence that encodes a native antibody or a portion thereof. Two polynucleotide or polypeptide sequences are said to be “identical” if the sequence of nucleotides or amino acids in the two sequences is the same when aligned for maximum correspondence as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, or 40 to about 50, in which a sequence can be compared to a reference sequence of the same number of contiguous positions after the two sequences are optimally aligned.

Optimal alignment of sequences for comparison can be conducted using the Megalign program in the Lasergene suite of bioinformatics software (DNASTAR, Inc., Madison, WI), using default parameters. This program embodies several alignment schemes described in the following references: Dayhoff, M. O., 1978, A model of evolutionary change in proteins—Matrices for detecting distant relationships. In Dayhoff, M. O. (ed.) Atlas of Protein Sequence and Structure, National Biomedical Research Foundation, Washington DC Vol. 5, Suppl. 3, pp. 345-358; Hein J., 1990, Unified Approach to Alignment and Phylogenes pp. 626-645 Methods in Enzymology vol. 183, Academic Press, Inc., San Diego, CA; Higgins, D. G. and Sharp, P. M., 1989, CABIOS 5:151-153; Myers, E. W. and Muller W., 1988, CABIOS 4:1 1-17; Robinson, E. D., 1971, Comb. Theor. 11:105; Santou, N., Nes, M., 1987, Mol. Biol. Evol. 4:406-425; Sneath, P. H. A. and Sokal, R. R., 1973, Numerical Taxonomy the Principles and Practice of Numerical Taxonomy, Freeman Press, San Francisco, CA; Wilbur, W. J. and Lipman, D. J., 1983, Proc. Natl. Acad. Sci. USA 80:726-730.

In some embodiments, the “percentage of sequence identity” is determined by comparing two optimally aligned sequences over a window of comparison of at least 20 positions, wherein the portion of the polynucleotide or polypeptide sequence in the comparison window can comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e. the window size) and multiplying the results by 100 to yield the percentage of sequence identity.

The present disclosure provides variant polynucleotides or variant polypeptides as compared to a reference polynucleotide or reference polypeptide, respectively. Variants can result from one or more insertions, one or more deletions, and/or one or more substitutions. In some embodiments, a variant polypeptide can contain one or more amino acid insertions, one or more amino acid deletions, and/or one or more amino acid substitutions as compared to a reference polypeptide. In some embodiments, the amino acid substitutions are conservative amino acid substitutions. Exemplary conservative amino acid residues are shown below:

TABLE A
Amino Acid Substitutions

| Original Residue | | |
|---|---|---|
| (naturally occurring | Conservative | Exemplary |
| amino acid) | Substitutions | Substitutions |
| Ala (A) | Val | Val; Leu; Ile |
| Arg (R) | Lys | Lys; Gln; Asn |
| Asn (N) | Gln | Gln; His; Asp, Lys; Arg |
| Asp (D) | Glu | Glu; Asn |
| Cys (C) | Ser | Ser; Ala |
| Gln (Q) | Asn | Asn; Glu |
| Glu (E) | Asp | Asp; Gln |
| Gly (G) | Ala | Ala |
| His (H) | Arg | Asn; Gln; Lys; Arg |
| Ile (I) | Leu | Leu; Val; Met; Ala; Phe; |
| | | Norleucine |
| Leu (L) | Ile | Norleucine; Ile; Val; Met; |
| | | Ala; Phe |
| Lys (K) | Arg | Arg; Gln; Asn |
| Met (M) | Leu | Leu; Phe; Ile |
| Phe (F) | Tyr | Leu; Val; Ile; Ala; Tyr |
| Pro (P) | Ala | Ala |
| Ser (S) | Thr | Thr |
| Thr (T) | Ser | Ser |
| Trp (W) | Tyr | Tyr; Phe |
| Tyr (Y) | Phe | Trp; Phe; Thr; Ser |
| Val (V) | Leu | Ile; Leu; Met; Phe; Ala; |
| | | Norleucine |

Variants can also, or alternatively, be substantially homologous to a native gene, or a portion or complement thereof. Such polynucleotide variants are capable of hybridizing under moderately stringent conditions to a naturally occurring DNA sequence encoding a native antibody (or a complementary sequence).

Suitable “moderately stringent conditions” include prewashing in a solution of 5×SSC, 0.5% SDS, 1.0 mM EDTA (pH 8.0); hybridizing at 50° C.-65° C., 5×SSC, overnight; followed by washing twice at 65° C. for 20 minutes with each of 2×, 0.5× and 0.2×SSC containing 0.1% SDS.

As used herein, “highly stringent conditions” or “high stringency conditions” are those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/m), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C. The skilled artisan will recognize how to adjust the temperature, ionic strength, etc. as necessary to accommodate factors such as probe length and the like.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the instant disclosure. Further, alleles of the genes comprising the polynucleotide sequences provided herein are within the scope of the instant disclosure. Alleles are endogenous genes that are altered as a result of one or more mutations, such as deletions, additions and/or substitutions of nucleotides. The resulting mRNA and protein can, but need not, have an altered structure or function. Alleles can be identified using standard techniques (such as hybridization, amplification and/or database sequence comparison).

The polynucleotides of the instant disclosure can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence.

For preparing polynucleotides using recombinant methods, a polynucleotide comprising a desired sequence can be inserted into a suitable vector, and the vector in turn can be introduced into a suitable host cell for replication and amplification, as further described herein. Polynucleotides can be inserted into host cells by any means known in the art. Cells are transformed by introducing an exogenous polynucleotide by direct uptake, endocytosis, transfection, F-mating or electroporation. Once introduced, the exogenous polynucleotide can be maintained within the cell as a non-integrated vector (such as a plasmid) or integrated into the host cell genome. The polynucleotide so amplified can be isolated from the host cell by methods well known within the art. See, e.g., Sambrook et al., 1989.

Alternatively, PCR allows reproduction of DNA sequences. PCR technology is well known in the art and is described in, e.g., U.S. Pat. Nos. 4,683,195, 4,800,159, 4,754,065 and 4,683,202, as well as PCR: The Polymerase Chain Reaction, Mullis et al. eds., Birkauswer Press, Boston, 1994.

RNA can be obtained by using the isolated DNA in an appropriate vector and inserting it into a suitable host cell. When the cell replicates and the DNA is transcribed into RNA, the RNA can then be isolated using methods well known to those of skill in the art, as set forth in Sambrook et al., 1989, supra, for example.

Suitable cloning vectors can be constructed according to standard techniques, or can be selected from a large number of cloning vectors available in the art. While the cloning vector selected can vary according to the host cell intended to be used, useful cloning vectors will generally have the ability to self-replicate, can possess a single target for a particular restriction endonuclease, and/or can carry genes for a marker that can be used in selecting clones containing the vector. Suitable examples include plasmids and bacterial viruses, e.g., pUC18, pUC19, Bluescript (e.g., pBS SK+) and its derivatives, mp18, mp19, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Expression vectors generally are replicable polynucleotide constructs that contain a polynucleotide according to the instant disclosure. It is implied that an expression vector must be replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO 87/04462. Vector components can generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons.

The vectors containing the polynucleotides of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell.

A polynucleotide encoding a polypeptide, e.g. a chimeric switch receptor, chimeric antigen receptor, or chimeric cytokine receptor, can exist in an expression cassette or expression vector (e.g., a plasmid for introduction into a bacterial host cell, or a viral vector such as a baculovirus vector for transfection of an insect host cell, or a plasmid or viral vector such as a lentivirus for transfection of a mammalian host cell). In some embodiments, a polynucleotide or vector can include a nucleic acid sequence encoding ribosomal skip sequences such as, for example without limitation, a sequence encoding a 2A peptide. 2A peptides, which were identified in the Aphthovirus subgroup of picornaviruses, cause a ribosomal “skip” from one codon to the next without the formation of a peptide bond between the two amino acids encoded by the codons (see, e.g., Donnelly and Elliott 2001; Atkins, Wills et al. 2007; Doronina, Wu et al. 2008). By “codon” is meant three nucleotides on an mRNA (or on the sense strand of a DNA molecule) that are translated by a ribosome into one amino acid residue. Thus, two polypeptides can be synthesized from a single, contiguous open reading frame within an imRNA when the polypeptides are separated by a 2A oligopeptide sequence (P2A sequence) that is in frame. Such ribosomal skip mechanisms are well known in the art and are known to be used by several vectors for the expression of several proteins encoded by a single messenger RNA. In some embodiments, a 2A coding nucleotide sequence is positioned between a nucleic acid sequence encoding a CAR as disclosed herein, e.g. a DLL3 CAR, and a CCR as disclosed herein, e.g. either an inducible CCR or a constitutive CCR; in some embodiments, the polynucleotide encoding the CAR and CCR is incorporated into the TRAC locus of an immune cell in the preparation of an engineered immune cell as disclosed herein. In some embodiments, the nucleic acid sequence encoding the CCR is 5′ to the nucleic acid sequence encoding the CAR. In some embodiments, the nucleic acid sequence encoding the CAR is 5′ to the nucleic acid sequence encoding the CCR.

To direct transmembrane polypeptides into the secretory pathway of a host cell, in some embodiments, a secretory signal sequence (also known as a leader sequence, signal peptide, prepro-sequence or pre-sequence) is provided in a polynucleotide sequence or vector sequence. The secretory signal sequence is operably linked to the transmembrane nucleic acid sequence, i.e., the two sequences are joined in the correct reading frame and positioned to direct the newly synthesized polypeptide into the secretory pathway of the host cell. Secretory signal sequences are commonly positioned 5′ to the nucleic acid sequence encoding the polypeptide of interest, although certain secretory signal sequences can be positioned elsewhere in the nucleic acid sequence of interest (see, e.g., Welch et al., U.S. Pat. No. 5,037,743; Holland et al., U.S. Pat. No. 5,143,830). Those skilled in the art will recognize that, in view of the degeneracy of the genetic code, considerable sequence variation is possible among these polynucleotide molecules. In some embodiments, nucleic acid sequences of the instant disclosure are codon-optimized for expression in mammalian cells, preferably for expression in human cells. Codon-optimization refers to the exchange in a sequence of interest of codons that are generally rare in highly expressed genes of a given species for codons that are generally frequent in highly expressed genes of such species, such codons encoding the same amino acids as the codons that are being exchanged.

Methods of preparing immune cells for use in immunotherapy are provided herein. In some embodiments, the methods comprise introducing a chimeric switch receptor as disclosed herein into one or more immune cells, or introducing a polynucleotide encoding the chimeric switch receptor, and expanding the cells. In some embodiments, the instant disclosure relates to a method of engineering an immune cell comprising: providing an immune cell and expressing at the surface of the cell at least one chimeric switch receptor. In some embodiments, the method comprises: transfecting the cell with at least one polynucleotide encoding a chimeric switch receptor, and expressing the at least one polynucleotide in the cell.

In some embodiments, the polynucleotides encoding the chimeric switch receptor are present in one or more expression vectors for stable expression in the cells. In some embodiments, the polynucleotides are present in viral vectors for stable expression in the cells. In some embodiments, the viral vectors can be for example, lentiviral vectors or adenoviral vectors.

In some embodiments, polynucleotides encoding polypeptides according to the present disclosure can be mRNA which is introduced directly into the cells, for example by electroporation. In some embodiments, CytoPulse technology can be used to transiently permeabilize living cells for delivery of material into the cells. Parameters can be modified in order to determine conditions for high transfection efficiency with minimal mortality.

Also provided herein are methods of transfecting an immune cell e.g. a T cell. In general, any conventional method known to the person of ordinary skill in the art can be used, such as introducing any of RNA, DNA or protein into a cell by means of electroporation. See, e.g., Luft and Ketteler, J. Biomolec Screening 20(8): 932 (2015) (DOI: 10.1177/1087057115579638). In some embodiments, the method comprises: contacting a T cell with RNA and applying to the T cell an agile pulse sequence consisting of: (a) an electrical pulse with a voltage range from about 2250 to 3000 V per centimeter; (b) a pulse width of 0.1 ms; (c) a pulse interval of about 0.2 to 10 ms between the electrical pulses of step (a) and (b); (d) an electrical pulse with a voltage range from about 2250 to 3000 V per centimeter with a pulse width of about 100 ms and a pulse interval of about 100 ms between the electrical pulse of step (b) and the first electrical pulse of step (c); and (e) four electrical pulses with a voltage of about 325 V with a pulse width of about 0.2 ms and a pulse interval of 2 ms between each of 4 electrical pulses. In some embodiments, a method of transfecting a T cell comprises contacting said T cell with RNA and applying to the T cell an agile pulse sequence comprising: (a) an electrical pulse with a voltage of about 1600, 2250, 2300, 2350, 2400, 2450, 2500, 2550, 2400, 2450, 2500, 2600, 2700, 2800, 2900 or 3000V per centimeter; (b) a pulse width of 0.1 ms; (c) and a pulse interval of about 0.2, 0.5, 1, 2, 3, 4, 5, 6, 7, 8, 9 or 10 ms between the electrical pulses of step (a) and (b); (d) one electrical pulse with a voltage range from about 2250 to 3000 V per centimeter, e.g. of 2250, 2300, 2350, 2400, 2450, 2500, 2550, 2400, 2450, 2500, 2600, 2700, 2800, 2900 or 3000V per centimeter with a pulse width of 100 ms and a pulse interval of 100 ms between the electrical pulse of step (b) and the first electrical pulse of step (c); and (e) 4 electrical pulses with a voltage of about 325 V with a pulse width of about 0.2 ms and a pulse interval of about 2 ms between each of 4 electrical pulses. Any values included in the value range described above are disclosed in the present application. Electroporation medium can be any suitable medium known in the art. In some embodiments, the electroporation medium has conductivity in a range spanning about 0.01 to about 1.0 milliSiemens.

In some embodiments, the method can further comprise a step of genetically modifying a cell by inactivating at least one gene expressing, for example without limitation, a component of the TCR, a target for an immunosuppressive agent, an HLA gene, and/or an immune checkpoint protein such as, for example, PDCD1 or CTLA-4. By inactivating a gene it is intended that the gene of interest is not expressed in a functional protein form. In some embodiments, the gene to be inactivated is selected from the group consisting of, for example without limitation, TCRα, TCRβ, CD52, GR, deoxycytidine kinase (DCK), TGF-B, and CTLA-4. In some embodiments the method comprises inactivating one or more genes by introducing into the cells a rare-cutting endonuclease able to selectively inactivate a gene by selective DNA cleavage. In some embodiments the rare-cutting endonuclease can be, for example, a transcription activator-like effector nuclease (TALE-nuclease) or CRISPR-based endonuclease (e.g Cas-9 or Cas12a).

In another aspect, a step of genetically modifying cells can comprise: modifying immune cells (e.g. T-cells) by inactivating at least one gene expressing a target for an immunosuppressive agent, and; expanding the cells, optionally in the presence of the immunosuppressive agent. An immunosuppressive agent is an agent that suppresses immune function by one of several mechanisms of action. An immunosuppressive agent can diminish the extent and/or voracity of an immune response. Non-limiting examples of immunosuppressive agents include calcineurin inhibitors, targets of rapamycin, interleukin-2 α-chain blockers, inhibitors of inosine monophosphate dehydrogenase, inhibitors of dihydrofolic acid reductase, corticosteroids, and immunosuppressive antimetabolites. Some cytotoxic immunosuppressants act by inhibiting DNA synthesis. Others can act through activation of T cells or by inhibiting the activation of helper cells. The methods according to the instant disclosure allow conferring immunosuppressive resistance to e.g. T cells for immunotherapy by inactivating the target of the immunosuppressive agent in the T cells. As non-limiting examples, targets for an immunosuppressive agent can be a receptor for an immunosuppressive agent such as for example without limitation CD52, glucocorticoid receptor (GR), FKBP family gene members, and cyclophilin family gene members.

The present disclosure provides a method of making an engineered immune cell disclosed herein e.g. an engineered immune cell that expresses a chimeric switch receptor and optionally further expresses a CAR or a CAR and a CCR. The present disclosure also provides compositions comprising such engineered immune cells made by the disclosed methods. The present disclosure further provides methods of treating comprising administering the cells and compositions that comprise the cells. The methods and compositions provided herein are useful for improving therapeutic efficacy of engineered immune cells e.g. engineered T cells such as CAR-T cells.

One or more proteins or polypeptides, such as a chimeric polypeptide disclosed herein e.g. a chimeric switch receptor, a CAR and a CCR, can be synthesized in situ in a cell after introduction of one or more polynucleotide constructs encoding the proteins into the cell. Alternatively, a polypeptide can be produced outside of cells, and then introduced into cells. Methods for introducing a polynucleotide construct into cells are known in the art. In some embodiments, stable transformation methods can be used to integrate the polynucleotide construct into the genome of the cell. In other embodiments, transient transformation methods can be used to transiently express the polynucleotide construct, and the polynucleotide construct not integrated into the genome of the cell. In other embodiments, virus-mediated methods can be used. The polynucleotides can be introduced into a cell by any suitable means such as for example, recombinant viral vectors (e.g. retroviruses, including lentiviruses, adenoviruses), liposomes, and the like. Transient transformation methods include, for example without limitation, microinjection, electroporation or particle bombardment. Polynucleotides can be included in vectors, such as for example plasmid vectors or viral vectors.

In some embodiments, an engineered immune cell e.g. T cell of the present disclosure comprises at least one chimeric polypeptide disclosed herein e.g. a chimeric switch receptor and other polypeptides of interest such as a CAR and a CCR. The engineered immune cell e.g. T cell may be further modified e.g. genetically engineered to express a reduced level of any one or more of CD70, CD52, PD1, TIM3, CISH, cbl-b, TIGIT and TCRa (such as the constant region of TCRa, TRAC). In some embodiments, the introduction into the cell of a polynucleotide or vector encoding the chimeric polypeptide e.g. chimeric switch receptor, CAR and/or CCR also functions to disrupt the expression of any one or more of CD70, CD52, PD1, TIM3, CISH, cbl-b, TIGIT and TCRa. In some embodiments, the polynucleotide or vector encoding the chimeric polypeptide e.g. chimeric switch receptor, CAR and/or CCR is introduced into the genome of the engineered immune cell by site-specific integration (SSI) at one or more specific genetic loci. In some embodiments, the one or more genetic loci can be one or more or, for example without limitation, CD70, CD52, PD1, TIM3, CISH, cbl-b, TIGIT and TCRa (such as TRAC). In certain embodiments, the polynucleotide or vector encoding the chimeric polypeptide, e.g., the chimeric switch receptor is introduced into the CD52 locus. In certain embodiments, the polynucleotide or vector encoding the chimeric switch receptor is introduced into any one of the PD1, TIM3, CISH, cbl-b, or TIGIT locus. In certain embodiments, the polynucleotide encoding the CAR and/or CCR is introduced into the TRAC locus. In certain embodiments, the polynucleotide encoding the CAR and/or CCR is introduced into the CD52 locus.

In some embodiments of an engineered immune cell e.g. T cell provided herein, a CAR that the cell expresses can comprise an extracellular ligand-binding domain (e.g., a single chain variable fragment (scFv)), a transmembrane domain, and an intracellular signaling domain. In some embodiments, the extracellular ligand-binding domain, transmembrane domain, and intracellular signaling domain are in one polypeptide, i.e., in a single chain. Multichain CARs and polypeptides are also provided herein. In some embodiments, the multichain CARs comprise: a first polypeptide comprising a transmembrane domain and at least one extracellular ligand-binding domain, and a second polypeptide comprising a transmembrane domain and at least one intracellular signaling domain, wherein the polypeptides assemble together to form a multichain CAR.

The extracellular ligand-binding domain of a CAR specifically binds to a target of interest. In some embodiments, the target of interest can be any molecule of interest, including, for example, without limitation, BCMA, EGFRvIII, Flt-3, WT-1, CD20, CD23, CD30, CD38, CD70, CD33, CD133, WT1, TSPAN10, MHC-PRAME, Livl, ADAM10, CHRNA2, LeY, NKG2D, CS1, CD44v6, ROR1, CD19, Claudin-18.2 (Claudin-18A2, or Claudin18 isoform 2), DLL3 (Delta-like protein 3, Drosophila Delta homolog 3, Delta3), Muc17, Muc3, Muc3, Muc16, FAP alpha (Fibroblast Activation Protein alpha), Ly6G6D (Lymphocyte antigen 6 complex locus protein G6d, c6orf23, G6D, MEGT1, NG25), RNF43 (E3 ubiquitin-protein ligase RNF43, RING finger protein 43), specifically including the human form of any of the listed exemplary targets.

In some embodiments, the extracellular ligand-binding domain of a CAR comprises an scFv comprising the light chain variable (VL) region and the heavy chain variable (VH) region of a target antigen-specific monoclonal antibody joined by a flexible linker. Single chain variable region fragments are made by linking light and/or heavy chain variable regions by using a short linking peptide (Bird et al., Science 242:423-426, 1988). An example of a linking peptide is the GS linker having the amino acid sequence (GGGGS)₄ (SEQ ID NO: 159), which bridges approximately 3.5 nm between the carboxy terminus of one variable region and the amino terminus of the other variable region. Linkers of other sequences have been designed and used (Bird et al., 1988, supra). In general, linkers can be short, flexible polypeptides and preferably comprised of about 20 or fewer amino acid residues. Linkers can in turn be modified for additional functions, such as attachment of drugs or attachment to solid supports. The single chain variants can be produced either recombinantly or synthetically. For synthetic production of scFv, an automated synthesizer can be used. For recombinant production of scFv, a suitable plasmid or other vector containing a polynucleotide that encodes the scFv can be introduced into a suitable host cell, either eukaryotic, such as yeast, plant, insect or mammalian cells, or prokaryotic, such as E. coli. Polynucleotides encoding the scFv of interest can be made by routine manipulations such as ligation of polynucleotides. The resultant scFv can be isolated using standard protein purification techniques known in the art.

The intracellular signaling domain of a CAR as disclosed herein is responsible for intracellular signaling following the binding of the CAR's extracellular ligand-binding domain to the target, resulting in the activation of the immune cell and immune response. The intracellular signaling domain has the ability to activate at least one of the normal effector functions of the immune cell in which the CAR is expressed. For example, the effector function of a T cell can be a cytolytic activity or helper activity including the secretion of cytokines.

In some embodiments, an intracellular signaling domain for use in a CAR can be the cytoplasmic sequences of, for example without limitation, the T cell receptor and co-receptors that act in concert to initiate signal transduction following antigen receptor engagement, as well as any derivative or variant of these sequences and any synthetic sequence that has the same functional capability. Intracellular signaling domains comprise two distinct classes of cytoplasmic signaling sequences: those that initiate antigen-dependent primary activation, and those that act in an antigen-independent manner to provide a secondary or co-stimulatory signal. Primary cytoplasmic signaling sequences can comprise signaling motifs which are known as immunoreceptor tyrosine-based activation motifs or ITAMs. ITAMs are well defined signaling motifs found in the intracytoplasmic tail of a variety of receptors that serve as binding sites for syk/zap70 class tyrosine kinases. Examples of ITAM used in the instant disclosure can include as non-limiting examples those derived from TCRζ, FcRγ, FcRβ, FcRε, CD3γ, CD3δ, CD3ε, CD5, CD22, CD79a, CD79b and CD66d. In some embodiments, the intracellular signaling domain of the CAR comprises a CD3ζ signaling domain. In some embodiments the intracellular signaling domain of the CAR of the instant disclosure comprises or further comprises a domain of a co-stimulatory molecule.

In some embodiments, the intracellular signaling domain of a CAR of the instant disclosure comprises a part of a co-stimulatory molecule selected from the group consisting of fragment of 4-1BB (GenBank: AAA53133) and CD28 (NP_006130 and isoforms thereof).

CARs are expressed on the surface membrane of the cell. Thus, the CAR can comprise a transmembrane domain. Suitable transmembrane domains for a CAR disclosed herein have the ability to (a) be expressed at the surface of a cell, for example an immune cell such as, for example without limitation, lymphocyte cells (e.g. T cells) or Natural killer (NK) cells, and (b) interact with the ligand-binding domain and intracellular signaling domain for directing a cellular response of an immune cell against a predefined target cell. The transmembrane domain can be derived either from a natural or from a synthetic source. The transmembrane domain can be derived from any membrane-bound or transmembrane protein. As non-limiting examples, the transmembrane polypeptide can be a domain of the T cell receptor such as a, β, γ or δ, polypeptide constituting CD3 complex, IL-2 receptor e.g. p55 (α chain), p75 (β chain or γ chain), subunit chain of Fc receptors, in particular Fcγ receptor III or CD proteins. Alternatively, the transmembrane domain can be synthetic and can comprise predominantly hydrophobic residues such as leucine and valine. In some embodiments said transmembrane domain is derived from the human CD8α chain (e.g., NP_001139345.1). The transmembrane domain can further comprise a stalk domain between the extracellular ligand-binding domain and said transmembrane domain. A stalk domain can comprise up to 300 amino acids, for example, from 10 to 100 amino acids or 25 to 50 amino acids. The stalk region can be derived from all or part of naturally occurring molecules, such as from all or part of the extracellular region of CD8, CD4, or CD28, or from all or part of an antibody constant region. Alternatively, the stalk domain can be a synthetic sequence that corresponds to a naturally occurring stalk sequence or can be an entirely synthetic stalk sequence. In some embodiments said stalk domain is a part of human CD8α chain (e.g., NP_001139345 and isoforms thereof). In another particular embodiment, the transmembrane domain comprises a part of the human CD8α chain. In some embodiments, CARs disclosed herein can comprise an extracellular ligand-binding domain that specifically binds BCMA, CD8α human stalk and transmembrane domains, the CD3ζ signaling domain, and 4-1BB signaling domain. In some embodiments, a CAR can be introduced into an immune cell as a transgene via a vector e.g. a plasmid vector. In some embodiments, the vector e.g. plasmid vector can also contain, for example, a selection marker which provides for identification and/or selection of cells which received the vector.

A chimeric switch receptor, CAR and CCR polypeptides can be synthesized in situ in the cell after introduction of polynucleotides encoding the polypeptides into the cell. Alternatively, one or more of the polypeptides can be produced outside of cells, and then introduced into cells. Methods for introducing a polynucleotide construct into cells are known in the art. In some embodiments, stable transformation methods can be used to integrate the polynucleotide construct into the genome of the cell. In other embodiments, transient transformation methods can be used to transiently express the polynucleotide construct, and the polynucleotide construct not integrated into the genome of the cell. In other embodiments, virus-mediated methods can be used. The polynucleotides can be introduced into a cell by any suitable means such as for example, recombinant viral vectors (e.g. retroviruses (e.g. lentiviruses), adenoviruses), liposomes, and the like. Transient transformation methods include, for example without limitation, microinjection, electroporation or particle bombardment. Polynucleotides can be included in vectors, such as for example plasmid vectors or viral vectors.

Also provided herein are immune cells e.g. T cells such as isolated T cells obtained according to any one of the methods described herein. Any immune cell capable of expressing heterologous DNAs can be used for the purpose of expressing the chimeric switch receptor, CAR and CCR polypeptides of interest and further for engineering to express a reduced level, or eliminating the expression of, for example, CD70, CD52, PD1, TIM3, CISH, cbl-b, TIGIT and/or TCRa. In some embodiments, the immune cell is a T cell. In some embodiments, an immune cell can be derived from, for example without limitation, a stem cell. The stem cells can be adult stem cells, non-human embryonic stem cells, more particularly non-human stem cells, cord blood stem cells, progenitor cells, bone marrow stem cells, induced pluripotent stem cells, totipotent stem cells or hematopoietic stem cells. Representative human cells are CD34+ cells. The isolated cell can also be a dendritic cell, killer dendritic cell, a mast cell, a NK-cell, a B-cell or a T cell selected from the group consisting of inflammatory T-lymphocytes, cytotoxic T-lymphocytes, regulatory T-lymphocytes or helper T-lymphocytes. In some embodiments, the cell can be derived from the group consisting of CD4+T-lymphocytes and CD8+T-lymphocytes. In some embodiments, the immune cells e.g. T cells such as isolated T cells are further modified e.g. genetically engineered by methods described herein (e.g. known gene editing techniques that employ, for example, TALENs, CRISPR/Cas9, or megaTAL nucleases to partially or wholly delete or disrupt one or more gene loci as desired) so that they express a reduced level of e.g. one or more of CD70, CD52, PD1, TIM3, CISH, cbl-b, TIGIT and TCRa relative to comparable cells not engineered to express a reduced or altered level of the corresponding protein.

Amino acid sequences of components of a chimeric switch receptor as disclosed herein are provided in Table 1.

TABLE 1
Exemplary Individual modules amino acid sequence

| SEQ ID | Module | |
|---|---|---|
| NO: | name | Amino acid sequence |
| 1 | CD8 signal | MALPVTALLLPLALLLHAARP |
| | sequence | |
| 2 | BR2 signal | MGRGLLRGLWPLHIVLWTRIAS |
| | sequence | |
| 3 | TpoR (S505N | SDPTRVETATETAWISLVTALHLVLGLNAVLGLLLLRKQFPAHYRRLRHALW |
| | W515K) | PSLPDLHRVLGQYLRDTAALSPPKATVSDTCEEVEPSLLEILPKSSERTPLP |
| | | L |
| 4 | IL2Rb-YY | DEGVAGAPTGSSPQPLQPLSGEDDAYCTFPSRDDLLLFSPSGQGEFRALNAR |
| | | LPLNTDAYLSLQELQGQDPTHLV |
| 5 | dn WT PD1 | PGWFLDSPDRPWNPPTFSPALLVVTEGDNATFTCSFSNTSESFVLNWYRMSP |
| | | SNQTDKLAAFPEDRSQPGQDCRFRVTQLPNGRDFHMSVVRARRNDSGTYLCG |
| | | AISLAPKAQIKESLRAELRVTERRAEVPTAHPSPSPRPAGQFQTLVVGVVGG |
| | | LLGSLVLLVWVLAVICSRAARGTIGARRTGQ |
| 6 | dnHACiPD1 | PGWFLDSPDRPWNPPTFSPALLVVTEGDNATFTCSFSNTSESFHVIWHRESP |
| | | SG |
| | | QTDTLAAFPEDRSQPGQDCRFRVTQLPNGRDFHMSVVRARRNDSGTYVCGVI |
| | | GSLVLLVWVLAVICSRAARGTIGARRTGQ |
| 7 | dn WT BR2 | TIPPHVQKSVNNDMIVTDNNGAVKFPQLCKFCDVRFSTCDNQKSCMSNCSIT |
| | | SICEKPQEVCVAVWRKNDENITLETVCHDPKLPYHDFILEDAASPKCIMKEK |
| | | KKPGETFFMCSCSSDECNDNIIFSEEYNTSNPDLLLVIFQVTGISLLPPLGV |
| | | AISVIIIFYCYRVNRQQKLSS |
| 8 | dN25 | QLCKFCDVRFSTCDNQKSCMSNCSITSICEKPQEVCVAVWRKNDENITLETV |
| | BR2(DNR) | CHDPKLPYHDFILEDAASPKCIMKEKKKPGETFFMCSCSSDECNDNIIFSEE |
| | | YNTSNPDLLLVIFQVTGISLLPPLGVAISVIIIFYCYRVNRQQKLSS |
| 9 | WT PD1 ECD | PGWFLDSPDRPWNPPTFSPALLVVTEGDNATFTCSFSNTSESFVLNWYRMSP |
| | | SNQTDKLAAFPEDRSQPGQDCRFRVTQLPNGRDFHMSVVRARRNDSGTYLCG |
| | | AISLAPKAQIKESLRAELRVTERRAEVPTAHPSPSPRPAGQFQTLV |
| 10 | HA PD1 ECD | PGWFLDSPDRPWNPPTFSPALLVVTEGDNATFTCSFSNTSESFHVIWHRESP |
| | | SGQTDTLAAFPEDRSQPGQDCRFRVTQLPNGRDFHMSVVRARRNDSGTYVCG |
| | | VISLAPKIQIKESLRAELRVTERRAEVPTAHPSPSPRPAGQFQTLV |
| 11 | PD-1 signal | MQIPQAPWPVVWAVLQLGWR |
| | peptide | |
| 12 | WT BR2 ECD | TIPPHVQKSVNNDMIVTDNNGAVKFPQLCKFCDVRFSTCDNQKSCMSNCSIT |
| | | SICEKPQEVCVAVWRKNDENITLETVCHDPKLPYHDFILEDAASPKCIMKEK |
| | | KKPGETFFMCSCSSDECNDNIIFSEEYNTSNPDLLLVIFQ |
| 13 | dN25 BR2 | QLCKFCDVRFSTCDNQKSCMSNCSITSICEKPQEVCVAVWRKNDENITLETV |
| | ECD | CHDPKLPYHDFILEDAASPKCIMKEKKKPGETFFMCSCSSDECNDNIIFSEE |
| | | YNTSNPDLLLVIFQ |
| 14 | PD1 TM | VGVVGGLLGSLVLLVWVLAVICSRAARGTIGARRTGQ |
| 15 | MyD88 | MAAGGPGAGSAAPVSSTSSLPLAALNMRVRRRLSLFLNVRTQVAADWTALAE |
| | | EMDFEYLEIRQLETQADPTGRLLDAWQGRPGASVGRLLDLLTKLGRDDVLLE |
| | | LGPSIEEDCQKYILKQQQEEAEKPLQVAAVDSSVPRTAELAGITTLDDPLGH |
| | | MPERFDAFICYCPSDI |
| 16 | CD28 ECD | CPSPLFPGPSKP |
| | (truncated) | |
| 17 | CD28 TM | FWVLVVVGGVLACYSLLVTVAFIIFWV |
| 18 | CD28 | RSKRSRLLHSDYMNMTPRRPGPTRKHYQPYAPPRDFAAYRS |
| | intracellular | |
| 19 | CD28.YMFM | RSKRSRLLHSDYMFMTPRRPGPTRKHYQPYAPPRDFAAYRS |
| | intracellular | |
| 20 | CD28.AYAA | RSKRSRLLHSDYMNMTPRRPGPTRKHYQAYAAPRDFAAYRS |
| | intracellular | |
| 21 | CD2 ECD | VEPVSCPEKGLD |
| | (truncated) | |
| 22 | CD2 TM | IYLIIGICGGGSLLMVFVALLVFYIT |
| 23 | CD2 | KRKKQRSRRNDEELETRAHRVATEERGRKPHQIPASTPQNPATSQHPPPPPG |
| | intracellular | HRSQAPSHRPPPPGHRVQHQPQKRPPAPSGTQVHQQKGPPLPRPRVQPKPPH |
| | (full); | GAAENSLSPSSN |
| | | GYF binding domain underlined, SH3 domain bolded |
| 24 | CD2 | KRKKQTPQNPATSQHPPPPPGHRSQAPSHRPPPPGHRVQHQPQKRPPAPSGT |
| | intracellular | QVHQQKGPPLPRPRVQPKPPHGAAENSLSPSSN |
| | (short) | |
| 25 | DAP10 ECD | QTTPGERSSLPAFYPGTSGSCSGCGSLSLP |
| 26 | DAP10 TM | LLAGLVAANAVASLLIVGAVF |
| | D57N | |
| 27 | DAP10 | LCARPRRSPAQEDGKVYINMPGRG |
| | intracellular | |
| 28 | ICOS TM | FWLPIGCAAFVVVCILGCILI |
| 29 | ICOS | CWLTKKKYSSSVHDPNGEYMFMRAVNTAKKSRLTDVTL |
| | intracellular | |
| 30 | CD40 TM | ALVVIPIIFGILFAILLVLVFI |
| 31 | CD40 | KKVAKKPTNKAPHPKQEPQEINFPDDLPGSNTAAPVQETLHGCQPVTQEDGK |
| | intracellular | ESRISVQERQ |
| 32 | OX40 TM | VAAILGLGLVLGLLGPLAILL |
| 33 | OX40 | ALYLLRRDQRLPPDAHKPPGGGSFRTPIQEEQADAHSTLAKI |
| | intracellular | |
| 34 | BAFFR TM | FGAPALLGLALVLALVLVGLV |
| 35 | BAFFR | SWRRRQRRLRGASSAEAPDGDKDAPEPLDKVIILSPGISDATAPAWPPPGED |
| | intracellular | PGTTPPGHSVPVPATELGSTELVTTKTAGPEQQ |
| 36 | CD8α hinge | TTTPAPRPPTPAPTIASQPLSLRPEACRPAAGGAVHTRGLDFACD |
| 159 | GS linker | GGGGSGGGGSGGGGSGGGGS |
| 168 | SH3 domain | PXXP |
| 169 | SH3 domain | PXXXP |
| 170 | SH3 domain | PPPP |
| 171 | SH3 domain | PPPPP |
| 172 | SH3 domain | PPAP |
| 173 | SH3 domain | PPLP |
| 174 | SH3 domain | PLPRP |
| 175 | SH3 domain | PKPP |
| 176 | GYF binding | PPPPGHR |
| | domain | |
| 177 | TPOR | SDPTRVETATETAWILVLISLVTALHLVLGLSAVLGLLLLRWQFPAHYRRLR |
| | transmembrane | HALWPSLPDLHRVLGQYLRDTAALSPPKATVSDTCEEVEPSLLEILPKSSER |
| | domain N + 4 | TPLPL |
| 180 | IL2Rb-YYY | QQDKVPEPASLSSNHSLTSCFTNQGYFFFHLPDALEIEACQDEG |
| | | VAGAPTGSSPQPLQPLSGEDDAYCTFPSRDDLLLFSPSGQGEFR |
| | | ALNARLPLNTDAYLSLQELQGQDPTHLV |

Nucleotide sequences of components of a chimeric switch receptor as disclosed herein are provided in Table 2.

TABLE 2
Exemplary Individual modules-DNA sequence

| SEQ ID | | |
|---|---|---|
| NO: | Module name | DNA Sequence |
| 37 | CD8 signal | ATGGCCCTGCCAGTGACCGCCCTGCTGCTGCCTCTGGCCCTGCTGCTGCACG |
| | sequence | CCGCTAGACCC |
| 38 | BR2 signal | ATGGGTCGGGGGCTGCTCAGGGGCCTGTGGCCGCTGCACATCGTCCTGTGGA |
| | sequence | CGCGTATCGCCAGC |
| 39 | TpoR (S505N | TCAGACCCTACTAGAGTCGAGACCGCTACCGAGACCGCTTGGATCTCTCTGG |
| | W515K) | TGACCGCCCTGCACCTGGTGCTGGGCCTGAACGCCGTGCTGGGCCTGCTGCT |
| | | GCTGAGGAAGCAGTTCCCAGCACACTACCGGAGACTGAGGCACGCACTGTGG |
| | | CCAAGCCTGCCCGACCTGCACAGGGTGCTGGGACAGTATCTGAGGGATACAG |
| | | CCGCCCTGAGCCCACCTAAGGCAACCGTGTCCGACACATGCGAGGAGGTGGA |
| | | ACCAAGTCTGCTGGAAATCCTGCCAAAATCCTCTGAGCGGACACCCCTGCCC |
| | | CTG |
| 40 | IL2Rb-YY | GACGAGGGAGTGGCAGGAGCACCAACCGGCAGCTCCCCCCAGCCTCTGCAGC |
| | | CACTGTCCGGAGAGGACGATGCATACTGCACATTCCCTTCTCGGGACGATCT |
| | | GCTGCTGTTCTCTCCAAGCGGACAGGGAGAGTTTCGGGCCCTGAACGCCAGA |
| | | CTGCCCCTGAATACCGACGCCTATCTGAGCCTGCAGGAGCTGCAGGGACAGG |
| | | ACCCCACACACCTGGTG |
| 41 | dn WT PD1 | CCCGGATGGTTTCTGGATAGCCCTGATAGGCCCTGGAACCCCCCAACTTTTT |
| | | CACCCGCCCTGCTGGTCGTCACCGAAGGAGACAACGCCACCTTCACATGCAG |
| | | CTTTTCCAACACCTCTGAGAGCTTCGTGCTGAATTGGTACAGGATGTCCCCA |
| | | TCTAACCAGACAGACAAGCTGGCAGCATTTCCTGAGGACCGCTCCCAGCCAG |
| | | GACAGGATTGCCGGTTCAGAGTGACCCAGCTGCCCAATGGCCGGGACTTTCA |
| | | CATGTCTGTGGTGAGAGCCCGGAGAAACGATAGCGGCACATACCTGTGCGGA |
| | | GCAATCTCCCTGGCACCAAAGGCACAGATCAAGGAGTCTCTGAGGGCAGAGC |
| | | TGAGGGTGACCGAGAGGAGGGCAGAGGTGCCTACAGCACACCCAAGCCCTTC |
| | | CCCACGGCCCGCAGGCCAGTTCCAGACCCTGGTGGTGGGAGTGGTGGGAGGC |
| | | CTGCTGGGCAGCCTGGTGCTGCTGGTGTGGGTGCTGGCAGTCATTTGTAGCA |
| | | GAGCCGCAAGAGGAACTATCGGAGCAAGACGGACAGGGCAG |
| 42 | dnHACiPD1 | CCTGGATGGTTTCTGGACTCCCCTGATAGGCCCTGGAATCCCCCAACTTTCT |
| | | CCCCTGCCCTGCTGGTGGTCACTGAAGGCGACAACGCCACCTTCACATGCAG |
| | | CTTTTCCAACACCTCTGAGAGCTTCCACGTGATCTGGCACAGGGAGTCCCCA |
| | | TCTGGCCAGACCGACACACTGGCAGCATTTCCTGAGGACCGCTCCCAGCCAG |
| | | GACAGGATTGCCGGTTCAGAGTGACCCAGCTGCCCAACGGCCGGGACTTTCA |
| | | CATGTCTGTGGTGAGAGCCCGGAGAAATGATAGCGGCACCTACGTGTGCGGC |
| | | GTGATCTCCCTGGCCCCCAAGATCCAGATCAAGGAGTCTCTGAGGGCAGAGC |
| | | TGAGGGTGACCGAGAGGAGGGCAGAGGTGCCTACAGCACACCCAAGCCCTTC |
| | | CCCACGGCCCGCAGGACAGTTCCAGACACTGGTGGTGGGAGTGGTGGGAGGC |
| | | CTGCTGGGCAGCCTGGTGCTGCTGGTGTGGGTGCTGGCTGTCATCTGTAGCA |
| | | GGGCCGCAAGAGGCACCATTGGGGCACGAAGGACTGGGCAG |
| 43 | dn WT BR2 | ACGATCCCACCGCACGTTCAGAAGTCGGTTAATAACGACATGATAGTCACTG |
| | | ACAACAACGGTGCAGTCAAGTTTCCACAACTGTGTAAATTTTGTGATGTGAG |
| | | ATTTTCCACCTGTGACAACCAGAAATCCTGCATGAGCAACTGCAGCATCACC |
| | | TCCATCTGTGAGAAGCCACAGGAAGTCTGTGTGGCTGTATGGAGAAAGAATG |
| | | ACGAGAACATAACACTAGAGACAGTTTGCCATGACCCCAAGCTCCCCTACCA |
| | | TGACTTTATTCTGGAAGATGCTGCTTCTCCAAAGTGCATTATGAAGGAAAAA |
| | | AAAAAGCCTGGTGAGACTTTCTTCATGTGTTCCTGTAGCTCTGATGAGTGCA |
| | | ATGACAACATCATCTTCTCAGAAGAATATAACACCAGCAATCCTGACTTGTT |
| | | GCTAGTCATATTTCAAGTGACAGGCATCAGCCTCCTGCCACCACTGGGAGTT |
| | | GCCATATCTGTCATCATCATCTTCTACTGCTACCGCGTTAACCGGCAGCAGA |
| | | AGCTGAGTTCA |
| 44 | dN25 | CAACTGTGTAAATTTTGTGATGTGAGATTTTCCACCTGTGACAACCAGAAAT |
| | BR2(DNR) | CCTGCATGAGCAACTGCAGCATCACCTCCATCTGTGAGAAGCCACAGGAAGT |
| | | CTGTGTGGCTGTATGGAGAAAGAATGACGAGAACATAACACTAGAGACAGTT |
| | | TGCCATGACCCCAAGCTCCCCTACCATGACTTTATTCTGGAAGATGCTGCTT |
| | | CTCCAAAGTGCATTATGAAGGAAAAAAAAAAGCCTGGTGAGACTTTCTTCAT |
| | | GTGTTCCTGTAGCTCTGATGAGTGCAATGACAACATCATCTTCTCAGAAGAA |
| | | TATAACACCAGCAATCCTGACTTGTTGCTAGTCATATTTCAAGTGACAGGCA |
| | | TCAGCCTCCTGCCACCACTGGGAGTTGCCATATCTGTCATCATCATCTTCTA |
| | | CTGCTACCGCGTTAACCGGCAGCAGAAGCTGAGTTCA |
| 45 | WT PD1 ECD | CCAGGCTGGTTCCTGGATAGCCCCGACAGACCTTGGAATCCCCCTACATTCA |
| | | GCCCTGCTCTGCTGGTCGTGACCGAGGGCGACAACGCCACCTTCACATGCAG |
| | | CTTCAGCAACACCAGCGAGTCTTTTGTGCTGAACTGGTATCGGATGAGCCCT |
| | | TCTAACCAGACAGATAAGCTGGCAGCCTTCCCCGAAGATAGAAGCCAACCTG |
| | | GCCAGGACTGCAGATTCAGAGTGACCCAGCTGCCTAACGGCCGGGACTTCCA |
| | | CATGTCTGTGGTGCGGGCCAGACGCAACGACAGCGGCACCTACCTGTGCGGC |
| | | GCCATCAGCCTGGCCCCTAAGGCCCAGATCAAGGAAAGCCTGAGAGCCGAGC |
| | | TGCGGGTGACAGAAAGAAGGGCCGAAGTGCCCACCGCCCACCCTTCCCCTTC |
| | | CCCCAGACCTGCCGGACAATTTCAGACCCTGGTT |
| 46 | HA PD1 ECD | CCCGGCTGGTTCCTGGATAGCCCTGACCGGCCATGGAATCCTCCTACCTTCA |
| | | GCCCCGCTCTGCTCGTGGTCACAGAGGGAGATAACGCCACATTCACCTGTAG |
| | | CTTCAGCAACACAAGCGAGTCTTTTCACGTGATTTGGCATCGGGAATCTCCT |
| | | TCCGGCCAGACCGACACCCTGGCCGCCTTCCCTGAAGATAGATCTCAACCTG |
| | | GACAGGACTGCAGATTCAGAGTGACCCAGCTGCCCAACGGCAGAGACTTCCA |
| | | CATGAGCGTGGTGCGGGCCAGACGGAACGACAGCGGCACCTACGTGTGCGGC |
| | | GTGATCAGCCTGGCTCCTAAGATCCAGATCAAGGAAAGCCTGAGAGCCGAGC |
| | | TGCGGGTGACCGAGCGGAGAGCTGAGGTGCCTACAGCCCACCCTAGCCCATC |
| | | TCCTAGACCTGCCGGCCAATTTCAGACACTGGTC |
| 47 | BR2 signal | ATGGGCAGAGGACTGCTGAGAGGCCTGTGGCCTCTGCATATCGTGCTGTGGA |
| | peptide | CCAGAATCGCCTCT |
| 48 | WT BR2 ECD | ACAATCCCCCCCCACGTGCAGAAGTCCGTGAACAATGACATGATCGTCACCG |
| | | ACAACAACGGCGCTGTGAAGTTTCCACAACTGTGCAAGTTCTGCGACGTGCG |
| | | GTTCAGCACATGCGATAACCAGAAAAGCTGTATGAGCAATTGCTCCATTACA |
| | | AGCATCTGTGAAAAACCTCAGGAGGTGTGCGTGGCCGTGTGGCGGAAGAACG |
| | | ACGAGAACATCACCCTGGAGACCGTGTGTCACGATCCTAAGCTGCCTTACCA |
| | | CGACTTCATCCTGGAAGATGCCGCCAGCCCTAAGTGCATCATGAAGGAAAAG |
| | | AAAAAGCCTGGCGAGACCTTCTTCATGTGCTCTTGTTCTAGCGACGAGTGCA |
| | | ACGATAATATCATCTTCAGCGAGGAATACAACACCAGCAACCCCGACCTGCT |
| | | GCTCGTGATCTTTCAG |
| 49 | dN25 BR2 | CAGCTGTGCAAGTTCTGCGACGTGCGGTTCAGCACCTGTGATAACCAGAAAA |
| | ECD | GCTGTATGAGCAATTGCTCTATCACCTCCATCTGCGAGAAGCCTCAGGAGGT |
| | | GTGCGTGGCCGTGTGGCGGAAGAACGACGAGAACATTACACTGGAAACCGTG |
| | | TGTCACGATCCTAAGCTGCCTTACCACGACTTCATCCTGGAAGATGCCGCCA |
| | | GCCCTAAGTGCATCATGAAGGAAAAGAAAAAGCCAGGCGAGACATTTTTCAT |
| | | GTGCTCCTGTAGCAGCGACGAGTGCAACGACAATATCATCTTTAGCGAGGAA |
| | | TACAACACCAGCAACCCCGACCTGCTCCTGGTCATCTTCCAA |
| 50 | PD1 TM | GTCGGCGTGGTGGGCGGACTGCTGGGCTCTCTGGTGCTGCTGGTGTGGGTGC |
| | | TGGCCGTGATCTGCAGCAGAGCCGCTAGAGGAACAATCGGCGCCAGACGGAC |
| | | CGGCCAG |
| 51 | MyD88 | ATGGCTGCTGGAGGACCTGGCGCTGGCAGCGCCGCTCCTGTGTCCAGCACCA |
| | | GCTCTCTGCCTCTGGCTGCACTTAATATGAGAGTGCGGCGGAGACTGAGCCT |
| | | CTTCCTGAATGTGCGCACCCAAGTGGCAGCTGATTGGACCGCCCTGGCCGAA |
| | | GAGATGGACTTCGAGTACCTGGAAATCAGACAGCTGGAAACCCAGGCCGACC |
| | | CTACAGGCAGACTGCTGGATGCCTGGCAGGGCAGACCGGGCGCCAGCGTTGG |
| | | AAGGCTGCTGGACCTCCTGACCAAGCTGGGCCGGGATGATGTGCTGCTGGAG |
| | | CTGGGTCCTAGCATCGAGGAAGATTGCCAGAAATACATCCTGAAACAGCAAC |
| | | AGGAGGAAGCCGAGAAGCCTCTGCAGGTGGCCGCCGTGGACAGCTCTGTGCC |
| | | TAGAACAGCCGAGCTGGCCGGCATCACCACCCTGGACGACCCCCTGGGCCAC |
| | | ATGCCTGAGCGGTTCGACGCCTTTATTTGTTATTGCCCCTCTGACATC |
| 52 | CD28 ECD | TGTCCTAGCCCCCTGTTCCCCGGTCCTAGCAAACCT |
| | (truncated) | |
| 53 | CD28 TM | ttctgggtgctggtggtggtgggcggcgtgctggcctgctacagcctgctgg |
| | | tcacagtggcctttatcatcttctgggtc |
| 54 | CD28 | AGATCCAAGCGGTCTAGACTGCTTCATAGCGACTACATGAACATGACACCTA |
| | intracellular | GAAGGCCTGGCCCCACAAGAAAGCACTACCAGCCCTACGCCCCTCCTAGAGA |
| | | TTTCGCCGCCTACAGAAGC |
| 55 | CD28.YMFM | agatctaagcggtccagactgctgcattctgattacatgttcatgaccccta |
| | intracellular | gaagacctggacctacaagaaagcactaccagccttacgcccctcctcggga |
| | | cttcgccgcttatagaagc |
| 56 | CD28.AYAA | AGATCTAAGCGGTCCAGACTGCTGCATTCTGATTACATGAACATGACCCCTA |
| | intracellular | GAAGACCTGGACCTACAAGAAAGCACTACCAGGCCTACGCCGCCCCTCGGGA |
| | | CTTCGCCGCTTATAGAAGC |
| 57 | CD2 ECD | GTGGAGCCTGTGTCCTGCCCTGAGAAGGGCCTGGAC |
| | (truncated) | |
| 58 | CD2 TM | ATCTACCTGATCATCGGCATCTGCGGAGGAGGCAGCCTGCTGATGGTGTTCG |
| | | TGGCCCTGCTGGTGTTCTACATCACC |
| 59 | CD2 | AAGCGGAAGAAGCAGCGGAGCAGACGGAATGACGAGGAACTCGAGACAAGAG |
| | intracellular | CCCATCGGGTCGCCACAGAGGAAAGAGGCAGAAAGCCCCACCAGATTCCTGC |
| | (full) | CAGCACACCTCAGAACCCTGCTACCAGCCAACACCCCCCCCCCCCTCCTGGC |
| | | CACAGATCTCAGGCCCCTAGCCACCGGCCCCCGCCACCTGGCCACCGGGTGC |
| | | AGCACCAGCCTCAAAAAAGACCCCCTGCTCCTAGCGGCACACAGGTGCACCA |
| | | GCAGAAAGGTCCTCCACTGCCTAGACCTCGGGTGCAGCCTAAGCCTCCACAT |
| | | GGCGCCGCTGAGAACAGCTTGTCTCCTAGTTCTAAT |
| 60 | CD2 | AAGAGAAAGAAGCAGACCCCTCAGAACCCCGCCACCAGCCAACACCCCCCCC |
| | intracellular | CTCCACCAGGCCACAGAAGCCAGGCCCCTTCCCACCGCCCCCCCCCTCCAGG |
| | (short) | ACATAGGGTTCAGCACCAGCCCCAGAAGCGGCCTCCTGCTCCTAGCGGAACA |
| | | CAGGTGCACCAGCAGAAAGGCCCTCCCCTCCCTAGACCCAGAGTGCAGCCTA |
| | | AACCTCCCCACGGCGCCGCCGAGAACAGCCTGTCCCCTTCTAGCAAT |
| 61 | DAP10 ECD | CAGACCACACCTGGAGAACGGAGCAGCCTCCCCGCTTTCTACCCCGGCACCA |
| | | GCGGCTCCTGCAGCGGATGTGGCAGCCTGAGCCTGCCT |
| 62 | DAP10 TM | CTGCTGGCCGGCCTGGTGGCCGCCAACGCCGTCGCCTCTCTGCTGATCGTGG |
| | D57N | GCGCCGTGTTC |
| 63 | DAP10 | CTGCTGGCCGGCCTGGTGGCCGCCAACGCCGTCGCCTCTCTGCTGATCGTGG |
| | intracellular | GCGCCGTGTTC |
| 64 | ICOS TM | TTCTGGCTGCCTATCGGCTGCGCCGCTTTTGTGGTGGTCTGCATCCTGGGAT |
| | | GTATCCTGATC |
| 65 | ICOS | TGCTGGCTGACCAAGAAGAAGTACAGCTCCAGCGTGCACGACCCCAACGGCG |
| | intracellular | AGTACATGTTCATGCGGGCCGTGAACACCGCCAAGAAATCTAGACTGACAGA |
| | | TGTGACCCTG |
| 66 | CD40 TM | GCCCTGGTGGTGATCCCCATCATCTTCGGCATCCTGTTCGCCATTCTGCTGG |
| | | TGCTGGTCTTTATC |
| 67 | CD40 | AAGAAGGTGGCCAAGAAACCTACAAACAAGGCCCCTCACCCCAAGCAGGAGC |
| | intracellular | CTCAGGAGATCAACTTCCCCGACGACCTGCCTGGAAGCAATACCGCCGCTCC |
| | | AGTGCAAGAAACCCTGCACGGCTGCCAGCCTGTGACCCAGGAAGATGGCAAA |
| | | GAGTCTAGAATCAGCGTGCAGGAGCGGCAG |
| 68 | OX40 TM | GTGGCCGCCATCCTGGGCCTGGGCCTGGTGCTGGGACTGCTGGGCCCTCTGG |
| | | CTATCCTGCTG |
| 69 | OX40 | GCCCTGTACCTGCTCAGACGGGACCAGAGACTGCCCCCCGACGCCCACAAGC |
| | intracellular | CTCCAGGCGGCGGATCTTTCAGAACCCCTATCCAGGAGGAACAGGCCGATGC |
| | | TCACAGCACACTGGCCAAGATC |
| 70 | BAFFR TM | TTCGGCGCTCCCGCTCTGCTGGGCCTCGCCCTGGTGCTGGCCCTGGTCCTGG |
| | | TGGGCCTGGTG |
| 71 | BAFFR | TCCTGGCGGCGGAGACAGAGAAGACTGAGAGGCGCCAGCAGCGCCGAGGCCC |
| | intracellular | CTGATGGCGATAAGGACGCCCCTGAGCCTCTGGACAAAGTGATCATCCTGAG |
| | | CCCCGGCATCAGCGACGCTACCGCCCCTGCCTGGCCTCCACCAGGCGAGGAC |
| | | CCCGGAACAACCCCTCCTGGCCACAGCGTGCCTGTGCCCGCCACCGAGCTGG |
| | | GATCTACAGAACTGGTGACCACAAAGACCGCCGGCCCTGAACAGCAG |
| 72 | CD8α hinge | ACAACCACACCTGCACCTAGGCCACCTACACCTGCACCAACCATCGCCAGCC |
| | | AGCCTCTGTCCCTGAGACCAGAGGCCTGTAGGCCAGCAGCAGGAGGAGCAGT |
| | | GCACACCCGGGGCCTGGACTTCGCCTGCGAT |
| 164 | (GGGGS)₄ | GGCGGCGGCGGCTCTGGAGGAGGAGGCAGCGGCGGAGGAGGCTCCGGAGGCG |
| | linker | GCGGCTCT |

Amino acid sequences of full-length chimeric switch receptors as disclosed herein are provided in Table 3.

TABLE 3
Exemplary full-length constructs-amino acid sequence

| SEQ ID | Construct | Polypeptide | |
|---|---|---|---|
| NO: | name | structure | Amino acid sequence |
| 73 | 2G1 | CD8 signal | MALPVTALLLPLALLLHAARPGGGGSCPYSNPSLCGGGG |
| | (DLL3 | sequence | SQLQLQESGPGLVKPSETLSLTCTVSGGSISSSSYYWGW |
| | CAR) | (underlined), | IRQPPGKGLEWIGSIYYSGNIYHNPSLKSRVSISVDTSK |
| | | rituximab | NQFSLRLSSVTAADTAVYYCAREIIVGATHFDYWGQGTL |
| | | mimotope RSR, | VTVSSGGGGSGGGGSGGGGSGGGGSAIQMTQSPSSLSAS |
| | | 2G1 scFv, | VGDRVTITCRASQGIRNDLGWYQQKPGKAPELLIYAASS |
| | | rituximab | LQSGVPSRFSGSGSGTDFTLTISSLQPEDFATYYCLQDY |
| | | mimotope, CD8α | NYPLTFGPGTKVDIKGGGGSCPYSNPSLCGGGGSTTTPA |
| | | hinge, CD8α | PRPPTPAPTIASQPLSLRPEACRPAAGGAVHTRGLDFAC |
| | | transmembrane, | DIYIWAPLAGTCGVLLLSLVITLYCKRGRKKLLYIFKQP |
| | | CD8α cytoplasmic | FMRPVQTTQEEDGCSCRFPEEEEGGCELRVKFSRSADAP |
| | | domain (truncated), | AYQQGQNQLYNELNLGRREEYDVLDKRRGRDPEMGGKPR |
| | | 4-1BB (TNFRSF9, | RKNPQEGLYNELQKDKMAEAYSEIGMKGERRRGKGHDGL |
| | | CD137) | YQGLSTATKDTYDALHMQALPPR |
| | | cytoplasmic | |
| | | domain, CD3ζ | |
| | | cytoplasmic | |
| | | domain | |
| 165 | 2G1 | rituximab | GGGGSCPYSNPSLCGGGGSQLQLQESGPGLVKPSETLSL |
| | (DLL3 | mimotope RSR, | TCTVSGGSISSSSYYWGWIRQPPGKGLEWIGSIYYSGNI |
| | CAR) | 2G1 scFv, | YHNPSLKSRVSISVDTSKNQFSLRLSSVTAADTAVYYCA |
| | | rituximab | REIIVGATHFDYWGQGTLVTVSSGGGGSGGGGSGGGGSG |
| | | mimotope, CD8α | GGGSAIQMTQSPSSLSASVGDRVTITCRASQGIRNDLGW |
| | | hinge, CD8α | YQQKPGKAPELLIYAASSLQSGVPSRFSGSGSGTDFTLT |
| | | transmembrane, | ISSLOPEDFATYYCLQDYNYPLTFGPGTKVDIKGGGGSC |
| | | CD8α cytoplasmic | PYSNPSLCGGGGSTTTPAPRPPTPAPTIASQPLSLRPEA |
| | | domain (truncated), | CRPAAGGAVHTRGLDFACDIYIWAPLAGTCGVLLLSLVI |
| | | 4-1BB (TNFRSF9, | TLYCKRGRKKLLYIFKQPFMRPVQTTQEEDGCSCRFPEE |
| | | CD137) | EEGGCELRVKFSRSADAPAYQQGQNQLYNELNLGRREEY |
| | | cytoplasmic | DVLDKRRGRDPEMGGKPRRKNPQEGLYNELQKDKMAEAY |
| | | domain, CD3ζ | SEIGMKGERRRGKGHDGLYQGLSTATKDTYDALHMQALP |
| | | cytoplasmic | PR |
| | | domain | |
| 74 | 2G1.15.1 | CD8 signal | MALPVTALLLPLALLLHAARPSDPTRVETATETAWISLV |
| | (CCR- | sequence | TALHLVLGLNAVLGLLLLRKQFPAHYRRLRHALWPSLPD |
| | P2A- | (underlined), TpoR | LHRVLGQYLRDTAALSPPKATVSDTCEEVEPSLLEILPK |
| | DLL3 | (S505N W515K), | SSERTPLPLLEDEGVAGAPTGSSPQPLQPLSGEDDAYCT |
| | CAR) | IL2Rb-YY, P2A, | FPSRDDLLLFSPSGQGEFRALNARLPLNTDAYLSLQELQ |
| | | CD8 signal | GQDPTHLVGSGATNFSLLKQAGDVEENPGPMALPVTALL |
| | | sequence, | LPLALLLHAARPGGGGSCPYSNPSLCGGGGSQLQLQESG |
| | | rituximab | PGLVKPSETLSLTCTVSGGSISSSSYYWGWIRQPPGKGL |
| | | mimotope, 2G1 | EWIGSIYYSGNIYHNPSLKSRVSISVDTSKNQFSLRLSS |
| | | scFv, rituximab | VTAADTAVYYCAREIIVGATHFDYWGQGTLVTVSSGGGG |
| | | mimotope, CD8α | SGGGGSGGGGSGGGGSAIQMTQSPSSLSASVGDRVTITC |
| | | hinge, CD8α | RASQGIRNDLGWYQQKPGKAPELLIYAASSLQSGVPSRF |
| | | transmembrane, | SGSGSGTDFTLTISSLQPEDFATYYCLQDYNYPLTFGPG |
| | | CD8α cytoplasmic | TKVDIKGGGGSCPYSNPSLCGGGGSTTTPAPRPPTPAPT |
| | | domain (truncated), | IASQPLSLRPEACRPAAGGAVHTRGLDFACDIYIWAPLA |
| | | 4-1BB (TNFRSF9, | GTCGVLLLSLVITLYCKRGRKKLLYIFKQPFMRPVQTTQ |
| | | CD137) | EEDGCSCRFPEEEEGGCELRVKFSRSADAPAYQQGQNQL |
| | | cytoplasmic | YNELNLGRREEYDVLDKRRGRDPEMGGKPRRKNPQEGLY |
| | | domain, CD3ζ | NELQKDKMAEAYSEIGMKGERRRGKGHDGLYQGLSTATK |
| | | cytoplasmic | DTYDALHMQALPPR |
| | | domain | |
| 75 | WT | WT PD1 ECD, | PGWFLDSPDRPWNPPTFSPALLVVTEGDNATFTCSFSNT |
| | PD1.CD28 | CD28 ECD | SESFVLNWYRMSPSNQTDKLAAFPEDRSQPGQDCRFRVT |
| | | (truncated), CD28 | QLPNGRDFHMSVVRARRNDSGTYLCGAISLAPKAQIKES |
| | | TM, CD28 | LRAELRVTERRAEVPTAHPSPSPRPAGQFQTLVCPSPLF |
| | | intracellular | PGPSKPFWVLVVVGGVLACYSLLVTVAFIIFWVRSKRSR |
| | | | LLHSDYMNMTPRRPGPTRKHYQPYAPPRDFAAYRS |
| 76 | WT | WT PD1 ECD, | PGWFLDSPDRPWNPPTFSPALLVVTEGDNATFTCSFSNT |
| | PD1.CD28. | CD28 ECD | SESFVLNWYRMSPSNQTDKLAAFPEDRSQPGQDCRFRVT |
| | YMFM | (truncated), CD28 | QLPNGRDFHMSVVRARRNDSGTYLCGAISLAPKAQIKES |
| | | TM, CD28.YMFM | LRAELRVTERRAEVPTAHPSPSPRPAGQFQTLVCPSPLF |
| | | intracellular | PGPSKPFWVLVVVGGVLACYSLLVTVAFIIFWVRSKRSR |
| | | | LLHSDYMFMTPRRPGPTRKHYQPYAPPRDFAAYRS |
| 77 | WT | WT PD1 ECD, | PGWFLDSPDRPWNPPTFSPALLVVTEGDNATFTCSFSNT |
| | PD1.CD28. | CD28 ECD | SESFVLNWYRMSPSNQTDKLAAFPEDRSQPGQDCRFRVT |
| | AYAA | (truncated), CD28 | QLPNGRDFHMSVVRARRNDSGTYLCGAISLAPKAQIKES |
| | | TM, CD28.AYAA | LRAELRVTERRAEVPTAHPSPSPRPAGQFQTLVCPSPLF |
| | | intracellular | PGPSKPFWVLVVVGGVLACYSLLVTVAFIIFWVRSKRSR |
| | | | LLHSDYMNMTPRRPGPTRKHYQAYAAPRDFAAYRS |
| 78 | WT | WT PD1 ECD, | PGWFLDSPDRPWNPPTFSPALLVVTEGDNATFTCSFSNT |
| | PD1.CD2 | CD2 ECD | SESFVLNWYRMSPSNQTDKLAAFPEDRSQPGQDCRFRVT |
| | (full) | (truncated), CD2 | QLPNGRDFHMSVVRARRNDSGTYLCGAISLAPKAQIKES |
| | | TM, CD2 | LRAELRVTERRAEVPTAHPSPSPRPAGQFQTLVVEPVSC |
| | | intracellular | PEKGLDIYLIIGICGGGSLLMVFVALLVFYITKRKKQRS |
| | | | RRNDEELETRAHRVATEERGRKPHQIPASTPQNPATSQH |
| | | | PPPPPGHRSQAPSHRPPPPGHRVQHQPQKRPPAPSGTQV |
| | | | HQQKGPPLPRPRVQPKPPHGAAENSLSPSSN |
| 79 | WT | WT PD1 ECD, | PGWFLDSPDRPWNPPTFSPALLVVTEGDNATFTCSFSNT |
| | PD1.CD2 | CD2 ECD | SESFVLNWYRMSPSNQTDKLAAFPEDRSQPGQDCRFRVT |
| | (short) | (truncated), CD2 | QLPNGRDFHMSVVRARRNDSGTYLCGAISLAPKAQIKES |
| | | TM, CD2 | LRAELRVTERRAEVPTAHPSPSPRPAGQFQTLVVEPVSC |
| | | intracellular | PEKGLDIYLIIGICGGGSLLMVFVALLVFYITKRKKQTP |
| | | (short) | QNPATSQHPPPPPGHRSQAPSHRPPPPGHRVQHQPQKRP |
| | | | PAPSGTQVHQQKGPPLPRPRVQPKPPHGAAENSLSPSSN |
| 80 | WT | WT PD1 ECD, | PGWFLDSPDRPWNPPTFSPALLVVTEGDNATFTCSFSNT |
| | PD1.DAP10. | DAP10 ECD, | SESFVLNWYRMSPSNQTDKLAAFPEDRSQPGQDCRFRVT |
| | D57N | DAP10 TM D57N, | QLPNGRDFHMSVVRARRNDSGTYLCGAISLAPKAQIKES |
| | | DAP10 | LRAELRVTERRAEVPTAHPSPSPRPAGQFQTLVQTTPGE |
| | | intracellular | RSSLPAFYPGTSGSCSGCGSLSLPLLAGLVAANAVASLL |
| | | | IVGAVFLCARPRRSPAQEDGKVYINMPGRG |
| 81 | WT | WT PD1 ECD, | PGWFLDSPDRPWNPPTFSPALLVVTEGDNATFTCSFSNT |
| | PD1.ICOS | ICOS TM, ICOS | SESFVLNWYRMSPSNQTDKLAAFPEDRSQPGQDCRFRVT |
| | | intracellular | QLPNGRDFHMSVVRARRNDSGTYLCGAISLAPKAQIKES |
| | | | LRAELRVTERRAEVPTAHPSPSPRPAGQFQTLVFWLPIG |
| | | | CAAFVVVCILGCILICWLTKKKYSSSVHDPNGEYMFMRA |
| | | | VNTAKKSRLTDVTL |
| 82 | WT | WT PD1 ECD, | PGWFLDSPDRPWNPPTFSPALLVVTEGDNATFTCSFSNT |
| | PD1.CD40 | CD40 TM, CD40 | SESFVLNWYRMSPSNQTDKLAAFPEDRSQPGQDCRFRVT |
| | | intracellular | QLPNGRDFHMSVVRARRNDSGTYLCGAISLAPKAQIKES |
| | | | LRAELRVTERRAEVPTAHPSPSPRPAGQFQTLVALVVIP |
| | | | IIFGILFAILLVLVFIKKVAKKPTNKAPHPKQEPQEINF |
| | | | PDDLPGSNTAAPVQETLHGCQPVTQEDGKESRISVQERQ |
| 83 | WT | WT PD1 ECD, | PGWFLDSPDRPWNPPTFSPALLVVTEGDNATFTCSFSNT |
| | PD1.OX40 | OX40 TM, OX40 | SESFVLNWYRMSPSNQTDKLAAFPEDRSQPGQDCRFRVT |
| | | intracellular | QLPNGRDFHMSVVRARRNDSGTYLCGAISLAPKAQIKES |
| | | | LRAELRVTERRAEVPTAHPSPSPRPAGQFQTLVVAAILG |
| | | | LGLVLGLLGPLAILLALYLLRRDQRLPPDAHKPPGGGSF |
| | | | RTPIQEEQADAHSTLAKI |
| 84 | HA | HA PD1 ECD, | PGWFLDSPDRPWNPPTFSPALLVVTEGDNATFTCSFSNT |
| | PD1.CD28 | CD28 ECD | SESFHVIWHRESPSGQTDTLAAFPEDRSQPGQDCRFRVT |
| | | (truncated), CD28 | QLPNGRDFHMSVVRARRNDSGTYVCGVISLAPKIQIKES |
| | | TM, CD28 | LRAELRVTERRAEVPTAHPSPSPRPAGQFQTLVCPSPLF |
| | | intracellular | PGPSKPFWVLVVVGGVLACYSLLVTVAFIIFWVRSKRSR |
| | | | LLHSDYMNMTPRRPGPTRKHYQPYAPPRDFAAYRS |
| 85 | HA | HA PD1 ECD, | PGWFLDSPDRPWNPPTFSPALLVVTEGDNATFTCSFSNT |
| | PD1.CD28. | CD28 ECD | SESFHVIWHRESPSGQTDTLAAFPEDRSQPGQDCRFRVT |
| | YMFM | (truncated), CD28 | QLPNGRDFHMSVVRARRNDSGTYVCGVISLAPKIQIKES |
| | | TM, CD28.YMFM | LRAELRVTERRAEVPTAHPSPSPRPAGQFQTLVCPSPLF |
| | | intracellular | PGPSKPFWVLVVVGGVLACYSLLVTVAFIIFWVRSKRSR |
| | | | LLHSDYMFMTPRRPGPTRKHYQPYAPPRDFAAYRS |
| 86 | HA | HA PD1 ECD, | PGWFLDSPDRPWNPPTFSPALLVVTEGDNATFTCSFSNT |
| | PD1.CD28. | CD28 ECD | SESFHVIWHRESPSGQTDTLAAFPEDRSQPGQDCRFRVT |
| | AYAA | (truncated), CD28 | QLPNGRDFHMSVVRARRNDSGTYVCGVISLAPKIQIKES |
| | | TM, CD28.AYAA | LRAELRVTERRAEVPTAHPSPSPRPAGQFQTLVCPSPLF |
| | | intracellular | PGPSKPFWVLVVVGGVLACYSLLVTVAFIIFWVRSKRSR |
| | | | LLHSDYMNMTPRRPGPTRKHYQAYAAPRDFAAYRS |
| 87 | HA | HA PD1 ECD, | PGWFLDSPDRPWNPPTFSPALLVVTEGDNATFTCSFSNT |
| | PD1.CD2 | CD2 ECD | SESFHVIWHRESPSGQTDTLAAFPEDRSQPGQDCRFRVT |
| | (full) | (truncated), CD2 | QLPNGRDFHMSVVRARRNDSGTYVCGVISLAPKIQIKES |
| | | TM, CD2 | LRAELRVTERRAEVPTAHPSPSPRPAGQFQTLVVEPVSC |
| | | intracellular | PEKGLDIYLIIGICGGGSLLMVFVALLVFYITKRKKQRS |
| | | | RRNDEELETRAHRVATEERGRKPHQIPASTPQNPATSQH |
| | | | PPPPPGHRSQAPSHRPPPPGHRVQHQPQKRPPAPSGTQV |
| | | | HQQKGPPLPRPRVQPKPPHGAAENSLSPSSN |
| 88 | HA | HA PD1 ECD, | PGWFLDSPDRPWNPPTFSPALLVVTEGDNATFTCSFSNT |
| | PD1.CD2 | CD2 ECD | SESFHVIWHRESPSGQTDTLAAFPEDRSQPGQDCRFRVT |
| | (short) | (truncated), CD2 | QLPNGRDFHMSVVRARRNDSGTYVCGVISLAPKIQIKES |
| | | TM, CD2 | LRAELRVTERRAEVPTAHPSPSPRPAGQFQTLV |
| | | intracellular | VEPVSCPEKGLDIYLIIGICGGGSLLMVFVALLVFYITK |
| | | (short) | RKKQTPQNPATSQHPPPPPGHRSQAPSHRPPPPGHRVQH |
| | | | QPQKRPPAPSGTQVHQQKGPPLPRPRVQPKPPHGAAENS |
| | | | LSPSSN |
| 89 | HA | HA PD1 ECD, | PGWFLDSPDRPWNPPTFSPALLVVTEGDNATFTCSFSNT |
| | PD1.DAP10. | DAP10 ECD, | SESFHVIWHRESPSGQTDTLAAFPEDRSQPGQDCRFRVT |
| | D57N | DAP10 TM D57N, | QLPNGRDFHMSVVRARRNDSGTYVCGVISLAPKIQIKES |
| | | DAP10 | LRAELRVTERRAEVPTAHPSPSPRPAGQFQTLVQTTPGE |
| | | intracellular | RSSLPAFYPGTSGSCSGCGSLSLPLLAGLVAANAVASLL |
| | | | IVGAVFLCARPRRSPAQEDGKVYINMPGRG |
| 90 | HA | HA PD1 ECD, | PGWFLDSPDRPWNPPTFSPALLVVTEGDNATFTCSFSNT |
| | PD1.ICOS | ICOS TM, ICOS | SESFHVIWHRESPSGQTDTLAAFPEDRSQPGQDCRFRVT |
| | | intracellular | QLPNGRDFHMSVVRARRNDSGTYVCGVISLAPKIQIKES |
| | | | LRAELRVTERRAEVPTAHPSPSPRPAGQFQTLVFWLPIG |
| | | | CAAFVVVCILGCILICWLTKKKYSSSVHDPNGEYMFMRA |
| | | | VNTAKKSRLTDVTL |
| 91 | HA | HA PD1 ECD, | PGWFLDSPDRPWNPPTFSPALLVVTEGDNATFTCSFSNT |
| | PD1.CD40 | CD40 TM, CD40 | SESFHVIWHRESPSGQTDTLAAFPEDRSQPGQDCRFRVT |
| | | intracellular | QLPNGRDFHMSVVRARRNDSGTYVCGVISLAPKIQIKES |
| | | | LRAELRVTERRAEVPTAHPSPSPRPAGQFQTLVALVVIP |
| | | | IIFGILFAILLVLVFIKKVAKKPTNKAPHPKQEPQEINF |
| | | | PDDLPGSNTAAPVQETLHGCQPVTQEDGKESRISVQERQ |
| 92 | HA | HA PD1 ECD, | PGWFLDSPDRPWNPPTFSPALLVVTEGDNATFTCSFSNT |
| | PD1.OX40 | OX40 TM, OX40 | SESFHVIWHRESPSGQTDTLAAFPEDRSQPGQDCRFRVT |
| | | intracellular | QLPNGRDFHMSVVRARRNDSGTYVCGVISLAPKIQIKES |
| | | | LRAELRVTERRAEVPTAHPSPSPRPAGQFQTLVVAAILG |
| | | | LGLVLGLLGPLAILLALYLLRRDQRLPPDAHKPPGGGSF |
| | | | RTPIQEEQADAHSTLAKI |
| 93 | HA | HA PD1 ECD, | PGWFLDSPDRPWNPPTFSPALLVVTEGDNATFTCSFSNT |
| | PD1.BAFFR | BAFFR TM, | SESFHVIWHRESPSGQTDTLAAFPEDRSQPGQDCRFRVT |
| | | BAFFR | QLPNGRDFHMSVVRARRNDSGTYVCGVISLAPKIQIKES |
| | | intracellular | LRAELRVTERRAEVPTAHPSPSPRPAGQFQTLVFGAPAL |
| | | | LGLALVLALVLVGLVSWRRRQRRLRGASSAEAPDGDKDA |
| | | | PEPLDKVIILSPGISDATAPAWPPPGEDPGTTPPGHSVP |
| | | | VPATELGSTELVTTKTAGPEQQ |
| 94 | HA | HA PD1 ECD, PD1 | PGWFLDSPDRPWNPPTFSPALLVVTEGDNATFTCSFSNT |
| | PD1.tm. | TM, MyD88 | SESFHVIWHRESPSGQTDTLAAFPEDRSQPGQDCRFRVT |
| | MyD88 | | QLPNGRDFHMSVVRARRNDSGTYVCGVISLAPKIQIKES |
| | | | LRAELRVTERRAEVPTAHPSPSPRPAGQFQTLVVGVVGG |
| | | | LLGSLVLLVWVLAVICSRAARGTIGARRTGQMAAGGPGA |
| | | | GSAAPVSSTSSLPLAALNMRVRRRLSLFLNVRTQVAADW |
| | | | TALAEEMDFEYLEIRQLETQADPTGRLLDAWQGRPGASV |
| | | | GRLLDLLTKLGRDDVLLELGPSIEEDCQKYILKQQQEEA |
| | | | EKPLQVAAVDSSVPRTAELAGITTLDDPLGHMPERFDAF |
| | | | ICYCPSDI |
| 95 | WT | WT BR2 ECD, | TIPPHVQKSVNNDMIVTDNNGAVKFPQLCKFCDVRFSTC |
| | BR2.CD28 | CD28 ECD | DNQKSCMSNCSITSICEKPQEVCVAVWRKNDENITLETV |
| | | (truncated), CD28 | CHDPKLPYHDFILEDAASPKCIMKEKKKPGETFFMCSCS |
| | | TM, CD28 | SDECNDNIIFSEEYNTSNPDLLLVIFQCPSPLFPGPSKP |
| | | intracellular | FWVLVVVGGVLACYSLLVTVAFIIFWVRSKRSRLLHSDY |
| | | | MNMTPRRPGPTRKHYQPYAPPRDFAAYRS |
| 96 | WT | WT BR2 ECD, | TIPPHVQKSVNNDMIVTDNNGAVKFPQLCKFCDVRFSTC |
| | BR2.CD28. | CD28 ECD | DNQKSCMSNCSITSICEKPQEVCVAVWRKNDENITLETV |
| | YMFM | (truncated), CD28 | CHDPKLPYHDFILEDAASPKCIMKEKKKPGETFFMCSCS |
| | | TM, CD28.YMFM | SDECNDNIIFSEEYNTSNPDLLLVIFQCPSPLFPGPSKP |
| | | intracellular | FWVLVVVGGVLACYSLLVTVAFIIFWVRSKRSRLLHSDY |
| | | | MFMTPRRPGPTRKHYQPYAPPRDFAAYRS |
| 97 | WT | WT BR2 ECD, | TIPPHVQKSVNNDMIVTDNNGAVKFPQLCKFCDVRFSTC |
| | BR2.CD28. | CD28 ECD | DNQKSCMSNCSITSICEKPQEVCVAVWRKNDENITLETV |
| | AYAA | (truncated), CD28 | CHDPKLPYHDFILEDAASPKCIMKEKKKPGETFFMCSCS |
| | | TM, CD28.AYAA | SDECNDNIIFSEEYNTSNPDLLLVIFQCPSPLFPGPSKP |
| | | intracellular | FWVLVVVGGVLACYSLLVTVAFIIFWVRSKRSRLLHSDY |
| | | | MNMTPRRPGPTRKHYQAYAAPRDFAAYRS |
| 98 | WT | WT BR2 ECD, | TIPPHVQKSVNNDMIVTDNNGAVKFPQLCKFCDVRFSTC |
| | BR2.CD2 | CD2 ECD | DNQKSCMSNCSITSICEKPQEVCVAVWRKNDENITLETV |
| | f(ull) | (truncated), CD2 | CHDPKLPYHDFILEDAASPKCIMKEKKKPGETFFMCSCS |
| | | TM, CD2 | SDECNDNIIFSEEYNTSNPDLLLVIFQVEPVSCPEKGLD |
| | | intracellular | IYLIIGICGGGSLLMVFVALLVFYITKRKKQRSRRNDEE |
| | | | LETRAHRVATEERGRKPHQIPASTPQNPATSQHPPPPPG |
| | | | HRSQAPSHRPPPPGHRVQHQPQKRPPAPSGTQVHQQKGP |
| | | | PLPRPRVQPKPPHGAAENSLSPSSN |
| 99 | WT | WT BR2 ECD, | TIPPHVQKSVNNDMIVTDNNGAVKFPQLCKFCDVRFSTC |
| | BR2.CD2 | CD2 ECD | DNQKSCMSNCSITSICEKPQEVCVAVWRKNDENITLETV |
| | (short) | (truncated), CD2 | CHDPKLPYHDFILEDAASPKCIMKEKKKPGETFFMCSCS |
| | | TM, CD2 | SDECNDNIIFSEEYNTSNPDLLLVIFQVEPVSCPEKGLD |
| | | intracellular | IYLIIGICGGGSLLMVFVALLVFYITKRKKQTPQNPATS |
| | | (short) | QHPPPPPGHRSQAPSHRPPPPGHRVQHQPQKRPPAPSGT |
| | | | QVHQQKGPPLPRPRVQPKPPHGAAENSLSPSSN |
| 100 | WT | WT BR2 ECD, | TIPPHVQKSVNNDMIVTDNNGAVKFPQLCKFCDVRFSTC |
| | BR2.DAP10. | DAP10 ECD, | DNQKSCMSNCSITSICEKPQEVCVAVWRKNDENITLETV |
| | D57N | DAP10 TM D57N, | CHDPKLPYHDFILEDAASPKCIMKEKKKPGETFFMCSCS |
| | | DAP10 | SDECNDNIIFSEEYNTSNPDLLLVIFQQTTPGERSSLPA |
| | | intracellular | FYPGTSGSCSGCGSLSLPLLAGLVAANAVASLLIVGAVF |
| | | | LCARPRRSPAQEDGKVYINMPGRG |
| 101 | WT | WT BR2 ECD, | TIPPHVQKSVNNDMIVTDNNGAVKFPQLCKFCDVRFSTC |
| | BR2.ICOS | ICOS TM, ICOS | DNQKSCMSNCSITSICEKPQEVCVAVWRKNDENITLETV |
| | | intracellular | CHDPKLPYHDFILEDAASPKCIMKEKKKPGETFFMCSCS |
| | | | SDECNDNIIFSEEYNTSNPDLLLVIFQFWLPIGCAAFVV |
| | | | VCILGCILICWLTKKKYSSSVHDPNGEYMFMRAVNTAKK |
| | | | SRLTDVTL |
| 102 | WT | WT BR2 ECD, | TIPPHVQKSVNNDMIVTDNNGAVKFPQLCKFCDVRFSTC |
| | BR2.CD40 | CD40 TM, CD40 | DNQKSCMSNCSITSICEKPQEVCVAVWRKNDENITLETV |
| | | intracellular | CHDPKLPYHDFILEDAASPKCIMKEKKKPGETFFMCSCS |
| | | | SDECNDNIIFSEEYNTSNPDLLLVIFQALVVIPIIFGIL |
| | | | FAILLVLVFIKKVAKKPTNKAPHPKQEPQEINFPDDLPG |
| | | | SNTAAPVQETLHGCQPVTQEDGKESRISVQERQ |
| 103 | WT | WT BR2 ECD, | TIPPHVQKSVNNDMIVTDNNGAVKFPQLCKFCDVRFSTC |
| | BR2.0X40 | OX40 TM, OX40 | DNQKSCMSNCSITSICEKPQEVCVAVWRKNDENITLETV |
| | | intracellular | CHDPKLPYHDFILEDAASPKCIMKEKKKPGETFFMCSCS |
| | | | SDECNDNIIFSEEYNTSNPDLLLVIFQVAAILGLGLVLG |
| | | | LLGPLAILLALYLLRRDQRLPPDAHKPPGGGSFRTPIQE |
| | | | EQADAHSTLAKI |
| 104 | WT | WT BR2 ECD, | TIPPHVQKSVNNDMIVTDNNGAVKFPQLCKFCDVRFSTC |
| | BR2.PD1tm. | PD1 TM, MyD88 | DNQKSCMSNCSITSICEKPQEVCVAVWRKNDENITLETV |
| | MyD88 | | CHDPKLPYHDFILEDAASPKCIMKEKKKPGETFFMCSCS |
| | | | SDECNDNIIFSEEYNTSNPDLLLVIFQVGVVGGLLGSLV |
| | | | LLVWVLAVICSRAARGTIGARRTGQMAAGGPGAGSAAPV |
| | | | SSTSSLPLAALNMRVRRRLSLFLNVRTQVAADWTALAEE |
| | | | MDFEYLEIRQLETQADPTGRLLDAWQGRPGASVGRLLDL |
| | | | LTKLGRDDVLLELGPSIEEDCQKYILKQQQEEAEKPLQV |
| | | | AAVDSSVPRTAELAGITTLDDPLGHMPERFDAFICYCPS |
| | | | DI |
| 105 | dN25 | dN BR2 ECD, | QLCKFCDVRFSTCDNQKSCMSNCSITSICEKPQEVCVAV |
| | BR2.CD28 | CD28 ECD | WRKNDENITLETVCHDPKLPYHDFILEDAASPKCIMKEK |
| | | (truncated), CD28 | KKPGETFFMCSCSSDECNDNIIFSEEYNTSNPDLLLVIF |
| | | TM, CD28 | QCPSPLFPGPSKPFWVLVVVGGVLACYSLLVTVAFIIFW |
| | | intracellular | VRSKRSRLLHSDYMNMTPRRPGPTRKHYQPYAPPRDFAA |
| | | | YRS |
| 106 | dN25 | dN25 BR2 ECD, | QLCKFCDVRFSTCDNQKSCMSNCSITSICEKPQEVCVAV |
| | BR2.CD28. | CD28 ECD | WRKNDENITLETVCHDPKLPYHDFILEDAASPKCIMKEK |
| | YMFM | (truncated), CD28 | KKPGETFFMCSCSSDECNDNIIFSEEYNTSNPDLLLVIF |
| | | TM, CD28.YMFM | QCPSPLFPGPSKPFWVLVVVGGVLACYSLLVTVAFIIFW |
| | | intracellular | VRSKRSRLLHSDYMFMTPRRPGPTRKHYQPYAPPRDFAA |
| | | | YRS |
| 107 | dN25 | dN25 BR2 ECD, | QLCKFCDVRFSTCDNQKSCMSNCSITSICEKPQEVCVAV |
| | BR2.CD28. | CD28 ECD | WRKNDENITLETVCHDPKLPYHDFILEDAASPKCIMKEK |
| | AYAA | (truncated), CD28 | KKPGETFFMCSCSSDECNDNIIFSEEYNTSNPDLLLVIF |
| | | TM, CD28.AYAA | QCPSPLFPGPSKPFWVLVVVGGVLACYSLLVTVAFIIFW |
| | | intracellular | VRSKRSRLLHSDYMNMTPRRPGPTRKHYQAYAAPRDFAA |
| | | | YRS |
| 108 | dN25 | dN25 BR2 ECD, | QLCKFCDVRFSTCDNQKSCMSNCSITSICEKPQEVCVAV |
| | BR2.CD2 | CD2 ECD | WRKNDENITLETVCHDPKLPYHDFILEDAASPKCIMKEK |
| | (full) | (truncated), CD2 | KKPGETFFMCSCSSDECNDNIIFSEEYNTSNPDLLLVIF |
| | | TM, CD2 | QVEPVSCPEKGLDIYLIIGICGGGSLLMVFVALLVFYIT |
| | | intracellular | KRKKQRSRRNDEELETRAHRVATEERGRKPHQIPASTPQ |
| | | | NPATSQHPPPPPGHRSQAPSHRPPPPGHRVQHQPQKRPP |
| | | | APSGTQVHQQKGPPLPRPRVQPKPPHGAAENSLSPSSN |
| 109 | dN25 | dN25 BR2 ECD, | QLCKFCDVRFSTCDNQKSCMSNCSITSICEKPQEVCVAV |
| | BR2.CD2 | CD2 ECD | WRKNDENITLETVCHDPKLPYHDFILEDAASPKCIMKEK |
| | (short) | (truncated), CD2 | KKPGETFFMCSCSSDECNDNIIFSEEYNTSNPDLLLVIF |
| | | TM, CD2 | QVEPVSCPEKGLDIYLIIGICGGGSLLMVFVALLVFYIT |
| | | intracellular | KRKKQTPQNPATSQHPPPPPGHRSQAPSHRPPPPGHRVQ |
| | | (short) | HQPQKRPPAPSGTQVHQQKGPPLPRPRVQPKPPHGAAEN |
| | | | SLSPSSN |
| 110 | dN25 | dN25 BR2 ECD, | QLCKFCDVRFSTCDNQKSCMSNCSITSICEKPQEVCVAV |
| | BR2.DAP10. | DAP10 ECD, | WRKNDENITLETVCHDPKLPYHDFILEDAASPKCIMKEK |
| | D57N | DAP10 TM D57N, | KKPGETFFMCSCSSDECNDNIIFSEEYNTSNPDLLLVIF |
| | | DAP10 | QQTTPGERSSLPAFYPGTSGSCSGCGSLSLPLLAGLVAA |
| | | intracellular | NAVASLLIVGAVFLCARPRRSPAQEDGKVYINMPGRG |
| 111 | dN25 | dN25 BR2 ECD, | QLCKFCDVRFSTCDNQKSCMSNCSITSICEKPQEVCVAV |
| | BR2.ICOS | ICOS TM, ICOS | WRKNDENITLETVCHDPKLPYHDFILEDAASPKCIMKEK |
| | | intracellular | KKPGETFFMCSCSSDECNDNIIFSEEYNTSNPDLLLVIF |
| | | | QFWLPIGCAAFVVVCILGCILICWLTKKKYSSSVHDPNG |
| | | | EYMFMRAVNTAKKSRLTDVTL |
| 112 | dN25 | dN25 BR2 ECD, | QLCKFCDVRFSTCDNQKSCMSNCSITSICEKPQEVCVAV |
| | BR2.CD40 | CD40 TM, CD40 | WRKNDENITLETVCHDPKLPYHDFILEDAASPKCIMKEK |
| | | intracellular | KKPGETFFMCSCSSDECNDNIIFSEEYNTSNPDLLLVIF |
| | | | QALVVIPIIFGILFAILLVLVFIKKVAKKPTNKAPHPKQ |
| | | | EPQEINFPDDLPGSNTAAPVQETLHGCQPVTQEDGKESR |
| | | | ISVQERQ |
| 113 | dN25 | dN25 BR2 ECD, | QLCKFCDVRFSTCDNQKSCMSNCSITSICEKPQEVCVAV |
| | BR2.OX40 | OX40 TM, OX40 | WRKNDENITLETVCHDPKLPYHDFILEDAASPKCIMKEK |
| | | intracellular | KKPGETFFMCSCSSDECNDNIIFSEEYNTSNPDLLLVIF |
| | | | QVAAILGLGLVLGLLGPLAILLALYLLRRDQRLPPDAHK |
| | | | PPGGGSFRTPIQEEQADAHSTLAKI |
| 114 | dN25 | dN25 BR2 ECD, | QLCKFCDVRFSTCDNQKSCMSNCSITSICEKPQEVCVAV |
| | BR2.BAFFR | BAFFR TM, | WRKNDENITLETVCHDPKLPYHDFILEDAASPKCIMKEK |
| | | BAFFR | KKPGETFFMCSCSSDECNDNIIFSEEYNTSNPDLLLVIF |
| | | intracellular | QFGAPALLGLALVLALVLVGLVSWRRRQRRLRGASSAEA |
| | | | PDGDKDAPEPLDKVIILSPGISDATAPAWPPPGEDPGTT |
| | | | PPGHSVPVPATELGSTELVTTKTAGPEQQ |
| 115 | dN25 | dN25 BR2 ECD, | QLCKFCDVRFSTCDNQKSCMSNCSITSICEKPQEVCVAV |
| | BR2.PD1tm. | PD1 TM, MyD88 | WRKNDENITLETVCHDPKLPYHDFILEDAASPKCIMKEK |
| | MyD88 | | KKPGETFFMCSCSSDECNDNIIFSEEYNTSNPDLLLVIF |
| | | | QVGVVGGLLGSLVLLVWVLAVICSRAARGTIGARRTGQM |
| | | | AAGGPGAGSAAPVSSTSSLPLAALNMRVRRRLSLFLNVR |
| | | | TQVAADWTALAEEMDFEYLEIRQLETQADPTGRLLDAWQ |
| | | | GRPGASVGRLLDLLTKLGRDDVLLELGPSIEEDCQKYIL |
| | | | KQQQEEAEKPLQVAAVDSSVPRTAELAGITTLDDPLGHM |
| | | | PERFDAFICYCPSDI |
| 162 | CCR 15.1 | TPOR TM, TPOR | SDPTRVETATETAWISLVTALHLVLGLNAVLGLLLLRKQ |
| CCR | | JAK-binding | FPAHYRRLRHALWPSLPDLHRVLGQYLRDTAALSPPKAT |
| 15.1 | | domain, IL2RbYY | VSDTCEEVEPSLLEILPKSSERTPLPLLEDEGVAGAPTG |
| | | signaling domain | SSPQPLQPLSGEDDAYCTFPSRDDLLLFSPSGQGEFRAL |
| | | | NARLPLNTDAYLSLQELQGQDPTHLV |
| 163 | dnHACi | CD8 signal | MALPVTALLLPLALLLHAARPPGWFLDSPDRPWNPPTFS |
| | PD1 | sequence | PALLVVTEGDNATFTCSFSNTSESFHVIWHRESPSGQTD |
| | | (underlined), HA | TLAAFPEDRSOPGQDCRFRVTQLPNGRDFHMSVVRARRN |
| | | PD1 ECD, PD1 | DSGTYVCGVISLAPKIQIKESLRAELRVTERRAEVPTAH |
| | | TM | PSPSPRPAGQFQTLVVGVVGGLLGSLVLLVWVLAVICSR |
| | | | AARGTIGARRTGQ |
| 166 | CCR 15.3 | TPOR TM, TPOR | SDPTRVETATETAWISLVTALLLVLGLNAVLGLLLLRKQ |
| | | JAK-binding | FPAHYRRLRHALWPSLPDLHRVLGQYLRDTAALSPPKAT |
| | | domain, | VSDTCEEVEPSLLEILPKSSERTPLPLLEQQDKVPEPAS |
| | | IL2RbYYY | LSSNHSLTSCFTNQGYFFFHLPDALEIEACQDEGVAGAP |
| | | signaling domain | TGSSPQPLQPLSGEDDAYCTFPSRDDLLLFSPSGQGEFR |
| | | | ALNARLPLNTDAYLSLQELQGQDPTHLV |
| 177 | TPOR | TPOR TM variant | SDPTRVETATETAWILVLISLVTALHLVLGLSAVLGLLL |
| | N + 4 | | LRWQFPAHYRRLRHALWPSLPDLHRVLGQYLRDTAALSP |
| | | | PKATVSDTCEEVEPSLLEILPKSSERTPLPL |
| 178 | CD70 | Exemplary CD70 | MALPVTALLLPLALLLHAARPQVTLKESGPVLVKPTE |
| | CAR | CAR | TLTLTCTVSGFSLSNARMGVTWIRQPPGKALEWLAHI |
| | | | FSNDEKSYSTSLKSRLTISKDTSKTQVVLTMTNMDPV |
| | | | DTATYYCARIRDYYDISSYYDYWGQGTLVSVSSGGGG |
| | | | SGGGGSGGGGSDIQMTQSPSAMSASVGDRVTITCRAS |
| | | | QDISNYLAWFQQKPGKVPKRLIYAASSLQSGVPSRFS |
| | | | GSGSGTEFTLTISSLLPEDFATYYCLQLNSFPFTFGG |
| | | | GTKVEINTTTPAPRPPTPAPTIASQPLSLRPEACRPA |
| | | | AGGAVHTRGLDFACDIYIWAPLAGTCGVLLLSLVITL |
| | | | YCKRGRKKLLYIFKQPFMRPVQTTQEEDGCSCRFPEE |
| | | | EEGGCELRVKFSRSADAPAYQQGQNQLYNELNLGRRE |
| | | | EYDVLDKRRGRDPEMGGKPRRKNPQEGLYNELQKDKM |
| | | | AEAYSEIGMKGERRRGKGHDGLYQGLSTATKDTYDAL |
| | | | HMQALPPR |
| 179 | CD70 | Exemplary CD70 | MALPVTALLLPLALLLHAARPGGGGSCPYSNPSLCSG |
| | CAR | CAR with a safety | GGGSGGGGSQVTLKESGPVLVKPTETLTLTCTVSGFS |
| | | switch | LSNARMGVTWIRQPPGKALEWLAHIFSNDEKSYSTSL |
| | | | KSRLTISKDTSKTQVVLTMTNMDPVDTATYYCARIRD |
| | | | YYDISSYYDYWGQGTLVSVSSGGGGSGGGGSGGGGSD |
| | | | IQMTQSPSAMSASVGDRVTITCRASQDISNYLAWFQQ |
| | | | KPGKVPKRLIYAASSLQSGVPSRFSGSGSGTEFTLTI |
| | | | SSLLPEDFATYYCLQLNSFPFTFGGGTKVEINGSGGG |
| | | | GSCPYSNPSLCSGGGGSELPTQGTFSNVSTNVSPAKP |
| | | | TTTACPYSNPSLCTTTPAPRPPTPAPTIASQPLSLRP |
| | | | EACRPAAGGAVHTRGLDFACDIYIWAPLAGTCGVLLL |
| | | | SLVITLYCKRGRKKLLYIFKQPFMRPVQTTQEEDGCS |
| | | | CRFPEEEEGGCELRVKFSRSADAPAYQQGQNQLYNEL |
| | | | NLGRREEYDVLDKRRGRDPEMGGKPRRKNPQEGLYNE |
| | | | LQKDKMAEAYSEIGMKGERRRGKGHDGLYQGLSTATK |
| | | | DTYDALHMQALPPR |

Nucleotide sequences of full-length chimeric switch receptors as disclosed herein are provided in Table 4.

TABLE 4

| SEQ | | | |
|---|---|---|---|
| ID | Construct | Polypeptide | |
| NO: | name | structure | DNA Sequence |
| 116 | 2G1-RSR | CD8 signal | ATGGCCCTGCCAGTGACCGCCCTGCTGCTGCCCCTGGCC |
| | | sequence | CTGCTGCTGCACGCAGCCAGACCCGGAGGAGGAGGCTCT |
| | | (underlined), | TGCCCCTACAGCAACCCTTCCCTGTGCGGAGGAGGAGGC |
| | | rituximab | TCTCAGCTGCAGCTGCAGGAGTCCGGCCCTGGCCTGGTG |
| | | mimotope, 2G1 | AAGCCATCCGAGACCCTGTCTCTGACCTGCACAGTGAGC |
| | | scFv, rituximab | GGCGGCTCCATCAGCTCCTCTAGCTACTATTGGGGCTGG |
| | | mimotope, CD8α | ATCAGACAGCCCCCTGGCAAGGGACTGGAGTGGATCGGC |
| | | hinge, CD8α | AGCATCTACTATTCCGGCAACATCTACCACAATCCTTCT |
| | | transmembrane, | CTGAAGAGCCGCGTGTCTATCAGCGTGGACACCTCCAAG |
| | | CD8α | AACCAGTTCTCTCTGAGGCTGTCCTCTGTGACCGCAGCA |
| | | cytoplasmic | GATACAGCCGTGTACTATTGCGCCAGGGAGATCATCGTG |
| | | domain | GGAGCAACCCACTTTGACTATTGGGGCCAGGGCACCCTG |
| | | (truncated), 4- | GTGACAGTGAGCTCCGGCGGCGGCGGCTCTGGAGGAGGA |
| | | 1BB (TNFRSF9, | GGCAGCGGCGGAGGAGGCTCCGGAGGCGGCGGCTCTGCC |
| | | CD137) | ATCCAGATGACACAGTCCCCATCTAGCCTGTCCGCCTCT |
| | | cytoplasmic | GTGGGCGACAGGGTGACCATCACATGTAGAGCCAGCCAG |
| | | domain, CD3ζ | GGCATCAGGAACGATCTGGGCTGGTACCAGCAGAAGCCA |
| | | cytoplasmic | GGCAAGGCCCCCGAGCTGCTGATCTATGCCGCCTCCTCT |
| | | domain | CTGCAGTCTGGCGTGCCAAGCAGATTCAGCGGCTCCGGC |
| | | | TCTGGCACCGACTTTACCCTGACAATCAGCTCCCTGCAG |
| | | | CCCGAGGACTTCGCCACATACTATTGTCTGCAGGATTAC |
| | | | AATTATCCCCTGACCTTTGGCCCTGGCACAAAGGTGGAT |
| | | | ATCAAGGGAGGAGGAGGCTCTTGCCCCTACAGCAACCCT |
| | | | TCCCTGTGCGGAGGAGGAGGCTCTACAACCACACCTGCA |
| | | | CCTAGGCCACCTACACCTGCACCAACCATCGCCAGCCAG |
| | | | CCTCTGTCCCTGAGACCAGAGGCCTGTAGGCCAGCAGCA |
| | | | GGAGGAGCAGTGCACACCCGGGGCCTGGACTTCGCCTGC |
| | | | GATATCTACATCTGGGCACCACTGGCAGGAACATGTGGC |
| | | | GTGCTGCTGCTGTCCCTGGTCATCACCCTGTACTGCAAG |
| | | | AGAGGCAGGAAGAAGCTGCTGTATATCTTCAAGCAGCCC |
| | | | TTCATGAGACCCGTGCAGACAACCCAGGAGGAGGACGGC |
| | | | TGCAGCTGTAGGTTCCCAGAGGAGGAGGAGGGAGGATGT |
| | | | GAGCTGCGCGTGAAGTTTTCCCGGTCTGCCGATGCACCT |
| | | | GCATACCAGCAGGGACAGAACCAGCTGTATAACGAGCTG |
| | | | AATCTGGGCCGGAGAGAGGAGTACGACGTGCTGGATAAG |
| | | | AGGAGGGGAAGGGACCCTGAGATGGGAGGCAAGCCTCGG |
| | | | AGAAAGAACCCACAGGAGGGCCTGTACAATGAGCTGCAG |
| | | | AAGGACAAGATGGCCGAGGCCTATAGCGAGATCGGCATG |
| | | | AAGGGAGAGAGGCGCCGGGGCAAGGGACACGATGGCCTG |
| | | | TATCAGGGCCTGTCAACCGCTACAAAAGATACCTACGAT |
| | | | GCTCTGCACATGCAGGCTCTGCCACCAAGA |
| 117 | 2G1.15.1 | CD8 signal | ATGGCCCTGCCAGTGACCGCCCTGCTGCTGCCACTGGCC |
| | | sequence | CTGCTGCTGCACGCAGCAAGGCCATCAGACCCTACTAGA |
| | | (underlined), | GTCGAGACCGCTACCGAGACCGCTTGGATCTCTCTGGTG |
| | | TpoR (S505N | ACCGCCCTGCACCTGGTGCTGGGCCTGAACGCCGTGCTG |
| | | W515K), IL2Rb- | GGCCTGCTGCTGCTGAGGAAGCAGTTCCCAGCACACTAC |
| | | YY, P2A, | CGGAGACTGAGGCACGCACTGTGGCCAAGCCTGCCCGAC |
| | | CD8 signal | CTGCACAGGGTGCTGGGACAGTATCTGAGGGATACAGCC |
| | | sequence, | GCCCTGAGCCCACCTAAGGCAACCGTGTCCGACACATGC |
| | | rituximab | GAGGAGGTGGAACCAAGTCTGCTGGAAATCCTGCCAAAA |
| | | mimotope, 2G1 | TCCTCTGAGCGGACACCCCTGCCCCTGCTCGAGGACGAG |
| | | scFv, rituximab | GGAGTGGCAGGAGCACCAACCGGCAGCTCCCCCCAGCCT |
| | | mimotope, CD8α | CTGCAGCCACTGTCCGGAGAGGACGATGCATACTGCACA |
| | | hinge, CD8α | TTCCCTTCTCGGGACGATCTGCTGCTGTTCTCTCCAAGC |
| | | transmembrane, | GGACAGGGAGAGTTTCGGGCCCTGAACGCCAGACTGCCC |
| | | CD8α | CTGAATACCGACGCCTATCTGAGCCTGCAGGAGCTGCAG |
| | | cytoplasmic | GGACAGGACCCCACACACCTGGTGGGATCCGGAGCCACC |
| | | domain | AACTTCTCCCTGCTGAAGCAGGCCGGCGATGTGGAGGAG |
| | | (truncated), 4- | AATCCAGGCCCCATGGCCCTGCCAGTGACCGCCCTGCTG |
| | | 1BB (TNFRSF9, | CTGCCCCTGGCCCTGCTGCTGCACGCAGCCAGACCCGGA |
| | | CD137) | GGAGGAGGCTCTTGCCCCTACAGCAACCCTTCCCTGTGC |
| | | cytoplasmic | GGAGGAGGAGGCTCTCAGCTGCAGCTGCAGGAGTCCGGC |
| | | domain, CD3ζ | CCTGGCCTGGTGAAGCCATCCGAGACCCTGTCTCTGACC |
| | | cytoplasmic | TGCACAGTGAGCGGCGGCTCCATCAGCTCCTCTAGCTAC |
| | | domain | TATTGGGGCTGGATCAGACAGCCCCCTGGCAAGGGACTG |
| | | | GAGTGGATCGGCAGCATCTACTATTCCGGCAACATCTAC |
| | | | CACAATCCTTCTCTGAAGAGCCGCGTGTCTATCAGCGTG |
| | | | GACACCTCCAAGAACCAGTTCTCTCTGAGGCTGTCCTCT |
| | | | GTGACCGCAGCAGATACAGCCGTGTACTATTGCGCCAGG |
| | | | GAGATCATCGTGGGAGCAACCCACTTTGACTATTGGGGC |
| | | | CAGGGCACCCTGGTGACAGTGAGCTCCGGCGGCGGCGGC |
| | | | TCTGGAGGAGGAGGCAGCGGCGGAGGAGGCTCCGGAGGC |
| | | | GGCGGCTCTGCCATCCAGATGACACAGTCCCCATCTAGC |
| | | | CTGTCCGCCTCTGTGGGCGACAGGGTGACCATCACATGT |
| | | | AGAGCCAGCCAGGGCATCAGGAACGATCTGGGCTGGTAC |
| | | | CAGCAGAAGCCAGGCAAGGCCCCCGAGCTGCTGATCTAT |
| | | | GCCGCCTCCTCTCTGCAGTCTGGCGTGCCAAGCAGATTC |
| | | | AGCGGCTCCGGCTCTGGCACCGACTTTACCCTGACAATC |
| | | | AGCTCCCTGCAGCCCGAGGACTTCGCCACATACTATTGT |
| | | | CTGCAGGATTACAATTATCCCCTGACCTTTGGCCCTGGC |
| | | | ACAAAGGTGGATATCAAGGGAGGAGGAGGCTCTTGCCCC |
| | | | TACAGCAACCCTTCCCTGTGCGGAGGAGGAGGCTCTACA |
| | | | ACCACACCTGCACCTAGGCCACCTACACCTGCACCAACC |
| | | | ATCGCCAGCCAGCCTCTGTCCCTGAGACCAGAGGCCTGT |
| | | | AGGCCAGCAGCAGGAGGAGCAGTGCACACCCGGGGCCTG |
| | | | GACTTCGCCTGCGATATCTACATCTGGGCACCACTGGCA |
| | | | GGAACATGTGGCGTGCTGCTGCTGTCCCTGGTCATCACC |
| | | | CTGTACTGCAAGAGAGGCAGGAAGAAGCTGCTGTATATC |
| | | | TTCAAGCAGCCCTTCATGAGACCCGTGCAGACAACCCAG |
| | | | GAGGAGGACGGCTGCAGCTGTAGGTTCCCAGAGGAGGAG |
| | | | GAGGGAGGATGTGAGCTGCGCGTGAAGTTTTCCCGGTCT |
| | | | GCCGATGCACCTGCATACCAGCAGGGACAGAACCAGCTG |
| | | | TATAACGAGCTGAATCTGGGCCGGAGAGAGGAGTACGAC |
| | | | GTGCTGGATAAGAGGAGGGGAAGGGACCCTGAGATGGGA |
| | | | GGCAAGCCTCGGAGAAAGAACCCACAGGAGGGCCTGTAC |
| | | | AATGAGCTGCAGAAGGACAAGATGGCCGAGGCCTATAGC |
| | | | GAGATCGGCATGAAGGGAGAGAGGCGCCGGGGCAAGGGA |
| | | | CACGATGGCCTGTATCAGGGCCTGTCAACCGCTACAAAA |
| | | | GATACCTACGATGCTCTGCACATGCAGGCTCTGCCACCA |
| | | | AGA |
| 118 | WT | WT PD1 ECD, | CCCGGCTGGTTCCTGGACAGCCCTGACCGGCCTTGGAAC |
| | PD1.CD28 | CD28 ECD | CCCCCCACCTTCTCTCCTGCTCTGCTGGTGGTGACAGAG |
| | | (truncated), CD28 | GGCGACAACGCCACCTTCACCTGCAGCTTCAGCAATACC |
| | | TM, CD28 | TCCGAGAGCTTTGTGCTGAACTGGTACCGGATGAGCCCC |
| | | intracellular | TCTAATCAGACAGATAAGCTGGCTGCTTTTCCGGAAGAT |
| | | | AGAAGCCAGCCTGGCCAAGACTGCCGCTTTAGAGTTACC |
| | | | CAGCTGCCTAACGGCAGAGATTTCCACATGTCTGTGGTG |
| | | | CGGGCCAGACGGAACGACAGCGGCACCTACCTGTGCGGC |
| | | | GCCATCAGCCTGGCCCCTAAGGCCCAGATCAAGGAAAGC |
| | | | CTGAGAGCCGAGCTGCGCGTGACCGAGAGAAGGGCCGAA |
| | | | GTGCCTACCGCCCACCCCAGCCCATCTCCTAGACCAGCC |
| | | | GGCCAGTTCCAGACCCTGGTGTGTCCTTCCCCTCTGTTC |
| | | | CCCGGCCCTAGCAAACCCTTCTGGGTGCTGGTGGTCGTG |
| | | | GGCGGAGTGCTGGCTTGCTACAGCCTGCTCGTGACCGTG |
| | | | GCCTTCATCATCTTCTGGGTCAGAAGCAAGCGGTCCAGA |
| | | | CTGCTGCACAGCGACTACATGAACATGACACCTAGAAGA |
| | | | CCTGGACCTACAAGAAAGCACTACCAGCCCTACGCCCCT |
| | | | CCTCGGGACTTCGCCGCTTATAGATCT |
| 119 | WT | WT PD1 ECD, | CCCGGCTGGTTCCTGGACAGCCCTGACCGGCCTTGGAAC |
| | PD1.CD28. | CD28 ECD | CCCCCCACCTTCTCTCCTGCTCTGCTGGTGGTGACAGAG |
| | YMFM | (truncated), CD28 | GGCGACAACGCCACCTTCACCTGCAGCTTCAGCAATACC |
| | | TM, | TCCGAGAGCTTTGTGCTGAACTGGTACCGGATGAGCCCC |
| | | CD28. YMFM | TCTAATCAGACAGATAAGCTGGCTGCTTTTCCGGAAGAT |
| | | intracellular | AGAAGCCAGCCTGGCCAAGACTGCCGCTTTAGAGTTACC |
| | | | CAGCTGCCTAACGGCAGAGATTTCCACATGTCTGTGGTG |
| | | | CGGGCCAGACGGAACGACAGCGGCACCTACCTGTGCGGC |
| | | | GCCATCAGCCTGGCCCCTAAGGCCCAGATCAAGGAAAGC |
| | | | CTGAGAGCCGAGCTGCGCGTGACCGAGAGAAGGGCCGAA |
| | | | GTGCCTACCGCCCACCCCAGCCCATCTCCTAGACCAGCC |
| | | | GGCCAGTTCCAGACCCTGGTGTGTCCTTCCCCTCTGTTC |
| | | | CCCGGCCCTAGCAAACCCTTCTGGGTGCTGGTGGTCGTG |
| | | | GGCGGAGTGCTGGCTTGCTACAGCCTGCTCGTGACCGTG |
| | | | GCCTTCATCATCTTCTGGGTCAGAAGCAAGCGGTCCAGA |
| | | | CTGCTGCACAGCGACTACATGTTCATGACACCTAGAAGA |
| | | | CCTGGACCTACAAGAAAGCACTACCAGCCCTACGCCCCT |
| | | | CCTCGGGACTTCGCCGCTTATAGATCT |
| 120 | WT | WT PD1 ECD, | CCCGGCTGGTTCCTGGACAGCCCTGACCGGCCTTGGAAC |
| | PD1.CD28. | CD28 ECD | CCCCCCACCTTCTCTCCTGCTCTGCTGGTGGTGACAGAG |
| | AYAA | (truncated), CD28 | GGCGACAACGCCACCTTCACCTGCAGCTTCAGCAATACC |
| | | TM, | TCCGAGAGCTTTGTGCTGAACTGGTACCGGATGAGCCCC |
| | | CD28.AYAA | TCTAATCAGACAGATAAGCTGGCTGCTTTTCCGGAAGAT |
| | | intracellular | AGAAGCCAGCCTGGCCAAGACTGCCGCTTTAGAGTTACC |
| | | | CAGCTGCCTAACGGCAGAGATTTCCACATGTCTGTGGTG |
| | | | CGGGCCAGACGGAACGACAGCGGCACCTACCTGTGCGGC |
| | | | GCCATCAGCCTGGCCCCTAAGGCCCAGATCAAGGAAAGC |
| | | | CTGAGAGCCGAGCTGCGCGTGACCGAGAGAAGGGCCGAA |
| | | | GTGCCTACCGCCCACCCCAGCCCATCTCCTAGACCAGCC |
| | | | GGCCAGTTCCAGACCCTGGTGTGTCCTTCCCCTCTGTTC |
| | | | CCCGGCCCTAGCAAACCCTTCTGGGTGCTGGTGGTCGTG |
| | | | GGCGGAGTGCTGGCTTGCTACAGCCTGCTCGTGACCGTG |
| | | | GCCTTCATCATCTTCTGGGTCAGAAGCAAGCGGTCCAGA |
| | | | CTGCTGCACAGCGACTACATGAACATGACACCTAGAAGA |
| | | | CCTGGACCTACAAGAAAGCACTACCAGGCCTACGCCGCC |
| | | | CCTCGGGACTTCGCCGCTTATAGATCT |
| 121 | WT | WT PD1 ECD, | CCCGGATGGTTCCTGGATTCCCCAGACAGACCATGGAAC |
| | PD1.CD2 | CD2 ECD | CCCCCCACCTTTTCTCCTGCTCTGCTGGTGGTGACAGAG |
| | (full) | (truncated), CD2 | GGCGACAACGCCACATTCACCTGTAGCTTCAGCAATACC |
| | | TM, CD2 | AGCGAGAGCTTCGTGCTGAACTGGTATAGAATGTCTCCT |
| | | intracellular | TCTAACCAGACCGACAAGCTGGCCGCTTTCCCCGAAGAT |
| | | | CGGAGCCAACCTGGCCAAGATTGCAGATTCAGAGTGACC |
| | | | CAGCTGCCTAACGGCCGGGACTTCCACATGAGCGTGGTC |
| | | | AGAGCCAGAAGGAACGACAGCGGCACCTACCTGTGCGGC |
| | | | GCCATCAGCCTGGCTCCTAAGGCCCAGATCAAGGAAAGC |
| | | | CTGCGCGCCGAGCTGCGGGTCACAGAGAGAAGAGCCGAG |
| | | | GTGCCTACCGCCCACCCTTCTCCGAGCCCCCGGCCAGCC |
| | | | GGCCAGTTCCAGACACTGGTGGTGGAGCCTGTGTCCTGC |
| | | | CCTGAGAAGGGCCTGGACATCTACCTGATCATCGGCATC |
| | | | TGCGGAGGCGGATCTCTGCTCATGGTGTTCGTGGCCCTG |
| | | | CTGGTGTTTTACATCACAAAGCGGAAGAAACAGAGAAGC |
| | | | AGACGGAACGACGAGGAACTGGAAACCAGAGCCCACAGA |
| | | | GTGGCCACCGAGGAACGGGGCAGAAAGCCTCATCAGATT |
| | | | CCTGCCAGCACCCCTCAGAACCCCGCTACCTCCCAGCAC |
| | | | CCCCCTCCTCCTCCTGGACACAGATCTCAGGCCCCTAGC |
| | | | CACCGGCCCCCTCCTCCTGGCCATCGGGTGCAGCACCAA |
| | | | CCCCAGAAAAGACCTCCAGCTCCTTCCGGCACCCAGGTG |
| | | | CACCAGCAGAAGGGCCCTCCTCTGCCTAGACCTAGGGTT |
| | | | CAGCCCAAGCCCCCCCACGGCGCAGCCGAAAACAGCCTG |
| | | | AGCCCCAGCAGCAAT |
| 122 | WT | WT PD1 ECD, | CCAGGCTGGTTCCTGGATAGCCCCGACAGACCTTGGAAT |
| | PD1.CD2 | CD2 ECD | CCCCCTACATTCAGCCCTGCTCTGCTGGTCGTGACCGAG |
| | (short) | (truncated), CD2 | GGCGACAACGCCACCTTCACATGCAGCTTCAGCAACACC |
| | | TM, CD2 | AGCGAGTCTTTTGTGCTGAACTGGTATCGGATGAGCCCT |
| | | intracellular | TCTAACCAGACAGATAAGCTGGCAGCCTTCCCCGAAGAT |
| | | (short) | AGAAGCCAACCTGGCCAGGACTGCAGATTCAGAGTGACC |
| | | | CAGCTGCCTAACGGCCGGGACTTCCACATGTCTGTGGTG |
| | | | CGGGCCAGACGCAACGACAGCGGCACCTACCTGTGCGGC |
| | | | GCCATCAGCCTGGCCCCTAAGGCCCAGATCAAGGAAAGC |
| | | | CTGAGAGCCGAGCTGCGGGTGACAGAAAGAAGGGCCGAA |
| | | | GTGCCCACCGCCCACCCTTCCCCTTCCCCCAGACCTGCC |
| | | | GGACAATTTCAGACCCTGGTTGTCGAGCCTGTGAGCTGC |
| | | | CCCGAGAAGGGGCTGGACATCTACCTGATCATCGGCATT |
| | | | TGTGGCGGCGGATCTCTGCTGATGGTGTTCGTGGCCCTG |
| | | | CTGGTGTTCTACATCACCAAGAGAAAGAAGCAGACCCCT |
| | | | CAGAACCCCGCCACAAGCCAGCATCCTCCACCACCTCCC |
| | | | GGCCACCGGAGCCAGGCCCCAAGTCACAGACCCCCACCT |
| | | | CCTGGCCACAGAGTGCAGCACCAGCCCCAGAAGCGGCCT |
| | | | CCAGCTCCTAGCGGAACCCAAGTGCACCAGCAGAAAGGC |
| | | | CCTCCTCTGCCTCGGCCTAGAGTGCAGCCTAAACCTCCG |
| | | | CACGGCGCTGCTGAGAACAGCTTGTCTCCCTCCAGCAAT |
| 123 | WT | WT PD1 ECD, | CCCGGATGGTTCCTGGATTCCCCAGACAGACCATGGAAC |
| | PD1.DAP10. | DAP10 ECD, | CCCCCCACCTTTTCTCCTGCTCTGCTGGTGGTGACAGAG |
| | D57N | DAP10 TM | GGCGACAACGCCACATTCACCTGTAGCTTCAGCAATACC |
| | | D57N, DAP10 | AGCGAGAGCTTCGTGCTGAACTGGTATAGAATGTCTCCT |
| | | intracellular | TCTAACCAGACCGACAAGCTGGCCGCTTTCCCCGAAGAT |
| | | | CGGAGCCAACCTGGCCAAGATTGCAGATTCAGAGTGACC |
| | | | CAGCTGCCTAACGGCCGGGACTTCCACATGAGCGTGGTC |
| | | | AGAGCCAGAAGGAACGACAGCGGCACCTACCTGTGCGGC |
| | | | GCCATCAGCCTGGCTCCTAAGGCCCAGATCAAGGAAAGC |
| | | | CTGCGCGCCGAGCTGCGGGTCACAGAGAGAAGAGCCGAG |
| | | | GTGCCTACCGCCCACCCTTCTCCGAGCCCCCGGCCAGCC |
| | | | GGCCAGTTCCAGACACTGGTGCAGACCACACCTGGAGAA |
| | | | CGGAGCAGCCTCCCCGCCTTCTACCCCGGCACCAGCGGC |
| | | | AGCTGCAGCGGATGTGGCAGCCTGTCTCTGCCTCTGCTG |
| | | | GCCGGCCTGGTCGCCGCCAACGCCGTGGCTTCTCTGCTG |
| | | | ATCGTGGGCGCCGTGTTCCTGTGCGCCAGACCTAGACGG |
| | | | TCCCCAGCTCAGGAGGACGGCAAGGTGTACATCAACATG |
| | | | CCTGGCAGAGGC |
| 124 | WT | WT PD1 ECD, | CCCGGATGGTTCCTGGATTCCCCAGACAGACCATGGAAC |
| | PD1.ICOS | ICOS TM, ICOS | CCCCCCACCTTTTCTCCTGCTCTGCTGGTGGTGACAGAG |
| | | intracellular | GGCGACAACGCCACATTCACCTGTAGCTTCAGCAATACC |
| | | | AGCGAGAGCTTCGTGCTGAACTGGTATAGAATGTCTCCT |
| | | | TCTAACCAGACCGACAAGCTGGCCGCTTTCCCCGAAGAT |
| | | | CGGAGCCAACCTGGCCAAGATTGCAGATTCAGAGTGACC |
| | | | CAGCTGCCTAACGGCCGGGACTTCCACATGAGCGTGGTC |
| | | | AGAGCCAGAAGGAACGACAGCGGCACCTACCTGTGCGGC |
| | | | GCCATCAGCCTGGCTCCTAAGGCCCAGATCAAGGAAAGC |
| | | | CTGCGCGCCGAGCTGCGGGTCACAGAGAGAAGAGCCGAG |
| | | | GTGCCTACCGCCCACCCTTCTCCGAGCCCCCGGCCAGCC |
| | | | GGCCAGTTCCAGACACTGGTGTTCTGGCTGCCTATCGGC |
| | | | TGCGCCGCTTTTGTGGTGGTCTGCATCCTGGGCTGTATC |
| | | | CTGATCTGCTGGCTGACCAAGAAGAAGTACAGCTCTTCC |
| | | | GTGCACGACCCCAACGGCGAGTACATGTTCATGCGGGCC |
| | | | GTGAACACCGCCAAGAAAAGCAGACTGACAGATGTGACC |
| | | | CTG |
| 125 | WT | WT PD1 ECD, | CCCGGATGGTTCCTGGATTCCCCAGACAGACCATGGAAC |
| | PD1.CD40 | CD40 TM, CD40 | CCCCCCACCTTTTCTCCTGCTCTGCTGGTGGTGACAGAG |
| | | intracellular | GGCGACAACGCCACATTCACCTGTAGCTTCAGCAATACC |
| | | | AGCGAGAGCTTCGTGCTGAACTGGTATAGAATGTCTCCT |
| | | | TCTAACCAGACCGACAAGCTGGCCGCTTTCCCCGAAGAT |
| | | | CGGAGCCAACCTGGCCAAGATTGCAGATTCAGAGTGACC |
| | | | CAGCTGCCTAACGGCCGGGACTTCCACATGAGCGTGGTC |
| | | | AGAGCCAGAAGGAACGACAGCGGCACCTACCTGTGCGGC |
| | | | GCCATCAGCCTGGCTCCTAAGGCCCAGATCAAGGAAAGC |
| | | | CTGCGCGCCGAGCTGCGGGTCACAGAGAGAAGAGCCGAG |
| | | | GTGCCTACCGCCCACCCTTCTCCGAGCCCCCGGCCAGCC |
| | | | GGCCAGTTCCAGACACTGGTGGCCCTGGTCGTGATCCCC |
| | | | ATCATCTTCGGCATCCTGTTTGCCATTCTGCTGGTGCTG |
| | | | GTGTTCATCAAGAAAGTGGCCAAGAAACCTACAAACAAG |
| | | | GCCCCTCACCCCAAGCAGGAGCCTCAGGAGATCAACTTC |
| | | | CCCGACGACCTGCCTGGATCTAATACCGCCGCTCCAGTG |
| | | | CAAGAAACCCTGCACGGCTGCCAGCCTGTGACCCAGGAG |
| | | | GATGGCAAGGAAAGCAGAATCAGCGTGCAGGAGCGGCAG |
| 126 | WT | WT PD1 ECD, | CCCGGATGGTTCCTGGATTCCCCAGACAGACCATGGAAC |
| | PD1.OX40 | OX40 TM, OX40 | CCCCCCACCTTTTCTCCTGCTCTGCTGGTGGTGACAGAG |
| | | intracellular | GGCGACAACGCCACATTCACCTGTAGCTTCAGCAATACC |
| | | | AGCGAGAGCTTCGTGCTGAACTGGTATAGAATGTCTCCT |
| | | | TCTAACCAGACCGACAAGCTGGCCGCTTTCCCCGAAGAT |
| | | | CGGAGCCAACCTGGCCAAGATTGCAGATTCAGAGTGACC |
| | | | CAGCTGCCTAACGGCCGGGACTTCCACATGAGCGTGGTC |
| | | | AGAGCCAGAAGGAACGACAGCGGCACCTACCTGTGCGGC |
| | | | GCCATCAGCCTGGCTCCTAAGGCCCAGATCAAGGAAAGC |
| | | | CTGCGCGCCGAGCTGCGGGTCACAGAGAGAAGAGCCGAG |
| | | | GTGCCTACCGCCCACCCTTCTCCGAGCCCCCGGCCAGCC |
| | | | GGCCAGTTCCAGACACTGGTGGTGGCCGCTATCCTGGGC |
| | | | CTGGGCCTCGTGCTGGGCCTGCTGGGACCTCTGGCCATC |
| | | | CTGCTGGCTCTGTACCTGCTGAGACGGGACCAGAGACTG |
| | | | CCTCCAGATGCCCACAAGCCCCCCGGCGGCGGATCTTTC |
| | | | AGAACCCCTATCCAGGAGGAACAGGCCGACGCCCACAGC |
| | | | ACACTGGCCAAGATC |
| 127 | HA | HA PD1 ECD, | CCCGGCTGGTTCCTGGACAGCCCCGACAGACCTTGGAAT |
| | PD1.CD28 | CD28 ECD | CCTCCAACCTTTAGCCCAGCCCTGCTGGTGGTGACAGAG |
| | | (truncated), CD28 | GGAGATAACGCCACCTTCACCTGCAGCTTCAGCAACACC |
| | | TM, CD28 | AGCGAGAGCTTTCACGTGATCTGGCACCGGGAATCCCCA |
| | | intracellular | TCTGGCCAGACCGACACCCTGGCTGCCTTCCCCGAAGAT |
| | | | AGAAGCCAGCCTGGCCAAGACTGCAGATTCCGGGTGACA |
| | | | CAGCTGCCCAACGGCAGAGACTTCCACATGTCTGTGGTG |
| | | | CGGGCTAGACGGAACGACAGCGGCACCTACGTGTGCGGC |
| | | | GTGATTTCTCTGGCTCCTAAGATCCAGATCAAGGAATCC |
| | | | CTGCGGGCCGAGCTGAGAGTGACAGAGAGAAGGGCCGAG |
| | | | GTGCCTACCGCCCACCCCAGCCCAAGCCCTAGGCCCGCC |
| | | | GGCCAGTTCCAGACCCTGGTTTGTCCTAGCCCCCTGTTC |
| | | | CCCGGTCCTAGCAAACCTTTCTGGGTGCTGGTGGTGGTG |
| | | | GGCGGCGTGCTGGCCTGCTACAGCCTGCTGGTCACAGTG |
| | | | GCCTTTATCATCTTCTGGGTCAGATCTAAGCGGTCCAGA |
| | | | CTGCTGCATTCTGATTACATGAACATGACCCCTAGAAGA |
| | | | CCTGGACCTACAAGAAAGCACTACCAGCCTTACGCCCCT |
| | | | CCTCGGGACTTCGCCGCTTATAGAAGC |
| 128 | HA | HA PD1 ECD, | CCCGGCTGGTTCCTGGACAGCCCCGACAGACCTTGGAAT |
| | PD1.CD28. | CD28 ECD | CCTCCAACCTTTAGCCCAGCCCTGCTGGTGGTGACAGAG |
| | YMFM | (truncated), CD28 | GGAGATAACGCCACCTTCACCTGCAGCTTCAGCAACACC |
| | | TM, | AGCGAGAGCTTTCACGTGATCTGGCACCGGGAATCCCCA |
| | | CD28.YMFM | TCTGGCCAGACCGACACCCTGGCTGCCTTCCCCGAAGAT |
| | | intracellular | AGAAGCCAGCCTGGCCAAGACTGCAGATTCCGGGTGACA |
| | | | CAGCTGCCCAACGGCAGAGACTTCCACATGTCTGTGGTG |
| | | | CGGGCTAGACGGAACGACAGCGGCACCTACGTGTGCGGC |
| | | | GTGATTTCTCTGGCTCCTAAGATCCAGATCAAGGAATCC |
| | | | CTGCGGGCCGAGCTGAGAGTGACAGAGAGAAGGGCCGAG |
| | | | GTGCCTACCGCCCACCCCAGCCCAAGCCCTAGGCCCGCC |
| | | | GGCCAGTTCCAGACCCTGGTTTGTCCTAGCCCCCTGTTC |
| | | | CCCGGTCCTAGCAAACCTTTCTGGGTGCTGGTGGTGGTG |
| | | | GGCGGCGTGCTGGCCTGCTACAGCCTGCTGGTCACAGTG |
| | | | GCCTTTATCATCTTCTGGGTCAGATCTAAGCGGTCCAGA |
| | | | CTGCTGCATTCTGATTACATGTTCATGACCCCTAGAAGA |
| | | | CCTGGACCTACAAGAAAGCACTACCAGCCTTACGCCCCT |
| | | | CCTCGGGACTTCGCCGCTTATAGAAGC |
| 129 | HA | HA PD1 ECD, | CCCGGCTGGTTCCTGGACAGCCCCGACAGACCTTGGAAT |
| | PD1.CD28. | CD28 ECD | CCTCCAACCTTTAGCCCAGCCCTGCTGGTGGTGACAGAG |
| | AYAA | (truncated), CD28 | GGAGATAACGCCACCTTCACCTGCAGCTTCAGCAACACC |
| | | TM, | AGCGAGAGCTTTCACGTGATCTGGCACCGGGAATCCCCA |
| | | CD28.AYAA | TCTGGCCAGACCGACACCCTGGCTGCCTTCCCCGAAGAT |
| | | intracellular | AGAAGCCAGCCTGGCCAAGACTGCAGATTCCGGGTGACA |
| | | | CAGCTGCCCAACGGCAGAGACTTCCACATGTCTGTGGTG |
| | | | CGGGCTAGACGGAACGACAGCGGCACCTACGTGTGCGGC |
| | | | GTGATTTCTCTGGCTCCTAAGATCCAGATCAAGGAATCC |
| | | | CTGCGGGCCGAGCTGAGAGTGACAGAGAGAAGGGCCGAG |
| | | | GTGCCTACCGCCCACCCCAGCCCAAGCCCTAGGCCCGCC |
| | | | GGCCAGTTCCAGACCCTGGTTTGTCCTAGCCCCCTGTTC |
| | | | CCCGGTCCTAGCAAACCTTTCTGGGTGCTGGTGGTGGTG |
| | | | GGCGGCGTGCTGGCCTGCTACAGCCTGCTGGTCACAGTG |
| | | | GCCTTTATCATCTTCTGGGTCAGATCTAAGCGGTCCAGA |
| | | | CTGCTGCATTCTGATTACATGAACATGACCCCTAGAAGA |
| | | | CCTGGACCTACAAGAAAGCACTACCAGGCCTACGCCGCC |
| | | | CCTCGGGACTTCGCCGCTTATAGAAGC |
| 130 | HA | HA PD1 ECD, | CCTGGATGGTTCCTGGATAGCCCTGATAGACCTTGGAAC |
| | PD1.CD2 | CD2 ECD | CCCCCTACCTTCAGCCCTGCACTTCTGGTCGTGACCGAA |
| | (full) | (truncated), CD2 | GGCGACAACGCCACCTTCACCTGCAGCTTTAGCAACACC |
| | | TM, CD2 | TCCGAGAGCTTCCACGTGATCTGGCACAGAGAGTCCCCT |
| | | intracellular | TCTGGCCAGACCGACACCCTGGCCGCCTTCCCCGAAGAT |
| | | | AGAAGCCAGCCCGGCCAGGACTGCAGATTCAGAGTGACA |
| | | | CAGCTGCCCAACGGCCGCGACTTCCACATGAGCGTGGTT |
| | | | AGAGCTAGAAGGAACGACAGCGGCACCTACGTGTGCGGC |
| | | | GTGATCAGCCTGGCTCCCAAGATCCAGATCAAGGAAAGC |
| | | | CTGAGAGCCGAACTGCGGGTGACCGAGCGGAGAGCCGAG |
| | | | GTGCCCACCGCCCACCCTTCCCCCTCTCCAAGACCCGCC |
| | | | GGCCAATTTCAGACACTGGTGGTGGAGCCTGTGTCCTGT |
| | | | CCTGAGAAGGGACTGGACATCTACCTGATCATCGGCATC |
| | | | TGCGGAGGAGGCAGCCTGCTGATGGTGTTCGTGGCCCTG |
| | | | CTGGTGTTCTACATCACCAAGCGGAAGAAGCAGCGGAGC |
| | | | AGACGGAATGACGAGGAACTCGAGACAAGAGCCCATCGG |
| | | | GTCGCCACAGAGGAAAGAGGCAGAAAGCCCCACCAGATT |
| | | | CCTGCCAGCACACCTCAGAACCCTGCTACCAGCCAACAC |
| | | | CCCCCCCCCCCTCCTGGCCACAGATCTCAGGCCCCTAGC |
| | | | CACCGGCCCCCGCCACCTGGCCACCGGGTGCAGCACCAG |
| | | | CCTCAAAAAAGACCCCCTGCTCCTAGCGGCACACAGGTG |
| | | | CACCAGCAGAAAGGTCCTCCACTGCCTAGACCTCGGGTG |
| | | | CAGCCTAAGCCTCCACATGGCGCCGCTGAGAACAGCTTG |
| | | | TCTCCTAGTTCTAAT |
| 131 | HA | HA PD1 ECD, | CCCGGCTGGTTCCTGGATAGCCCTGACCGGCCATGGAAT |
| | PD1.CD2 | CD2 ECD | CCTCCTACCTTCAGCCCCGCTCTGCTCGTGGTCACAGAG |
| | (short) | (truncated), CD2 | GGAGATAACGCCACATTCACCTGTAGCTTCAGCAACACA |
| | | TM, CD2 | AGCGAGTCTTTTCACGTGATTTGGCATCGGGAATCTCCT |
| | | intracellular | TCCGGCCAGACCGACACCCTGGCCGCCTTCCCTGAAGAT |
| | | (short) | AGATCTCAACCTGGACAGGACTGCAGATTCAGAGTGACC |
| | | | CAGCTGCCCAACGGCAGAGACTTCCACATGAGCGTGGTG |
| | | | CGGGCCAGACGGAACGACAGCGGCACCTACGTGTGCGGC |
| | | | GTGATCAGCCTGGCTCCTAAGATCCAGATCAAGGAAAGC |
| | | | CTGAGAGCCGAGCTGCGGGTGACCGAGCGGAGAGCTGAG |
| | | | GTGCCTACAGCCCACCCTAGCCCATCTCCTAGACCTGCC |
| | | | GGCCAATTTCAGACACTGGTCGTGGAACCTGTGTCCTGC |
| | | | CCCGAGAAGGGCCTGGACATCTACCTGATCATCGGCATC |
| | | | TGCGGCGGCGGCAGCCTGCTGATGGTGTTCGTGGCCCTG |
| | | | CTGGTGTTCTACATCACCAAGAGAAAGAAGCAGACCCCT |
| | | | CAGAACCCCGCCACCAGCCAACACCCCCCCCCTCCACCA |
| | | | GGCCACAGAAGCCAGGCCCCTTCCCACCGCCCCCCCCCT |
| | | | CCAGGACATAGGGTTCAGCACCAGCCCCAGAAGCGGCCT |
| | | | CCTGCTCCTAGCGGAACACAGGTGCACCAGCAGAAAGGC |
| | | | CCTCCCCTCCCTAGACCCAGAGTGCAGCCTAAACCTCCC |
| | | | CACGGCGCCGCCGAGAACAGCCTGTCCCCTTCTAGCAAT |
| 132 | HA | HA PD1 ECD, | CCCGGCTGGTTCCTGGACAGCCCCGACAGACCTTGGAAT |
| | PD1.DAP10. | DAP10 ECD, | CCTCCAACCTTTAGCCCAGCCCTGCTGGTGGTGACAGAG |
| | D57N | DAP10 TM | GGAGATAACGCCACCTTCACCTGCAGCTTCAGCAACACC |
| | | D57N, DAP10 | AGCGAGAGCTTTCACGTGATCTGGCACCGGGAATCCCCA |
| | | intracellular | TCTGGCCAGACCGACACCCTGGCTGCCTTCCCCGAAGAT |
| | | | AGAAGCCAGCCTGGCCAAGACTGCAGATTCCGGGTGACA |
| | | | CAGCTGCCCAACGGCAGAGACTTCCACATGTCTGTGGTG |
| | | | CGGGCTAGACGGAACGACAGCGGCACCTACGTGTGCGGC |
| | | | GTGATTTCTCTGGCTCCTAAGATCCAGATCAAGGAATCC |
| | | | CTGCGGGCCGAGCTGAGAGTGACAGAGAGAAGGGCCGAG |
| | | | GTGCCTACCGCCCACCCCAGCCCAAGCCCTAGGCCCGCC |
| | | | GGCCAGTTCCAGACCCTGGTTCAGACCACACCTGGAGAA |
| | | | CGGAGCAGCCTCCCCGCCTTCTACCCCGGCACCAGCGGC |
| | | | AGCTGCAGCGGATGTGGCAGCCTGTCTCTGCCTCTGCTG |
| | | | GCCGGCCTGGTCGCCGCCAACGCCGTGGCTTCTCTGCTG |
| | | | ATCGTGGGCGCCGTGTTCCTGTGCGCCAGACCTAGACGG |
| | | | TCCCCAGCTCAGGAGGACGGCAAGGTGTACATCAACATG |
| | | | CCTGGCAGAGGC |
| 133 | HA | HA PD1 ECD, | CCCGGCTGGTTCCTGGACAGCCCCGACAGACCTTGGAAT |
| | PD1.ICOS | ICOS TM, ICOS | CCTCCAACCTTTAGCCCAGCCCTGCTGGTGGTGACAGAG |
| | | intracellular | GGAGATAACGCCACCTTCACCTGCAGCTTCAGCAACACC |
| | | | AGCGAGAGCTTTCACGTGATCTGGCACCGGGAATCCCCA |
| | | | TCTGGCCAGACCGACACCCTGGCTGCCTTCCCCGAAGAT |
| | | | AGAAGCCAGCCTGGCCAAGACTGCAGATTCCGGGTGACA |
| | | | CAGCTGCCCAACGGCAGAGACTTCCACATGTCTGTGGTG |
| | | | CGGGCTAGACGGAACGACAGCGGCACCTACGTGTGCGGC |
| | | | GTGATTTCTCTGGCTCCTAAGATCCAGATCAAGGAATCC |
| | | | CTGCGGGCCGAGCTGAGAGTGACAGAGAGAAGGGCCGAG |
| | | | GTGCCTACCGCCCACCCCAGCCCAAGCCCTAGGCCCGCC |
| | | | GGCCAGTTCCAGACCCTGGTTTTCTGGCTGCCTATCGGC |
| | | | TGCGCCGCTTTTGTGGTGGTCTGCATCCTGGGCTGTATC |
| | | | CTGATCTGCTGGCTGACCAAGAAGAAGTACAGCTCTTCC |
| | | | GTGCACGACCCCAACGGCGAGTACATGTTCATGCGGGCC |
| | | | GTGAACACCGCCAAGAAAAGCAGACTGACAGATGTGACC |
| | | | CTG |
| 134 | HA | HA PD1 ECD, | CCCGGCTGGTTCCTGGACAGCCCCGACAGACCTTGGAAT |
| | PD1.CD40 | CD40 TM, CD40 | CCTCCAACCTTTAGCCCAGCCCTGCTGGTGGTGACAGAG |
| | | intracellular | GGAGATAACGCCACCTTCACCTGCAGCTTCAGCAACACC |
| | | | AGCGAGAGCTTTCACGTGATCTGGCACCGGGAATCCCCA |
| | | | TCTGGCCAGACCGACACCCTGGCTGCCTTCCCCGAAGAT |
| | | | AGAAGCCAGCCTGGCCAAGACTGCAGATTCCGGGTGACA |
| | | | CAGCTGCCCAACGGCAGAGACTTCCACATGTCTGTGGTG |
| | | | CGGGCTAGACGGAACGACAGCGGCACCTACGTGTGCGGC |
| | | | GTGATTTCTCTGGCTCCTAAGATCCAGATCAAGGAATCC |
| | | | CTGCGGGCCGAGCTGAGAGTGACAGAGAGAAGGGCCGAG |
| | | | GTGCCTACCGCCCACCCCAGCCCAAGCCCTAGGCCCGCC |
| | | | GGCCAGTTCCAGACCCTGGTTGCCCTGGTGGTGATCCCC |
| | | | ATCATCTTCGGCATCCTGTTCGCCATTCTGCTGGTGCTG |
| | | | GTCTTTATCAAGAAGGTGGCCAAGAAACCTACAAACAAG |
| | | | GCCCCTCACCCCAAGCAGGAGCCTCAGGAGATCAACTTC |
| | | | CCCGACGACCTGCCTGGAAGCAATACCGCCGCTCCAGTG |
| | | | CAAGAAACCCTGCACGGCTGCCAGCCTGTGACCCAGGAA |
| | | | GATGGCAAAGAGTCTAGAATCAGCGTGCAGGAGCGGCAG |
| 135 | HA | HA PD1 ECD, | CCCGGCTGGTTCCTGGACAGCCCCGACAGACCTTGGAAT |
| | PD1.OX40 | OX40 TM, OX40 | CCTCCAACCTTTAGCCCAGCCCTGCTGGTGGTGACAGAG |
| | | intracellular | GGAGATAACGCCACCTTCACCTGCAGCTTCAGCAACACC |
| | | | AGCGAGAGCTTTCACGTGATCTGGCACCGGGAATCCCCA |
| | | | TCTGGCCAGACCGACACCCTGGCTGCCTTCCCCGAAGAT |
| | | | AGAAGCCAGCCTGGCCAAGACTGCAGATTCCGGGTGACA |
| | | | CAGCTGCCCAACGGCAGAGACTTCCACATGTCTGTGGTG |
| | | | CGGGCTAGACGGAACGACAGCGGCACCTACGTGTGCGGC |
| | | | GTGATTTCTCTGGCTCCTAAGATCCAGATCAAGGAATCC |
| | | | CTGCGGGCCGAGCTGAGAGTGACAGAGAGAAGGGCCGAG |
| | | | GTGCCTACCGCCCACCCCAGCCCAAGCCCTAGGCCCGCC |
| | | | GGCCAGTTCCAGACCCTGGTTGTGGCCGCCATCCTGGGC |
| | | | CTGGGCCTGGTGCTGGGACTGCTGGGCCCTCTGGCTATC |
| | | | CTGCTGGCCCTGTACCTGCTCAGACGGGACCAGAGACTG |
| | | | CCCCCCGACGCCCACAAGCCTCCAGGCGGCGGATCTTTC |
| | | | AGAACCCCTATCCAGGAGGAACAGGCCGATGCTCACAGC |
| | | | ACACTGGCCAAGATC |
| 136 | HA | HA PD1 ECD, | CCCGGCTGGTTCCTGGACAGCCCCGACAGACCTTGGAAT |
| | PD1.BAFFR | BAFFR TM, | CCTCCAACCTTTAGCCCAGCCCTGCTGGTGGTGACAGAG |
| | | BAFFR | GGAGATAACGCCACCTTCACCTGCAGCTTCAGCAACACC |
| | | intracellular | AGCGAGAGCTTTCACGTGATCTGGCACCGGGAATCCCCA |
| | | | TCTGGCCAGACCGACACCCTGGCTGCCTTCCCCGAAGAT |
| | | | AGAAGCCAGCCTGGCCAAGACTGCAGATTCCGGGTGACA |
| | | | CAGCTGCCCAACGGCAGAGACTTCCACATGTCTGTGGTG |
| | | | CGGGCTAGACGGAACGACAGCGGCACCTACGTGTGCGGC |
| | | | GTGATTTCTCTGGCTCCTAAGATCCAGATCAAGGAATCC |
| | | | CTGCGGGCCGAGCTGAGAGTGACAGAGAGAAGGGCCGAG |
| | | | GTGCCTACCGCCCACCCCAGCCCAAGCCCTAGGCCCGCC |
| | | | GGCCAGTTCCAGACCCTGGTTTTCGGCGCTCCCGCTCTG |
| | | | CTGGGCCTCGCCCTGGTGCTGGCCCTGGTCCTGGTGGGC |
| | | | CTGGTGTCCTGGCGGCGGAGACAGAGAAGACTGAGAGGC |
| | | | GCCAGCAGCGCCGAGGCCCCTGATGGCGATAAGGACGCC |
| | | | CCTGAGCCTCTGGACAAAGTGATCATCCTGAGCCCCGGC |
| | | | ATCAGCGACGCTACCGCCCCTGCCTGGCCTCCACCAGGC |
| | | | GAGGACCCCGGAACAACCCCTCCTGGCCACAGCGTGCCT |
| | | | GTGCCCGCCACCGAGCTGGGATCTACAGAACTGGTGACC |
| | | | ACAAAGACCGCCGGCCCTGAACAGCAG |
| 137 | HA | HA PD1 ECD, | CCTGGCTGGTTCCTGGACTCCCCTGACAGACCTTGGAAC |
| | PD1.tm. | PD1 TM, MyD88 | CCCCCCACCTTCAGCCCAGCCCTGCTGGTGGTCACCGAG |
| | MyD88 | | GGCGACAACGCTACATTCACCTGCAGCTTCAGCAACACC |
| | | | AGCGAGAGCTTCCACGTGATCTGGCACCGGGAATCTCCT |
| | | | TCTGGCCAGACAGACACCTTGGCAGCTTTTCCAGAGGAT |
| | | | AGAAGCCAGCCTGGCCAGGACTGCAGATTCAGAGTGACC |
| | | | CAGCTGCCCAACGGCCGGGACTTCCACATGAGCGTGGTG |
| | | | CGGGCCAGACGGAACGACAGCGGCACCTACGTGTGCGGC |
| | | | GTGATCTCTCTGGCCCCAAAGATCCAGATCAAGGAAAGC |
| | | | CTGAGAGCCGAACTGCGGGTGACGGAGAGAAGAGCCGAG |
| | | | GTGCCAACAGCCCACCCCAGCCCTTCCCCCAGACCCGCC |
| | | | GGACAATTTCAGACCCTGGTGGTCGGCGTGGTGGGCGGA |
| | | | CTGCTGGGCTCTCTGGTGCTGCTGGTGTGGGTGCTGGCC |
| | | | GTGATCTGCAGCAGAGCCGCTAGAGGAACAATCGGCGCC |
| | | | AGACGGACCGGCCAGATGGCTGCTGGAGGACCTGGCGCT |
| | | | GGCAGCGCCGCTCCTGTGTCCAGCACCAGCTCTCTGCCT |
| | | | CTGGCTGCACTTAATATGAGAGTGCGGCGGAGACTGAGC |
| | | | CTCTTCCTGAATGTGCGCACCCAAGTGGCAGCTGATTGG |
| | | | ACCGCCCTGGCCGAAGAGATGGACTTCGAGTACCTGGAA |
| | | | ATCAGACAGCTGGAAACCCAGGCCGACCCTACAGGCAGA |
| | | | CTGCTGGATGCCTGGCAGGGCAGACCGGGCGCCAGCGTT |
| | | | GGAAGGCTGCTGGACCTCCTGACCAAGCTGGGCCGGGAT |
| | | | GATGTGCTGCTGGAGCTGGGTCCTAGCATCGAGGAAGAT |
| | | | TGCCAGAAATACATCCTGAAACAGCAACAGGAGGAAGCC |
| | | | GAGAAGCCTCTGCAGGTGGCCGCCGTGGACAGCTCTGTG |
| | | | CCTAGAACAGCCGAGCTGGCCGGCATCACCACCCTGGAC |
| | | | GACCCCCTGGGCCACATGCCTGAGCGGTTCGACGCCTTT |
| | | | ATTTGTTATTGCCCCTCTGACATC |
| 138 | WT | WT BR2 ECD, | ACAATCCCCCCCCACGTGCAGAAGTCCGTGAACAATGAC |
| | BR2.CD28 | CD28 ECD | ATGATCGTCACCGACAACAACGGCGCTGTGAAGTTTCCA |
| | | (truncated), CD28 | CAACTGTGCAAGTTCTGCGACGTGCGGTTCAGCACATGC |
| | | TM, CD28 | GATAACCAGAAAAGCTGTATGAGCAATTGCTCCATTACA |
| | | intracellular | AGCATCTGTGAAAAACCTCAGGAGGTGTGCGTGGCCGTG |
| | | | TGGCGGAAGAACGACGAGAACATCACCCTGGAGACCGTG |
| | | | TGTCACGATCCTAAGCTGCCTTACCACGACTTCATCCTG |
| | | | GAAGATGCCGCCAGCCCTAAGTGCATCATGAAGGAAAAG |
| | | | AAAAAGCCTGGCGAGACCTTCTTCATGTGCTCTTGTTCT |
| | | | AGCGACGAGTGCAACGATAATATCATCTTCAGCGAGGAA |
| | | | TACAACACCAGCAACCCCGACCTGCTGCTCGTGATCTTT |
| | | | CAGTGTCCTAGCCCCCTGTTCCCCGGTCCTAGCAAACCT |
| | | | TTCTGGGTGCTGGTGGTGGTGGGCGGCGTGCTGGCCTGC |
| | | | TACAGCCTGCTGGTCACAGTGGCCTTTATCATCTTCTGG |
| | | | GTCAGATCTAAGCGGTCCAGACTGCTGCATTCTGATTAC |
| | | | ATGAACATGACCCCTAGAAGACCTGGACCTACAAGAAAG |
| | | | CACTACCAGCCTTACGCCCCTCCTCGGGACTTCGCCGCT |
| | | | TATAGAAGC |
| 139 | WT | WT BR2 ECD, | ACAATCCCCCCCCACGTGCAGAAGTCCGTGAACAATGAC |
| | BR2.CD28. | CD28 ECD | ATGATCGTCACCGACAACAACGGCGCTGTGAAGTTTCCA |
| | YMFM | (truncated), CD28 | CAACTGTGCAAGTTCTGCGACGTGCGGTTCAGCACATGC |
| | | TM, | GATAACCAGAAAAGCTGTATGAGCAATTGCTCCATTACA |
| | | CD28.YMFM | AGCATCTGTGAAAAACCTCAGGAGGTGTGCGTGGCCGTG |
| | | intracellular | TGGCGGAAGAACGACGAGAACATCACCCTGGAGACCGTG |
| | | | TGTCACGATCCTAAGCTGCCTTACCACGACTTCATCCTG |
| | | | GAAGATGCCGCCAGCCCTAAGTGCATCATGAAGGAAAAG |
| | | | AAAAAGCCTGGCGAGACCTTCTTCATGTGCTCTTGTTCT |
| | | | AGCGACGAGTGCAACGATAATATCATCTTCAGCGAGGAA |
| | | | TACAACACCAGCAACCCCGACCTGCTGCTCGTGATCTTT |
| | | | CAGTGTCCTAGCCCCCTGTTCCCCGGTCCTAGCAAACCT |
| | | | TTCTGGGTGCTGGTGGTGGTGGGCGGCGTGCTGGCCTGC |
| | | | TACAGCCTGCTGGTCACAGTGGCCTTTATCATCTTCTGG |
| | | | GTCAGATCTAAGCGGTCCAGACTGCTGCATTCTGATTAC |
| | | | ATGTTCATGACCCCTAGAAGACCTGGACCTACAAGAAAG |
| | | | CACTACCAGCCTTACGCCCCTCCTCGGGACTTCGCCGCT |
| | | | TATAGAAGC |
| 140 | WT | WT BR2 ECD, | ACAATCCCCCCCCACGTGCAGAAGTCCGTGAACAATGAC |
| | BR2.CD28. | CD28 ECD | ATGATCGTCACCGACAACAACGGCGCTGTGAAGTTTCCA |
| | AYAA | (truncated), CD28 | CAACTGTGCAAGTTCTGCGACGTGCGGTTCAGCACATGC |
| | | TM, | GATAACCAGAAAAGCTGTATGAGCAATTGCTCCATTACA |
| | | CD28.AYAA | AGCATCTGTGAAAAACCTCAGGAGGTGTGCGTGGCCGTG |
| | | intracellular | TGGCGGAAGAACGACGAGAACATCACCCTGGAGACCGTG |
| | | | TGTCACGATCCTAAGCTGCCTTACCACGACTTCATCCTG |
| | | | GAAGATGCCGCCAGCCCTAAGTGCATCATGAAGGAAAAG |
| | | | AAAAAGCCTGGCGAGACCTTCTTCATGTGCTCTTGTTCT |
| | | | AGCGACGAGTGCAACGATAATATCATCTTCAGCGAGGAA |
| | | | TACAACACCAGCAACCCCGACCTGCTGCTCGTGATCTTT |
| | | | CAGTGTCCTTCCCCTCTGTTCCCCGGCCCTAGCAAACCC |
| | | | TTCTGGGTGCTGGTGGTCGTGGGCGGAGTGCTGGCTTGC |
| | | | TACAGCCTGCTCGTGACCGTGGCCTTCATCATCTTCTGG |
| | | | GTCAGAAGCAAGCGGTCCAGACTGCTGCACAGCGACTAC |
| | | | ATGAACATGACACCTAGAAGACCTGGACCTACAAGAAAG |
| | | | CACTACCAGGCCTACGCCGCCCCTCGGGACTTCGCCGCT |
| | | | TATAGATCT |
| 141 | WT | WT BR2 ECD, | ACAATCCCCCCCCACGTGCAGAAGTCCGTGAACAATGAC |
| | BR2.CD2 | CD2 ECD | ATGATCGTCACCGACAACAACGGCGCTGTGAAGTTTCCA |
| | (full) | (truncated), CD2 | CAACTGTGCAAGTTCTGCGACGTGCGGTTCAGCACATGC |
| | | TM, CD2 | GATAACCAGAAAAGCTGTATGAGCAATTGCTCCATTACA |
| | | intracellular | AGCATCTGTGAAAAACCTCAGGAGGTGTGCGTGGCCGTG |
| | | | TGGCGGAAGAACGACGAGAACATCACCCTGGAGACCGTG |
| | | | TGTCACGATCCTAAGCTGCCTTACCACGACTTCATCCTG |
| | | | GAAGATGCCGCCAGCCCTAAGTGCATCATGAAGGAAAAG |
| | | | AAAAAGCCTGGCGAGACCTTCTTCATGTGCTCTTGTTCT |
| | | | AGCGACGAGTGCAACGATAATATCATCTTCAGCGAGGAA |
| | | | TACAACACCAGCAACCCCGACCTGCTGCTCGTGATCTTT |
| | | | CAGGTGGAGCCTGTGTCCTGTCCTGAGAAGGGACTGGAC |
| | | | ATCTACCTGATCATCGGCATCTGCGGAGGAGGCAGCCTG |
| | | | CTGATGGTGTTCGTGGCCCTGCTGGTGTTCTACATCACC |
| | | | AAGCGGAAGAAGCAGCGGAGCAGACGGAATGACGAGGAA |
| | | | CTCGAGACAAGAGCCCATCGGGTCGCCACAGAGGAAAGA |
| | | | GGCAGAAAGCCCCACCAGATTCCTGCCAGCACACCTCAG |
| | | | AACCCTGCTACCAGCCAACACCCCCCCCCCCCTCCTGGC |
| | | | CACAGATCTCAGGCCCCTAGCCACCGGCCCCCGCCACCT |
| | | | GGCCACCGGGTGCAGCACCAGCCTCAAAAAAGACCCCCT |
| | | | GCTCCTAGCGGCACACAGGTGCACCAGCAGAAAGGTCCT |
| | | | CCACTGCCTAGACCTCGGGTGCAGCCTAAGCCTCCACAT |
| | | | GGCGCCGCTGAGAACAGCTTGTCTCCTAGTTCTAAT |
| 142 | WT | WT BR2 ECD, | ACAATCCCCCCCCACGTGCAGAAGTCCGTGAACAATGAC |
| | BR2.CD2 | CD2 ECD | ATGATCGTCACCGACAACAACGGCGCTGTGAAGTTTCCA |
| | (short) | (truncated), CD2 | CAACTGTGCAAGTTCTGCGACGTGCGGTTCAGCACATGC |
| | | TM, CD2 | GATAACCAGAAAAGCTGTATGAGCAATTGCTCCATTACA |
| | | intracellular | AGCATCTGTGAAAAACCTCAGGAGGTGTGCGTGGCCGTG |
| | | (short) | TGGCGGAAGAACGACGAGAACATCACCCTGGAGACCGTG |
| | | | TGTCACGATCCTAAGCTGCCTTACCACGACTTCATCCTG |
| | | | GAAGATGCCGCCAGCCCTAAGTGCATCATGAAGGAAAAG |
| | | | AAAAAGCCTGGCGAGACCTTCTTCATGTGCTCTTGTTCT |
| | | | AGCGACGAGTGCAACGATAATATCATCTTCAGCGAGGAA |
| | | | TACAACACCAGCAACCCCGACCTGCTGCTCGTGATCTTT |
| | | | CAGGTCGAGCCTGTGAGCTGCCCCGAGAAGGGGCTGGAC |
| | | | ATCTACCTGATCATCGGCATTTGTGGCGGCGGATCTCTG |
| | | | CTGATGGTGTTCGTGGCCCTGCTGGTGTTCTACATCACC |
| | | | AAGAGAAAGAAGCAGACCCCTCAGAACCCCGCCACAAGC |
| | | | CAGCATCCTCCACCACCTCCCGGCCACCGGAGCCAGGCC |
| | | | CCAAGTCACAGACCCCCACCTCCTGGCCACAGAGTGCAG |
| | | | CACCAGCCCCAGAAGCGGCCTCCAGCTCCTAGCGGAACC |
| | | | CAAGTGCACCAGCAGAAAGGCCCTCCTCTGCCTCGGCCT |
| | | | AGAGTGCAGCCTAAACCTCCGCACGGCGCTGCTGAGAAC |
| | | | AGCTTGTCTCCCTCCAGCAAT |
| 143 | WT | WT BR2 ECD, | ACAATCCCCCCCCACGTGCAGAAGTCCGTGAACAATGAC |
| | BR2.DAP10. | DAP10 ECD, | ATGATCGTCACCGACAACAACGGCGCTGTGAAGTTTCCA |
| | D57N | DAP10 TM | CAACTGTGCAAGTTCTGCGACGTGCGGTTCAGCACATGC |
| | | | GATAACCAGAAAAGCTGTATGAGCAATTGCTCCATTACA |
| | | D57N, DAP10 | AGCATCTGTGAAAAACCTCAGGAGGTGTGCGTGGCCGTG |
| | | intracellular | TGGCGGAAGAACGACGAGAACATCACCCTGGAGACCGTG |
| | | | TGTCACGATCCTAAGCTGCCTTACCACGACTTCATCCTG |
| | | | GAAGATGCCGCCAGCCCTAAGTGCATCATGAAGGAAAAG |
| | | | AAAAAGCCTGGCGAGACCTTCTTCATGTGCTCTTGTTCT |
| | | | AGCGACGAGTGCAACGATAATATCATCTTCAGCGAGGAA |
| | | | TACAACACCAGCAACCCCGACCTGCTGCTCGTGATCTTT |
| | | | CAGCAGACCACACCTGGAGAACGGAGCAGCCTCCCCGCC |
| | | | TTCTACCCCGGCACCAGCGGCAGCTGCAGCGGATGTGGC |
| | | | AGCCTGTCTCTGCCTCTGCTGGCCGGCCTGGTCGCCGCC |
| | | | AACGCCGTGGCTTCTCTGCTGATCGTGGGCGCCGTGTTC |
| | | | CTGTGCGCCAGACCTAGACGGTCCCCAGCTCAGGAGGAC |
| | | | GGCAAGGTGTACATCAACATGCCTGGCAGAGGC |
| 144 | WT | WT BR2 ECD, | ACAATCCCCCCCCACGTGCAGAAGTCCGTGAACAATGAC |
| | BR2.ICOS | ICOS TM, ICOS | ATGATCGTCACCGACAACAACGGCGCTGTGAAGTTTCCA |
| | | intracellular | CAACTGTGCAAGTTCTGCGACGTGCGGTTCAGCACATGC |
| | | | GATAACCAGAAAAGCTGTATGAGCAATTGCTCCATTACA |
| | | | AGCATCTGTGAAAAACCTCAGGAGGTGTGCGTGGCCGTG |
| | | | TGGCGGAAGAACGACGAGAACATCACCCTGGAGACCGTG |
| | | | TGTCACGATCCTAAGCTGCCTTACCACGACTTCATCCTG |
| | | | GAAGATGCCGCCAGCCCTAAGTGCATCATGAAGGAAAAG |
| | | | AAAAAGCCTGGCGAGACCTTCTTCATGTGCTCTTGTTCT |
| | | | AGCGACGAGTGCAACGATAATATCATCTTCAGCGAGGAA |
| | | | TACAACACCAGCAACCCCGACCTGCTGCTCGTGATCTTT |
| | | | CAGTTCTGGCTGCCTATCGGCTGCGCCGCTTTTGTGGTG |
| | | | GTCTGCATCCTGGGCTGTATCCTGATCTGCTGGCTGACC |
| | | | AAGAAGAAGTACAGCTCTTCCGTGCACGACCCCAACGGC |
| | | | GAGTACATGTTCATGCGGGCCGTGAACACCGCCAAGAAA |
| | | | AGCAGACTGACAGATGTGACCCTG |
| 145 | WT | WT BR2 ECD, | ACAATCCCCCCCCACGTGCAGAAGTCCGTGAACAATGAC |
| | BR2.CD40 | CD40 TM, CD40 | ATGATCGTCACCGACAACAACGGCGCTGTGAAGTTTCCA |
| | | intracellular | CAACTGTGCAAGTTCTGCGACGTGCGGTTCAGCACATGC |
| | | | GATAACCAGAAAAGCTGTATGAGCAATTGCTCCATTACA |
| | | | AGCATCTGTGAAAAACCTCAGGAGGTGTGCGTGGCCGTG |
| | | | TGGCGGAAGAACGACGAGAACATCACCCTGGAGACCGTG |
| | | | TGTCACGATCCTAAGCTGCCTTACCACGACTTCATCCTG |
| | | | GAAGATGCCGCCAGCCCTAAGTGCATCATGAAGGAAAAG |
| | | | AAAAAGCCTGGCGAGACCTTCTTCATGTGCTCTTGTTCT |
| | | | AGCGACGAGTGCAACGATAATATCATCTTCAGCGAGGAA |
| | | | TACAACACCAGCAACCCCGACCTGCTGCTCGTGATCTTT |
| | | | CAGGCCCTGGTGGTGATCCCCATCATCTTCGGCATCCTG |
| | | | TTCGCCATTCTGCTGGTGCTGGTCTTTATCAAGAAGGTG |
| | | | GCCAAGAAACCTACAAACAAGGCCCCTCACCCCAAGCAG |
| | | | GAGCCTCAGGAGATCAACTTCCCCGACGACCTGCCTGGA |
| | | | AGCAATACCGCCGCTCCAGTGCAAGAAACCCTGCACGGC |
| | | | TGCCAGCCTGTGACCCAGGAAGATGGCAAAGAGTCTAGA |
| | | | ATCAGCGTGCAGGAGCGGCAG |
| 146 | WT | WT BR2 ECD, | ACAATCCCCCCCCACGTGCAGAAGTCCGTGAACAATGAC |
| | BR2.OX40 | OX40 TM, OX40 | ATGATCGTCACCGACAACAACGGCGCTGTGAAGTTTCCA |
| | | intracellular | CAACTGTGCAAGTTCTGCGACGTGCGGTTCAGCACATGC |
| | | | GATAACCAGAAAAGCTGTATGAGCAATTGCTCCATTACA |
| | | | AGCATCTGTGAAAAACCTCAGGAGGTGTGCGTGGCCGTG |
| | | | TGGCGGAAGAACGACGAGAACATCACCCTGGAGACCGTG |
| | | | TGTCACGATCCTAAGCTGCCTTACCACGACTTCATCCTG |
| | | | GAAGATGCCGCCAGCCCTAAGTGCATCATGAAGGAAAAG |
| | | | AAAAAGCCTGGCGAGACCTTCTTCATGTGCTCTTGTTCT |
| | | | AGCGACGAGTGCAACGATAATATCATCTTCAGCGAGGAA |
| | | | TACAACACCAGCAACCCCGACCTGCTGCTCGTGATCTTT |
| | | | CAGGTGGCCGCCATCCTGGGCCTGGGCCTGGTGCTGGGA |
| | | | CTGCTGGGCCCTCTGGCTATCCTGCTGGCCCTGTACCTG |
| | | | CTCAGACGGGACCAGAGACTGCCCCCCGACGCCCACAAG |
| | | | CCTCCAGGCGGCGGATCTTTCAGAACCCCTATCCAGGAG |
| | | | GAACAGGCCGATGCTCACAGCACACTGGCCAAGATC |
| 147 | WT | WT BR2 ECD, | ACAATCCCCCCCCACGTGCAGAAGTCCGTGAACAATGAC |
| | BR2.PD1tm. | PD1 TM, MyD88 | ATGATCGTCACCGACAACAACGGCGCTGTGAAGTTTCCA |
| | MyD88 | | CAACTGTGCAAGTTCTGCGACGTGCGGTTCAGCACATGC |
| | | | GATAACCAGAAAAGCTGTATGAGCAATTGCTCCATTACA |
| | | | AGCATCTGTGAAAAACCTCAGGAGGTGTGCGTGGCCGTG |
| | | | TGGCGGAAGAACGACGAGAACATCACCCTGGAGACCGTG |
| | | | TGTCACGATCCTAAGCTGCCTTACCACGACTTCATCCTG |
| | | | GAAGATGCCGCCAGCCCTAAGTGCATCATGAAGGAAAAG |
| | | | AAAAAGCCTGGCGAGACCTTCTTCATGTGCTCTTGTTCT |
| | | | AGCGACGAGTGCAACGATAATATCATCTTCAGCGAGGAA |
| | | | TACAACACCAGCAACCCCGACCTGCTGCTCGTGATCTTT |
| | | | CAGGTCGGCGTGGTGGGCGGACTGCTGGGCTCTCTGGTG |
| | | | CTGCTGGTGTGGGTGCTGGCCGTGATCTGCAGCAGAGCC |
| | | | GCTAGAGGAACAATCGGCGCCAGACGGACCGGCCAGATG |
| | | | GCCGCCGGAGGCCCTGGCGCTGGAAGCGCCGCACCTGTG |
| | | | TCCTCTACATCTAGTCTGCCTCTGGCCGCTCTTAATATG |
| | | | AGAGTGCGGAGAAGACTGAGCCTGTTCCTGAACGTGCGC |
| | | | ACACAAGTGGCCGCTGATTGGACTGCCCTGGCTGAAGAG |
| | | | ATGGACTTCGAGTACCTGGAAATCAGACAGCTGGAAACC |
| | | | CAGGCCGACCCCACAGGCCGGCTGCTGGACGCCTGGCAG |
| | | | GGCAGACCTGGAGCCAGCGTGGGCAGACTGCTGGACCTG |
| | | | CTGACCAAGCTGGGACGGGACGACGTGCTGCTGGAACTG |
| | | | GGCCCCTCTATTGAGGAAGATTGCCAGAAATACATCCTG |
| | | | AAACAGCAGCAGGAGGAGGCCGAAAAGCCTCTGCAGGTG |
| | | | GCCGCCGTGGACAGCAGCGTGCCCAGAACCGCCGAGCTG |
| | | | GCTGGCATCACCACACTGGATGATCCTCTGGGCCACATG |
| | | | CCTGAAAGATTCGACGCCTTCATCTGCTACTGTCCTAGC |
| | | | GACATC |
| 148 | dN25 | dN BR2 ECD, | CAGCTGTGCAAGTTCTGCGACGTGCGGTTCAGCACCTGT |
| | BR2.CD28 | CD28 ECD | GATAACCAGAAAAGCTGTATGAGCAATTGCTCTATCACC |
| | | (truncated), CD28 | TCCATCTGCGAGAAGCCTCAGGAGGTGTGCGTGGCCGTG |
| | | TM, CD28 | TGGCGGAAGAACGACGAGAACATTACACTGGAAACCGTG |
| | | intracellular | TGTCACGATCCTAAGCTGCCTTACCACGACTTCATCCTG |
| | | | GAAGATGCCGCCAGCCCTAAGTGCATCATGAAGGAAAAG |
| | | | AAAAAGCCAGGCGAGACATTTTTCATGTGCTCCTGTAGC |
| | | | AGCGACGAGTGCAACGACAATATCATCTTTAGCGAGGAA |
| | | | TACAACACCAGCAACCCCGACCTGCTCCTGGTCATCTTC |
| | | | CAATGTCCTAGCCCCCTGTTCCCCGGTCCTAGCAAACCT |
| | | | TTCTGGGTGCTGGTGGTGGTGGGCGGCGTGCTGGCCTGC |
| | | | TACAGCCTGCTGGTCACAGTGGCCTTTATCATCTTCTGG |
| | | | GTCAGATCTAAGCGGTCCAGACTGCTGCATTCTGATTAC |
| | | | ATGAACATGACCCCTAGAAGACCTGGACCTACAAGAAAG |
| | | | CACTACCAGCCTTACGCCCCTCCTCGGGACTTCGCCGCT |
| | | | TATAGAAGC |
| 149 | dN25 | dN25 BR2 ECD, | CAGCTGTGCAAGTTCTGCGACGTGCGGTTCAGCACCTGT |
| | BR2.CD28. | CD28 ECD | GATAACCAGAAAAGCTGTATGAGCAATTGCTCTATCACC |
| | YMFM | (truncated), CD28 | TCCATCTGCGAGAAGCCTCAGGAGGTGTGCGTGGCCGTG |
| | | TM, | TGGCGGAAGAACGACGAGAACATTACACTGGAAACCGTG |
| | | CD28.YMFM | TGTCACGATCCTAAGCTGCCTTACCACGACTTCATCCTG |
| | | intracellular | GAAGATGCCGCCAGCCCTAAGTGCATCATGAAGGAAAAG |
| | | | AAAAAGCCAGGCGAGACATTTTTCATGTGCTCCTGTAGC |
| | | | AGCGACGAGTGCAACGACAATATCATCTTTAGCGAGGAA |
| | | | TACAACACCAGCAACCCCGACCTGCTCCTGGTCATCTTC |
| | | | CAATGTCCTAGCCCCCTGTTCCCCGGTCCTAGCAAACCT |
| | | | TTCTGGGTGCTGGTGGTGGTGGGCGGCGTGCTGGCCTGC |
| | | | TACAGCCTGCTGGTCACAGTGGCCTTTATCATCTTCTGG |
| | | | GTCAGATCTAAGCGGTCCAGACTGCTGCATTCTGATTAC |
| | | | ATGTTCATGACCCCTAGAAGACCTGGACCTACAAGAAAG |
| | | | CACTACCAGCCTTACGCCCCTCCTCGGGACTTCGCCGCT |
| | | | TATAGAAGC |
| 150 | dN25 | dN25 BR2 ECD, | CAGCTGTGCAAGTTCTGCGACGTGCGGTTCAGCACCTGT |
| | BR2.CD28. | CD28 ECD | GATAACCAGAAAAGCTGTATGAGCAATTGCTCTATCACC |
| | AYAA | (truncated), CD28 | TCCATCTGCGAGAAGCCTCAGGAGGTGTGCGTGGCCGTG |
| | | TM, | TGGCGGAAGAACGACGAGAACATTACACTGGAAACCGTG |
| | | CD28.AYAA | TGTCACGATCCTAAGCTGCCTTACCACGACTTCATCCTG |
| | | intracellular | GAAGATGCCGCCAGCCCTAAGTGCATCATGAAGGAAAAG |
| | | | AAAAAGCCAGGCGAGACATTTTTCATGTGCTCCTGTAGC |
| | | | AGCGACGAGTGCAACGACAATATCATCTTTAGCGAGGAA |
| | | | TACAACACCAGCAACCCCGACCTGCTCCTGGTCATCTTC |
| | | | CAATGTCCTAGCCCCCTGTTCCCCGGTCCTAGCAAACCT |
| | | | TTCTGGGTGCTGGTGGTGGTGGGCGGCGTGCTGGCCTGC |
| | | | TACAGCCTGCTGGTCACAGTGGCCTTTATCATCTTCTGG |
| | | | GTCAGATCTAAGCGGTCCAGACTGCTGCATTCTGATTAC |
| | | | ATGAACATGACCCCTAGAAGACCTGGACCTACAAGAAAG |
| | | | CACTACCAGGCCTACGCCGCCCCTCGGGACTTCGCCGCT |
| | | | TATAGAAGC |
| 151 | dN25 | dN25 BR2 ECD, | CAGCTGTGCAAGTTCTGCGACGTGCGGTTCAGCACCTGT |
| | BR2.CD2 | CD2 ECD | GATAACCAGAAAAGCTGTATGAGCAATTGCTCTATCACC |
| | (full) | (truncated), CD2 | TCCATCTGCGAGAAGCCTCAGGAGGTGTGCGTGGCCGTG |
| | | TM, CD2 | TGGCGGAAGAACGACGAGAACATTACACTGGAAACCGTG |
| | | intracellular | TGTCACGATCCTAAGCTGCCTTACCACGACTTCATCCTG |
| | | | GAAGATGCCGCCAGCCCTAAGTGCATCATGAAGGAAAAG |
| | | | AAAAAGCCAGGCGAGACATTTTTCATGTGCTCCTGTAGC |
| | | | AGCGACGAGTGCAACGACAATATCATCTTTAGCGAGGAA |
| | | | TACAACACCAGCAACCCCGACCTGCTCCTGGTCATCTTC |
| | | | CAAGTGGAGCCTGTGTCCTGTCCTGAGAAGGGACTGGAC |
| | | | ATCTACCTGATCATCGGCATCTGCGGAGGAGGCAGCCTG |
| | | | CTGATGGTGTTCGTGGCCCTGCTGGTGTTCTACATCACC |
| | | | AAGCGGAAGAAGCAGCGGAGCAGACGGAATGACGAGGAA |
| | | | CTCGAGACAAGAGCCCATCGGGTCGCCACAGAGGAAAGA |
| | | | GGCAGAAAGCCCCACCAGATTCCTGCCAGCACACCTCAG |
| | | | AACCCTGCTACCAGCCAACACCCCCCCCCCCCTCCTGGC |
| | | | CACAGATCTCAGGCCCCTAGCCACCGGCCCCCGCCACCT |
| | | | GGCCACCGGGTGCAGCACCAGCCTCAAAAAAGACCCCCT |
| | | | GCTCCTAGCGGCACACAGGTGCACCAGCAGAAAGGTCCT |
| | | | CCACTGCCTAGACCTCGGGTGCAGCCTAAGCCTCCACAT |
| | | | GGCGCCGCTGAGAACAGCTTGTCTCCTAGTTCTAAT |
| 152 | dN25 | dN25 BR2 ECD, | CAGCTGTGCAAGTTCTGCGACGTGCGGTTCAGCACCTGT |
| | BR2.CD2 | CD2 ECD | GATAACCAGAAAAGCTGTATGAGCAATTGCTCTATCACC |
| | (short) | (truncated), CD2 | TCCATCTGCGAGAAGCCTCAGGAGGTGTGCGTGGCCGTG |
| | | TM, CD2 | TGGCGGAAGAACGACGAGAACATTACACTGGAAACCGTG |
| | | intracellular | TGTCACGATCCTAAGCTGCCTTACCACGACTTCATCCTG |
| | | (short) | GAAGATGCCGCCAGCCCTAAGTGCATCATGAAGGAAAAG |
| | | | AAAAAGCCAGGCGAGACATTTTTCATGTGCTCCTGTAGC |
| | | | AGCGACGAGTGCAACGACAATATCATCTTTAGCGAGGAA |
| | | | TACAACACCAGCAACCCCGACCTGCTCCTGGTCATCTTC |
| | | | CAAGTCGAGCCTGTGAGCTGCCCCGAGAAGGGGCTGGAC |
| | | | ATCTACCTGATCATCGGCATTTGTGGCGGCGGATCTCTG |
| | | | CTGATGGTGTTCGTGGCCCTGCTGGTGTTCTACATCACC |
| | | | AAGAGAAAGAAGCAGACCCCTCAGAACCCCGCCACAAGC |
| | | | CAGCATCCTCCACCACCTCCCGGCCACCGGAGCCAGGCC |
| | | | CCAAGTCACAGACCCCCACCTCCTGGCCACAGAGTGCAG |
| | | | CACCAGCCCCAGAAGCGGCCTCCAGCTCCTAGCGGAACC |
| | | | CAAGTGCACCAGCAGAAAGGCCCTCCTCTGCCTCGGCCT |
| | | | AGAGTGCAGCCTAAACCTCCGCACGGCGCTGCTGAGAAC |
| | | | AGCTTGTCTCCCTCCAGCAAT |
| 153 | dN25 | dN25 BR2 ECD, | CAGCTGTGCAAGTTCTGCGACGTGCGGTTCAGCACCTGT |
| | BR2.DAP10. | DAP10 ECD, | GATAACCAGAAAAGCTGTATGAGCAATTGCTCTATCACC |
| | D57N | DAP10 TM | TCCATCTGCGAGAAGCCTCAGGAGGTGTGCGTGGCCGTG |
| | | D57N, DAP10 | TGGCGGAAGAACGACGAGAACATTACACTGGAAACCGTG |
| | | intracellular | TGTCACGATCCTAAGCTGCCTTACCACGACTTCATCCTG |
| | | | GAAGATGCCGCCAGCCCTAAGTGCATCATGAAGGAAAAG |
| | | | AAAAAGCCAGGCGAGACATTTTTCATGTGCTCCTGTAGC |
| | | | AGCGACGAGTGCAACGACAATATCATCTTTAGCGAGGAA |
| | | | TACAACACCAGCAACCCCGACCTGCTCCTGGTCATCTTC |
| | | | CAACAGACCACACCTGGAGAACGGAGCAGCCTCCCCGCC |
| | | | TTCTACCCCGGCACCAGCGGCAGCTGCAGCGGATGTGGC |
| | | | AGCCTGTCTCTGCCTCTGCTGGCCGGCCTGGTCGCCGCC |
| | | | AACGCCGTGGCTTCTCTGCTGATCGTGGGCGCCGTGTTC |
| | | | CTGTGCGCCAGACCTAGACGGTCCCCAGCTCAGGAGGAC |
| | | | GGCAAGGTGTACATCAACATGCCTGGCAGAGGC |
| 154 | dN25 | dN25 BR2 ECD, | CAGCTGTGCAAGTTCTGCGACGTGCGGTTCAGCACCTGT |
| | BR2.ICOS | ICOS TM, ICOS | GATAACCAGAAAAGCTGTATGAGCAATTGCTCTATCACC |
| | | intracellular | TCCATCTGCGAGAAGCCTCAGGAGGTGTGCGTGGCCGTG |
| | | | TGGCGGAAGAACGACGAGAACATTACACTGGAAACCGTG |
| | | | TGTCACGATCCTAAGCTGCCTTACCACGACTTCATCCTG |
| | | | GAAGATGCCGCCAGCCCTAAGTGCATCATGAAGGAAAAG |
| | | | AAAAAGCCAGGCGAGACATTTTTCATGTGCTCCTGTAGC |
| | | | AGCGACGAGTGCAACGACAATATCATCTTTAGCGAGGAA |
| | | | TACAACACCAGCAACCCCGACCTGCTCCTGGTCATCTTC |
| | | | CAATTCTGGCTGCCTATCGGCTGCGCCGCTTTTGTGGTG |
| | | | GTCTGCATCCTGGGCTGTATCCTGATCTGCTGGCTGACC |
| | | | AAGAAGAAGTACAGCTCTTCCGTGCACGACCCCAACGGC |
| | | | GAGTACATGTTCATGCGGGCCGTGAACACCGCCAAGAAA |
| | | | AGCAGACTGACAGATGTGACCCTG |
| 155 | dN25 | dN25 BR2 ECD, | CAGCTGTGCAAGTTCTGCGACGTGCGGTTCAGCACCTGT |
| | BR2.CD40 | CD40 TM, CD40 | GATAACCAGAAAAGCTGTATGAGCAATTGCTCTATCACC |
| | | intracellular | TCCATCTGCGAGAAGCCTCAGGAGGTGTGCGTGGCCGTG |
| | | | TGGCGGAAGAACGACGAGAACATTACACTGGAAACCGTG |
| | | | TGTCACGATCCTAAGCTGCCTTACCACGACTTCATCCTG |
| | | | GAAGATGCCGCCAGCCCTAAGTGCATCATGAAGGAAAAG |
| | | | AAAAAGCCAGGCGAGACATTTTTCATGTGCTCCTGTAGC |
| | | | AGCGACGAGTGCAACGACAATATCATCTTTAGCGAGGAA |
| | | | TACAACACCAGCAACCCCGACCTGCTCCTGGTCATCTTC |
| | | | CAAGCCCTGGTGGTGATCCCCATCATCTTCGGCATCCTG |
| | | | TTCGCCATTCTGCTGGTGCTGGTCTTTATCAAGAAGGTG |
| | | | GCCAAGAAACCTACAAACAAGGCCCCTCACCCCAAGCAG |
| | | | GAGCCTCAGGAGATCAACTTCCCCGACGACCTGCCTGGA |
| | | | AGCAATACCGCCGCTCCAGTGCAAGAAACCCTGCACGGC |
| | | | TGCCAGCCTGTGACCCAGGAAGATGGCAAAGAGTCTAGA |
| | | | ATCAGCGTGCAGGAGCGGCAG |
| 156 | dN25 | dN25 BR2 ECD, | CAGCTGTGCAAGTTCTGCGACGTGCGGTTCAGCACCTGT |
| | BR2.OX40 | OX40 TM, OX40 | GATAACCAGAAAAGCTGTATGAGCAATTGCTCTATCACC |
| | | intracellular | TCCATCTGCGAGAAGCCTCAGGAGGTGTGCGTGGCCGTG |
| | | | TGGCGGAAGAACGACGAGAACATTACACTGGAAACCGTG |
| | | | TGTCACGATCCTAAGCTGCCTTACCACGACTTCATCCTG |
| | | | GAAGATGCCGCCAGCCCTAAGTGCATCATGAAGGAAAAG |
| | | | AAAAAGCCAGGCGAGACATTTTTCATGTGCTCCTGTAGC |
| | | | AGCGACGAGTGCAACGACAATATCATCTTTAGCGAGGAA |
| | | | TACAACACCAGCAACCCCGACCTGCTCCTGGTCATCTTC |
| | | | CAAGTGGCCGCCATCCTGGGCCTGGGCCTGGTGCTGGGA |
| | | | CTGCTGGGCCCTCTGGCTATCCTGCTGGCCCTGTACCTG |
| | | | CTCAGACGGGACCAGAGACTGCCCCCCGACGCCCACAAG |
| | | | CCTCCAGGCGGCGGATCTTTCAGAACCCCTATCCAGGAG |
| | | | GAACAGGCCGATGCTCACAGCACACTGGCCAAGATC |
| 157 | dN25 | dN25 BR2 ECD, | CAGCTGTGCAAGTTCTGCGACGTGCGGTTCAGCACCTGT |
| | BR2.BAFFR | BAFFR | GATAACCAGAAAAGCTGTATGAGCAATTGCTCTATCACC |
| | | intracellular | TCCATCTGCGAGAAGCCTCAGGAGGTGTGCGTGGCCGTG |
| | | dN25 BR2 ECD, | TGGCGGAAGAACGACGAGAACATTACACTGGAAACCGTG |
| | | PD1 TM, MyD88 | TGTCACGATCCTAAGCTGCCTTACCACGACTTCATCCTG |
| | | | GAAGATGCCGCCAGCCCTAAGTGCATCATGAAGGAAAAG |
| | | | AAAAAGCCAGGCGAGACATTTTTCATGTGCTCCTGTAGC |
| | | | AGCGACGAGTGCAACGACAATATCATCTTTAGCGAGGAA |
| | | | TACAACACCAGCAACCCCGACCTGCTCCTGGTCATCTTC |
| | | | CAATTCGGCGCTCCCGCTCTGCTGGGCCTCGCCCTGGTG |
| | | | CTGGCCCTGGTCCTGGTGGGCCTGGTGTCCTGGCGGCGG |
| | | | AGACAGAGAAGACTGAGAGGCGCCAGCAGCGCCGAGGCC |
| | | | CCTGATGGCGATAAGGACGCCCCTGAGCCTCTGGACAAA |
| | | | GTGATCATCCTGAGCCCCGGCATCAGCGACGCTACCGCC |
| | | | CCTGCCTGGCCTCCACCAGGCGAGGACCCCGGAACAACC |
| | | | CCTCCTGGCCACAGCGTGCCTGTGCCCGCCACCGAGCTG |
| | | | GGATCTACAGAACTGGTGACCACAAAGACCGCCGGCCCT |
| | | | GAACAGCAG |
| 158 | dN25 | BAFFR TM, | CAGCTGTGCAAGTTCTGCGACGTGCGGTTCAGCACCTGT |
| | BR2.PD1tm. | | GATAACCAGAAAAGCTGTATGAGCAATTGCTCTATCACC |
| | MyD88 | | TCCATCTGCGAGAAGCCTCAGGAGGTGTGCGTGGCCGTG |
| | | | TGGCGGAAGAACGACGAGAACATTACACTGGAAACCGTG |
| | | | TGTCACGATCCTAAGCTGCCTTACCACGACTTCATCCTG |
| | | | GAAGATGCCGCCAGCCCTAAGTGCATCATGAAGGAAAAG |
| | | | AAAAAGCCAGGCGAGACATTTTTCATGTGCTCCTGTAGC |
| | | | AGCGACGAGTGCAACGACAATATCATCTTTAGCGAGGAA |
| | | | TACAACACCAGCAACCCCGACCTGCTCCTGGTCATCTTC |
| | | | CAAGTCGGCGTGGTGGGCGGACTGCTGGGCTCTCTGGTG |
| | | | CTGCTGGTGTGGGTGCTGGCCGTGATCTGCAGCAGAGCC |
| | | | GCTAGAGGAACAATCGGCGCCAGACGGACCGGCCAGATG |
| | | | GCCGCCGGAGGCCCTGGCGCTGGAAGCGCCGCACCTGTG |
| | | | TCCTCTACATCTAGTCTGCCTCTGGCCGCTCTTAATATG |
| | | | AGAGTGCGGAGAAGACTGAGCCTGTTCCTGAACGTGCGC |
| | | | ACACAAGTGGCCGCTGATTGGACTGCCCTGGCTGAAGAG |
| | | | ATGGACTTCGAGTACCTGGAAATCAGACAGCTGGAAACC |
| | | | CAGGCCGACCCCACAGGCCGGCTGCTGGACGCCTGGCAG |
| | | | GGCAGACCTGGAGCCAGCGTGGGCAGACTGCTGGACCTG |
| | | | CTGACCAAGCTGGGACGGGACGACGTGCTGCTGGAACTG |
| | | | GGCCCCTCTATTGAGGAAGATTGCCAGAAATACATCCTG |
| | | | AAACAGCAGCAGGAGGAGGCCGAAAAGCCTCTGCAGGTG |
| | | | GCCGCCGTGGACAGCAGCGTGCCCAGAACCGCCGAGCTG |
| | | | GCTGGCATCACCACACTGGATGATCCTCTGGGCCACATG |
| | | | CCTGAAAGATTCGACGCCTTCATCTGCTACTGTCCTAGC |
| | | | GACATC |
| 160 | CCR 15.1 | TPOR TM, TPOR | TCAGACCCTACTAGAGTCGAGACCGCTACCGAGACCGCT |
| | | JAK-binding | TGGATCTCTCTGGTGACCGCCCTGCACCTGGTGCTGGGC |
| | | domain, IL2RbYY | CTGAACGCCGTGCTGGGCCTGCTGCTGCTGAGGAAGCAG |
| | | signaling domain | TTCCCAGCACACTACCGGAGACTGAGGCACGCACTGTGG |
| | | | CCAAGCCTGCCCGACCTGCACAGGGTGCTGGGACAGTAT |
| | | | CTGAGGGATACAGCCGCCCTGAGCCCACCTAAGGCAACC |
| | | | GTGTCCGACACATGCGAGGAGGTGGAACCAAGTCTGCTG |
| | | | GAAATCCTGCCAAAATCCTCTGAGCGGACACCCCTGCCC |
| | | | CTGCTCGAGGACGAGGGAGTGGCAGGAGCACCAACCGGC |
| | | | AGCTCCCCCCAGCCTCTGCAGCCACTGTCCGGAGAGGAC |
| | | | GATGCATACTGCACATTCCCTTCTCGGGACGATCTGCTG |
| | | | CTGTTCTCTCCAAGCGGACAGGGAGAGTTTCGGGCCCTG |
| | | | AACGCCAGACTGCCCCTGAATACCGACGCCTATCTGAGC |
| | | | CTGCAGGAGCTGCAGGGACAGGACCCCACACACCTGGTG |
| 161 | dnHACi | CD8 signal | ATGGCCCTGCCAGTGACCGCCCTGCTGCTGCCACTGGCC |
| | PD1 | sequence | CTGCTGCTGCACGCAGCAAGGCCACCTGGATGGTTTCTG |
| | | (underlined), HA | GACTCCCCTGATAGGCCCTGGAATCCCCCAACTTTCTCC |
| | | PD1 ECD, PD1 TM | CCTGCCCTGCTGGTGGTCACTGAAGGCGACAACGCCACC |
| | | | TTCACATGCAGCTTTTCCAACACCTCTGAGAGCTTCCAC |
| | | | GTGATCTGGCACAGGGAGTCCCCATCTGGCCAGACCGAC |
| | | | ACACTGGCAGCATTTCCTGAGGACCGCTCCCAGCCAGGA |
| | | | CAGGATTGCCGGTTCAGAGTGACCCAGCTGCCCAACGGC |
| | | | CGGGACTTTCACATGTCTGTGGTGAGAGCCCGGAGAAAT |
| | | | GATAGCGGCACCTACGTGTGCGGCGTGATCTCCCTGGCC |
| | | | CCCAAGATCCAGATCAAGGAGTCTCTGAGGGCAGAGCTG |
| | | | AGGGTGACCGAGAGGAGGGCAGAGGTGCCTACAGCACAC |
| | | | CCAAGCCCTTCCCCACGGCCCGCAGGACAGTTCCAGACA |
| | | | CTGGTGGTGGGAGTGGTGGGAGGCCTGCTGGGCAGCCTG |
| | | | GTGCTGCTGGTGTGGGTGCTGGCTGTCATCTGTAGCAGG |
| | | | GCCGCAAGAGGCACCATTGGGGCACGAAGGACTGGGCAG |
| 167 | CCR 15.3 | TPOR TM, TPOR | TCAGACCCTACTAGAGTCGAGACCGCTACCGAGACCGCT |
| | | JAK-binding | TGGATCTCTCTGGTGACCGCCCTGCTGCTGGTGCTGGGC |
| | | domain, | CTGAACGCCGTGCTGGGCCTGCTGCTGCTGAGGAAGCAG |
| | | IL2RbYYY | TTCCCAGCACACTACCGGAGACTGAGGCACGCACTGTGG |
| | | signaling domain | CCAAGCCTGCCCGACCTGCACAGGGTGCTGGGACAGTAT |
| | | | CTGAGGGATACAGCCGCCCTGAGCCCACCTAAGGCAACC |
| | | | GTGTCCGACACATGCGAGGAGGTGGAACCAAGTCTGCTG |
| | | | GAAATCCTGCCAAAATCCTCTGAGCGGACACCCCTGCCC |
| | | | CTGCTCGAGCAGCAGGACAAGGTGCCCGAGCCTGCCTCC |
| | | | CTGAGCTCCAACCACAGCCTGACCTCCTGCTTTACAAAT |
| | | | CAGGGCTACTTCTTTTTCCACCTGCCTGACGCCCTGGAG |
| | | | ATCGAGGCCTGTCAGGATGAGGGAGTGGCAGGAGCACCT |
| | | | ACCGGCTCTAGCCCACAGCCACTGCAGCCACTGTCTGGA |
| | | | GAGGACGATGCCTACTGCACATTCCCCAGCCGGGACGAT |
| | | | CTGCTGCTGTTTTCCCCTTCTGGACAGGGAGAGTTCCGG |
| | | | GCCCTGAACGCAAGACTGCCACTGAATACCGACGCCTAT |
| | | | CTGTCTCTGCAGGAGCTGCAGGGCCAGGACCCCACACAC |
| | | | CTGGTG |

The engineered immune cells provided herein can comprise one or more mimotope sequences that enable sorting of cells to enrich a population for cells engineered as described herein, e.g. cells that express the antigen binding protein, and/or that provide a safety switch mechanism to inactivate the immune cell after the cells have been administered to the patient or recipient, e.g. to limit adverse effects. Such mimotope sequences and their application in cell sorting and as safety switches are known in the art and described, for example, in US2018/0002435, which is incorporated herein by reference in its entirety.

Prior to expansion and genetic modification, a source of cells can be obtained from a subject through a variety of non-limiting methods. Cells can be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. In some embodiments, any number of T cell lines available and known to those skilled in the art, can be used. In some embodiments, cells can be derived from a healthy donor, from a subject diagnosed with cancer or from a subject diagnosed with an infection. In some embodiments, cells can be part of a mixed population of cells which present different phenotypic characteristics.

Also provided herein are cell lines obtained from a modified e.g. transformed or engineered immune cell e.g. engineered T cell according to any of the methods described herein. In some embodiments, the cell line prepared from or derived from an engineered immune cell e.g. engineered T cell according to the instant disclosure comprises a polynucleotide encoding a chimeric polypeptide of the invention e.g. a chimeric switch receptor, and optionally a CAR and/or CCR; the cell line optionally is also modified or engineered e.g. genetically modified to express e.g. functionally express no or a reduced level of one or more of CD70, CD52, PD1, TIM3, CISH, cbl-b, TIGIT and TCRa.

The immune cells, e.g. T cells of the instant disclosure, can be activated and expanded, either prior to or after modification of the cells, using methods as generally described, for example without limitation, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005. Immune cells e.g. T cells can be expanded in vitro or in vivo. Generally, the immune cells of the instant disclosure can be expanded, for example, by contact with an agent that stimulates a CD3 TCR complex and a co-stimulatory molecule on the surface of the immune cells to create an activation signal for the cell. For example, chemicals such as calcium ionophore A23187, phorbol 12-myristate 13-acetate (PMA), or mitogenic lectins like phytohemagglutinin (PHA) can be used to create an activation signal for the immune cell, e.g., a T cell.

In some embodiments, T cell populations can be stimulated in vitro by contact with, for example, an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. Conditions appropriate for T cell culture include an appropriate medium (e.g., Minimal Essential Media, RPMI Media 1640 or, X-VIVO™ 5, (Lonza)) that can contain factors necessary for proliferation and viability, including serum (e.g., fetal bovine or human serum), interleukin-2 (IL-2), insulin, IFN-7, IL-4, IL-7, GM-CSF, IL-10, IL-2, IL-15, a TGFβ, and TNF, or any other additives for the growth of cells known to the skilled artisan. Other additives for the growth of cells include, but are not limited to, surfactant, Plasmanate®, and reducing agents such as N-acetyl-cysteine and 2-mercaptoethanol. Media can include RPMI 1640 (as noted herein), AIM V, DMEM, MEM, α-MEM, F-12, X-VIVO™ 10, X-VIVO™ 15 and X-VIVO™ 20, OpTmizer™, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂). Immune cells e.g. T cells that have been exposed to varied stimulation times can exhibit different characteristics.

In some embodiments, the cells of the instant disclosure can be expanded by co-culturing with tissue or cells. The cells can also be expanded in vivo, for example in the subject's blood after administrating the cell into the subject.

In another aspect, the instant disclosure provides compositions (such as pharmaceutical compositions) comprising any of the cells e.g. engineered immune cells of the instant disclosure. In some embodiments, the composition comprises an engineered immune cell e.g. an engineered T cell comprising a polynucleotide encoding a chimeric polypeptide as disclosed herein e.g. a chimeric switch receptor, or a population of cells that comprise such an engineered immune cell, e.g. a population of cells that comprise between about 1×10⁴ and about 1×10¹⁰ engineered immune cells provided herein, and one or more pharmaceutically acceptable carriers or excipients.

In some embodiments, primary cells isolated from a donor are engineered as described herein to provide a population of cells of which a subpopulation (e.g., a proportion less than 100%, such as 10%, 20%, 30%, 40%, 50%, 60%, 70% 80% or 90%) of the resulting cells comprise all of the desired modifications. Such a resulting population, comprising a mixture of cells that comprise all of the modifications and cells that do not, can be used in the methods of treatment of the instant disclosure and to prepare the compositions of the instant disclosure. Alternatively, this population of cells (the “starting population”) can be manipulated by known methods e.g. cell sorting and/or expansion of cells that have the desired modifications, to provide a population of cells that is enriched for those cells comprising one or more of the desired modifications (e.g. enriched for cells that express the desired chimeric switch receptor protein and/or enriched for cells that express one or more of a CAR and a CCR, wherein a polynucleotide encoding the chimeric switch receptor is inserted at a CD70 locus, CD52 locus, PD1 locus, TIM3 locus, CISH locus, TIGIT locus, or cbl-b locus, and wherein a polynucleotide encoding a CAR and optionally a CCR is inserted at a TRAC locus), that is, that comprises a higher percentage of such modified or engineered cells than did the starting population. The population enriched for the modified cells can then be used in the methods of treatment of the instant disclosure and to prepare the compositions of the instant disclosure, for example. In some embodiments, the enriched population of cells contains, or contains at least, for example, 20%, 30%, 40%, 50%, 60%, 70%, 80%, 90%, 95%, or 99% cells that have one or more of the modifications. In other embodiments, the proportion of cells of the enriched population of cells that comprise one or more of the modifications is at least 30% higher than the proportion of cells of the starting population of cells that comprise the desired modifications.

### Methods of Treating

Engineered immune cells, e.g. engineered T cells obtained by the methods described herein, or cell lines derived from such immune cells or T cells, can be used as a medicament or to prepare a medicament. In some embodiments, such a medicament can be used for treating a disorder such as for example a viral disease, a bacterial disease, a cancer, an inflammatory disease, an immune disease, or an aging-associated disease. In some embodiments, the cancer can be selected from the group consisting of gastric cancer, sarcoma, lymphoma (including Non-Hodgkin's lymphoma), leukemia, head and neck cancer, thymic cancer, epithelial cancer, salivary cancer, liver cancer, stomach cancer, thyroid cancer, lung cancer, ovarian cancer, breast cancer, prostate cancer, esophageal cancer, pancreatic cancer, glioma, leukemia, multiple myeloma, renal cell carcinoma, bladder cancer, cervical cancer, choriocarcinoma, colon cancer, oral cancer, skin cancer, and melanoma. In some embodiments, the subject is a previously treated adult subject with locally advanced or metastatic melanoma, squamous cell head and neck cancer (SCHNC), ovarian carcinoma, sarcoma, or relapsed or refractory classic Hodgkin's Lymphoma (cHL).

In some embodiments, engineered immune cells e.g., engineered T cells according to the instant disclosure, or a cell line derived from the engineered immune cells e.g., engineered T cells, can be used in the manufacture of a medicament for treatment of a disorder in a subject in need thereof. In some embodiments, the disorder can be, for example, a cancer, an autoimmune disorder, or an infection.

Also provided herein are methods for treating subjects. In some embodiments the method comprises administering or providing an engineered immune cell e.g., an engineered T cell of the instant disclosure to a subject in need thereof. In some embodiments, the method comprises a step of administering the engineered immune cells e.g., engineered T cells of the instant disclosure, to a subject in need thereof.

In some embodiments, engineered immune cells e.g., engineered T cells of the instant disclosure can undergo robust in vivo cell expansion and can persist for an extended amount of time. Methods of treatment of the instant disclosure can be ameliorating, curative or prophylactic. The method of the instant disclosure can be either part of an autologous immunotherapy or part of an allogeneic immunotherapy treatment. The instant disclosure is particularly suitable for allogeneic immunotherapy. Engineered immune cells e.g., engineered T cells prepared from cells provided by a donor, can be transformed into non-alloreactive cells using standard protocols and reproduced as needed, thereby producing e.g. CAR-T cells expressing a chimeric switch receptor which can be administered to one or several subjects. Such CAR-T cell therapy can be made available as an allogeneic therapeutic product e.g. an ALLO CAR T™ therapeutic product.

In another aspect, the instant disclosure provides a method of inhibiting tumor growth or progression in a subject who has a tumor, comprising administering to the subject an effective amount of engineered immune cells e.g. engineered T cells as described herein. In another aspect, the present disclosure provides a method of inhibiting or preventing metastasis of cancer cells in a subject, comprising administering to the subject in need thereof an effective amount of engineered immune cells e.g. engineered T cells as described herein. In another aspect, the instant disclosure provides a method of inducing tumor regression in a subject who has a tumor, comprising administering to the subject an effective amount of engineered immune cells, e.g., engineered T cells as described herein.

In some embodiments, the immune cells, e.g., T cells provided herein can be administered parenterally to a subject. In some embodiments, the subject is a human.

In some embodiments, the method can further comprise administering an effective amount of a second therapeutic agent. In some embodiments, the second therapeutic agent is, for example, crizotinib, palbociclib, an anti-CTLA4 antibody, an anti-4-1 BB antibody, a PD-1 antibody, or a PD-L1 antibody.

Also provided is the use of any of the engineered immune cells e.g. T cells provided herein in the manufacture of a medicament for the treatment of cancer or for inhibiting tumor growth or progression in a subject in need thereof.

In certain embodiments, the functional expression level of one or more of CD70, CD52, PD1, TIM3, CISH, cbl-b, TIGIT and TCRa in an engineered immune cell of the instant disclosure is decreased by or by at least about 25%, 30%, 35%, 40%, 45%, 50%, 55%, 60%, 65%, 70%, 75%, 80%, 85%, 90%, 95%, 99%, or 100% relative to the corresponding expression level in a comparable but not so genetically-modified engineered immune cell. Expression levels can be determined by any known method, such as FACS or MACs. In some embodiments, the engineered immune cell disclosed herein functionally expresses any one or more of CD70, CD52, PD1, TIM3, CISH, cbl-b, TIGIT and TCRa at a level not greater than 75%, not greater than 50%, not greater than 25%, not greater than 10% or at a level of 0% of the expression level in non-engineered immune cells that otherwise are the same as the engineered immune cells, e.g. comprise the same components as the engineered immune cells. In some embodiments, both alleles of one gene are knocked out, so that gene's expression level in the engineered immune cell disclosed herein is 0% of that of a corresponding non-engineered cell. In some embodiments, one of the two alleles of a gene is knocked out, so that gene's expression level in the engineered immune cell disclosed herein is 50% or about 50% (e.g. if a compensatory mechanism causes greater than normal expression of the remaining allele) of that of a corresponding non-engineered cell. Intermediate levels of expression can be observed if, for example, expression is reduced by some means other than knock-out, as described herein.

In some embodiments, the expression level of one or more of CD70, CD52, PD1, TIM3, CISH, cbl-b, TIGIT and TCRa in the engineered cells of the present disclosure can be measured directly by assaying the cells for gene products and their properties using standard techniques known to those of skill in the art (e.g. RT-qPCR, nucleic acid sequencing, antibody staining, or some combination of techniques). In some embodiments, the functional expression level of one or more of CD70, CD52, PD1, TIM3, CISH, cbl-b, TIGIT and TCRa is measured by standard techniques known in the art, e.g. flow cytometry. These measurements can be compared to corresponding measurements made on comparable cells that have not been engineered to reduce the corresponding functional expression level. In a population of cells that comprises an engineered cell e.g. engineered immune cell of the invention, a pooled sample of the material being measured, e.g. RNA or protein or cells, will reflect the fact that some of the cells do not express the gene of interest, having had both alleles knocked out, for example, some of the cells express the gene of interest at 50% or about 50%, having had only one allele knocked out, and, if the population comprises non-engineered cells, that some of the cells express a normal level of the gene of interest.

The functional expression level of one or more of CD70, CD52, PD1, TIM3, CISH, cbl-b, TIGIT and TCRa expression in engineered immune cells of the present disclosure can also be assayed, for example, by measuring the degree to which the engineered immune cells survive in the presence of effector cells e.g. T cells or NK cells, in comparison to the degree to which non-engineered, but otherwise comparable e.g. identical, immune cells survive under the same conditions.

In some embodiments, the treatment disclosed herein can be in combination with one or more therapies against cancer selected from the group of surgery, antibodies therapy, chemotherapy, cytokines therapy, dendritic cell therapy, gene therapy, hormone therapy, laser light therapy and radiation therapy.

In some embodiments, treatment can be administered to subjects undergoing an immunosuppressive treatment. Indeed, the instant disclosure can rely on cells or a population of cells which have been made resistant to at least one immunosuppressive agent due to the inactivation of a gene encoding a receptor for such immunosuppressive agent. In this aspect, the immunosuppressive treatment can help the selection and expansion of the T cells according to the instant disclosure within the subject.

The administration of the cells or population of cells according to the instant disclosure can be carried out in any convenient manner, including by aerosol inhalation, injection, ingestion, transfusion, implantation or transplantation. The compositions described herein can be administered to a subject subcutaneously, intradermally, intratumorally, intranodally, intramedullary, intramuscularly, by intravenous or intralymphatic injection, or intraperitoneally. In one embodiment, the cell compositions of the instant disclosure are administered by intravenous injection.

In some embodiments, the administration of the cells or population of cells according to the instant disclosure can comprise administration of, for example, from about 10³ or 10⁴ to about 10⁹ cells per kg body weight including all integer values of cell numbers within those ranges, or a composition as disclosed herein comprising engineered immune cells as disclosed herein. In some embodiments the administration of the cells or population of cells can comprise administration of about 10⁵ to about 10⁶ cells per kg body weight including all integer values of cell numbers within those range, or administration of between 0.1×10⁶ and 5×10⁶ engineered immune cells of the invention per kg body weight, or a total of between 0.1×10⁸ and 5×10⁸ engineered immune cells. The cells or population of cells can be administered in one or more doses. In some embodiments, an effective amount of cells can be administered as a single dose. In some embodiments, an effective amount of cells can be administered as more than one dose over a period time. Timing of administration is within the judgment of the managing physician and depends on the clinical condition of the subject. The cells or population of cells can be obtained from any source, such as a blood bank or a donor. While individual needs vary, determination of optimal ranges of effective amounts of a given cell type for a particular disease or conditions is within the skill of the art. An effective amount means an amount which provides a therapeutic or prophylactic benefit. The dosage administered will be dependent upon the age, health and weight of the recipient, the kind of concurrent treatment, if any, the frequency of treatment and the nature of the effect desired. In some embodiments, an effective amount of cells or composition comprising those cells are administered parenterally. In some embodiments, administration can be an intravenous administration. In some embodiments, administration can be directly done by injection within a tumor.

In some embodiments of the instant disclosure, cells e.g. engineered immune cells as disclosed herein are administered to a subject in conjunction with (e.g., before, simultaneously or following) any number of relevant treatment modalities, including but not limited to treatment with agents such as monoclonal antibody therapy, CCR2 antagonist (e.g., INC-8761), antiviral therapy, cidofovir and interleukin-2, Cytarabine (also known as ARA-C) or nataliziimab treatment for MS subjects or efaliztimab treatment for psoriasis subjects or other treatments for PML subjects. In some embodiments, BCMA specific CAR-T cells are administered to a subject in conjunction with one or more of the following: an anti-PD-1 antibody (e.g., nivolumab, pembrolizumab), an anti-PD-L1 antibody (e.g., avelumab, atezolizumab, or durvalumab), an anti-OX40 antibody, an anti-4-1 BB antibody (e.g., Utolimumab), an anti-MCSF antibody, an anti-GITR antibody, and/or an anti-TIGIT antibody. In further embodiments, the immune cells, e.g. T cells, of the instant disclosure can be used in combination with surgery, chemotherapy, radiation, immunosuppressive agents, such as cyclosporin, azathioprine, methotrexate, mycophenolate, and FK506, antibodies, or other immunoablative agents such as CAMPATH (alemtuzumab), anti-CD3 antibodies or other antibody therapies, cytoxan, fludarabine, cyclophosphamide, cyclosporin, FK506, rapamycin, mycoplienolic acid, steroids, FR901228, cytokines, and/or irradiation. These drugs inhibit either the calcium dependent phosphatase calcineurin (cyclosporine and FK506) or inhibit the p70S6 kinase that is important for growth factor induced signaling (rapamycin) (Henderson, Naya et al. Immunology. 1991 July; 73(3): 316-321; Liu, Albers et al. Biochemistry 1992 Apr. 28; 31(16):3896-901; Bierer, Hollander et al. Curr Opin Immunol. 1993 October; 5(5):763-73). The interval between different treatment modalities can range from minutes (e.g. from 1 to 360 minutes) up to hours (e.g. up to 6, 12, 18 or 24 hours), days (e.g. up to between 1 and 7 days), or weeks e.g. up to 1, 2, 4, 8, 16 or 52 weeks.

In a further embodiment, the cell compositions of the instant disclosure are administered to a subject in conjunction with (e.g., before, simultaneously or following) bone marrow transplantation, T cell ablative therapy using either chemotherapy agents such as fludarabine, external-beam radiation therapy (XRT), cyclophosphamide, or antibodies such as CAMPATH. In some embodiments, the cell compositions of the instant disclosure are administered following B-cell ablative therapy such as agents that react with CD20, e.g., Rituxan. For example, in one embodiment, subjects can undergo standard treatment with high dose chemotherapy followed by peripheral blood stem cell transplantation. In certain embodiments, following the transplant, subjects receive an infusion of expanded immune cells of the instant disclosure. In some embodiments, expanded cells are administered before or following surgery.

### Kits

The instant disclosure also provides kits for use in the instant methods. Kits of the instant disclosure include one or more containers comprising a composition of the instant disclosure or an immune cell, e.g., a T cell of the instant disclosure or a population of cells comprising an immune cell, e.g., an engineered T cell of the instant disclosure. In various embodiments, the immune cell, e.g., T cell comprises one or more polynucleotide(s) encoding the desired chimeric switch receptor protein and one or more of a CAR and a CCR as described herein, and further is engineered to express a reduced level of one or more of CD70, CD52, PD1, TIM3, CISH, cbl-b, TIGIT and TCRa as described herein. The kit further comprises instructions for use in accordance with any of the methods of the instant disclosure described herein. Generally, these instructions comprise a description of administration of the composition, immune cell, e.g., a T cell or population of cells for the above described therapeutic treatments.

The instructions relating to the use of the kit components generally include information as to dosage, dosing schedule, and route of administration for the intended treatment. The containers can be unit doses, bulk packages (e.g., multi-dose packages) or subunit doses. Instructions supplied in the kits of the instant disclosure are typically written instructions on a label or package insert (e.g., a paper sheet included in the kit), but machine-readable instructions (e.g., instructions carried on a magnetic or optical storage disk) are also acceptable.

The kits of the present disclosure are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit can have a sterile access port (for example the container can be an intravenous solution bag or a vial having a stopper pierceable by a hypodermic injection needle). The container can also have a sterile access port (for example the container can be an intravenous solution bag or a vial having a stopper pierceable by a hypodermic injection needle). At least one active agent in the composition is an immune cell e.g. T cell according to the instant disclosure. The container can further comprise a second pharmaceutically active agent.

Kits can optionally provide additional components such as buffers and interpretive information. Normally, the kit comprises a container and a label or package insert(s) on or associated with the container.

Methods of Sorting and Depletion

In some embodiments, provided are methods for in vitro sorting of a population of immune cells, wherein a subset of the population of immune cells comprises immune cells engineered as described herein to express one or more of CD70, CD52, PD1, TIM3, CISH, cbl-b, TIGIT and TCRa at a reduced level and/or express a chimeric switch receptor and an antigen binding protein, e.g., a CAR. In various embodiments the method comprises contacting the population of immune cells with a monoclonal antibody specific for an epitope (e.g., a mimotope such as those provided in US2018/0002435) unique to the engineered cell, e.g. an epitope of the antigen binding protein or a mimotope incorporated into the antigen binding protein, and selecting the immune cells that bind to the monoclonal antibody to obtain a population of cells enriched in engineered immune cells that express the antigen binding protein.

In some embodiments, the monoclonal antibody specific for the epitope is optionally conjugated to a fluorophore. In this embodiment, the step of selecting the cells that bind to the monoclonal antibody can be done by Fluorescence Activated Cell Sorting (FACS).

In some embodiments, said monoclonal antibody specific for said epitope is optionally conjugated to a magnetic particle. In this embodiment, the step of selecting the cells that bind to the monoclonal antibody can be done by Magnetic Activated Cell Sorting (MACS).

In some embodiments, the mAb used in the method for sorting immune cells expressing the chimeric switch receptor of the present disclosure and an antigen binding protein, e.g., a CAR is chosen from alemtuzumab, ibritumomab tiuxetan, muromonab-CD3, tositumomab, abciximab, basiliximab, brentuximab vedotin, cetuximab, infliximab, rituximab, bevacizumab, certolizumab pegol, daclizumab, eculizumab, efalizumab, gemtuzumab, natalizumab, omalizumab, palivizumab, ranibizumab, tocilizumab, trastuzumab, vedolizumab, adalimumab, belimumab, canakinumab, denosumab, golimumab, ipilimumab, ofatumumab, panitumumab, QBEND-10 and/or ustekinumab. In some embodiments, said mAb is rituximab. In another embodiment, said mAb is QBEND-10.

In some embodiments, the population of CAR-expressing immune cells obtained when using the method for in vitro sorting of CAR-expressing immune cells described above, comprises at least 70%, 75%, 80%, 85%, 90%, 95% of CAR-expressing immune cells. In some embodiments, the population of CAR-expressing immune cells obtained when using the method for in vitro sorting CAR-expressing immune cells, comprises at least 85% CAR-expressing immune cells.

In some embodiments, the population of CAR-expressing immune cells obtained when using the method for in vitro sorting CAR-expressing immune cells described above shows increased cytotoxic activity in vitro compared with the initial (non-sorted) cell population. In some embodiments, said cytotoxic activity in vitro is increased by 10%, 20%, 30% or 50%. In some embodiments, the immune cells are T-cells.

The chimeric switch receptor- and CAR-expressing immune cells to be administered to the recipient can be enriched in vitro from the source population. Methods of expanding source populations can include selecting cells that express an antigen such as CD34 antigen, using combinations of density centrifugation, immuno-magnetic bead purification, affinity chromatography, and fluorescent activated cell sorting.

Flow cytometry can be used to quantify specific cell types within a population of cells. In general, flow cytometry is a method for quantitating components or structural features of cells primarily by optical means. Since different cell types can be distinguished by quantitating structural features, flow cytometry and cell sorting can be used to count and sort cells of different phenotypes in a mixture.

A flow cytometry analysis involves two primary steps: 1) labeling selected cell types with one or more labeled markers, and 2) determining the number of labeled cells relative to the total number of cells in the population. In some embodiments, the method of labeling cell types includes binding labeled antibodies to markers expressed by the specific cell type. The antibodies can be either directly labeled with a fluorescent compound or indirectly labeled using, for example, a fluorescent-labeled second antibody which recognizes the first antibody.

In some embodiments, the method used for sorting T cells expressing a CAR is the Magnetic-Activated Cell Sorting (MACS) method. MACS is a method for separation of various cell populations depending on their surface antigens (CD molecules) by using superparamagnetic nanoparticles and columns. MACS can be used to obtain a pure cell population. Cells in a single-cell suspension can be magnetically labeled with microbeads. The sample is applied to a column composed of ferromagnetic spheres, which are covered with a cell-friendly coating allowing fast and gentle separation of cells. The unlabeled cells pass through while the magnetically labeled cells are retained within the column. The flow-through can be collected as the unlabeled cell fraction. After a washing step, the column is removed from the separator, and the magnetically labeled cells are eluted from the column.

A detailed protocol for the purification of a specific cell population such as T-cells can be found in Basu S et al. (2010). (Basu S, Campbell H M, Dittel B N, Ray A. Purification of specific cell population by fluorescence activated cell sorting (FACS). J Vis Exp. (41): 1546).

### EXAMPLES

### Example 1: Design of Chimeric Switch Receptors

Chimeric switch receptors can be fusion proteins comprising an ectodomain and/or transmembrane domain derived from an inhibitory receptor (e.g. PD1 or TGFbR2) fused to the transmembrane domain and/or intracellular signaling domain derived from one or more costimulatory proteins (e.g. CD2, CD28, MyD88, DAP10 or ICOS). Chimeric switch receptors can compete with endogenous inhibitory receptors (e.g. PD1 or TGFbR) for ligand binding to subvert immunesuppression, and transmit a costimulatory signal in PDL1/2- or TGFb-enriched environments

In chimeric switch receptors containing an ectodomain derived from PD1, the ectodomain may, for example, either be the wildtype sequence, or be modified to bind PD1 ligands with higher affinity than wildtype PD1.

In chimeric switch receptors containing an ectodomain derived from TGFbR2 (also referred to herein as “BR2”), the ectodomain may, for example, either be the wildtype sequence, or be modified, for example, to have 25 residues deleted from the N-terminus of the wildtype sequence (dN25). The TGFbR2 N25 peptide can mediate the recruitment of TGFbR1, which may in turn interfere with costimulatory signaling of chimeric switch receptors. Deleting the N25 peptide abolishes TGFbR1 recruitment and may enhance chimeric switch receptor signaling.

In the case where an intracellular signaling domain does not have a transmembrane domain (e.g. MyD88), the PD1 transmembrane domain, which does not dimerize, may be used to minimize tonic signaling of the chimeric switch receptor.

Individual intracellular costimulatory domains may be optimized to reduce vector cargo size and/or enhance or modulate functional activity by the removal of non-signaling intervening sequences or negative regulatory sequences (e.g. CD2short) or by mutating key residues involved in signal transduction (e.g. CD28.YMFM and CD28.AYAA, which are amino acid substitution variants from YMNM and PYAP of the CD28 intracellular domain, respectively). See Boucher, et al., 2021, Cancer Immunology Res. 9:62.

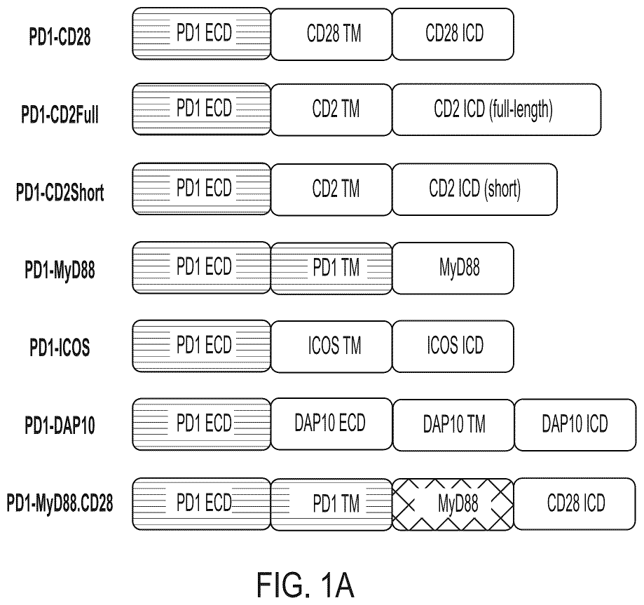

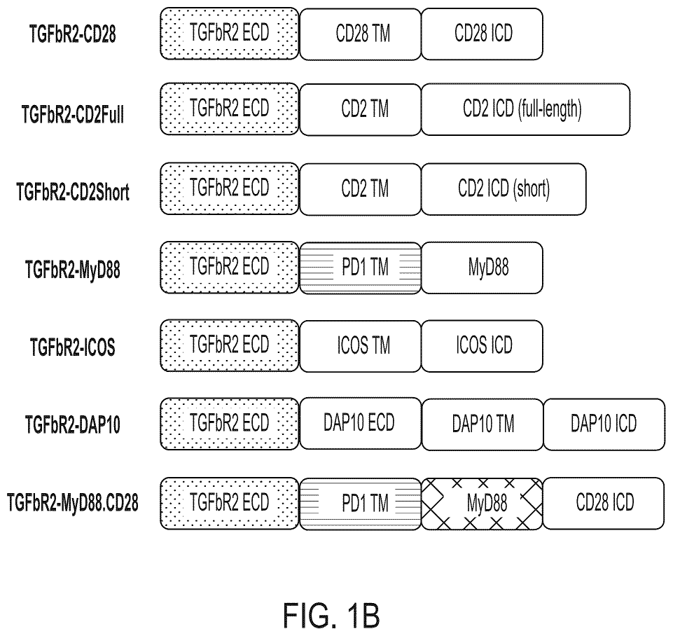

FIG. 1A and FIG. 1B show domain structure of representative PD1 chimeric switch receptors and TGFbR2 chimeric switch receptors, respectively.

### Example 2: Production of T Cells Expressing Chimeric Switch Receptors Alone or Together with a DLL3 CAR Using Lentivirus Co-Transduction

To generate T cells expressing chimeric switch receptors alone or together with a DLL3 CAR, primary human T cells were first purified from LeukoPak (StemCell Technologies) using EasySep™ Human T Cell Isolation Kit (StemCell).

The activities of different chimeric switch receptors were tested either alone or in conjunction with a CAR, e.g., a DLL3 CAR. The activities of CAR T cells expressing the DLL3 CAR clone 2G1 and co-expressing either a CCR of the 15.1 construct or a CCR of the 15.3 construct were first evaluated. In a low stringency SHP-77 subcutaneous tumor model, 5×10⁶ SHP-77 cells were injected to NSG mice (n=8/group) subcutaneously on day 0, and 5×10⁶ CAR T cells were injected intravenously on day 14. In these experiments, peripheral blood T cells numbers were analyzed by flow cytometry on days 71, 85, and 99 post implant. Next, the CAR T cells were tested in a high stringency subcutaneous tumor mode. In this model, 5×10⁶ NCI-H82 cells were injected subcutaneously to NSG mice (n=10/group) on day 0, and 3×10⁶ or 6×10⁶ CAR T cells were injected intravenously on day 11. Peripheral blood T cells numbers were analyzed by flow cytometry once weekly (on days 6, 13, 20 and 27 post CAR T infusion). Serum cytokine levels were measured using MSD Multi-Spot Assay T-plex human cytokine assay kit on week 1 and 4.

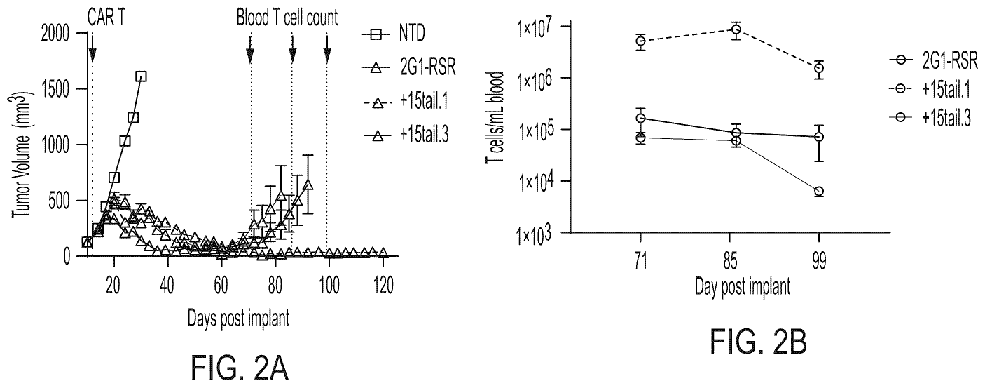

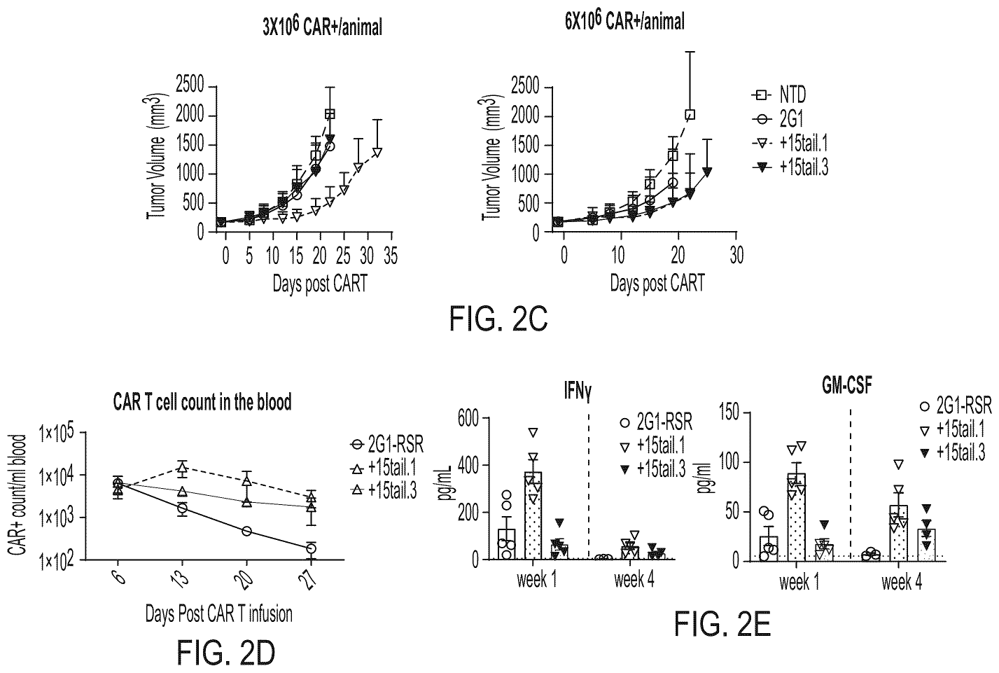

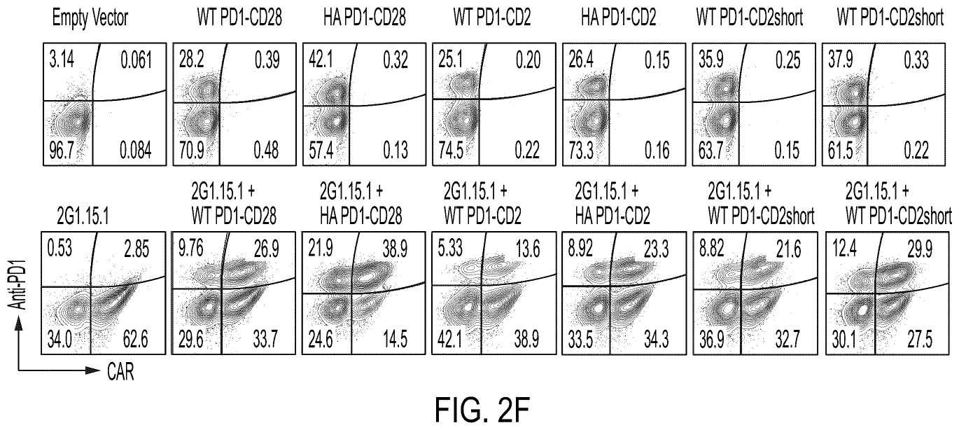

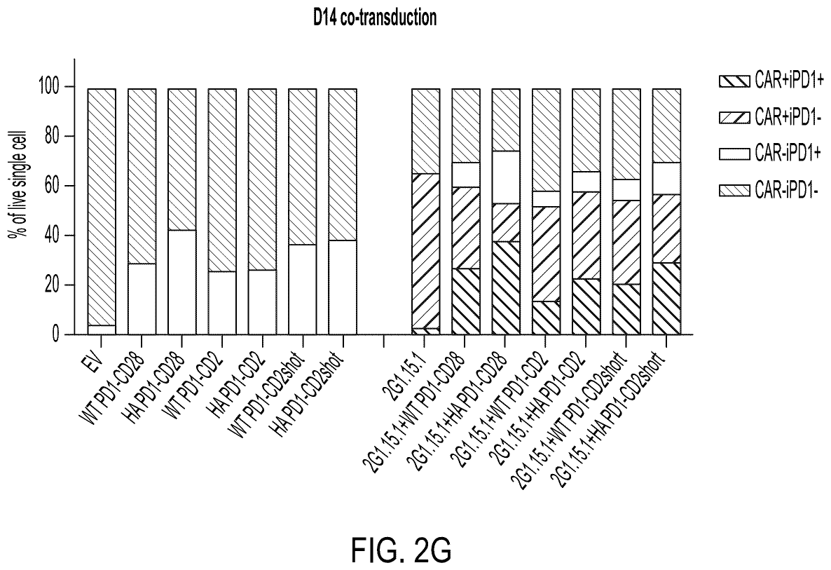

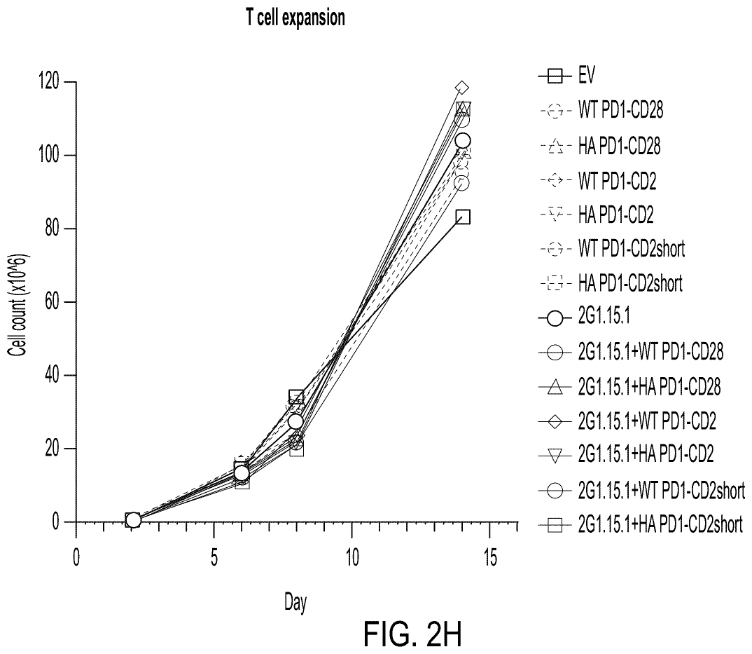

As shown in FIGS. 2A-2B, the 2G1 CAR T expressing the 15.1 CCR construct outperformed the same CAR T cells expressing the 15.3 CCR construct, in the low stringency SHP-77 subcutaneous tumor model. DLL3 2G1.15.1 CAR (“+15tail.1”) exhibited the most durable anti-tumor response than DLL3 2G1.15.3 CAR (“+15tail.3”) or the parental CAR without a CCR (“2G1-RSR”) (FIG. 2A). The DLL3 2G1.15.1 CAR also demonstrated highest amount of CAR T cells in blood post injection, i.e., evidence of in vivo persistence among the three (FIG. 2B). Similarly, in a high stringency animal model, DLL3 2G1.15.1 CAR at a suboptimal CAR T dose (3×10⁶/animal), i.e., a more stringent condition, outperformed the DLL3 2G1.15.3 CAR (FIG. 2C), although the difference is less pronounced when 6×10⁶/animal CAR T cells were injected, i.e., a less stringent condition. The DLL3 2G1.15.1 CAR also exhibited superior in vivo persistence (FIG. 2D) and secreted higher level of serum cytokine in the high stringency animal model (FIG. 2E). As the DLL3 2G1.15.1 CAR showed the best activities, CAR T cells expressing the 2G1 CAR and 15.1 CCR were used in the following experiments.

To make lentivirus encoding the DLL3 CAR alone, DLL3 CAR and a CCR, and/or chimeric switch receptors, HEK-293T cells were plated at 0.75 million cells per mL in 2 mL of DMEM (Gibco) supplemented with 10% FBS (Hyclone) per well of a 6-well plate on Day 0. On Day 1, the lentivirus was prepared by mixing together lentiviral packaging vectors 1.5 ug psPAX2, 0.5 ug pMD2G, and 0.5 ug of the appropriate CAR vector or chimeric switch receptor vector in 250 uL Opti-MEM (Gibco) per well of the 6-well plate (“DNA mix”). 10 uL Lipofectamine 2000 (Invitrogen) in 250 uL Opti-MEM was incubated at room temperature for 5 minutes and then added to the DNA mix. The mixture was incubated at room temperature for 20 minutes and the total volume of 500 uL was slowly added to the sides of the wells containing HEK-293T. Purified T cells were activated in X-Vivo-15 medium (Lonza) supplemented with 100 IU/mL human IL-2 (Miltenyi Biotec), 10% FBS (Hyclone), and human T TransAct (Miltenyi Biotec, Cat #130-111-160, 1:100 dilution). On Day 2, the media from each well of the 6-well plate was replaced with 2 mL per well of T cell transduction media, i.e., X-Vivo-15 supplemented with 10% FBS. On Day 3, T cells were resuspended at 0.75 million cells per mL in 1 mL of T cell transduction media per well of a Grex-24 plate (Wilson Wolf, cat #80192M). The lentiviral supernatants from HEK293T cells were harvested and passed through a 0.45 micron filter (EID Millipore) to remove cell debris, and then mixed with the Lenti-X Concentrator (Clontech) and incubated for 30 minutes at 4° C. The mixture was then centrifuged 1,500×g for 45 minutes at 4° C. to obtain a high-titer virus-containing pellet. After the supernatant was removed, the pellet was resuspended in 1/25 of the original volume using T cell transduction media and 150 ul to 350 ul of concentrated virus was added to the T cells along with 100 IU/mL human IL-2. On Day 5, 4.5 mL of T cell expansion media, i.e., X-Vivo-15 supplemented with 5% human AB serum (Gemini Bio) was added to each well of a Grex-24 plate. On Day 9 and Day 13, CAR transduction efficiency and chimeric switch receptor transduction efficacy were determined. CAR transduction was measured by staining the T cells first with 1 μg/ml Flag tagged recombinant DLL3 (Adipogen) in PBS+1% BSA for 20 minutes at 4° C. and then with PE labelled anti-Flag antibodies (Biolegend, Cat #637310). Chimeric switch receptor was determined by staining the T cells first with 10 μg/ml anti-PD1 antibody nivolumab (Selleckchem, Cat #A2002) and then with anti-human IgG-APC (Jackson ImmunoResearch) at 1:200 dilution. T Cells were expanded into larger flasks or G-Rex vessels (Wilson Wolf) as needed using T cell expansion media. On Day 14 or Day 16, DLL3 CAR-T cells were cryopreserved. Percentage of cells stained with recombinant DLL3 was normalized across clones right before cryopreservation.

FIG. 2F presents experimental data showing that PD1 chimeric switch receptors were expressed alone or together with DLL3 CAR and a CCR (2G1.15.1) on the surface of primary T-cells. Percentage of T cells that express one or both of DLL3 CAR and chimeric switch receptor was determined by staining with recombinant DLL3 and nivolumab. The plots were gated on live single T cell. FIG. 2G presents a bar graph summarizing the percentage of T cells that were transduced with one vector or both vectors. FIG. 2H presents experimental data showing that all T cells expanded well during production, with about 90 to 120 fold increase in cell number from day 2 to day 14.

### Example 3: Cytotoxic Activity of DLL3 CAR T Cells with or without Expressing Chimeric Switch Receptors Using Lentivirus Co-Transduction

Cytotoxicity of T cells produced according to the methods in Example 2 was determined in serial killing assays. The serial killing assay involved repeated exposure of T cells to their target, causing the T cells to undergo proliferation and in certain cases, differentiation and exhaustion. This assay was used to select optimal clones with high target cell killing and proliferative abilities after several rounds of exposure to target cells.

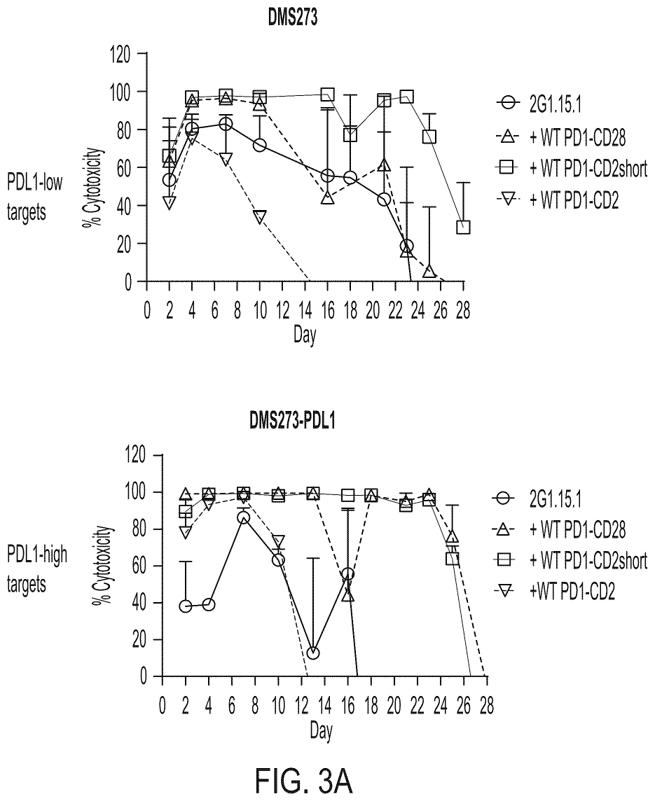

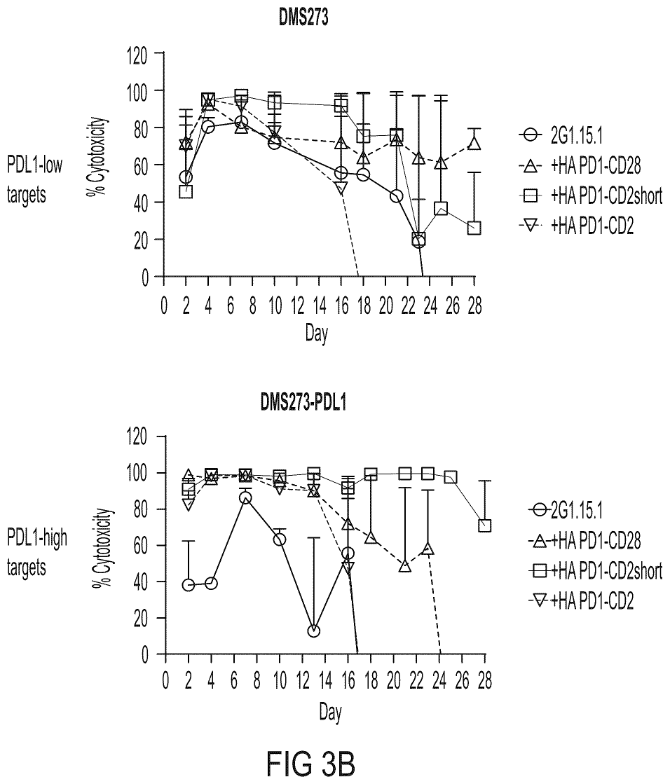

On the first day of the assay, 5,000 firefly luciferase labelled DMS 273 (DLL3-low, PDL1-low) or DMS 273-PDL1(DLL3-low, PDL1-high) cells were seeded in 96-well plates with white wall and flat clear bottom in 100 ul RPMI medium with 10% FBS. After target cells attached to the bottom of the plates, T cells expressing PD1 chimeric switch receptors alone or together with the DLL3 CAR were thawed and added to plated target cells at an effector:target (E:T) ratio of 9:1 or 3:1 in 100 ul RPMI medium with 10% FBS. Every 2 to 3 days thereafter, 100 μl medium containing T cells was transferred to freshly plated target cells and the percentage lysis of previously plated target cells was determined using the one-glo assay system or CellTiter-glo system (Promega). Each condition was assayed in 3 to 6 replicates. Average percentage of lysis and standard deviation were plotted in FIGS. 3A and 3B. FIG. 3A presents experimental data of the serial killing assay showing cytotoxicity of T cells co-expressing the DLL3 CAR and PD1 chimeric switch receptors with the wildtype PD1 ectodomain against DMS 273 (PDL1-low) or DMS 273-PDL1 (PDL1-high) cells. The data of high affinity PD1 chimeric switch receptor are shown in FIG. 3B. DLL3 CAR T cells expressing WT PD1-CD28, WT PD1-CD2short, HA PD1-CD28, or HA-CD2short showed comparable or slightly better activity than DLL3 CAR T cells without expressing chimeric switch receptors against DMS 273 cells, but showed dramatically increased activity against DMS 273-PDL1-high target cells, suggesting high levels of PDL1 on DMS 273-PDL1 was sufficient to cluster and induce signaling of PD1 chimeric switch receptors. T cells expressing PD1 chimeric switch receptors alone did not show cytotoxicity against any target cells tested (data not shown).

### Example 4: Cytokines Secreted from DLL3 CAR T Cells with or without Expressing Chimeric Switch Receptors Using Lentivirus Co-Transduction

Cytokines secreted from T cells produced according to the methods in Example 2 were measured using Human ProInflammatory 9-Plex Tissue Culture Kit (Meso Scale Discovery, 15007B). On the first day of the assay, 5,000 DMS 273 (DLL3-low, PDL1-low) or DMS 273-PDL1(DLL3-low, PDL1-high) cells were seeded in 96-well plates in 100 ul RPMI medium with 10% FBS. After target cells attached to the bottom of the plates, T cells expressing PD1 chimeric switch receptors alone or together with DLL3 CAR and CCR (2G1.15.1) were thawed and added to plated target cells at an effector:target (E:T) ratio of 1:1 in 100 ul RPMI medium with 10% FBS. Twenty-four hours later, medium from the co-culture was collected from each well and spun down to pellet T cells. The supernatant was then frozen at −80° C. and then thawed for cytokine analysis using Meso Scale Discovery analysis according to manufacture's protocol.

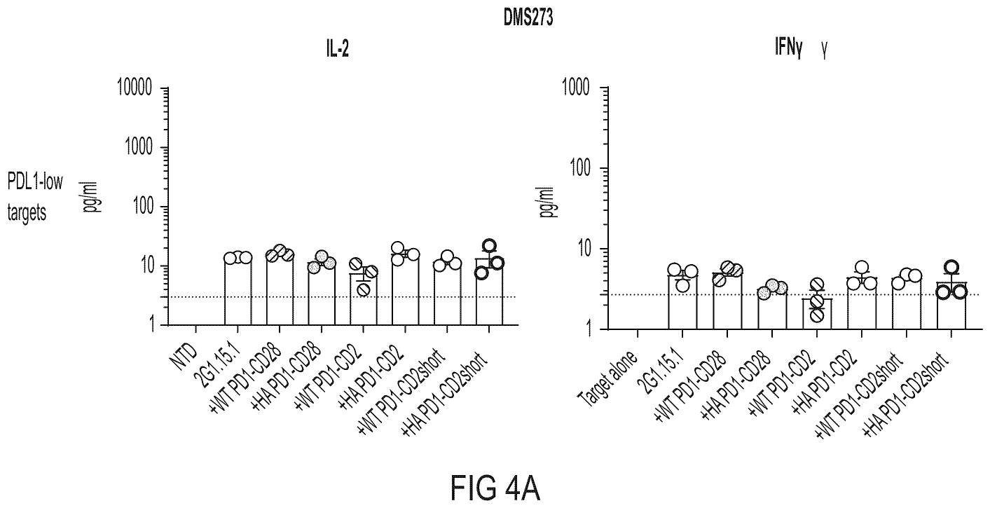

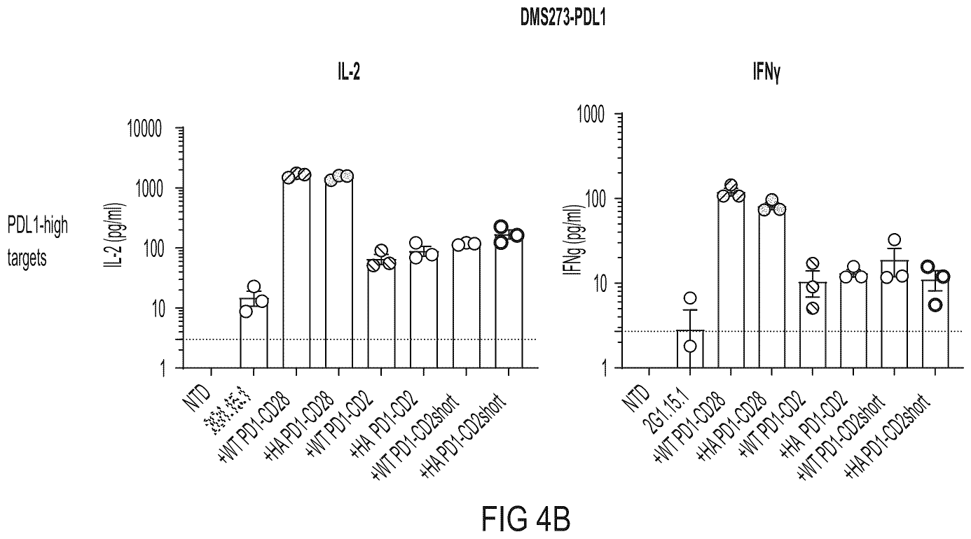

FIGS. 4A-4B present experimental data showing T cells expressing both DLL3 CAR and CCR (2G1.15.1) together with PD1 chimeric switch receptors secreted cytokines when co-cultured with DLL3 positive target cells. When the T cells were co-cultured with PDL1-low DMS 273 cells, all T cells secreted a very small amount of cytokines (IL-2 and IFNγ were shown as representative cytokines) (FIG. 4A). When the T cells were co-cultured with PDL1-high DMS 273-PDL1 cells, PD1 chimeric switch receptors with CD28, CD2 or CD2short signaling domains showed increased cytokine secretion, with CD28 showing the most significant increase (more than 10 fold) (FIG. 4B). When co-cultured with DMS 273-PDL1 cells, all CAR T with a PD1 chimeric switch receptor secreted higher levels of IL-2 and IFNγ than CAR T without a PD1 chimeric switch receptor (FIG. 4B).

### Example 5: Production of DLL3 CAR T Cells with or without Expressing Chimeric Switch Receptors Using Multiplexed Site Specific Integration (SSI)

To generate DLL3 CAR T cells (or CD70 CAR T cells) with or without expressing chimeric switch receptors using multiplexed site specific integration (SSI), primary human T cells were first purified from LeukoPak (StemCell Technologies) using EasySep™ Human T Cell Isolation Kit (StemCell).

On Day 0, purified T cells were activated in X-Vivo-15 medium (Lonza) supplemented with 100 IU/mL human IL-2 (Miltenyi Biotec), 10% FBS (Hyclone), and human T TransAct (Miltenyi Biotec, Cat #130-111-160, 1:100 dilution). On Day 2, electroporation was performed with TALEN targeting TRAC and CD52 loci using P3 Primary Cell 4D-Nucleofector™ X Kit (Lonza, Cat #V4XP-3024). Activated T cells were pelleted, washed twice with PBS, and resuspended at 5 to 10 million cells per 100 ul Lonza electroporation buffer (prepared by mixing 18 ul supplement with 82 ul of Nucleofector™ Solution from P3 Primary Cell 4D-Nucleofector™ X Kit). TALEN mRNAs targeting TRAC and CD52 loci were added to resuspended T cells at 10 ug per TALEN mRNA per 10 million cells and the mixture was loaded in Nucleocuvette (Lonza) for electroporation using DS115 program (for stimulated human T cells) on Amaxa™ 4D-Nucleofector (Lonza, AAF-1002X). Electroporated T cells were taken out of the cuvette and plated in 96-well V bottom plate at 3 million cells per well with 50 ul X-Vivo-15 medium (Lonza) supplemented with 5% human serum (Gemini Bio) and 100 IU/mL human IL-2 (Miltenyi Biotec). T cells were then transduced with AAVs encoding the DLL3 CAR (or CD70 CAR) and/or chimeric switch receptors at multiplicity of infection (MOI) of 5000 to 10,000 and incubated at 30° C. for 1 hour. After the 1-hour incubation, T cells were transferred to a Grex-24 plate (Wilson Wolf, cat #80192M) pre-filled with 1 ml of X-Vivo-15 medium supplemented with 5% human serum and 100 IU/mL human IL-2 and incubated at 30° C. overnight. On Day 3, the Grex-24 plate containing transduced T cells were moved to 37° C. incubator and 1 ml of X-Vivo-15 medium supplemented with 5% human serum and 100 IU/mL human IL-2 was added to each well. On Day 5, 6 mL of X-Vivo-15 supplemented with 5% human AB serum and 100 IU/mL human IL-2 was added to each well of a Grex-24 plate. From day 5 to the day cells were cryopreserved, cells were split when the density exceeded 5 million per ml and 100 IU/mL human IL-2 was added to the medium every 2 to 3 days.

During production, TRAC and CD52 knockout efficiency were determined on Day 6. CAR (2G1.15.1) transduction efficiency and chimeric switch receptor transduction efficiency were determined on Day 6, Day 9 and Day 13/14. CAR transduction was measured by staining the T cells first with 1 μg/ml Flag tagged recombinant DLL3 (Adipogen) in PBS+1% BSA for 20 minutes at 4° C. and then with 3 μg/ml PE labelled anti-Flag antibodies (Biolegend, Cat #637310). Chimeric switch receptor was determined by staining the T cells first with 10 μg/ml nivolumab (Selleckchem, Cat #A2002) for 20 minutes at 4° C. and then with anti-human IgG-APC (Jackson ImmunoResearch) at 1:200 dilution for 20 minutes at 4° C. T Cells were expanded into larger flasks or G-Rex vessels (Wilson Wolf) as needed using T cell expansion media. On Day 14 or Day16, DLL3 CAR-T cells (or CD70 CAR T cells) were cryopreserved. Percentage of cells stained with recombinant DLL3 was normalized across clones right before cryopreservation.

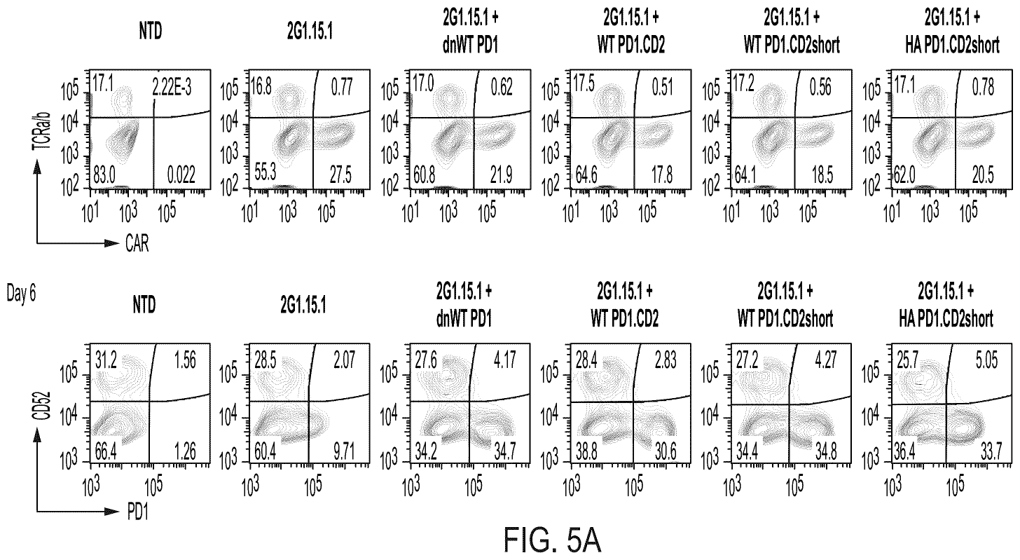

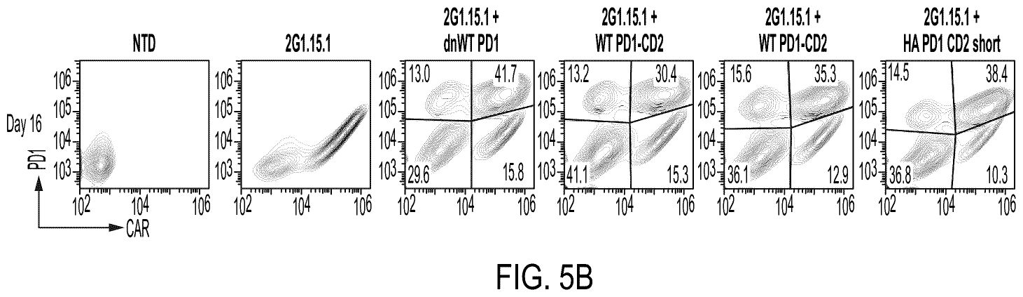

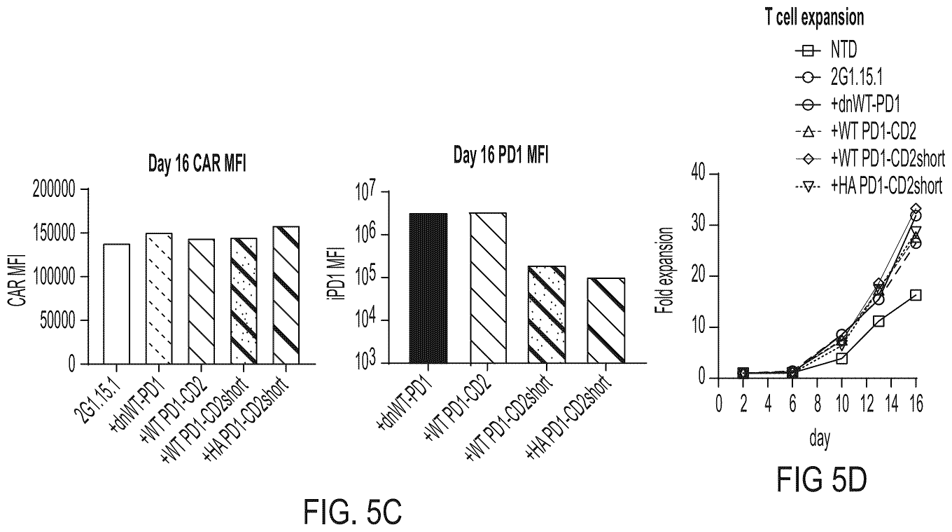

FIGS. 5A-5D show production of T cells expressing DLL3 CAR and chimeric switch receptors using SSI from one human T cell donor. FIG. 5A shows experimental data showing TALEN mediated gene knockout efficiency and AAV mediated transduction efficiency on day 6 of production. Top panel of FIG. 5A shows ˜83% of T cells lost TCRa/b expression and a subset of TCRa/b negative T cells express DLL3 CAR. Bottom panel of FIG. 5A shows ˜70% of T cells lost CD52 expression and a subset of CD52 negative T cells express PD1 chimeric switch receptor. FIG. 5B shows flow cytometry plots depicting percentage of T cells expressing the DLL3 CAR and/or chimeric switch receptor on day 16 of production. The plots are gated on live single T cells. FIG. 5C shows bar graphs demonstrating mean fluorescence intensity of DLL3 CAR (left panel) and PD1 chimeric switch receptors (right panel). FIG. 5D shows T cell expansion during production.

Regarding the constructs expressed, dnWT-PD1 (dominant negative wild-type PD1) has the extracellular and TM domain of wild-type PD1 but no intracellular domain. It therefore binds to PDL1 (on target cells), thereby competing with endogenous PD1 (on T cells) to block signaling by PDL1. WT PD1-CD28 or WT or HA PD1-CD2 construct has a wild-type or high affinity PD1 extracellular domain and an intracellular domain of CD28 or CD2; it therefore signals via the intracellular domain upon binding to PDL1.

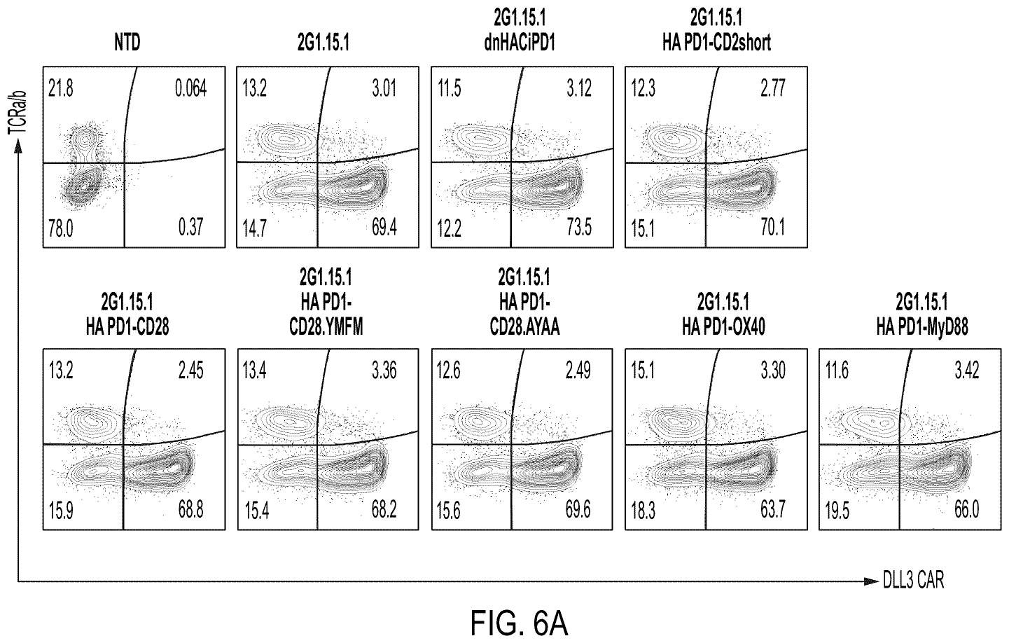

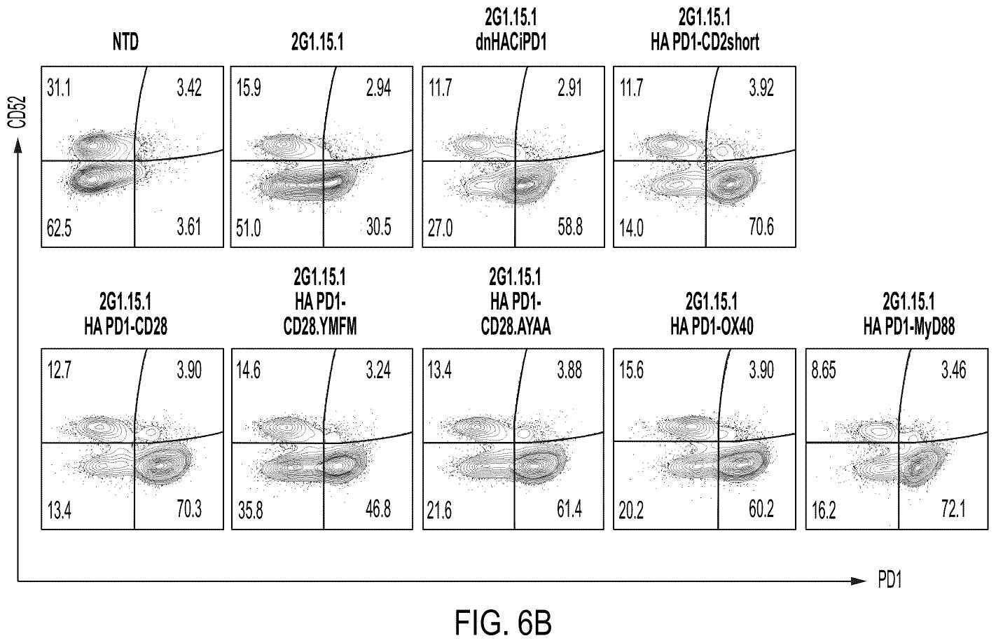

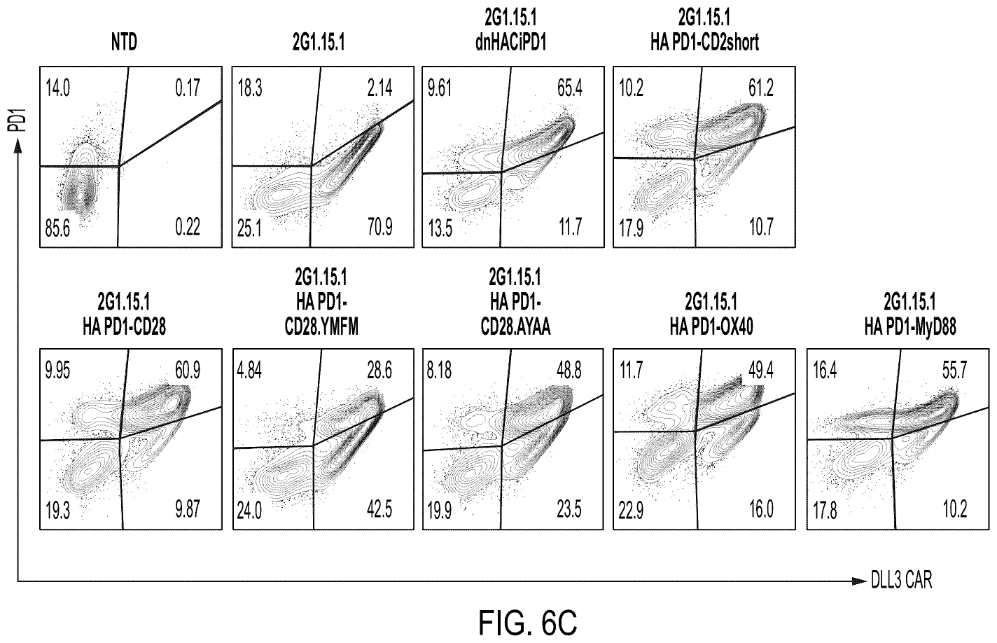

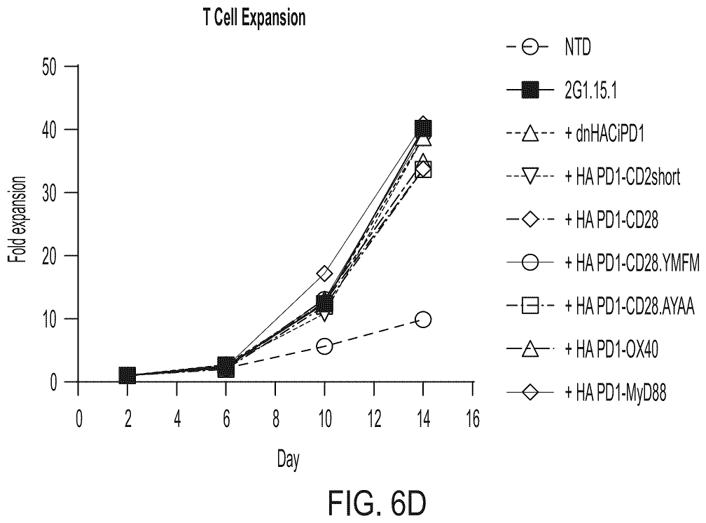

FIGS. 6A-6D show production of T cells expressing DLL3 CAR and chimeric switch receptors using SSI from a human T cell donor different from the donor shown in FIGS. 5A-5D. FIG. 6A shows experimental data showing TALEN mediated gene knockout efficiency and AAV mediated transduction efficiency on day 14 of production. FIG. 6A shows more than 75% of T cells lost TCRa/b expression and most TCRa/b negative T cells expressed DLL3 CAR. FIG. 6B shows a majority of T cells lost CD52 expression and a subset of CD52 negative T cells expressed PD1 chimeric switch receptor. FIG. 6C shows flow cytometry plots depicting percentage of T cells expressing DLL3 CAR or chimeric switch receptor alone, or both DLL3 CAR and chimeric switch receptor on day 14 of production. The plots are gated on live single T cells. FIG. 6D shows T cell expansion during production.

### Example 6: Cytotoxic Activity of T Cells Expressing Chimeric Switch Receptors Using Multiplexed SSI

Cytotoxicity of T cells produced according to the methods in Example 5 was determined in a serial killing assay described in Example 3, using DMS 273 (DLL3-low, PDL1-low), DMS 273-PDL1(DLL3-low, PDL1-high), DMS 273-DLL3 (DLL3-high, PDL1-low), or DMS 273-DLL3-PDL1(DLL3-high, PDL1-high) as target cells.

In addition to the serial killing assay described in Example 3, a single stimulation long-term killing assay was also used to assess the potency of T cell products against high tumor burden. In this assay, different T cell effector activity parameters were simultaneously examined, including target cell killing, CAR T cell expansion and memory phenotypes. On the first day of the assay, 0.25×10⁶ CAR-T cells were mixed with PDL1-high NCI-H82 cells in a Grex-24 culture vessel at an effector:target ratio of 1:5 in 4 mL RPMI medium with 10% FBS. Every 2 to 3 days thereafter, 100 ul of mixed cells were analyzed by flow cytometry. The absolute number of viable target cells (hCD45−) and CAR T cells (hCD45⁺CAR⁺) were assessed using 123count eBeads (Thermofisher). Each condition was assayed in duplicate.

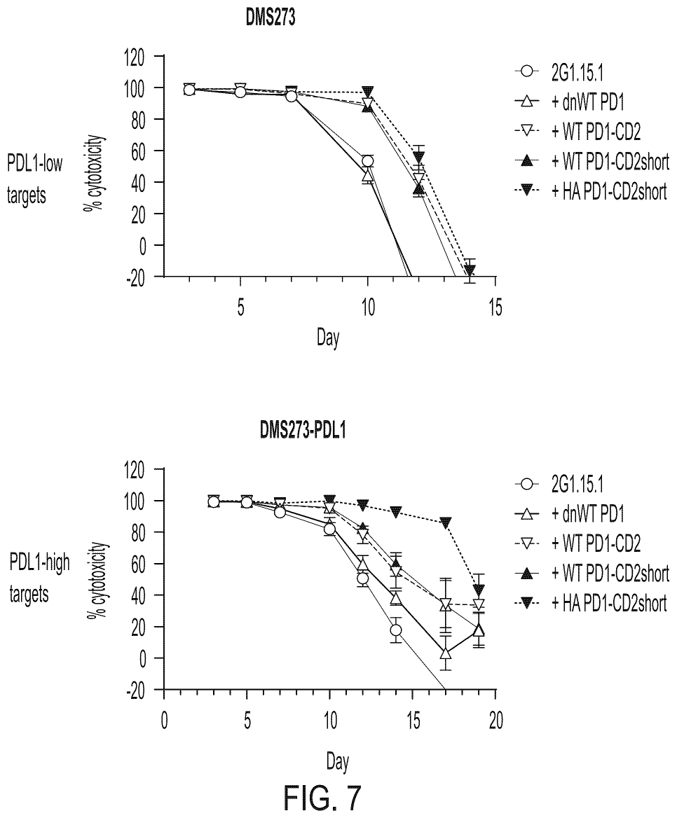

FIG. 7 shows experimental data from a serial killing assay showing cytotoxicity of T cells co-expressing DLL3 CAR and PD1 chimeric switch receptors (either with wildtype PD1 ectodomain or high affinity PD1 ectodomain) against DLL3 positive cells with low or high levels of PD-L1 expression. Dominant negative PD1 (dnWT PD1) alone did not confer benefits (e.g. did not enhance cytotoxicity of the DLL3 CAR T cells) in the assay, while chimeric switch receptors with intracellular signaling domains from CD28 or CD2 enhanced the activities of DLL3 CAR T cells (e.g. cytotoxicity).

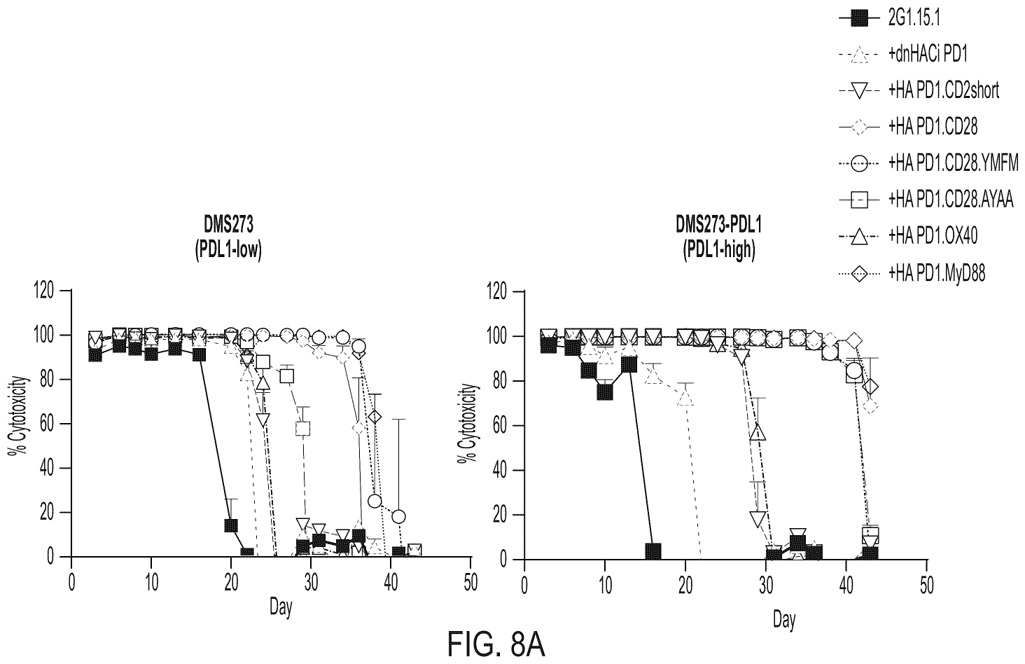

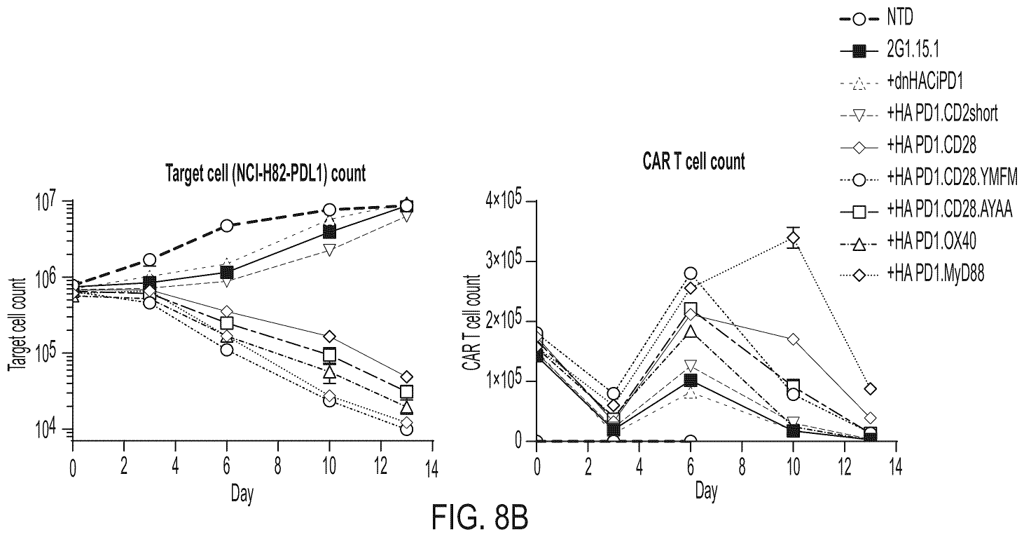

FIGS. 8A-B show results of CAR T cells expressing a CAR (2G1.15.1) co-expressing additional PD1 chimeric switch receptors with different intracellular domains produced by site-specific integration. Results or reporter assay in FIG. 8A showed that the additional PD1 chimeric switch receptors also increased cytotoxic activity as compared to CAR T without the PD1 chimeric switch receptor. The data in FIG. 8A show that multiple PD1 chimeric switch receptors demonstrated enhanced cytotoxic activity, as compared to control DLL3 CAR T cells, while dominant negative high affinity PD1 (dnHACiPD1) alone had little to modest effect on enhancing cytotoxicity. In FIG. 8B, a single stimulation long-term killing assay was conducted to evaluate target cell killing and CAR T expansion. In this assay, the target cells were suspension NCI-H82 cells overexpressing PDL1 (in contrast to the adherent target cells as in FIG. 8A), and the cell killing was done in a stringent condition, i.e., low E:T of 1:5 (as opposed to E:T of 9:1 in FIG. 8A). The remaining target cells and expansion of CAR T cells were analyzed by flow cytometry. The data in FIG. 8B show enhanced cytotoxicity of CAR T cells co-expressing several PD1 chimeric switch receptors as compared to the control CAR T cells and concomitant expansion of CAR-T cells co-expressing different PD1 chimeric switch receptors. Under the stringent conditions, CAR T cells with HA PD1 CD28, HA PD1 CD28.YMFM, HA PD1 CD28.AYAA or HA PD1 MyD88 chimeric switch receptor exhibited better cytotoxicity, while CAR T cells with HA PD1 CD2short chimeric switch receptor performed similarly to the control CAR T cells. In the high target-expressing cells, for example in this case high DLL3 expressing NCI-H82 cells, the effects of CD2 signaling from the PD1-CD2 chimeric switch receptor may be less pronounced. In the stringent experiment condition, the superior activities of chimeric switch receptors with a CD28 intracellular domain, e.g., the CD28 variants YMFM and AYAA, or the OX40 intracellular domain, are of particular interest. See FIG. 8B.

### Example 7: Cytokine Secretion from T Cells Expressing Chimeric Switch Receptors Using Multiplexed SSI

Cytokine secretion of T cells produced according to the methods in Example 5 were determined in the Meso Scale Discovery assay described in Example 4, using DMS273 cells (DLL3-low, PDL1-low), or DMS 273-PDL1 cells (DLL3-low, PDL1-high) as target cells.

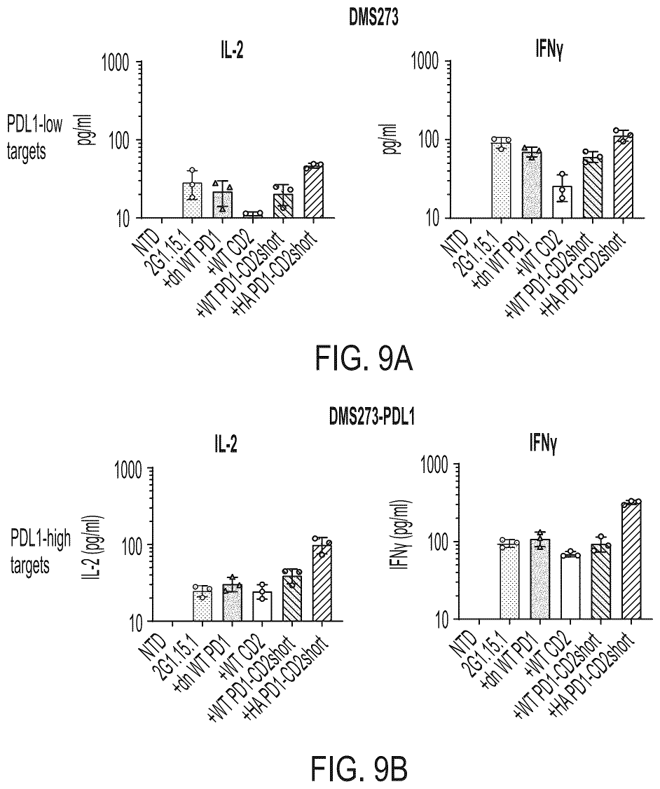

FIGS. 9A-B depict experimental data showing that T cells expressing both DLL3 CAR/CCR (2G1.15.1) and a PD1 chimeric switch receptor secreted cytokines when co-cultured with DLL3 positive target cells. When the T cells were co-cultured with PDL1-high DMS 273-PDL1 cells, WT PD1-CD2short and HA PD1-CD2short chimeric switch receptors (CSR) showed higher levels of cytokine secretion (for example, higher IL-2 secretion and to a less extent IFNγ secretion) than when CAR T cell were co-cultured with PDL1-low DMS 273 cells. Dominant negative PD1 (dnWT PD1) alone did not increase cytokine secretion in any condition tested. In this assay, the cells comprised a mixture of CAR+ and CAR− cells and a mixture of CSR+ and CSR-cells. The T cells were normalized (by adding NTD cells) at the end of CAR T production to have the same % of CAR+ cells, but normalization was not performed for the CSR+ cells. Since the CAR+% is the same across the samples, it is concluded that DLL3 CAR T cells (2G1.15.1) plus PD1-chimeric switch receptor secreted more cytokines than the parental CAR T cells without the PD1 switch receptor.

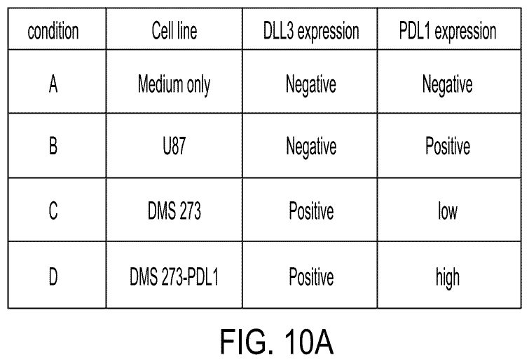

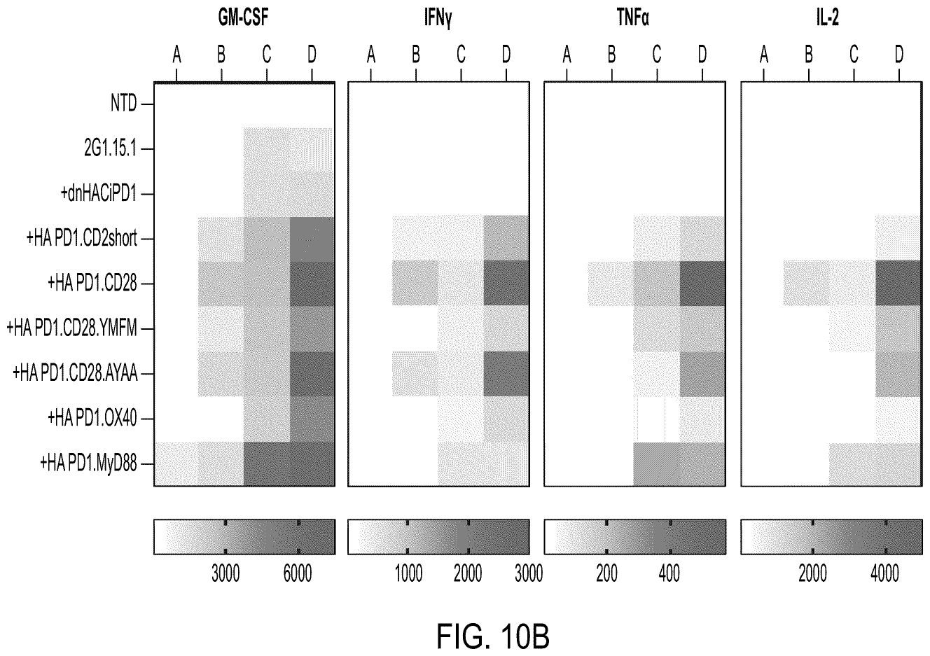

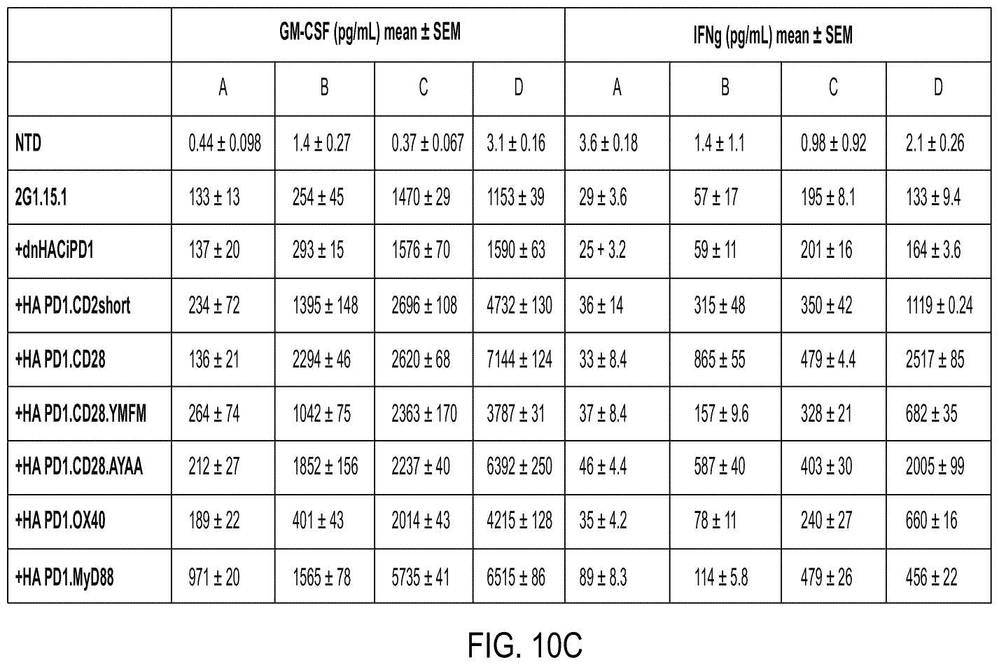

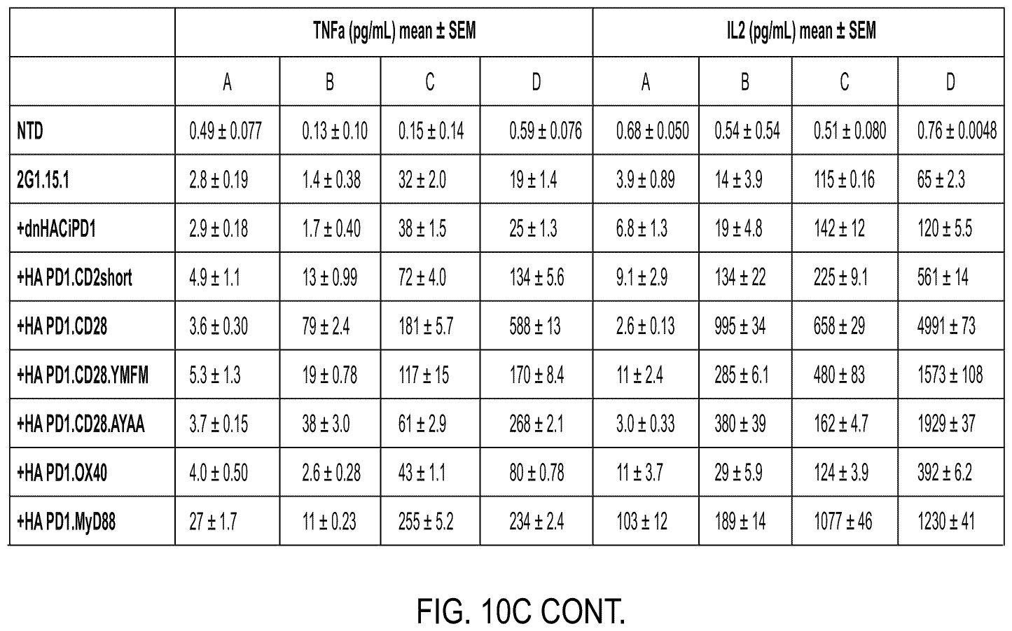

Next, we examined cytokine secretion of CAR T cells co-expressing different PD1 chimeric switch receptor when co-cultured with cell lines that express high or low levels of PDL1, and are target positive or negative (FIG. 10A). FIG. 10B presents data showing cytokine secretion from CAR T cells expressing chimeric switch receptors produced by site specific integration. The data values are shown in FIG. 10C. When the CAR T cells were co-cultured with PDL1-high cells (condition D), many chimeric switch receptors induced higher levels of cytokine secretion than when the same CAR T cells were co-cultured with PDL1-low DMS 273 cells (condition C), suggesting higher levels of PDL1 effectively trigger signaling through PD1 chimeric antigen receptors. HA PD1-CD28 induced very high levels of all cytokines tested, which might lead to severe adverse events in vivo or in patients (e.g. cytokine release syndrome or Immune Effector Cell-Associated Neurotoxicity Syndrome). HA PD1-MyD88 also induced very high level of GM-CSF, which can potentially lead to macrophage activation and toxicities in the clinic. CAR T co-expressing another chimeric switch receptor, for example, HA PD1 CD2short, HA PD1 CD28.YMFM, HA PD1 CD28.AYAA, and HA PD1 OX40 chimeric switch receptor secreted moderate levels of cytokines and may have better safety profile in vivo.

### Example 8: Growth Factor Independent Growth

To understand whether any of the chimeric switch receptors will result in aberrant CAR T proliferation, T cells co-expressing DLL3 CAR and PD1 chimeric switch receptors were cultured for 6 weeks in the absence of target cells or IL-2. On the first day of the assay, 0.4×10⁶ CAR-T cells were resuspended in T cell culture medium (RPMI with 10% FBS, lx Non-Essential Amino Acids, 1 mM Sodium Pyruvate and 25 mM HEPES) in a Grex-24 culture vessel. Every week thereafter, 100 ul of cell suspension was analyzed by flow cytometry. The absolute number of viable cells were assessed using 123count eBeads (Thermofisher). Each condition was assayed in duplicate. CAR T cells supplemented with IL-2 (100 IU/mL, 2×/week) served as a positive control. Half medium change was performed during the assay only when medium turned yellow.

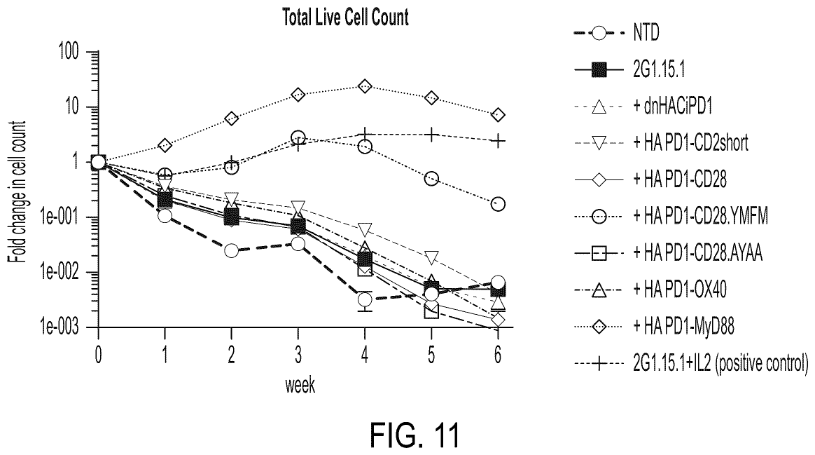

FIG. 11 presents data showing the expansion of CAR T cells expressing one of various PD1 chimeric switch receptors in the absence of target cells or growth factors. Total live cell count was quantified using flow cytometry. HA PD1-MyD88 induced aberrant growth of T cells in this assay, which may present a safety concern. Other chimeric switch receptors behaved similarly as the control CAR T or only induce transient acceptable levels of expansion of T cells.

### Example 9 Production and Cytotoxic Activity of T Cells Expressing DLL3 CAR without a CCR, with or without Chimeric Switch Receptors

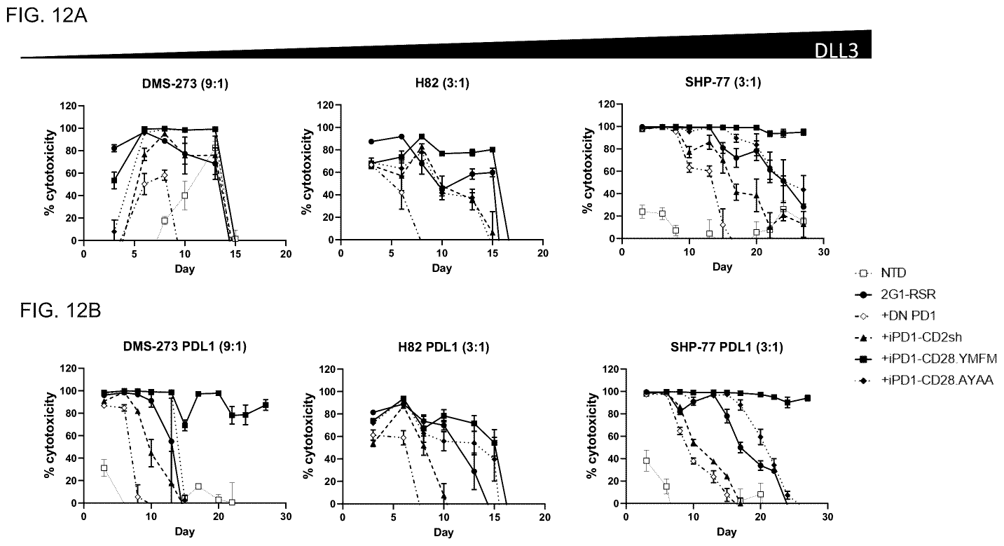

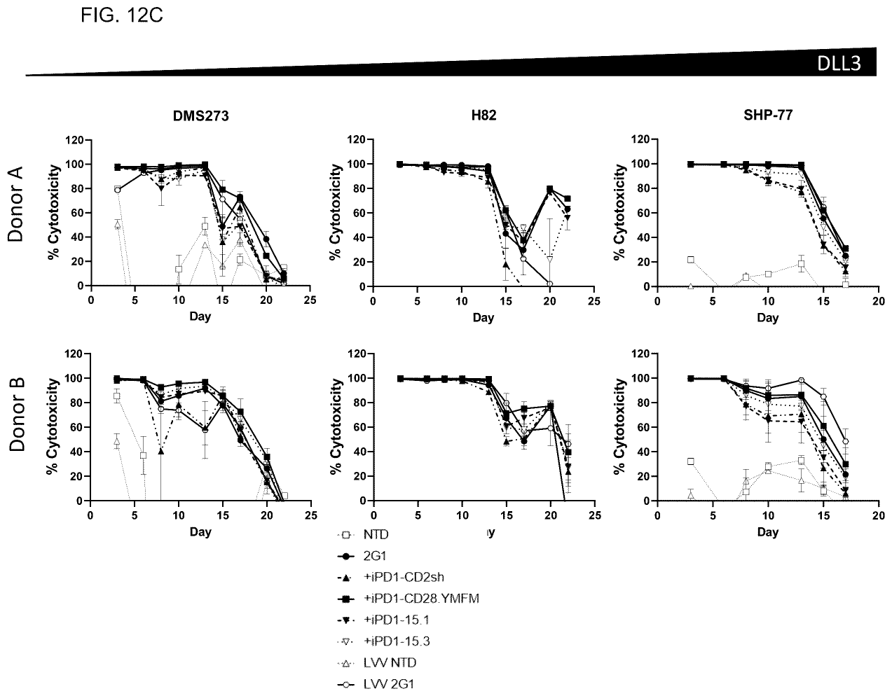

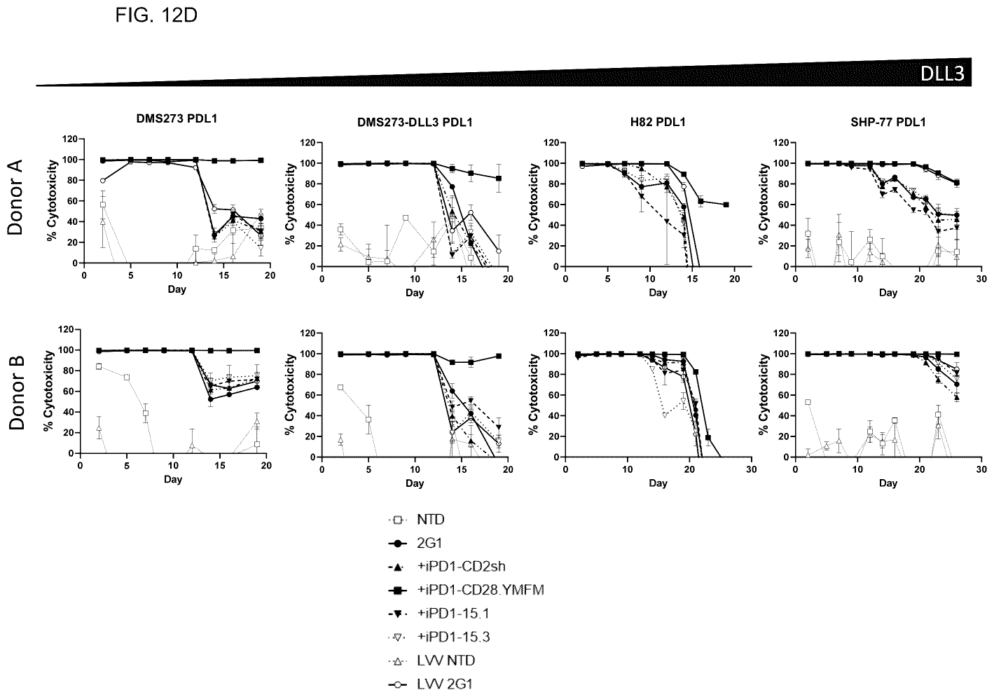

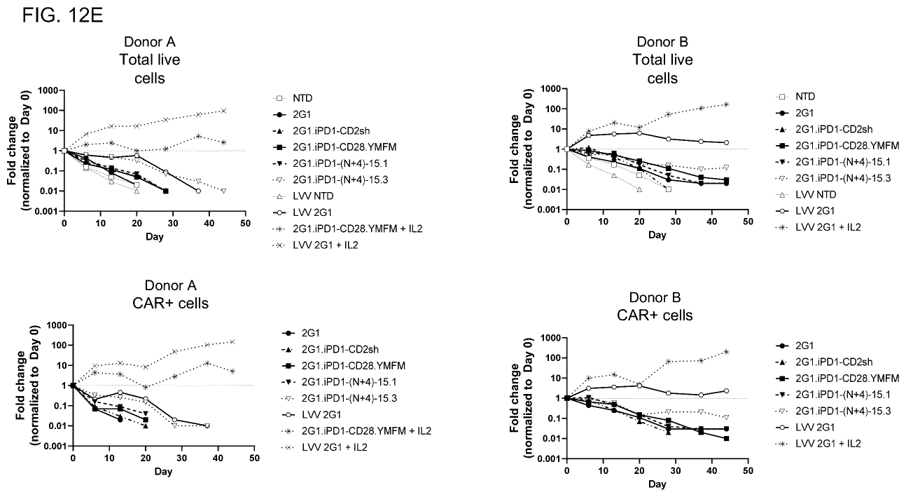

We next produced CAR T cells expressing a DLL3 CAR alone without a CCR, with or without a chimeric switch receptor, either by LVV transduction or by site-specific integration as described in Example 2 and Example 5 above, respectively. Cytotoxicity activity of the different CAR T cells produced by LVV transduction was analyzed in a long-term killing assay as described in Example 3. As shown in FIGS. 12A-12B, DLL3 CAR T expressing the high affinity PD1 chimeric switch receptor with the CD28 YMFM variant intracellular domain outperformed other constructs using either the PDL1-low (FIG. 12A) or PDL1-high (FIG. 12B) target cells.

Cytotoxic activity of CAR T cells generated by site-specific integration were analyzed as described in Example 6, either against PDL1-low or -high target cells. For comparison, inducible chimeric cytokine receptors comprising the high affinity PD1 extracellular domain, fused to a TPOR transmembrane domain (TPOR N+4, SEQ ID NO: 177) and an intracellular signaling domain containing the IL2Rb intracellular signaling domain in the form of SEQ ID NO: 4 (FIG. 12C) as in the construct iPD1-15.1, or SEQ ID NO 180 (FIG. 12C) as in the construct iPD1-15.3. Minimal positive effects of chimeric switch receptor were observed when PDL1-low target cells were used as compared to DLL3 CAR T cells without co-expressing the high affinity PD1 chimeric switch receptor or CAR T cells produced by LVV transduction (FIG. 12C, results from two different donors). In contrast, when PDL1-high target cells were used, DLL3 CAR T cells expressing the high affinity PD1 CD28 YMFM chimeric switch receptor outperformed the CAR T cells without the chimeric switch receptor or CAR T cells produced by LVV transduction (FIG. 12D, results from two different donors). The CAR T cells expressing the high affinity PD1-switch receptor signaling through the CD28 YMFM intracellular domain showed better activity than the construct signaling through the IL2Rb intracellular signaling domain in the constructs tested (FIG. 12D).

### Example 10 In Vitro and In Vivo Cytotoxicity of CAR T Cells Co-Expressing a Chimeric Switch Receptor

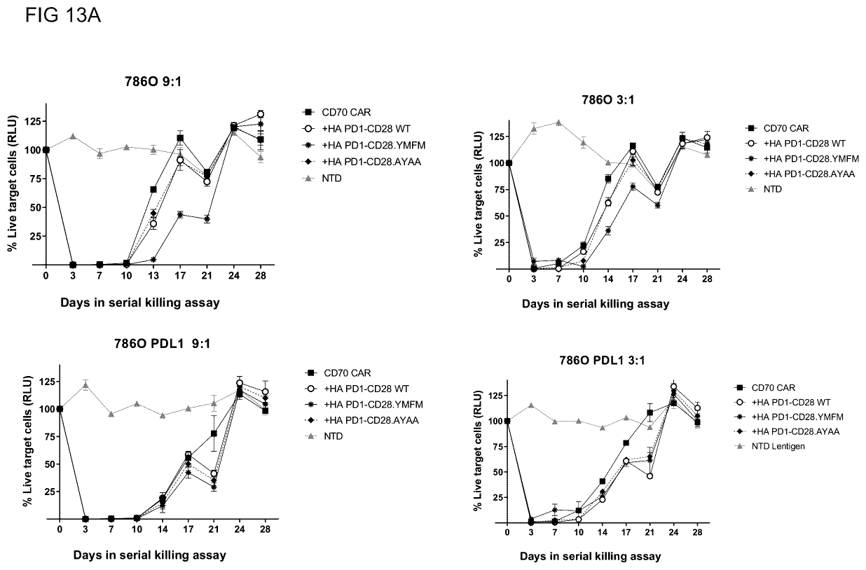

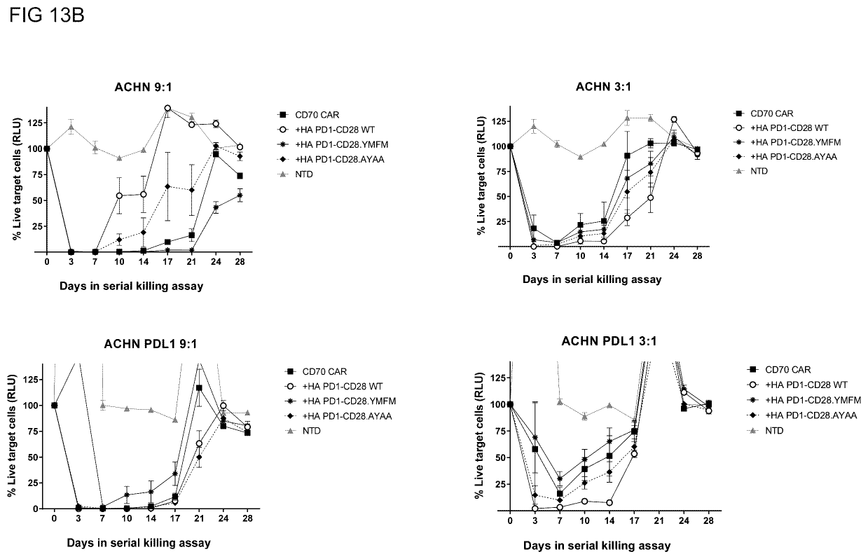

The PD1 CD28 switch receptor constructs were also tested in CAR T cells expressing an exemplary anti-CD70 CAR (SEQ ID NO: 179). The CAR T cells were generated by site-specific integration (SSI) as described above in Example 5. As shown in FIGS. 13A-13B, CD70 CAR T cells co-expressing a high affinity PD1-wild-type CD28 or variant CD28 chimeric switch receptors showed modest improvement as compared to CD70 CAR T control cells, especially at a low E:T ratio using either PDL1-low or PDL1-high, CD70-high 7860 target cells (FIG. 13A) or PDL1-low or PDL1-high, CD70-low ACHN target cells (FIG. 13B).

The PD1 chimeric switch receptor with the wild-type CD28 intracellular signaling domain elicited stronger cytokine secretion, as compared to variant CD28 intracellular signaling domains (FIGS. 10B-10C). Thus, the PD1 switch receptor with the variant CD28 signaling domain can be advantageous in providing increased activities in the context of a PD1 chimeric switch receptor with moderate cytokine secretion to ease any potential safety concerns. We next test CD70 CAR T cells co-expressing PD1-CD28 chimeric switch receptors in the mouse model to evaluate activity and assess toxicity. CAR T cells are generated using SSI as described in Example 5 with insertion of both CAR and PD1-CD28 chimeric switch receptor transgenes into the TRAC locus using bicistronic AAV vectors. NOD scid gamma (NSG) mice are implanted subcutaneously with tumor cells 786-0 overexpressing PDL1 and once the tumors attain a volume of 200 mm³, the mice are treated with CAR T cells at a suboptimal dose of 1×10⁶ to 5×10⁶ CAR+ T cells intravenously via tail vein injection to evaluate the benefit of PD1-CD28 chimeric switch receptors in vivo. Preliminary data show that mice dosed with a suboptimal low dose of CAR T cells that express a PD1-CD28 wild-type chimeric switch receptor, but not the PD1-CD28 YMFM variant chimeric switch receptor, exhibited weight losses, even though CAR T cytotoxicity was not observed in all animals at this low dose (data not shown). The data suggest that variant CD28 intracellular domain may confer the benefit of improved safety profile in vivo.

All references cited herein, including patents, patent applications, papers, text books, and the like, and the references cited therein, to the extent that they are not already, are hereby incorporated by reference in their entirety.

Although the disclosed teachings have been described with reference to various applications, methods, kits, and compositions, it will be appreciated that various changes and modifications can be made without departing from the teachings herein and the claimed invention below. The foregoing examples are provided to better illustrate the disclosed teachings and are not intended to limit the scope of the teachings presented herein. While the present teachings have been described in terms of these exemplary embodiments, the skilled artisan will readily understand that numerous variations and modifications of these exemplary embodiments are possible without undue experimentation. All such variations and modifications are within the scope of the current teachings.

1. A polynucleotide encoding a chimeric polypeptide comprising an extracellular domain, a transmembrane domain, and one or more intracellular domains, wherein the extracellular domain comprises a PD-1 extracellular domain or a TGFβ receptor extracellular domain or an antibody or an antigen-binding portion of an antibody that specifically recognizes and binds to PDL1 or TGFβ, optionally wherein:
(a) the PD-1 extracellular domain comprises the amino acid sequence of SEQ ID NO:9 or a variant thereof, or
(b) the TGFβ receptor extracellular domain comprises the amino acid sequence of SEQ ID NO: 12 or a variant thereof, or
(c) the antibody or antigen-binding portion of an antibody specifically recognizes and binds to PDL1, TGFβ or any one or more of TGFβ1, TGFβ2 and TGFβ3.

2. (canceled)

3. The polynucleotide of claim 1, wherein the extracellular domain comprises a PD-1 extracellular domain variant, wherein the PD-1 extracellular domain variant comprises the amino acid sequence of SEQ ID NO:10.

4. (canceled)

5. The polynucleotide of claim 1, wherein the extracellular domain comprises the amino acid sequence of SEQ ID NO:12 or the amino acid sequence of SEQ ID NO:13.

6. (canceled)

7. (canceled)

8. (canceled)

9. The polynucleotide of claim 1, wherein the one or more intracellular domains comprise one or more intracellular signalling domains, optionally wherein the one or more intracellular signalling domains are selected from the group consisting of a CD28 intracellular signalling domain, CD2 intracellular signalling domain, MyD88 intracellular signalling domain, ICOS intracellular signalling domain, DAP10 intracellular signalling domain, OX40 intracellular signalling domain, BAFFR intracellular signalling domain, and CD40 intracellular signalling domain, any variant thereof, and any combination thereof.

10. The polynucleotide of claim 1, wherein the one or more intracellular domains comprise the amino acid sequence of one or more of SEQ ID NO:19, SEQ ID NO:20, SEQ ID NO:15, SEQ ID NO:18, SEQ ID NO:23, SEQ ID NO:24, SEQ ID NO:27, SEQ ID NO:29, SEQ ID NO:33, SEQ ID NO:31, SEQ ID NO:35, and any combination thereof.

11. The polynucleotide of claim 1, wherein the transmembrane domain comprises a CD28 transmembrane domain, CD2 transmembrane domain, PD-1 transmembrane domain, ICOS transmembrane domain, DAP10 transmembrane domain, OX40 transmembrane domain, BAFFR transmembrane domain or CD40 transmembrane domain.

12. The polynucleotide of claim 1, wherein the transmembrane domain comprises the amino acid sequence of SEQ ID NO:17, SEQ ID NO:22, SEQ ID NO:26, SEQ ID NO:28, SEQ ID NO:30, SEQ ID NO:32, or SEQ ID NO:34.

13. The polynucleotide of claim 1, wherein the intracellular domain comprises a variant CD28 intracellular domain, and optionally further wherein the intracellular domain comprises the amino acid sequence of SEQ ID NO:19 or SEQ ID NO:20.

14. The polynucleotide of claim 1, wherein the intracellular domain comprises a CD2 intracellular domain or a variant thereof, optionally further wherein the transmembrane domain comprises a CD2 transmembrane domain.

15. (canceled)

16. (canceled)

17. (canceled)

18. The polynucleotide of claim 1, wherein the intracellular domain comprises a variant CD2 intracellular domain that comprises the amino acid sequences of SEQ ID NO: 170 and SEQ ID NO: 176 or the amino acid sequences of SEQ ID NO: 174 and SEQ ID NO: 175, and optionally the variant CD2 intracellular domain is a truncated CD2 intracellular domain.

19. (canceled)

20. (canceled)

21. The polynucleotide of claim 1, wherein the intracellular domain comprises a variant CD2 intracellular domain that comprises the amino acid sequences of SEQ ID NOs: 170, 171, 173, 174 and 175 or the amino acid sequences of SEQ ID NOs: 170-176, and optionally the variant CD2 intracellular domain is a truncated CD2 intracellular domain.

22. The polynucleotide of claim 21, wherein the intracellular domain comprises a truncated CD2 intracellular domain, wherein the truncated CD2 intracellular domain comprises the amino acid sequence of SEQ ID NO:23 or 24 or an amino acid sequence that is at least about 90% identical to the amino acid sequence of SEQ ID NO:23 or 24.

23. (canceled)

24. (canceled)

25. The polynucleotide of claim 1, wherein the polypeptide further comprises a hinge domain located between the extracellular domain and the transmembrane domain.

26. The polynucleotide of claim 1, wherein the polypeptide comprises the amino acid sequence of any one of SEQ ID NOS: 75-115 or an amino acid sequence that is at least 90% identical to any one of SEQ ID NOS: 75-115, optionally the polypeptide further comprises a signal peptide.

27. The polynucleotide of claim 1, wherein the polypeptide further comprises a signal peptide.

28. The polynucleotide of claim 27, wherein the signal peptide comprises a CD8α signal peptide or a TGFβ receptor signal peptide, optionally wherein the signal peptide comprises the amino acid sequence of SEQ ID NO:1 or 2.

29. The polynucleotide of claim 1, wherein the polynucleotide further encodes a second polypeptide, optionally wherein the second polypeptide comprises a chimeric cytokine receptor (CCR) or a chimeric antigen receptor (CAR).

30. The polynucleotide of claim 29, wherein the CCR is constitutively active or inducible.

31. A vector comprising the polynucleotide of claim 1.

32. The vector of claim 31, wherein the vector is a viral vector, optionally wherein the vector is a lentiviral vector.

33. A chimeric polypeptide encoded by the polynucleotide of claim 1.

34. A chimeric polypeptide comprising an extracellular domain, a transmembrane domain, and one or more intracellular domains, wherein the extracellular domain comprises a PD-1 extracellular domain or a TGFβ receptor extracellular domain or an antibody or an antigen-binding portion of an antibody that specifically recognizes and binds to PDL1 or TGFβ, optionally wherein:
(a) the PD-1 extracellular domain comprises the amino acid sequence of SEQ ID NO:9 or a variant thereof, or
(b) the TGFβ receptor extracellular domain comprises the amino acid sequence of SEQ ID NO: 12 or a variant thereof.

35. (canceled)

36. The chimeric polypeptide of claim 34, wherein the PD-1 extracellular domain variant comprises the amino acid sequence of SEQ ID NO:10.

37. (canceled)

38. The chimeric polypeptide of claim 34, wherein the extracellular domain comprises the amino acid sequence of SEQ ID NO:12 or the amino acid sequence of SEQ ID NO:13.

39. The chimeric polypeptide of claim 34, wherein the one or more intracellular domains comprise one or more intracellular signalling domains, optionally wherein the one or more intracellular signalling domains are selected from the group consisting of a CD28 intracellular signalling domain, CD2 intracellular signalling domain, MyD88 intracellular signalling domain, ICOS intracellular signalling domain, DAP10 intracellular signalling domain, OX40 intracellular signalling domain, BAFFR intracellular signalling domain, and CD40 intracellular signalling domain, any variant thereof, and any combination thereof.

40. The chimeric polypeptide of claim 34, wherein the one or more intracellular domains comprise the amino acid sequence of one or more of SEQ ID NO:19, SEQ ID NO:20, SEQ ID NO:15, SEQ ID NO:18, SEQ ID NO:23, SEQ ID NO:24, SEQ ID NO:15, SEQ ID NO:29, SEQ ID NO:27, SEQ ID NO:33, SEQ ID NO:31, SEQ ID NO:35, and any combination thereof.

41. The chimeric polypeptide of claim 34, wherein the transmembrane domain comprises a CD28 transmembrane domain, CD2 transmembrane domain, PD-1 transmembrane domain, ICOS transmembrane domain, DAP10 transmembrane domain, OX40 transmembrane domain, BAFFR transmembrane domain or CD40 transmembrane domain.

42. The chimeric polypeptide of claim 34, wherein the transmembrane domain comprises the amino acid sequence of SEQ ID NO:17, SEQ ID NO:22, SEQ ID NO:26, SEQ ID NO:28, SEQ ID NO:30, SEQ ID NO:32, or SEQ ID NO:34.

43. The chimeric polypeptide of claim 34, wherein the one or more intracellular domains comprises a variant CD28 intracellular domain, and optionally further wherein the variant CD28 intracellular domain comprises the amino acid sequence of SEQ ID NO:19 or SEQ ID NO:20.

44. The chimeric polypeptide of claim 34, wherein the one or more intracellular domains comprises a CD2 intracellular domain or a variant thereof, optionally further wherein the transmembrane domain comprises a CD2 transmembrane domain.

45. (canceled)

46. (canceled)

47. The chimeric polypeptide of claim 44, wherein the intracellular domain comprises the amino acid sequence of at least one or at least two of SEQ ID NOs: 170, 171, 172, 173, 174, 175 and 176, and optionally the variant CD2 intracellular domain is a truncated CD2 intracellular domain.

48. (canceled)

49. The chimeric polypeptide of claim 44, wherein the intracellular domain comprises a variant CD2 intracellular domain that comprises the amino acid sequences of SEQ ID NO: 170 and 176, or the amino acid sequences of SEQ ID NO: 174 and 175, and optionally the variant CD2 intracellular domain is a truncated CD2 intracellular domain.

50. (canceled)

51. (canceled)

52. The chimeric polypeptide of claim 44, wherein the intracellular domain comprises a variant of a CD2 intracellular domain that comprises the amino acid sequences of SEQ ID NOs: 170, 171, 173, 174 and 175 or SEQ ID NOs: 170-176, and optionally the variant CD2 intracellular domain is a truncated CD2 intracellular domain.

53. The chimeric polypeptide of claim 44, wherein the one or more intracellular domains comprises a truncated CD2 intracellular domain, wherein the truncated CD2 intracellular domain comprises the amino acid sequence of SEQ ID NO:24 or an amino acid sequence that is at least about 90% identical to the amino acid sequence of SEQ ID NO:24.

54. (canceled)

55. The chimeric polypeptide of claim 34, wherein the transmembrane domain comprises a CD28 transmembrane domain or a CD2 transmembrane domain, optionally wherein the CD28 transmembrane domain comprises the amino acid sequence of SEQ ID NO: 17 or an amino acid sequence that is at least about 90% identical to the amino acid sequence of SEQ ID NO: 17, or the CD2 transmembrane domain comprises the amino acid sequence of SEQ ID NO:22 or an amino acid sequence that is at least about 90% identical to the amino acid sequence of SEQ ID NO:22.

56. The chimeric polypeptide of claim 34, wherein the chimeric polypeptide further comprises a signal peptide.

57. The chimeric polypeptide of claim 34, wherein the polypeptide further comprises a hinge domain located between the extracellular domain and the transmembrane domain.

58. The chimeric polypeptide of claim 34, wherein the polypeptide comprises the amino acid sequence of any one of SEQ ID NOs:75-115 or an amino acid sequence that is at least 90% identical to any one of SEQ ID NOs:75-115.

59. (canceled)

60. The chimeric polypeptide of claim 56, wherein the signal peptide comprises a CD8α signal peptide or a TGFβ receptor signal peptide, optionally wherein the signal peptide comprises the amino acid sequence of SEQ ID NO:1 or 2.

61. An engineered immune cell comprising the polynucleotide of claim 1.

62. An engineered immune cell comprising the vector of claim 31.

63. An engineered immune cell comprising or expressing the chimeric polypeptide of claim 34.

64. The engineered immune cell of claim 63, wherein the cell further comprises or expresses a CAR, wherein the CAR comprises an extracellular ligand-binding domain, a transmembrane domain, and an intracellular signaling domain.

65. The engineered immune cell of claim 64, wherein the CAR intracellular signaling domain comprises any one or more of a CD3ζ signaling domain, a CD28 signaling domain, and a 4-1 BB signaling domain.

66. The engineered immune cell of claim 64, wherein the CAR extracellular ligand-binding domain specifically recognizes or binds to DLL3, CD370, BCMA, Claudin 18.2, Muc16 or Flt3.

67. The engineered immune cell of claim 64, wherein the cell further comprises or expresses a chimeric cytokine receptor (CCR).

68. The engineered immune cell of claim 67, wherein the CCR is constitutively active or inducible.

69. The engineered immune cell of claim 63, wherein the chimeric polypeptide comprises the intracellular domain comprising the amino acid sequence of SEQ ID NO: 19, 20, or 24.

70. The engineered immune cell of claim 69, wherein the chimeric polypeptide comprises a PD1 extracellular domain or a variant thereof, wherein the immune cell secrets reduced levels of cytokine as compared to an engineered immune cell that comprises a chimeric polypeptide comprising the same extracellular domain and an intracellular domain comprising the amino acid sequence of SEQ ID NO: 18 or 23, and optionally wherein the reduced level of cytokine is about a 1.5 fold, 2 fold, 2.5 fold, 3 fold, 3.5 fold, 4 fold, 4.5 fold, and 5 fold reduction.

71. An engineered immune cell comprising or expressing a DLL3 CAR that comprises the amino acid sequence of SEQ ID NO: 73 or 165, and a CCR that comprises the amino acid sequence of SEQ ID NO:162.

72. The engineered immune cell of claim 71, wherein the engineered immune cell comprises or expresses a polypeptide comprising the amino acid sequence of SEQ ID NO: 74, with or without the signal peptide.

73. The engineered immune cell of claim 63, wherein the immune cell is a T cell, tumor infiltrating lymphocyte (TIL), NK cell, dendritic cell, or NK-T cell.

74. The engineered immune cell of claim 63, wherein the cell is an autologous T cell.

75. The engineered immune cell of claim 63, wherein the cell is an allogeneic T cell.

76. A population of cells comprising at least about 1×10⁴, 1×10⁵, 1×10⁶, 1×10⁷ or 1×10⁸ cells of claim 63.

77. A composition comprising the cell of claim 63 and a pharmaceutically acceptable carrier.

78. A method of treating a disease or condition in a patient comprising administering to the patient the cell of claim 64.

79. The method of claim 78, wherein the subject is a human and the condition is a cancer.

80. The method of claim 79, wherein the cancer is a hematological malignancy or a solid cancer.

81. The method of claim 80, wherein the cancer is a hematological malignancy optionally selected from acute lymphoblastic leukemia (ALL), acute myeloid leukemia (AML), chronic myelogenous leukemia (CML), chronic eosinophilic leukemia (CEL), myelodysplasia syndrome (MDS), non-Hodgkin's lymphoma (NHL), and multiple myeloma (MN).

82. The method of claim 80, wherein the cancer is a solid cancer optionally selected from biliary cancer, bladder cancer, bone and soft tissue carcinoma, brain tumor, breast cancer, cervical cancer, colon cancer, colorectal adenocarcinoma, colorectal cancer, desmoid tumor, embryonal cancer, endometrial cancer, esophageal cancer, gastric cancer, gastric adenocarcinoma, glioblastoma multiforme, gynecological tumor, head and neck squamous cell carcinoma, hepatic cancer, lung cancer, malignant melanoma, osteosarcoma, ovarian cancer, pancreatic cancer, pancreatic ductal adenocarcinoma, primary astrocytic tumor, primary thyroid cancer, prostate cancer, renal cancer, renal cell carcinoma, rhabdomyosarcoma, skin cancer, soft tissue sarcoma, testicular germ-cell tumor, urothelial cancer, uterine sarcoma, and uterine cancer.

83. A method of making an engineered immune cell comprising the step of introducing the polynucleotide of claim 1, into an immune cell.

84. (canceled)

85. (canceled)

86. (canceled)

87. (canceled)

88. (canceled)

89. (canceled)

90. (canceled)


SEQUENCE LISTING (180 sequences)

SEQ ID NO: 1 - protein, 21 aa, synthetic construct
  REGION 1..21: note = Description of sequence: CD8 signal sequence
MALPVTALLLPLALLLHAARP

SEQ ID NO: 2 - protein, 22 aa, synthetic construct
  REGION 1..22: note = Description of sequence: BR2 signal sequence
MGRGLLRGLWPLHIVLWTRIAS

SEQ ID NO: 3 - protein, 105 aa, synthetic construct
  REGION 1..105: note = Description of sequence: TpoR (S505N W515K)
SDPTRVETATETAWISLVTALHLVLGLNAVLGLLLLRKQFPAHYRRLRHALWPSLPDLHRVLGQYLRDTAALSPPKATVSDTCEEVEPSLLEILPKSSERTPLPL

SEQ ID NO: 4 - protein, 75 aa, synthetic construct
  REGION 1..75: note = Description of sequence: IL2Rb-YY
DEGVAGAPTGSSPQPLQPLSGEDDAYCTFPSRDDLLLFSPSGQGEFRALNARLPLNTDAYLSLQELQGQDPTHLV

SEQ ID NO: 5 - protein, 187 aa, synthetic construct
  REGION 1..187: note = Description of sequence: dnWT PD1
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQ

SEQ ID NO: 6 - protein, 187 aa, synthetic construct
  REGION 1..187: note = Description of sequence: dnHACiPD1
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVVGVVGGLLGSLVLLVWVLAVICSRAARGTIGARRTGQ

SEQ ID NO: 7 - protein, 177 aa, synthetic construct
  REGION 1..177: note = Description of sequence: dnWT BR2
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSS

SEQ ID NO: 8 - protein, 151 aa, synthetic construct
  REGION 1..151: note = Description of sequence: dN25 BR2(DNR)
QLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSS

SEQ ID NO: 9 - protein, 150 aa, synthetic construct [sequence omitted]

SEQ ID NO: 10 - protein, 150 aa, synthetic construct
  REGION 1..150: note = Description of sequence: HA PD1 ECD
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLV

SEQ ID NO: 11 - protein, 20 aa, synthetic construct
  REGION 1..20: note = Description of sequence: PD-1 signal peptide
MQIPQAPWPVVWAVLQLGWR

SEQ ID NO: 12 - protein, 144 aa, synthetic construct
  REGION 1..144: note = Description of sequence: WT BR2 ECD
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQ

SEQ ID NO: 13 - protein, 118 aa, synthetic construct
  REGION 1..118: note = Description of sequence: dN25 BR2 ECD
QLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQ

SEQ ID NO: 14 - protein, 37 aa, synthetic construct
  REGION 1..37: note = Description of sequence: PD1 TM
VGVVGGLLGSLVLLVWVLAVICSRAARGTIGARRTGQ

SEQ ID NO: 15 - protein, 172 aa, synthetic construct
  REGION 1..172: note = Description of sequence: MyD88
MAAGGPGAGSAAPVSSTSSLPLAALNMRVRRRLSLFLNVRTQVAADWTALAEEMDFEYLEIRQLETQADPTGRLLDAWQGRPGASVGRLLDLLTKLGRDDVLLELGPSIEEDCQKYILKQQQEEAEKPLQVAAVDSSVPRTAELAGITTLDDPLGHMPERFDAFICYCPSDI

SEQ ID NO: 16 - protein, 12 aa, synthetic construct
  REGION 1..12: note = Description of sequence: CD28 ECD (truncated)
CPSPLFPGPSKP

SEQ ID NO: 17 - protein, 27 aa, synthetic construct
  REGION 1..27: note = Description of sequence: CD28 TM
FWVLVVVGGVLACYSLLVTVAFIIFWV

SEQ ID NO: 18 - protein, 41 aa, synthetic construct
  REGION 1..41: note = Description of sequence: CD28 intracellular
RSKRSRLLHSDYMNMTPRRPGPTRKHYQPYAPPRDFAAYRS

SEQ ID NO: 19 - protein, 41 aa, synthetic construct
  REGION 1..41: note = Description of sequence: CD28.YMFM intracellular
RSKRSRLLHSDYMFMTPRRPGPTRKHYQPYAPPRDFAAYRS

SEQ ID NO: 20 - protein, 41 aa, synthetic construct
  REGION 1..41: note = Description of sequence: CD28.AYAA intracellular
RSKRSRLLHSDYMNMTPRRPGPTRKHYQAYAAPRDFAAYRS

SEQ ID NO: 21 - protein, 12 aa, synthetic construct
  REGION 1..12: note = Description of sequence: CD2 ECD (truncated)
VEPVSCPEKGLD

SEQ ID NO: 22 - protein, 26 aa, synthetic construct
  REGION 1..26: note = Description of sequence: CD2 TM
IYLIIGICGGGSLLMVFVALLVFYIT

SEQ ID NO: 23 - protein, 119 aa, synthetic construct
  REGION 1..119: note = Description of sequence: CD2 intracellular (full);
KRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSNGYF

SEQ ID NO: 24 - protein, 85 aa, synthetic construct
  REGION 1..85: note = Description of sequence: CD2 intracellular (short)
KRKKQTPQNPATSQHPPPPPGHRSQAPSHRPPPPGHRVQHQPQKRPPAPSGTQVHQQKGPPLPRPRVQPKPPHGAAENSLSPSSN

SEQ ID NO: 25 - protein, 30 aa, synthetic construct
  REGION 1..30: note = Description of sequence: DAP10 ECD
QTTPGERSSLPAFYPGTSGSCSGCGSLSLP

SEQ ID NO: 26 - protein, 21 aa, synthetic construct
  REGION 1..21: note = Description of sequence: DAP10 TM D57N
LLAGLVAANAVASLLIVGAVF

SEQ ID NO: 27 - protein, 24 aa, synthetic construct
  REGION 1..24: note = Description of sequence: DAP10 intracellular
LCARPRRSPAQEDGKVYINMPGRG

SEQ ID NO: 28 - protein, 21 aa, synthetic construct
  REGION 1..21: note = Description of sequence: ICOS TM
FWLPIGCAAFVVVCILGCILI

SEQ ID NO: 29 - protein, 38 aa, synthetic construct
  REGION 1..38: note = Description of sequence: ICOS intracellular
CWLTKKKYSSSVHDPNGEYMFMRAVNTAKKSRLTDVTL

SEQ ID NO: 30 - protein, 22 aa, synthetic construct
  REGION 1..22: note = Description of sequence: CD40 TM
ALVVIPIIFGILFAILLVLVFI

SEQ ID NO: 31 - protein, 62 aa, synthetic construct
  REGION 1..62: note = Description of sequence: CD40 intracellular
KKVAKKPTNKAPHPKQEPQEINFPDDLPGSNTAAPVQETLHGCQPVTQEDGKESRISVQERQ

SEQ ID NO: 32 - protein, 21 aa, synthetic construct
  REGION 1..21: note = Description of sequence: OX40 TM
VAAILGLGLVLGLLGPLAILL

SEQ ID NO: 33 - protein, 42 aa, synthetic construct
  REGION 1..42: note = Description of sequence: OX40 intracellular
ALYLLRRDQRLPPDAHKPPGGGSFRTPIQEEQADAHSTLAKI

SEQ ID NO: 34 - protein, 21 aa, synthetic construct
  REGION 1..21: note = Description of sequence: BAFFR TM
FGAPALLGLALVLALVLVGLV

SEQ ID NO: 35 - protein, 85 aa, synthetic construct
  REGION 1..85: note = Description of sequence: BAFFR intracellular
SWRRRQRRLRGASSAEAPDGDKDAPEPLDKVIILSPGISDATAPAWPPPGEDPGTTPPGHSVPVPATELGSTELVTTKTAGPEQQ

SEQ ID NO: 36 - protein, 45 aa, synthetic construct
  REGION 1..45: note = Description of sequence: CD8-Alpha hinge
TTTPAPRPPTPAPTIASQPLSLRPEACRPAAGGAVHTRGLDFACD

SEQ ID NO: 37 - DNA, 63 bp, synthetic construct
  misc_feature 1..63: note = Description of sequence: CD8 signal sequence
  source 1..63: mol_type = other DNA
atggccctgccagtgaccgccctgctgctgcctctggccctgctgctgcacgccgctagaccc

SEQ ID NO: 38 - DNA, 66 bp, synthetic construct
  misc_feature 1..66: note = Description of sequence: BR2 signal sequence
  source 1..66: mol_type = other DNA
atgggtcgggggctgctcaggggcctgtggccgctgcacatcgtcctgtggacgcgtatcgccagc

SEQ ID NO: 39 - DNA, 315 bp, synthetic construct
  misc_feature 1..315: note = Description of sequence: TpoR (S505N W515K)
  source 1..315: mol_type = other DNA
tcagaccctactagagtcgagaccgctaccgagaccgcttggatctctctggtgaccgccctgcacctggtgctgggcctgaacgccgtgctgggcctgctgctgctgaggaagcagttcccagcacactaccggagactgaggcacgcactgtggccaagcctgcccgacctgcacagggtgctgggacagtatctgagggatacagccgccctgagcccacctaaggcaaccgtgtccgacacatgcgaggaggtggaaccaagtctgctggaaatcctgccaaaatcctctgagcggacacccctgcccctg

SEQ ID NO: 40 - DNA, 225 bp, synthetic construct
  misc_feature 1..225: note = Description of sequence: IL2Rb-YY
  source 1..225: mol_type = other DNA
gacgagggagtggcaggagcaccaaccggcagctccccccagcctctgcagccactgtccggagaggacgatgcatactgcacattcccttctcgggacgatctgctgctgttctctccaagcggacagggagagtttcgggccctgaacgccagactgcccctgaataccgacgcctatctgagcctgcaggagctgcagggacaggaccccacacacctggtg

SEQ ID NO: 41 - DNA, 561 bp, synthetic construct
  misc_feature 1..561: note = Description of sequence: dnWT PD1
  source 1..561: mol_type = other DNA
cccggatggtttctggatagccctgataggccctggaaccccccaactttttcacccgccctgctggtcgtcaccgaaggagacaacgccaccttcacatgcagcttttccaacacctctgagagcttcgtgctgaattggtacaggatgtccccatctaaccagacagacaagctggcagcatttcctgaggaccgctcccagccaggacaggattgccggttcagagtgacccagctgcccaatggccgggactttcacatgtctgtggtgagagcccggagaaacgatagcggcacatacctgtgcggagcaatctccctggcaccaaaggcacagatcaaggagtctctgagggcagagctgagggtgaccgagaggagggcagaggtgcctacagcacacccaagcccttccccacggcccgcaggccagttccagaccctggtggtgggagtggtgggaggcctgctgggcagcctggtgctgctggtgtgggtgctggcagtcatttgtagcagagccgcaagaggaactatcggagcaagacggacagggcag

SEQ ID NO: 42 - DNA, 561 bp, synthetic construct
  misc_feature 1..561: note = Description of sequence: dnHACiPD1
  source 1..561: mol_type = other DNA
cctggatggtttctggactcccctgataggccctggaatcccccaactttctcccctgccctgctggtggtcactgaaggcgacaacgccaccttcacatgcagcttttccaacacctctgagagcttccacgtgatctggcacagggagtccccatctggccagaccgacacactggcagcatttcctgaggaccgctcccagccaggacaggattgccggttcagagtgacccagctgcccaacggccgggactttcacatgtctgtggtgagagcccggagaaatgatagcggcacctacgtgtgcggcgtgatctccctggcccccaagatccagatcaaggagtctctgagggcagagctgagggtgaccgagaggagggcagaggtgcctacagcacacccaagcccttccccacggcccgcaggacagttccagacactggtggtgggagtggtgggaggcctgctgggcagcctggtgctgctggtgtgggtgctggctgtcatctgtagcagggccgcaagaggcaccattggggcacgaaggactgggcag

SEQ ID NO: 43 - DNA, 531 bp, synthetic construct
  misc_feature 1..531: note = Description of sequence: dnWT BR2
  source 1..531: mol_type = other DNA
acgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctaccgcgttaaccggcagcagaagctgagttca

SEQ ID NO: 44 - DNA, 453 bp, synthetic construct
  misc_feature 1..453: note = Description of sequence: dN25 BR2(DNR)
  source 1..453: mol_type = other DNA
caactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctaccgcgttaaccggcagcagaagctgagttca

SEQ ID NO: 45 - DNA, 450 bp, synthetic construct
  misc_feature 1..450: note = Description of sequence: WT PD1 ECD
  source 1..450: mol_type = other DNA
ccaggctggttcctggatagccccgacagaccttggaatccccctacattcagccctgctctgctggtcgtgaccgagggcgacaacgccaccttcacatgcagcttcagcaacaccagcgagtcttttgtgctgaactggtatcggatgagcccttctaaccagacagataagctggcagccttccccgaagatagaagccaacctggccaggactgcagattcagagtgacccagctgcctaacggccgggacttccacatgtctgtggtgcgggccagacgcaacgacagcggcacctacctgtgcggcgccatcagcctggcccctaaggcccagatcaaggaaagcctgagagccgagctgcgggtgacagaaagaagggccgaagtgcccaccgcccacccttccccttcccccagacctgccggacaatttcagaccctggtt

SEQ ID NO: 46 - DNA, 450 bp, synthetic construct
  misc_feature 1..450: note = Description of sequence: HA PD1 ECD
  source 1..450: mol_type = other DNA
cccggctggttcctggatagccctgaccggccatggaatcctcctaccttcagccccgctctgctcgtggtcacagagggagataacgccacattcacctgtagcttcagcaacacaagcgagtcttttcacgtgatttggcatcgggaatctccttccggccagaccgacaccctggccgccttccctgaagatagatctcaacctggacaggactgcagattcagagtgacccagctgcccaacggcagagacttccacatgagcgtggtgcgggccagacggaacgacagcggcacctacgtgtgcggcgtgatcagcctggctcctaagatccagatcaaggaaagcctgagagccgagctgcgggtgaccgagcggagagctgaggtgcctacagcccaccctagcccatctcctagacctgccggccaatttcagacactggtc

SEQ ID NO: 47 - DNA, 66 bp, synthetic construct
  misc_feature 1..66: note = Description of sequence: BR2 signal peptide
  source 1..66: mol_type = other DNA
atgggcagaggactgctgagaggcctgtggcctctgcatatcgtgctgtggaccagaatcgcctct

SEQ ID NO: 48 - DNA, 432 bp, synthetic construct
  misc_feature 1..432: note = Description of sequence: WT BR2 ECD
  source 1..432: mol_type = other DNA
acaatccccccccacgtgcagaagtccgtgaacaatgacatgatcgtcaccgacaacaacggcgctgtgaagtttccacaactgtgcaagttctgcgacgtgcggttcagcacatgcgataaccagaaaagctgtatgagcaattgctccattacaagcatctgtgaaaaacctcaggaggtgtgcgtggccgtgtggcggaagaacgacgagaacatcaccctggagaccgtgtgtcacgatcctaagctgccttaccacgacttcatcctggaagatgccgccagccctaagtgcatcatgaaggaaaagaaaaagcctggcgagaccttcttcatgtgctcttgttctagcgacgagtgcaacgataatatcatcttcagcgaggaatacaacaccagcaaccccgacctgctgctcgtgatctttcag

SEQ ID NO: 49 - DNA, 354 bp, synthetic construct
  misc_feature 1..354: note = Description of sequence: dN25 BR2 ECD
  source 1..354: mol_type = other DNA
cagctgtgcaagttctgcgacgtgcggttcagcacctgtgataaccagaaaagctgtatgagcaattgctctatcacctccatctgcgagaagcctcaggaggtgtgcgtggccgtgtggcggaagaacgacgagaacattacactggaaaccgtgtgtcacgatcctaagctgccttaccacgacttcatcctggaagatgccgccagccctaagtgcatcatgaaggaaaagaaaaagccaggcgagacatttttcatgtgctcctgtagcagcgacgagtgcaacgacaatatcatctttagcgaggaatacaacaccagcaaccccgacctgctcctggtcatcttccaa

SEQ ID NO: 50 - DNA, 111 bp, synthetic construct
  misc_feature 1..111: note = Description of sequence: PD1 TM
  source 1..111: mol_type = other DNA
gtcggcgtggtgggcggactgctgggctctctggtgctgctggtgtgggtgctggccgtgatctgcagcagagccgctagaggaacaatcggcgccagacggaccggccag

SEQ ID NO: 51 - DNA, 516 bp, synthetic construct
  misc_feature 1..516: note = Description of sequence: MyD88
  source 1..516: mol_type = other DNA
atggctgctggaggacctggcgctggcagcgccgctcctgtgtccagcaccagctctctgcctctggctgcacttaatatgagagtgcggcggagactgagcctcttcctgaatgtgcgcacccaagtggcagctgattggaccgccctggccgaagagatggacttcgagtacctggaaatcagacagctggaaacccaggccgaccctacaggcagactgctggatgcctggcagggcagaccgggcgccagcgttggaaggctgctggacctcctgaccaagctgggccgggatgatgtgctgctggagctgggtcctagcatcgaggaagattgccagaaatacatcctgaaacagcaacaggaggaagccgagaagcctctgcaggtggccgccgtggacagctctgtgcctagaacagccgagctggccggcatcaccaccctggacgaccccctgggccacatgcctgagcggttcgacgcctttatttgttattgcccctctgacatc

SEQ ID NO: 52 - DNA, 36 bp, synthetic construct
  misc_feature 1..36: note = Description of sequence: CD28 ECD (truncated)
  source 1..36: mol_type = other DNA
tgtcctagccccctgttccccggtcctagcaaacct

SEQ ID NO: 53 - DNA, 81 bp, synthetic construct
  misc_feature 1..81: note = Description of sequence: CD28 TM
  source 1..81: mol_type = other DNA
ttctgggtgctggtggtggtgggcggcgtgctggcctgctacagcctgctggtcacagtggcctttatcatcttctgggtc

SEQ ID NO: 54 - DNA, 123 bp, synthetic construct
  misc_feature 1..123: note = Description of sequence: CD28 intracellular
  source 1..123: mol_type = other DNA
agatccaagcggtctagactgcttcatagcgactacatgaacatgacacctagaaggcctggccccacaagaaagcactaccagccctacgcccctcctagagatttcgccgcctacagaagc

SEQ ID NO: 55 - DNA, 123 bp, synthetic construct
  misc_feature 1..123: note = Description of sequence: CD28.YMFM intracellular
  source 1..123: mol_type = other DNA
agatctaagcggtccagactgctgcattctgattacatgttcatgacccctagaagacctggacctacaagaaagcactaccagccttacgcccctcctcgggacttcgccgcttatagaagc

SEQ ID NO: 56 - DNA, 123 bp, synthetic construct
  misc_feature 1..123: note = Description of sequence: CD28.AYAA intracellular
  source 1..123: mol_type = other DNA
agatctaagcggtccagactgctgcattctgattacatgaacatgacccctagaagacctggacctacaagaaagcactaccaggcctacgccgcccctcgggacttcgccgcttatagaagc

SEQ ID NO: 57 - DNA, 36 bp, synthetic construct
  misc_feature 1..36: note = Description of sequence: CD2 ECD (truncated)
  source 1..36: mol_type = other DNA
gtggagcctgtgtcctgccctgagaagggcctggac

SEQ ID NO: 58 - DNA, 78 bp, synthetic construct
  misc_feature 1..78: note = Description of sequence: CD2 TM
  source 1..78: mol_type = other DNA
atctacctgatcatcggcatctgcggaggaggcagcctgctgatggtgttcgtggccctgctggtgttctacatcacc

SEQ ID NO: 59 - DNA, 348 bp, synthetic construct
  misc_feature 1..348: note = Description of sequence: CD2 intracellular (full)
  source 1..348: mol_type = other DNA
aagcggaagaagcagcggagcagacggaatgacgaggaactcgagacaagagcccatcgggtcgccacagaggaaagaggcagaaagccccaccagattcctgccagcacacctcagaaccctgctaccagccaacacccccccccccctcctggccacagatctcaggcccctagccaccggcccccgccacctggccaccgggtgcagcaccagcctcaaaaaagaccccctgctcctagcggcacacaggtgcaccagcagaaaggtcctccactgcctagacctcgggtgcagcctaagcctccacatggcgccgctgagaacagcttgtctcctagttctaat

SEQ ID NO: 60 - DNA, 255 bp, synthetic construct
  misc_feature 1..255: note = Description of sequence: CD2 intracellular (short)
  source 1..255: mol_type = other DNA
aagagaaagaagcagacccctcagaaccccgccaccagccaacaccccccccctccaccaggccacagaagccaggccccttcccaccgccccccccctccaggacatagggttcagcaccagccccagaagcggcctcctgctcctagcggaacacaggtgcaccagcagaaaggccctcccctccctagacccagagtgcagcctaaacctccccacggcgccgccgagaacagcctgtccccttctagcaat

SEQ ID NO: 61 - DNA, 90 bp, synthetic construct
  misc_feature 1..90: note = Description of sequence: DAP10 ECD
  source 1..90: mol_type = other DNA
cagaccacacctggagaacggagcagcctccccgctttctaccccggcaccagcggctcctgcagcggatgtggcagcctgagcctgcct

SEQ ID NO: 62 - DNA, 63 bp, synthetic construct
  misc_feature 1..63: note = Description of sequence: DAP10 TM D57N
  source 1..63: mol_type = other DNA
ctgctggccggcctggtggccgccaacgccgtcgcctctctgctgatcgtgggcgccgtgttc

SEQ ID NO: 63 - DNA, 63 bp, synthetic construct; identical to SEQ ID NO: 62
  misc_feature 1..63: note = Description of sequence: DAP10 intracellular
  source 1..63: mol_type = other DNA

SEQ ID NO: 64 - DNA, 63 bp, synthetic construct
  misc_feature 1..63: note = Description of sequence: ICOS TM
  source 1..63: mol_type = other DNA
ttctggctgcctatcggctgcgccgcttttgtggtggtctgcatcctgggatgtatcctgatc

SEQ ID NO: 65 - DNA, 114 bp, synthetic construct
  misc_feature 1..114: note = Description of sequence: ICOS intracellular
  source 1..114: mol_type = other DNA
tgctggctgaccaagaagaagtacagctccagcgtgcacgaccccaacggcgagtacatgttcatgcgggccgtgaacaccgccaagaaatctagactgacagatgtgaccctg

SEQ ID NO: 66 - DNA, 66 bp, synthetic construct
  misc_feature 1..66: note = Description of sequence: CD40 TM
  source 1..66: mol_type = other DNA
gccctggtggtgatccccatcatcttcggcatcctgttcgccattctgctggtgctggtctttatc

SEQ ID NO: 67 - DNA, 186 bp, synthetic construct
  misc_feature 1..186: note = Description of sequence: CD40 intracellular
  source 1..186: mol_type = other DNA
aagaaggtggccaagaaacctacaaacaaggcccctcaccccaagcaggagcctcaggagatcaacttccccgacgacctgcctggaagcaataccgccgctccagtgcaagaaaccctgcacggctgccagcctgtgacccaggaagatggcaaagagtctagaatcagcgtgcaggagcggcag

SEQ ID NO: 68 - DNA, 63 bp, synthetic construct
  misc_feature 1..63: note = Description of sequence: OX40 TM
  source 1..63: mol_type = other DNA
gtggccgccatcctgggcctgggcctggtgctgggactgctgggccctctggctatcctgctg

SEQ ID NO: 69 - DNA, 126 bp, synthetic construct
  misc_feature 1..126: note = Description of sequence: OX40 intracellular
  source 1..126: mol_type = other DNA
gccctgtacctgctcagacgggaccagagactgccccccgacgcccacaagcctccaggcggcggatctttcagaacccctatccaggaggaacaggccgatgctcacagcacactggccaagatc

SEQ ID NO: 70 - DNA, 63 bp, synthetic construct
  misc_feature 1..63: note = Description of sequence: BAFFR TM
  source 1..63: mol_type = other DNA
ttcggcgctcccgctctgctgggcctcgccctggtgctggccctggtcctggtgggcctggtg

SEQ ID NO: 71 - DNA, 255 bp, synthetic construct
  misc_feature 1..255: note = Description of sequence: BAFFR intracellular
  source 1..255: mol_type = other DNA
tcctggcggcggagacagagaagactgagaggcgccagcagcgccgaggcccctgatggcgataaggacgcccctgagcctctggacaaagtgatcatcctgagccccggcatcagcgacgctaccgcccctgcctggcctccaccaggcgaggaccccggaacaacccctcctggccacagcgtgcctgtgcccgccaccgagctgggatctacagaactggtgaccacaaagaccgccggccctgaacagcag

SEQ ID NO: 72 - DNA, 135 bp, synthetic construct
  misc_feature 1..135: note = Description of sequence: CD8-Alpha hinge
  source 1..135: mol_type = other DNA
acaaccacacctgcacctaggccacctacacctgcaccaaccatcgccagccagcctctgtccctgagaccagaggcctgtaggccagcagcaggaggagcagtgcacacccggggcctggacttcgcctgcgat

SEQ ID NO: 73 - protein, 530 aa, synthetic construct
  REGION 1..530: note = Description of sequence: Construct name: 2G1 (DLL3 CAR); Polypeptide structure: CD8 signal sequence (underlined), rituximab mimotope RSR, 2G1 scFv, rituximab mimotope, CD8-Alpha hinge, CD8-Alpha transmembrane, CD8-Alpha cytoplasmic domain (truncated), 4-1BB (TNFRSF9, CD137) cytoplasmic domain, CD3 cytoplasmic domain
MALPVTALLLPLALLLHAARPGGGGSCPYSNPSLCGGGGSQLQLQESGPGLVKPSETLSLTCTVSGGSISSSSYYWGWIRQPPGKGLEWIGSIYYSGNIYHNPSLKSRVSISVDTSKNQFSLRLSSVTAADTAVYYCAREIIVGATHFDYWGQGTLVTVSSGGGGSGGGGSGGGGSGGGGSAIQMTQSPSSLSASVGDRVTITCRASQGIRNDLGWYQQKPGKAPELLIYAASSLQSGVPSRFSGSGSGTDFTLTISSLQPEDFATYYCLQDYNYPLTFGPGTKVDIKGGGGSCPYSNPSLCGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 74 - protein, 755 aa, synthetic construct
  REGION 1..755: note = Description of sequence: Construct name: 2G1.15.1 (CCR-P2A-DLL3 CAR); Polypeptide structure: CD8 signal sequence (underlined), TpoR (S505N W515K), IL2Rb-YY, P2A,
MALPVTALLLPLALLLHAARPSDPTRVETATETAWISLVTALHLVLGLNAVLGLLLLRKQFPAHYRRLRHALWPSLPDLHRVLGQYLRDTAALSPPKATVSDTCEEVEPSLLEILPKSSERTPLPLLEDEGVAGAPTGSSPQPLQPLSGEDDAYCTFPSRDDLLLFSPSGQGEFRALNARLPLNTDAYLSLQELQGQDPTHLVGSGATNFSLLKQAGDVEENPGPMALPVTALLLPLALLLHAARPGGGGSCPYSNPSLCGGGGSQLQLQESGPGLVKPSETLSLTCTVSGGSISSSSYYWGWIRQPPGKGLEWIGSIYYSGNIYHNPSLKSRVSISVDTSKNQFSLRLSSVTAADTAVYYCAREIIVGATHFDYWGQGTLVTVSSGGGGSGGGGSGGGGSGGGGSAIQMTQSPSSLSASVGDRVTITCRASQGIRNDLGWYQQKPGKAPELLIYAASSLQSGVPSRFSGSGSGTDFTLTISSLQPEDFATYYCLQDYNYPLTFGPGTKVDIKGGGGSCPYSNPSLCGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 75 - protein, 230 aa, synthetic construct
  REGION 1..230: note = Description of sequence: Construct name: WT PD1.CD28; Polypeptide structure: WT PD1 ECD, CD28 ECD (truncated), CD28 TM, CD28 intracellular
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 76 - protein, 230 aa, synthetic construct
  REGION 1..230: note = Description of sequence: Construct name: WT PD1.CD28.YMFM; Polypeptide structure: WT PD1 ECD, CD28 ECD (truncated), CD28 TM, CD28.YMFM intracellular
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVCPSPLFPGPSKPFWVLVVVGGVLACYSLLVTVAFIIFWVRSKRSRLLHSDYMFMTPRRPGPTRKHYQPYAPPRDFAAYRS

SEQ ID NO: 77 - protein, 230 aa, synthetic construct
  REGION 1..230: note = Description of sequence: Construct name: WT PD1.CD28.AYAA; Polypeptide structure: WT PD1 ECD, CD28 ECD (truncated), CD28 TM, CD28.AYAA intracellular
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVCPSPLFPGPSKPFWVLVVVGGVLACYSLLVTVAFIIFWVRSKRSRLLHSDYMNMTPRRPGPTRKHYQAYAAPRDFAAYRS

SEQ ID NO: 78 - protein, 304 aa, synthetic construct
  REGION 1..304: note = Description of sequence: Construct name: WT PD1.CD2(full); Polypeptide structure: WT PD1 ECD, CD2 ECD (truncated), CD2 TM, CD2 intracellular
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVEPVSCPEKGLDIYLIIGICGGGSLLMVFVALLVFYITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 79 - protein, 273 aa, synthetic construct
  REGION 1..273: note = Description of sequence: Construct name: WT PD1.CD2(short); Polypeptide structure: WT PD1 ECD, CD2 ECD (truncated), CD2 TM, CD2 intracellular (short)
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVEPVSCPEKGLDIYLIIGICGGGSLLMVFVALLVFYITKRKKQTPQNPATSQHPPPPPGHRSQAPSHRPPPPGHRVQHQPQKRPPAPSGTQVHQQKGPPLPRPRVQPKPPHGAAENSLSPSSN

SEQ ID NO: 80 - protein, 225 aa, synthetic construct
  REGION 1..225: note = Description of sequence: Construct name: WT PD1.DAP10.D57N; Polypeptide structure: WT PD1 ECD, DAP10 ECD, DAP10 TM D57N, DAP10 intracellular
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVQTTPGERSSLPAFYPGTSGSCSGCGSLSLPLLAGLVAANAVASLLIVGAVFLCARPRRSPAQEDGKVYINMPGRG

SEQ ID NO: 81 - protein, 209 aa, synthetic construct
  REGION 1..209: note = Description of sequence: Construct name: WT PD1.ICOS; Polypeptide structure: WT PD1 ECD, ICOS TM, ICOS intracellular
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVFWLPIGCAAFVVVCILGCILICWLTKKKYSSSVHDPNGEYMFMRAVNTAKKSRLTDVTL

SEQ ID NO: 82 - protein, 234 aa, synthetic construct
  REGION 1..234: note = Description of sequence: Construct name: WT PD1.CD40; Polypeptide structure: WT PD1 ECD, CD40 TM, CD40 intracellular
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVALVVIPIIFGILFAILLVLVFIKKVAKKPTNKAPHPKQEPQEINFPDDLPGSNTAAPVQETLHGCQPVTQEDGKESRISVQERQ

SEQ ID NO: 83 - protein, 213 aa, synthetic construct
  REGION 1..213: note = Description of sequence: Construct name: WT PD1.OX40; Polypeptide structure: WT PD1 ECD, OX40 TM, OX40 intracellular
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVAAILGLGLVLGLLGPLAILLALYLLRRDQRLPPDAHKPPGGGSFRTPIQEEQADAHSTLAKI

SEQ ID NO: 84 - protein, 230 aa, synthetic construct
  REGION 1..230: note = Description of sequence: Construct name: HA PD1.CD28; Polypeptide structure: HA PD1 ECD, CD28 ECD (truncated), CD28 TM, CD28 intracellular
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 85 - protein, 230 aa, synthetic construct
  REGION 1..230: note = Description of sequence: Construct name: HA PD1.CD28.YMFM; Polypeptide structure: HA PD1 ECD, CD28 ECD (truncated), CD28 TM, CD28.YMFM intracellular
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVCPSPLFPGPSKPFWVLVVVGGVLACYSLLVTVAFIIFWVRSKRSRLLHSDYMFMTPRRPGPTRKHYQPYAPPRDFAAYRS

SEQ ID NO: 86 - protein, 230 aa, synthetic construct
  REGION 1..230: note = Description of sequence: Construct name: HA PD1.CD28.AYAA; Polypeptide structure: HA PD1 ECD, CD28 ECD (truncated), CD28 TM, CD28.AYAA intracellular
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVCPSPLFPGPSKPFWVLVVVGGVLACYSLLVTVAFIIFWVRSKRSRLLHSDYMNMTPRRPGPTRKHYQAYAAPRDFAAYRS

SEQ ID NO: 87 - protein, 304 aa, synthetic construct
  REGION 1..304: note = Description of sequence: Construct name: HA PD1.CD2(full); Polypeptide structure: HA PD1 ECD, CD2 ECD (truncated), CD2 TM, CD2 intracellular
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVVEPVSCPEKGLDIYLIIGICGGGSLLMVFVALLVFYITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 88 - protein, 273 aa, synthetic construct
  REGION 1..273: note = Description of sequence: Construct name: HA PD1.CD2(short); Polypeptide structure: HA PD1 ECD, CD2 ECD (truncated), CD2 TM, CD2 intracellular (short)
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVVEPVSCPEKGLDIYLIIGICGGGSLLMVFVALLVFYITKRKKQTPQNPATSQHPPPPPGHRSQAPSHRPPPPGHRVQHQPQKRPPAPSGTQVHQQKGPPLPRPRVQPKPPHGAAENSLSPSSN

SEQ ID NO: 89 - protein, 225 aa, synthetic construct
  REGION 1..225: note = Description of sequence: Construct name: HA PD1.DAP10.D57N; Polypeptide structure: HA PD1 ECD, DAP10 ECD, DAP10 TM D57N, DAP10 intracellular
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVQTTPGERSSLPAFYPGTSGSCSGCGSLSLPLLAGLVAANAVASLLIVGAVFLCARPRRSPAQEDGKVYINMPGRG

SEQ ID NO: 90 - protein, 209 aa, synthetic construct
  REGION 1..209: note = Description of sequence: Construct name: HA PD1.ICOS; Polypeptide structure: HA PD1 ECD, ICOS TM, ICOS intracellular
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVFWLPIGCAAFVVVCILGCILICWLTKKKYSSSVHDPNGEYMFMRAVNTAKKSRLTDVTL

SEQ ID NO: 91 - protein, 234 aa, synthetic construct
  REGION 1..234: note = Description of sequence: Construct name: HA PD1.CD40; Polypeptide structure: HA PD1 ECD, CD40 TM, CD40 intracellular
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVALVVIPIIFGILFAILLVLVFIKKVAKKPTNKAPHPKQEPQEINFPDDLPGSNTAAPVQETLHGCQPVTQEDGKESRISVQERQ

SEQ ID NO: 92 - protein, 213 aa, synthetic construct
  REGION 1..213: note = Description of sequence: Construct name: HA PD1.OX40; Polypeptide structure: HA PD1 ECD, OX40 TM, OX40 intracellular
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVVAAILGLGLVLGLLGPLAILLALYLLRRDQRLPPDAHKPPGGGSFRTPIQEEQADAHSTLAKI

SEQ ID NO: 93 - protein, 256 aa, synthetic construct
  REGION 1..256: note = Description of sequence: Construct name: HA PD1.BAFFR; Polypeptide structure: HA PD1 ECD, BAFFR TM, BAFFR intracellular
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVFGAPALLGLALVLALVLVGLVSWRRRQRRLRGASSAEAPDGDKDAPEPLDKVIILSPGISDATAPAWPPPGEDPGTTPPGHSVPVPATELGSTELVTTKTAGPEQQ

SEQ ID NO: 94 - protein, 359 aa, synthetic construct
  REGION 1..359: note = Description of sequence: Construct name: HA PD1.tm.MyD88; Polypeptide structure: HA PD1 ECD, PD1 TM, MyD88
PGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVVGVVGGLLGSLVLLVWVLAVICSRAARGTIGARRTGQMAAGGPGAGSAAPVSSTSSLPLAALNMRVRRRLSLFLNVRTQVAADWTALAEEMDFEYLEIRQLETQADPTGRLLDAWQGRPGASVGRLLDLLTKLGRDDVLLELGPSIEEDCQKYILKQQQEEAEKPLQVAAVDSSVPRTAELAGITTLDDPLGHMPERFDAFICYCPSDI

SEQ ID NO: 95 - protein, 224 aa, synthetic construct
  REGION 1..224: note = Description of sequence: Construct name: WT BR2.CD28; Polypeptide structure: WT BR2 ECD, CD28 ECD (truncated), CD28 TM, CD28 intracellular
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 96 - protein, 224 aa, synthetic construct
  REGION 1..224: note = Description of sequence: Construct name: WT BR2.CD28.YMFM; Polypeptide structure: WT BR2 ECD, CD28 ECD (truncated), CD28 TM, CD28.YMFM intracellular
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQCPSPLFPGPSKPFWVLVVVGGVLACYSLLVTVAFIIFWVRSKRSRLLHSDYMFMTPRRPGPTRKHYQPYAPPRDFAAYRS

SEQ ID NO: 97 - protein, 224 aa, synthetic construct
  REGION 1..224: note = Description of sequence: Construct name: WT BR2.CD28.AYAA; Polypeptide structure: WT BR2 ECD, CD28 ECD (truncated), CD28 TM, CD28.AYAA intracellular
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQCPSPLFPGPSKPFWVLVVVGGVLACYSLLVTVAFIIFWVRSKRSRLLHSDYMNMTPRRPGPTRKHYQAYAAPRDFAAYRS

SEQ ID NO: 98 - protein, 298 aa, synthetic construct
  REGION 1..298: note = Description of sequence: Construct name: WT BR2.CD2(full); Polypeptide structure: WT BR2 ECD, CD2 ECD (truncated), CD2 TM, CD2 intracellular
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVEPVSCPEKGLDIYLIIGICGGGSLLMVFVALLVFYITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 99 - protein, 267 aa, synthetic construct
  REGION 1..267: note = Description of sequence: Construct name: WT BR2.CD2(short); Polypeptide structure: WT BR2 ECD, CD2 ECD (truncated), CD2 TM, CD2 intracellular (short)
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVEPVSCPEKGLDIYLIIGICGGGSLLMVFVALLVFYITKRKKQTPQNPATSQHPPPPPGHRSQAPSHRPPPPGHRVQHQPQKRPPAPSGTQVHQQKGPPLPRPRVQPKPPHGAAENSLSPSSN

SEQ ID NO: 100 - protein, 219 aa, synthetic construct
  REGION 1..219: note = Description of sequence: Construct name: WT BR2.DAP10.D57N; Polypeptide structure: WT BR2 ECD, DAP10 ECD, DAP10 TM D57N, DAP10 intracellular
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQQTTPGERSSLPAFYPGTSGSCSGCGSLSLPLLAGLVAANAVASLLIVGAVFLCARPRRSPAQEDGKVYINMPGRG

SEQ ID NO: 101 - protein, 203 aa, synthetic construct
  REGION 1..203: note = Description of sequence: Construct name: WT BR2.ICOS; Polypeptide structure: WT BR2 ECD, ICOS TM, ICOS intracellular
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQFWLPIGCAAFVVVCILGCILICWLTKKKYSSSVHDPNGEYMFMRAVNTAKKSRLTDVTL

SEQ ID NO: 102 - protein, 228 aa, synthetic construct
  REGION 1..228: note = Description of sequence: Construct name: WT BR2.CD40; Polypeptide structure: WT BR2 ECD, CD40 TM, CD40 intracellular
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQALVVIPIIFGILFAILLVLVFIKKVAKKPTNKAPHPKQEPQEINFPDDLPGSNTAAPVQETLHGCQPVTQEDGKESRISVQERQ

SEQ ID NO: 103 - protein, 207 aa, synthetic construct
  REGION 1..207: note = Description of sequence: Construct name: WT BR2.OX40; Polypeptide structure: WT BR2 ECD, OX40 TM, OX40 intracellular
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVAAILGLGLVLGLLGPLAILLALYLLRRDQRLPPDAHKPPGGGSFRTPIQEEQADAHSTLAKI

SEQ ID NO: 104 - protein, 353 aa, synthetic construct
  REGION 1..353: note = Description of sequence: Construct name: WT BR2.PD1 tm.MyD88; Polypeptide structure: WT BR2 ECD, PD1 TM, MyD88
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVGVVGGLLGSLVLLVWVLAVICSRAARGTIGARRTGQMAAGGPGAGSAAPVSSTSSLPLAALNMRVRRRLSLFLNVRTQVAADWTALAEEMDFEYLEIRQLETQADPTGRLLDAWQGRPGASVGRLLDLLTKLGRDDVLLELGPSIEEDCQKYILKQQQEEAEKPLQVAAVDSSVPRTAELAGITTLDDPLGHMPERFDAFICYCPSDI

SEQ ID NO: 105 - protein, 198 aa, synthetic construct
  REGION 1..198: note = Description of sequence: Construct name: dN25 BR2.CD28; Polypeptide structure: dN BR2 ECD, CD28 ECD (truncated), CD28 TM, CD28 intracellular
QLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 106 - protein, 198 aa, synthetic construct
  REGION 1..198: note = Description of sequence: Construct name: dN25 BR2.CD28.YMFM; Polypeptide structure: dN25 BR2 ECD, CD28 ECD (truncated), CD28 TM, CD28.YMFM intracellular
QLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQCPSPLFPGPSKPFWVLVVVGGVLACYSLLVTVAFIIFWVRSKRSRLLHSDYMFMTPRRPGPTRKHYQPYAPPRDFAAYRS

SEQ ID NO: 107 - protein, 198 aa, synthetic construct
  REGION 1..198: note = Description of sequence: Construct name: dN25 BR2.CD28.AYAA; Polypeptide structure: dN25 BR2 ECD, CD28 ECD (truncated), CD28 TM, CD28.AYAA intracellular
QLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQCPSPLFPGPSKPFWVLVVVGGVLACYSLLVTVAFIIFWVRSKRSRLLHSDYMNMTPRRPGPTRKHYQAYAAPRDFAAYRS

SEQ ID NO: 108 - protein, 272 aa, synthetic construct
  REGION 1..272: note = Description of sequence: Construct name: dN25 BR2.CD2(full); Polypeptide structure: dN25 BR2 ECD, CD2 ECD (truncated), CD2 TM, CD2 intracellular
QLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVEPVSCPEKGLDIYLIIGICGGGSLLMVFVALLVFYITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 109 - protein, 241 aa, synthetic construct
  REGION 1..241: note = Description of sequence: Construct name: dN25 BR2.CD2(short); Polypeptide structure: dN25 BR2 ECD, CD2 ECD (truncated), CD2 TM, CD2 intracellular (short)
QLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVEPVSCPEKGLDIYLIIGICGGGSLLMVFVALLVFYITKRKKQTPQNPATSQHPPPPPGHRSQAPSHRPPPPGHRVQHQPQKRPPAPSGTQVHQQKGPPLPRPRVQPKPPHGAAENSLSPSSN

SEQ ID NO: 110 - protein, 193 aa, synthetic construct
  REGION 1..193: note = Description of sequence: Construct name: dN25 BR2.DAP10.D57N; Polypeptide structure: dN25 BR2 ECD, DAP10 ECD, DAP10 TM D57N, DAP10 intracellular
QLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQQTTPGERSSLPAFYPGTSGSCSGCGSLSLPLLAGLVAANAVASLLIVGAVFLCARPRRSPAQEDGKVYINMPGRG

SEQ ID NO: 111 - protein, 177 aa, synthetic construct
  REGION 1..177: note = Description of sequence: Construct name: dN25 BR2.ICOS; Polypeptide structure: dN25 BR2 ECD, ICOS TM, ICOS intracellular
QLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQFWLPIGCAAFVVVCILGCILICWLTKKKYSSSVHDPNGEYMFMRAVNTAKKSRLTDVTL

SEQ ID NO: 112 - protein, 202 aa, synthetic construct
  REGION 1..202: note = Description of sequence: Construct name: dN25 BR2.CD40; Polypeptide structure: dN25 BR2 ECD, CD40 TM, CD40 intracellular
QLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQALVVIPIIFGILFAILLVLVFIKKVAKKPTNKAPHPKQEPQEINFPDDLPGSNTAAPVQETLHGCQPVTQEDGKESRISVQERQ

SEQ ID NO: 113 - protein, 181 aa, synthetic construct
  REGION 1..181: note = Description of sequence: Construct name: dN25 BR2.OX40; Polypeptide structure: dN25 BR2 ECD, OX40 TM, OX40 intracellular
QLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVAAILGLGLVLGLLGPLAILLALYLLRRDQRLPPDAHKPPGGGSFRTPIQEEQADAHSTLAKI

SEQ ID NO: 114 - protein, 224 aa, synthetic construct
  REGION 1..224: note = Description of sequence: Construct name: dN25 BR2.BAFFR; Polypeptide structure: dN25 BR2 ECD, BAFFR TM, BAFFR intracellular
QLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQFGAPALLGLALVLALVLVGLVSWRRRQRRLRGASSAEAPDGDKDAPEPLDKVIILSPGISDATAPAWPPPGEDPGTTPPGHSVPVPATELGSTELVTTKTAGPEQQ

SEQ ID NO: 115 - protein, 327 aa, synthetic construct
  REGION 1..327: note = Description of sequence: Construct name: dN25 BR2.PD1 tm.MyD88; Polypeptide structure: dN25 BR2 ECD, PD1 TM, MyD88
QLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVGVVGGLLGSLVLLVWVLAVICSRAARGTIGARRTGQMAAGGPGAGSAAPVSSTSSLPLAALNMRVRRRLSLFLNVRTQVAADWTALAEEMDFEYLEIRQLETQADPTGRLLDAWQGRPGASVGRLLDLLTKLGRDDVLLELGPSIEEDCQKYILKQQQEEAEKPLQVAAVDSSVPRTAELAGITTLDDPLGHMPERFDAFICYCPSDI

SEQ ID NO: 116 - DNA, 1590 bp, synthetic construct
  misc_feature 1..1590: note = Description of sequence: Construt: 2G1-RSR; Polypeptide structure: CD8 signal sequence (underlined), rituximab mimotope, 2G1 scFv, rituximab mimotope, CD8-Alpha hinge, CD8-Alpha transmembrane, CD8-Alpha cytoplasmic domain (truncated), 4-1BB (TNFRSF9, CD137) cytoplasmic domain, CD3 cytoplasmic domain
  source 1..1590: mol_type = other DNA
atggccctgccagtgaccgccctgctgctgcccctggccctgctgctgcacgcagccagacccggaggaggaggctcttgcccctacagcaacccttccctgtgcggaggaggaggctctcagctgcagctgcaggagtccggccctggcctggtgaagccatccgagaccctgtctctgacctgcacagtgagcggcggctccatcagctcctctagctactattggggctggatcagacagccccctggcaagggactggagtggatcggcagcatctactattccggcaacatctaccacaatccttctctgaagagccgcgtgtctatcagcgtggacacctccaagaaccagttctctctgaggctgtcctctgtgaccgcagcagatacagccgtgtactattgcgccagggagatcatcgtgggagcaacccactttgactattggggccagggcaccctggtgacagtgagctccggcggcggcggctctggaggaggaggcagcggcggaggaggctccggaggcggcggctctgccatccagatgacacagtccccatctagcctgtccgcctctgtgggcgacagggtgaccatcacatgtagagccagccagggcatcaggaacgatctgggctggtaccagcagaagccaggcaaggcccccgagctgctgatctatgccgcctcctctctgcagtctggcgtgccaagcagattcagcggctccggctctggcaccgactttaccctgacaatcagctccctgcagcccgaggacttcgccacatactattgtctgcaggattacaattatcccctgacctttggccctggcacaaaggtggatatcaagggaggaggaggctcttgcccctacagcaacccttccctgtgcggaggaggaggctctacaaccacacctgcacctaggccacctacacctgcaccaaccatcgccagccagcctctgtccctgagaccagaggcctgtaggccagcagcaggaggagcagtgcacacccggggcctggacttcgcctgcgatatctacatctgggcaccactggcaggaacatgtggcgtgctgctgctgtccctggtcatcaccctgtactgcaagagaggcaggaagaagctgctgtatatcttcaagcagcccttcatgagacccgtgcagacaacccaggaggaggacggctgcagctgtaggttcccagaggaggaggagggaggatgtgagctgcgcgtgaagttttcccggtctgccgatgcacctgcataccagcagggacagaaccagctgtataacgagctgaatctgggccggagagaggagtacgacgtgctggataagaggaggggaagggaccctgagatgggaggcaagcctcggagaaagaacccacaggagggcctgtacaatgagctgcagaaggacaagatggccgaggcctatagcgagatcggcatgaagggagagaggcgccggggcaagggacacgatggcctgtatcagggcctgtcaaccgctacaaaagatacctacgatgctctgcacatgcaggctctgccaccaaga

SEQ ID NO: 117 - DNA, 2265 bp, synthetic construct
  misc_feature 1..2265: note = Description of sequence: Construt: 2G1.15.1; Polypeptide structure: CD8 signal sequence (underlined), TpoR (S505N W515K), IL2Rb-YY, P2A,CD8 signal sequence, rituximab mimotope, 2G1 scFv, rituximab mimotope, CD8-Alpha hinge, CD8-Alpha transmembrane, CD8-Alpha cytoplasmic domain (truncated), 4-1BB (TNFRSF9, CD137) cytoplasmic domain, CD3 cytoplasmic domain
  source 1..2265: mol_type = other DNA
atggccctgccagtgaccgccctgctgctgccactggccctgctgctgcacgcagcaaggccatcagaccctactagagtcgagaccgctaccgagaccgcttggatctctctggtgaccgccctgcacctggtgctgggcctgaacgccgtgctgggcctgctgctgctgaggaagcagttcccagcacactaccggagactgaggcacgcactgtggccaagcctgcccgacctgcacagggtgctgggacagtatctgagggatacagccgccctgagcccacctaaggcaaccgtgtccgacacatgcgaggaggtggaaccaagtctgctggaaatcctgccaaaatcctctgagcggacacccctgcccctgctcgaggacgagggagtggcaggagcaccaaccggcagctccccccagcctctgcagccactgtccggagaggacgatgcatactgcacattcccttctcgggacgatctgctgctgttctctccaagcggacagggagagtttcgggccctgaacgccagactgcccctgaataccgacgcctatctgagcctgcaggagctgcagggacaggaccccacacacctggtgggatccggagccaccaacttctccctgctgaagcaggccggcgatgtggaggagaatccaggccccatggccctgccagtgaccgccctgctgctgcccctggccctgctgctgcacgcagccagacccggaggaggaggctcttgcccctacagcaacccttccctgtgcggaggaggaggctctcagctgcagctgcaggagtccggccctggcctggtgaagccatccgagaccctgtctctgacctgcacagtgagcggcggctccatcagctcctctagctactattggggctggatcagacagccccctggcaagggactggagtggatcggcagcatctactattccggcaacatctaccacaatccttctctgaagagccgcgtgtctatcagcgtggacacctccaagaaccagttctctctgaggctgtcctctgtgaccgcagcagatacagccgtgtactattgcgccagggagatcatcgtgggagcaacccactttgactattggggccagggcaccctggtgacagtgagctccggcggcggcggctctggaggaggaggcagcggcggaggaggctccggaggcggcggctctgccatccagatgacacagtccccatctagcctgtccgcctctgtgggcgacagggtgaccatcacatgtagagccagccagggcatcaggaacgatctgggctggtaccagcagaagccaggcaaggcccccgagctgctgatctatgccgcctcctctctgcagtctggcgtgccaagcagattcagcggctccggctctggcaccgactttaccctgacaatcagctccctgcagcccgaggacttcgccacatactattgtctgcaggattacaattatcccctgacctttggccctggcacaaaggtggatatcaagggaggaggaggctcttgcccctacagcaacccttccctgtgcggaggaggaggctctacaaccacacctgcacctaggccacctacacctgcaccaaccatcgccagccagcctctgtccctgagaccagaggcctgtaggccagcagcaggaggagcagtgcacacccggggcctggacttcgcctgcgatatctacatctgggcaccactggcaggaacatgtggcgtgctgctgctgtccctggtcatcaccctgtactgcaagagaggcaggaagaagctgctgtatatcttcaagcagcccttcatgagacccgtgcagacaacccaggaggaggacggctgcagctgtaggttcccagaggaggaggagggaggatgtgagctgcgcgtgaagttttcccggtctgccgatgcacctgcataccagcagggacagaaccagctgtataacgagctgaatctgggccggagagaggagtacgacgtgctggataagaggaggggaagggaccctgagatgggaggcaagcctcggagaaagaacccacaggagggcctgtacaatgagctgcagaaggacaagatggccgaggcctatagcgagatcggcatgaagggagagaggcgccggggcaagggacacgatggcctgtatcagggcctgtcaaccgctacaaaagatacctacgatgctctgcacatgcaggctctgccaccaaga

SEQ ID NO: 118 - DNA, 690 bp, synthetic construct
  misc_feature 1..690: note = Description of sequence: Construt: WT PD1.CD28; Polypeptide structure: WT PD1 ECD, CD28 ECD (truncated), CD28 TM, CD28 intracellular
  source 1..690: mol_type = other DNA
cccggctggttcctggacagccctgaccggccttggaacccccccaccttctctcctgctctgctggtggtgacagagggcgacaacgccaccttcacctgcagcttcagcaatacctccgagagctttgtgctgaactggtaccggatgagcccctctaatcagacagataagctggctgcttttccggaagatagaagccagcctggccaagactgccgctttagagttacccagctgcctaacggcagagatttccacatgtctgtggtgcgggccagacggaacgacagcggcacctacctgtgcggcgccatcagcctggcccctaaggcccagatcaaggaaagcctgagagccgagctgcgcgtgaccgagagaagggccgaagtgcctaccgcccaccccagcccatctcctagaccagccggccagttccagaccctggtgtgtccttcccctctgttccccggccctagcaaacccttctgggtgctggtggtcgtgggcggagtgctggcttgctacagcctgctcgtgaccgtggccttcatcatcttctgggtcagaagcaagcggtccagactgctgcacagcgactacatgaacatgacacctagaagacctggacctacaagaaagcactaccagccctacgcccctcctcgggacttcgccgcttatagatct

SEQ ID NO: 119 - DNA, 690 bp, synthetic construct
  misc_feature 1..690: note = Description of sequence: Construt: WT PD1.CD28.YMFM; Polypeptide structure: WT PD1 ECD, CD28 ECD (truncated), CD28 TM, CD28.YMFM intracellular
  source 1..690: mol_type = other DNA
cccggctggttcctggacagccctgaccggccttggaacccccccaccttctctcctgctctgctggtggtgacagagggcgacaacgccaccttcacctgcagcttcagcaatacctccgagagctttgtgctgaactggtaccggatgagcccctctaatcagacagataagctggctgcttttccggaagatagaagccagcctggccaagactgccgctttagagttacccagctgcctaacggcagagatttccacatgtctgtggtgcgggccagacggaacgacagcggcacctacctgtgcggcgccatcagcctggcccctaaggcccagatcaaggaaagcctgagagccgagctgcgcgtgaccgagagaagggccgaagtgcctaccgcccaccccagcccatctcctagaccagccggccagttccagaccctggtgtgtccttcccctctgttccccggccctagcaaacccttctgggtgctggtggtcgtgggcggagtgctggcttgctacagcctgctcgtgaccgtggccttcatcatcttctgggtcagaagcaagcggtccagactgctgcacagcgactacatgttcatgacacctagaagacctggacctacaagaaagcactaccagccctacgcccctcctcgggacttcgccgcttatagatct

SEQ ID NO: 120 - DNA, 690 bp, synthetic construct
  misc_feature 1..690: note = Description of sequence: Construt: WT PD1.CD28.AYAA; Polypeptide structure: WT PD1 ECD, CD28 ECD (truncated), CD28 TM, CD28.AYAA intracellular
  source 1..690: mol_type = other DNA
cccggctggttcctggacagccctgaccggccttggaacccccccaccttctctcctgctctgctggtggtgacagagggcgacaacgccaccttcacctgcagcttcagcaatacctccgagagctttgtgctgaactggtaccggatgagcccctctaatcagacagataagctggctgcttttccggaagatagaagccagcctggccaagactgccgctttagagttacccagctgcctaacggcagagatttccacatgtctgtggtgcgggccagacggaacgacagcggcacctacctgtgcggcgccatcagcctggcccctaaggcccagatcaaggaaagcctgagagccgagctgcgcgtgaccgagagaagggccgaagtgcctaccgcccaccccagcccatctcctagaccagccggccagttccagaccctggtgtgtccttcccctctgttccccggccctagcaaacccttctgggtgctggtggtcgtgggcggagtgctggcttgctacagcctgctcgtgaccgtggccttcatcatcttctgggtcagaagcaagcggtccagactgctgcacagcgactacatgaacatgacacctagaagacctggacctacaagaaagcactaccaggcctacgccgcccctcgggacttcgccgcttatagatct

SEQ ID NO: 121 - DNA, 912 bp, synthetic construct
  misc_feature 1..912: note = Description of sequence: Construt: WT PD1.CD2(full); Polypeptide structure: WT PD1 ECD, CD2 ECD (truncated), CD2 TM, CD2 intracellular
  source 1..912: mol_type = other DNA
cccggatggttcctggattccccagacagaccatggaacccccccaccttttctcctgctctgctggtggtgacagagggcgacaacgccacattcacctgtagcttcagcaataccagcgagagcttcgtgctgaactggtatagaatgtctccttctaaccagaccgacaagctggccgctttccccgaagatcggagccaacctggccaagattgcagattcagagtgacccagctgcctaacggccgggacttccacatgagcgtggtcagagccagaaggaacgacagcggcacctacctgtgcggcgccatcagcctggctcctaaggcccagatcaaggaaagcctgcgcgccgagctgcgggtcacagagagaagagccgaggtgcctaccgcccacccttctccgagcccccggccagccggccagttccagacactggtggtggagcctgtgtcctgccctgagaagggcctggacatctacctgatcatcggcatctgcggaggcggatctctgctcatggtgttcgtggccctgctggtgttttacatcacaaagcggaagaaacagagaagcagacggaacgacgaggaactggaaaccagagcccacagagtggccaccgaggaacggggcagaaagcctcatcagattcctgccagcacccctcagaaccccgctacctcccagcacccccctcctcctcctggacacagatctcaggcccctagccaccggccccctcctcctggccatcgggtgcagcaccaaccccagaaaagacctccagctccttccggcacccaggtgcaccagcagaagggccctcctctgcctagacctagggttcagcccaagcccccccacggcgcagccgaaaacagcctgagccccagcagcaat

SEQ ID NO: 122 - DNA, 819 bp, synthetic construct
  misc_feature 1..819: note = Description of sequence: Construt: WT PD1.CD2(short); Polypeptide structure: WT PD1 ECD, CD2 ECD (truncated), CD2 TM, CD2 intracellular (short)
  source 1..819: mol_type = other DNA
ccaggctggttcctggatagccccgacagaccttggaatccccctacattcagccctgctctgctggtcgtgaccgagggcgacaacgccaccttcacatgcagcttcagcaacaccagcgagtcttttgtgctgaactggtatcggatgagcccttctaaccagacagataagctggcagccttccccgaagatagaagccaacctggccaggactgcagattcagagtgacccagctgcctaacggccgggacttccacatgtctgtggtgcgggccagacgcaacgacagcggcacctacctgtgcggcgccatcagcctggcccctaaggcccagatcaaggaaagcctgagagccgagctgcgggtgacagaaagaagggccgaagtgcccaccgcccacccttccccttcccccagacctgccggacaatttcagaccctggttgtcgagcctgtgagctgccccgagaaggggctggacatctacctgatcatcggcatttgtggcggcggatctctgctgatggtgttcgtggccctgctggtgttctacatcaccaagagaaagaagcagacccctcagaaccccgccacaagccagcatcctccaccacctcccggccaccggagccaggccccaagtcacagacccccacctcctggccacagagtgcagcaccagccccagaagcggcctccagctcctagcggaacccaagtgcaccagcagaaaggccctcctctgcctcggcctagagtgcagcctaaacctccgcacggcgctgctgagaacagcttgtctccctccagcaat

SEQ ID NO: 123 - DNA, 675 bp, synthetic construct
  misc_feature 1..675: note = Description of sequence: Construt: WT PD1.DAP10.D57N; Polypeptide structure: WT PD1 ECD, DAP10 ECD, DAP10 TM D57N, DAP10 intracellular
  source 1..675: mol_type = other DNA
cccggatggttcctggattccccagacagaccatggaacccccccaccttttctcctgctctgctggtggtgacagagggcgacaacgccacattcacctgtagcttcagcaataccagcgagagcttcgtgctgaactggtatagaatgtctccttctaaccagaccgacaagctggccgctttccccgaagatcggagccaacctggccaagattgcagattcagagtgacccagctgcctaacggccgggacttccacatgagcgtggtcagagccagaaggaacgacagcggcacctacctgtgcggcgccatcagcctggctcctaaggcccagatcaaggaaagcctgcgcgccgagctgcgggtcacagagagaagagccgaggtgcctaccgcccacccttctccgagcccccggccagccggccagttccagacactggtgcagaccacacctggagaacggagcagcctccccgccttctaccccggcaccagcggcagctgcagcggatgtggcagcctgtctctgcctctgctggccggcctggtcgccgccaacgccgtggcttctctgctgatcgtgggcgccgtgttcctgtgcgccagacctagacggtccccagctcaggaggacggcaaggtgtacatcaacatgcctggcagaggc

SEQ ID NO: 124 - DNA, 627 bp, synthetic construct
  misc_feature 1..627: note = Description of sequence: Construt: WT PD1.ICOS; Polypeptide structure: WT PD1 ECD, ICOS TM, ICOS intracellular
  source 1..627: mol_type = other DNA
cccggatggttcctggattccccagacagaccatggaacccccccaccttttctcctgctctgctggtggtgacagagggcgacaacgccacattcacctgtagcttcagcaataccagcgagagcttcgtgctgaactggtatagaatgtctccttctaaccagaccgacaagctggccgctttccccgaagatcggagccaacctggccaagattgcagattcagagtgacccagctgcctaacggccgggacttccacatgagcgtggtcagagccagaaggaacgacagcggcacctacctgtgcggcgccatcagcctggctcctaaggcccagatcaaggaaagcctgcgcgccgagctgcgggtcacagagagaagagccgaggtgcctaccgcccacccttctccgagcccccggccagccggccagttccagacactggtgttctggctgcctatcggctgcgccgcttttgtggtggtctgcatcctgggctgtatcctgatctgctggctgaccaagaagaagtacagctcttccgtgcacgaccccaacggcgagtacatgttcatgcgggccgtgaacaccgccaagaaaagcagactgacagatgtgaccctg

SEQ ID NO: 125 - DNA, 702 bp, synthetic construct
  misc_feature 1..702: note = Description of sequence: Construt: WT PD1.CD40; Polypeptide structure: WT PD1 ECD, CD40 TM, CD40 intracellular
  source 1..702: mol_type = other DNA
cccggatggttcctggattccccagacagaccatggaacccccccaccttttctcctgctctgctggtggtgacagagggcgacaacgccacattcacctgtagcttcagcaataccagcgagagcttcgtgctgaactggtatagaatgtctccttctaaccagaccgacaagctggccgctttccccgaagatcggagccaacctggccaagattgcagattcagagtgacccagctgcctaacggccgggacttccacatgagcgtggtcagagccagaaggaacgacagcggcacctacctgtgcggcgccatcagcctggctcctaaggcccagatcaaggaaagcctgcgcgccgagctgcgggtcacagagagaagagccgaggtgcctaccgcccacccttctccgagcccccggccagccggccagttccagacactggtggccctggtcgtgatccccatcatcttcggcatcctgtttgccattctgctggtgctggtgttcatcaagaaagtggccaagaaacctacaaacaaggcccctcaccccaagcaggagcctcaggagatcaacttccccgacgacctgcctggatctaataccgccgctccagtgcaagaaaccctgcacggctgccagcctgtgacccaggaggatggcaaggaaagcagaatcagcgtgcaggagcggcag

SEQ ID NO: 126 - DNA, 639 bp, synthetic construct
  misc_feature 1..639: note = Description of sequence: Construt: WT PD1.OX40; Polypeptide structure: WT PD1 ECD, OX40 TM, OX40 intracellular
  source 1..639: mol_type = other DNA
cccggatggttcctggattccccagacagaccatggaacccccccaccttttctcctgctctgctggtggtgacagagggcgacaacgccacattcacctgtagcttcagcaataccagcgagagcttcgtgctgaactggtatagaatgtctccttctaaccagaccgacaagctggccgctttccccgaagatcggagccaacctggccaagattgcagattcagagtgacccagctgcctaacggccgggacttccacatgagcgtggtcagagccagaaggaacgacagcggcacctacctgtgcggcgccatcagcctggctcctaaggcccagatcaaggaaagcctgcgcgccgagctgcgggtcacagagagaagagccgaggtgcctaccgcccacccttctccgagcccccggccagccggccagttccagacactggtggtggccgctatcctgggcctgggcctcgtgctgggcctgctgggacctctggccatcctgctggctctgtacctgctgagacgggaccagagactgcctccagatgcccacaagccccccggcggcggatctttcagaacccctatccaggaggaacaggccgacgcccacagcacactggccaagatc

SEQ ID NO: 127 - DNA, 690 bp, synthetic construct
  misc_feature 1..690: note = Description of sequence: Construt: HA PD1.CD28; Polypeptide structure: HA PD1 ECD, CD28 ECD (truncated), CD28 TM, CD28 intracellular
  source 1..690: mol_type = other DNA
cccggctggttcctggacagccccgacagaccttggaatcctccaacctttagcccagccctgctggtggtgacagagggagataacgccaccttcacctgcagcttcagcaacaccagcgagagctttcacgtgatctggcaccgggaatccccatctggccagaccgacaccctggctgccttccccgaagatagaagccagcctggccaagactgcagattccgggtgacacagctgcccaacggcagagacttccacatgtctgtggtgcgggctagacggaacgacagcggcacctacgtgtgcggcgtgatttctctggctcctaagatccagatcaaggaatccctgcgggccgagctgagagtgacagagagaagggccgaggtgcctaccgcccaccccagcccaagccctaggcccgccggccagttccagaccctggtttgtcctagccccctgttccccggtcctagcaaacctttctgggtgctggtggtggtgggcggcgtgctggcctgctacagcctgctggtcacagtggcctttatcatcttctgggtcagatctaagcggtccagactgctgcattctgattacatgaacatgacccctagaagacctggacctacaagaaagcactaccagccttacgcccctcctcgggacttcgccgcttatagaagc

SEQ ID NO: 128 - DNA, 690 bp, synthetic construct
  misc_feature 1..690: note = Description of sequence: Construt: HA PD1.CD28.YMFM; Polypeptide structure: HA PD1 ECD, CD28 ECD (truncated), CD28 TM, CD28.YMFM intracellular
  source 1..690: mol_type = other DNA
cccggctggttcctggacagccccgacagaccttggaatcctccaacctttagcccagccctgctggtggtgacagagggagataacgccaccttcacctgcagcttcagcaacaccagcgagagctttcacgtgatctggcaccgggaatccccatctggccagaccgacaccctggctgccttccccgaagatagaagccagcctggccaagactgcagattccgggtgacacagctgcccaacggcagagacttccacatgtctgtggtgcgggctagacggaacgacagcggcacctacgtgtgcggcgtgatttctctggctcctaagatccagatcaaggaatccctgcgggccgagctgagagtgacagagagaagggccgaggtgcctaccgcccaccccagcccaagccctaggcccgccggccagttccagaccctggtttgtcctagccccctgttccccggtcctagcaaacctttctgggtgctggtggtggtgggcggcgtgctggcctgctacagcctgctggtcacagtggcctttatcatcttctgggtcagatctaagcggtccagactgctgcattctgattacatgttcatgacccctagaagacctggacctacaagaaagcactaccagccttacgcccctcctcgggacttcgccgcttatagaagc

SEQ ID NO: 129 - DNA, 690 bp, synthetic construct
  misc_feature 1..690: note = Description of sequence: Construt: HA PD1.CD28.AYAA; Polypeptide structure: HA PD1 ECD, CD28 ECD (truncated), CD28 TM, CD28.AYAA intracellular
  source 1..690: mol_type = other DNA
cccggctggttcctggacagccccgacagaccttggaatcctccaacctttagcccagccctgctggtggtgacagagggagataacgccaccttcacctgcagcttcagcaacaccagcgagagctttcacgtgatctggcaccgggaatccccatctggccagaccgacaccctggctgccttccccgaagatagaagccagcctggccaagactgcagattccgggtgacacagctgcccaacggcagagacttccacatgtctgtggtgcgggctagacggaacgacagcggcacctacgtgtgcggcgtgatttctctggctcctaagatccagatcaaggaatccctgcgggccgagctgagagtgacagagagaagggccgaggtgcctaccgcccaccccagcccaagccctaggcccgccggccagttccagaccctggtttgtcctagccccctgttccccggtcctagcaaacctttctgggtgctggtggtggtgggcggcgtgctggcctgctacagcctgctggtcacagtggcctttatcatcttctgggtcagatctaagcggtccagactgctgcattctgattacatgaacatgacccctagaagacctggacctacaagaaagcactaccaggcctacgccgcccctcgggacttcgccgcttatagaagc

SEQ ID NO: 130 - DNA, 912 bp, synthetic construct
  misc_feature 1..912: note = Description of sequence: Construt: HA PD1.CD2(full); Polypeptide structure: HA PD1 ECD, CD2 ECD (truncated), CD2 TM, CD2 intracellular
  source 1..912: mol_type = other DNA
cctggatggttcctggatagccctgatagaccttggaacccccctaccttcagccctgcacttctggtcgtgaccgaaggcgacaacgccaccttcacctgcagctttagcaacacctccgagagcttccacgtgatctggcacagagagtccccttctggccagaccgacaccctggccgccttccccgaagatagaagccagcccggccaggactgcagattcagagtgacacagctgcccaacggccgcgacttccacatgagcgtggttagagctagaaggaacgacagcggcacctacgtgtgcggcgtgatcagcctggctcccaagatccagatcaaggaaagcctgagagccgaactgcgggtgaccgagcggagagccgaggtgcccaccgcccacccttccccctctccaagacccgccggccaatttcagacactggtggtggagcctgtgtcctgtcctgagaagggactggacatctacctgatcatcggcatctgcggaggaggcagcctgctgatggtgttcgtggccctgctggtgttctacatcaccaagcggaagaagcagcggagcagacggaatgacgaggaactcgagacaagagcccatcgggtcgccacagaggaaagaggcagaaagccccaccagattcctgccagcacacctcagaaccctgctaccagccaacacccccccccccctcctggccacagatctcaggcccctagccaccggcccccgccacctggccaccgggtgcagcaccagcctcaaaaaagaccccctgctcctagcggcacacaggtgcaccagcagaaaggtcctccactgcctagacctcgggtgcagcctaagcctccacatggcgccgctgagaacagcttgtctcctagttctaat

SEQ ID NO: 131 - DNA, 819 bp, synthetic construct
  misc_feature 1..819: note = Description of sequence: Construt: HA PD1.CD2(short); Polypeptide structure: HA PD1 ECD, CD2 ECD (truncated), CD2 TM, CD2 intracellular (short)
  source 1..819: mol_type = other DNA
cccggctggttcctggatagccctgaccggccatggaatcctcctaccttcagccccgctctgctcgtggtcacagagggagataacgccacattcacctgtagcttcagcaacacaagcgagtcttttcacgtgatttggcatcgggaatctccttccggccagaccgacaccctggccgccttccctgaagatagatctcaacctggacaggactgcagattcagagtgacccagctgcccaacggcagagacttccacatgagcgtggtgcgggccagacggaacgacagcggcacctacgtgtgcggcgtgatcagcctggctcctaagatccagatcaaggaaagcctgagagccgagctgcgggtgaccgagcggagagctgaggtgcctacagcccaccctagcccatctcctagacctgccggccaatttcagacactggtcgtggaacctgtgtcctgccccgagaagggcctggacatctacctgatcatcggcatctgcggcggcggcagcctgctgatggtgttcgtggccctgctggtgttctacatcaccaagagaaagaagcagacccctcagaaccccgccaccagccaacaccccccccctccaccaggccacagaagccaggccccttcccaccgccccccccctccaggacatagggttcagcaccagccccagaagcggcctcctgctcctagcggaacacaggtgcaccagcagaaaggccctcccctccctagacccagagtgcagcctaaacctccccacggcgccgccgagaacagcctgtccccttctagcaat

SEQ ID NO: 132 - DNA, 675 bp, synthetic construct
  misc_feature 1..675: note = Description of sequence: Construt: HA PD1.DAP10.D57N; Polypeptide structure: HA PD1 ECD, DAP10 ECD, DAP10 TM D57N, DAP10 intracellular
  source 1..675: mol_type = other DNA
cccggctggttcctggacagccccgacagaccttggaatcctccaacctttagcccagccctgctggtggtgacagagggagataacgccaccttcacctgcagcttcagcaacaccagcgagagctttcacgtgatctggcaccgggaatccccatctggccagaccgacaccctggctgccttccccgaagatagaagccagcctggccaagactgcagattccgggtgacacagctgcccaacggcagagacttccacatgtctgtggtgcgggctagacggaacgacagcggcacctacgtgtgcggcgtgatttctctggctcctaagatccagatcaaggaatccctgcgggccgagctgagagtgacagagagaagggccgaggtgcctaccgcccaccccagcccaagccctaggcccgccggccagttccagaccctggttcagaccacacctggagaacggagcagcctccccgccttctaccccggcaccagcggcagctgcagcggatgtggcagcctgtctctgcctctgctggccggcctggtcgccgccaacgccgtggcttctctgctgatcgtgggcgccgtgttcctgtgcgccagacctagacggtccccagctcaggaggacggcaaggtgtacatcaacatgcctggcagaggc

SEQ ID NO: 133 - DNA, 627 bp, synthetic construct
  misc_feature 1..627: note = Description of sequence: Construt: HA PD1.ICOS; Polypeptide structure: HA PD1 ECD, ICOS TM, ICOS intracellular
  source 1..627: mol_type = other DNA
cccggctggttcctggacagccccgacagaccttggaatcctccaacctttagcccagccctgctggtggtgacagagggagataacgccaccttcacctgcagcttcagcaacaccagcgagagctttcacgtgatctggcaccgggaatccccatctggccagaccgacaccctggctgccttccccgaagatagaagccagcctggccaagactgcagattccgggtgacacagctgcccaacggcagagacttccacatgtctgtggtgcgggctagacggaacgacagcggcacctacgtgtgcggcgtgatttctctggctcctaagatccagatcaaggaatccctgcgggccgagctgagagtgacagagagaagggccgaggtgcctaccgcccaccccagcccaagccctaggcccgccggccagttccagaccctggttttctggctgcctatcggctgcgccgcttttgtggtggtctgcatcctgggctgtatcctgatctgctggctgaccaagaagaagtacagctcttccgtgcacgaccccaacggcgagtacatgttcatgcgggccgtgaacaccgccaagaaaagcagactgacagatgtgaccctg

SEQ ID NO: 134 - DNA, 702 bp, synthetic construct
  misc_feature 1..702: note = Description of sequence: Construt: HA PD1.CD40; Polypeptide structure: HA PD1 ECD, CD40 TM, CD40 intracellular
  source 1..702: mol_type = other DNA
cccggctggttcctggacagccccgacagaccttggaatcctccaacctttagcccagccctgctggtggtgacagagggagataacgccaccttcacctgcagcttcagcaacaccagcgagagctttcacgtgatctggcaccgggaatccccatctggccagaccgacaccctggctgccttccccgaagatagaagccagcctggccaagactgcagattccgggtgacacagctgcccaacggcagagacttccacatgtctgtggtgcgggctagacggaacgacagcggcacctacgtgtgcggcgtgatttctctggctcctaagatccagatcaaggaatccctgcgggccgagctgagagtgacagagagaagggccgaggtgcctaccgcccaccccagcccaagccctaggcccgccggccagttccagaccctggttgccctggtggtgatccccatcatcttcggcatcctgttcgccattctgctggtgctggtctttatcaagaaggtggccaagaaacctacaaacaaggcccctcaccccaagcaggagcctcaggagatcaacttccccgacgacctgcctggaagcaataccgccgctccagtgcaagaaaccctgcacggctgccagcctgtgacccaggaagatggcaaagagtctagaatcagcgtgcaggagcggcag

SEQ ID NO: 135 - DNA, 639 bp, synthetic construct
  misc_feature 1..639: note = Description of sequence: Construt: HA PD1.OX40; Polypeptide structure: HA PD1 ECD, OX40 TM, OX40 intracellular
  source 1..639: mol_type = other DNA
cccggctggttcctggacagccccgacagaccttggaatcctccaacctttagcccagccctgctggtggtgacagagggagataacgccaccttcacctgcagcttcagcaacaccagcgagagctttcacgtgatctggcaccgggaatccccatctggccagaccgacaccctggctgccttccccgaagatagaagccagcctggccaagactgcagattccgggtgacacagctgcccaacggcagagacttccacatgtctgtggtgcgggctagacggaacgacagcggcacctacgtgtgcggcgtgatttctctggctcctaagatccagatcaaggaatccctgcgggccgagctgagagtgacagagagaagggccgaggtgcctaccgcccaccccagcccaagccctaggcccgccggccagttccagaccctggttgtggccgccatcctgggcctgggcctggtgctgggactgctgggccctctggctatcctgctggccctgtacctgctcagacgggaccagagactgccccccgacgcccacaagcctccaggcggcggatctttcagaacccctatccaggaggaacaggccgatgctcacagcacactggccaagatc

SEQ ID NO: 136 - DNA, 768 bp, synthetic construct
  misc_feature 1..768: note = Description of sequence: Construt: HA PD1.BAFFR; Polypeptide structure: HA PD1 ECD, BAFFR TM, BAFFR intracellular
  source 1..768: mol_type = other DNA
cccggctggttcctggacagccccgacagaccttggaatcctccaacctttagcccagccctgctggtggtgacagagggagataacgccaccttcacctgcagcttcagcaacaccagcgagagctttcacgtgatctggcaccgggaatccccatctggccagaccgacaccctggctgccttccccgaagatagaagccagcctggccaagactgcagattccgggtgacacagctgcccaacggcagagacttccacatgtctgtggtgcgggctagacggaacgacagcggcacctacgtgtgcggcgtgatttctctggctcctaagatccagatcaaggaatccctgcgggccgagctgagagtgacagagagaagggccgaggtgcctaccgcccaccccagcccaagccctaggcccgccggccagttccagaccctggttttcggcgctcccgctctgctgggcctcgccctggtgctggccctggtcctggtgggcctggtgtcctggcggcggagacagagaagactgagaggcgccagcagcgccgaggcccctgatggcgataaggacgcccctgagcctctggacaaagtgatcatcctgagccccggcatcagcgacgctaccgcccctgcctggcctccaccaggcgaggaccccggaacaacccctcctggccacagcgtgcctgtgcccgccaccgagctgggatctacagaactggtgaccacaaagaccgccggccctgaacagcag

SEQ ID NO: 137 - DNA, 1077 bp, synthetic construct
  misc_feature 1..1077: note = Description of sequence: Construt: HA PD1.tm.MyD88; Polypeptide structure: HA PD1 ECD, PD1 TM, MyD88
  source 1..1077: mol_type = other DNA
cctggctggttcctggactcccctgacagaccttggaacccccccaccttcagcccagccctgctggtggtcaccgagggcgacaacgctacattcacctgcagcttcagcaacaccagcgagagcttccacgtgatctggcaccgggaatctccttctggccagacagacaccttggcagcttttccagaggatagaagccagcctggccaggactgcagattcagagtgacccagctgcccaacggccgggacttccacatgagcgtggtgcgggccagacggaacgacagcggcacctacgtgtgcggcgtgatctctctggccccaaagatccagatcaaggaaagcctgagagccgaactgcgggtgacggagagaagagccgaggtgccaacagcccaccccagcccttcccccagacccgccggacaatttcagaccctggtggtcggcgtggtgggcggactgctgggctctctggtgctgctggtgtgggtgctggccgtgatctgcagcagagccgctagaggaacaatcggcgccagacggaccggccagatggctgctggaggacctggcgctggcagcgccgctcctgtgtccagcaccagctctctgcctctggctgcacttaatatgagagtgcggcggagactgagcctcttcctgaatgtgcgcacccaagtggcagctgattggaccgccctggccgaagagatggacttcgagtacctggaaatcagacagctggaaacccaggccgaccctacaggcagactgctggatgcctggcagggcagaccgggcgccagcgttggaaggctgctggacctcctgaccaagctgggccgggatgatgtgctgctggagctgggtcctagcatcgaggaagattgccagaaatacatcctgaaacagcaacaggaggaagccgagaagcctctgcaggtggccgccgtggacagctctgtgcctagaacagccgagctggccggcatcaccaccctggacgaccccctgggccacatgcctgagcggttcgacgcctttatttgttattgcccctctgacatc

SEQ ID NO: 138 - DNA, 672 bp, synthetic construct
  misc_feature 1..672: note = Description of sequence: Construt: WT BR2.CD28; Polypeptide structure: WT BR2 ECD, CD28 ECD (truncated), CD28 TM, CD28 intracellular
  source 1..672: mol_type = other DNA
acaatccccccccacgtgcagaagtccgtgaacaatgacatgatcgtcaccgacaacaacggcgctgtgaagtttccacaactgtgcaagttctgcgacgtgcggttcagcacatgcgataaccagaaaagctgtatgagcaattgctccattacaagcatctgtgaaaaacctcaggaggtgtgcgtggccgtgtggcggaagaacgacgagaacatcaccctggagaccgtgtgtcacgatcctaagctgccttaccacgacttcatcctggaagatgccgccagccctaagtgcatcatgaaggaaaagaaaaagcctggcgagaccttcttcatgtgctcttgttctagcgacgagtgcaacgataatatcatcttcagcgaggaatacaacaccagcaaccccgacctgctgctcgtgatctttcagtgtcctagccccctgttccccggtcctagcaaacctttctgggtgctggtggtggtgggcggcgtgctggcctgctacagcctgctggtcacagtggcctttatcatcttctgggtcagatctaagcggtccagactgctgcattctgattacatgaacatgacccctagaagacctggacctacaagaaagcactaccagccttacgcccctcctcgggacttcgccgcttatagaagc

SEQ ID NO: 139 - DNA, 672 bp, synthetic construct
  misc_feature 1..672: note = Description of sequence: Construt: WT BR2.CD28.YMFM; Polypeptide structure: WT BR2 ECD, CD28 ECD (truncated), CD28 TM, CD28.YMFM intracellular
  source 1..672: mol_type = other DNA
acaatccccccccacgtgcagaagtccgtgaacaatgacatgatcgtcaccgacaacaacggcgctgtgaagtttccacaactgtgcaagttctgcgacgtgcggttcagcacatgcgataaccagaaaagctgtatgagcaattgctccattacaagcatctgtgaaaaacctcaggaggtgtgcgtggccgtgtggcggaagaacgacgagaacatcaccctggagaccgtgtgtcacgatcctaagctgccttaccacgacttcatcctggaagatgccgccagccctaagtgcatcatgaaggaaaagaaaaagcctggcgagaccttcttcatgtgctcttgttctagcgacgagtgcaacgataatatcatcttcagcgaggaatacaacaccagcaaccccgacctgctgctcgtgatctttcagtgtcctagccccctgttccccggtcctagcaaacctttctgggtgctggtggtggtgggcggcgtgctggcctgctacagcctgctggtcacagtggcctttatcatcttctgggtcagatctaagcggtccagactgctgcattctgattacatgttcatgacccctagaagacctggacctacaagaaagcactaccagccttacgcccctcctcgggacttcgccgcttatagaagc

SEQ ID NO: 140 - DNA, 672 bp, synthetic construct
  misc_feature 1..672: note = Description of sequence: Construt: WT BR2.CD28.AYAA; Polypeptide structure: WT BR2 ECD, CD28 ECD (truncated), CD28 TM, CD28.AYAA intracellular
  source 1..672: mol_type = other DNA
acaatccccccccacgtgcagaagtccgtgaacaatgacatgatcgtcaccgacaacaacggcgctgtgaagtttccacaactgtgcaagttctgcgacgtgcggttcagcacatgcgataaccagaaaagctgtatgagcaattgctccattacaagcatctgtgaaaaacctcaggaggtgtgcgtggccgtgtggcggaagaacgacgagaacatcaccctggagaccgtgtgtcacgatcctaagctgccttaccacgacttcatcctggaagatgccgccagccctaagtgcatcatgaaggaaaagaaaaagcctggcgagaccttcttcatgtgctcttgttctagcgacgagtgcaacgataatatcatcttcagcgaggaatacaacaccagcaaccccgacctgctgctcgtgatctttcagtgtccttcccctctgttccccggccctagcaaacccttctgggtgctggtggtcgtgggcggagtgctggcttgctacagcctgctcgtgaccgtggccttcatcatcttctgggtcagaagcaagcggtccagactgctgcacagcgactacatgaacatgacacctagaagacctggacctacaagaaagcactaccaggcctacgccgcccctcgggacttcgccgcttatagatct

SEQ ID NO: 141 - DNA, 894 bp, synthetic construct
  misc_feature 1..894: note = Description of sequence: Construt: WT BR2.CD2(full); Polypeptide structure: WT BR2 ECD, CD2 ECD (truncated), CD2 TM, CD2 intracellular
  source 1..894: mol_type = other DNA
acaatccccccccacgtgcagaagtccgtgaacaatgacatgatcgtcaccgacaacaacggcgctgtgaagtttccacaactgtgcaagttctgcgacgtgcggttcagcacatgcgataaccagaaaagctgtatgagcaattgctccattacaagcatctgtgaaaaacctcaggaggtgtgcgtggccgtgtggcggaagaacgacgagaacatcaccctggagaccgtgtgtcacgatcctaagctgccttaccacgacttcatcctggaagatgccgccagccctaagtgcatcatgaaggaaaagaaaaagcctggcgagaccttcttcatgtgctcttgttctagcgacgagtgcaacgataatatcatcttcagcgaggaatacaacaccagcaaccccgacctgctgctcgtgatctttcaggtggagcctgtgtcctgtcctgagaagggactggacatctacctgatcatcggcatctgcggaggaggcagcctgctgatggtgttcgtggccctgctggtgttctacatcaccaagcggaagaagcagcggagcagacggaatgacgaggaactcgagacaagagcccatcgggtcgccacagaggaaagaggcagaaagccccaccagattcctgccagcacacctcagaaccctgctaccagccaacacccccccccccctcctggccacagatctcaggcccctagccaccggcccccgccacctggccaccgggtgcagcaccagcctcaaaaaagaccccctgctcctagcggcacacaggtgcaccagcagaaaggtcctccactgcctagacctcgggtgcagcctaagcctccacatggcgccgctgagaacagcttgtctcctagttctaat

SEQ ID NO: 142 - DNA, 801 bp, synthetic construct
  misc_feature 1..801: note = Description of sequence: Construt: WT BR2.CD2(short); Polypeptide structure: WT BR2 ECD, CD2 ECD (truncated), CD2 TM, CD2 intracellular (short)
  source 1..801: mol_type = other DNA
acaatccccccccacgtgcagaagtccgtgaacaatgacatgatcgtcaccgacaacaacggcgctgtgaagtttccacaactgtgcaagttctgcgacgtgcggttcagcacatgcgataaccagaaaagctgtatgagcaattgctccattacaagcatctgtgaaaaacctcaggaggtgtgcgtggccgtgtggcggaagaacgacgagaacatcaccctggagaccgtgtgtcacgatcctaagctgccttaccacgacttcatcctggaagatgccgccagccctaagtgcatcatgaaggaaaagaaaaagcctggcgagaccttcttcatgtgctcttgttctagcgacgagtgcaacgataatatcatcttcagcgaggaatacaacaccagcaaccccgacctgctgctcgtgatctttcaggtcgagcctgtgagctgccccgagaaggggctggacatctacctgatcatcggcatttgtggcggcggatctctgctgatggtgttcgtggccctgctggtgttctacatcaccaagagaaagaagcagacccctcagaaccccgccacaagccagcatcctccaccacctcccggccaccggagccaggccccaagtcacagacccccacctcctggccacagagtgcagcaccagccccagaagcggcctccagctcctagcggaacccaagtgcaccagcagaaaggccctcctctgcctcggcctagagtgcagcctaaacctccgcacggcgctgctgagaacagcttgtctccctccagcaat

SEQ ID NO: 143 - DNA, 657 bp, synthetic construct
  misc_feature 1..657: note = Description of sequence: Construt: WT BR2.DAP10.D57N; Polypeptide structure: WT BR2 ECD, DAP10 ECD, DAP10 TM D57N, DAP10 intracellular
  source 1..657: mol_type = other DNA
acaatccccccccacgtgcagaagtccgtgaacaatgacatgatcgtcaccgacaacaacggcgctgtgaagtttccacaactgtgcaagttctgcgacgtgcggttcagcacatgcgataaccagaaaagctgtatgagcaattgctccattacaagcatctgtgaaaaacctcaggaggtgtgcgtggccgtgtggcggaagaacgacgagaacatcaccctggagaccgtgtgtcacgatcctaagctgccttaccacgacttcatcctggaagatgccgccagccctaagtgcatcatgaaggaaaagaaaaagcctggcgagaccttcttcatgtgctcttgttctagcgacgagtgcaacgataatatcatcttcagcgaggaatacaacaccagcaaccccgacctgctgctcgtgatctttcagcagaccacacctggagaacggagcagcctccccgccttctaccccggcaccagcggcagctgcagcggatgtggcagcctgtctctgcctctgctggccggcctggtcgccgccaacgccgtggcttctctgctgatcgtgggcgccgtgttcctgtgcgccagacctagacggtccccagctcaggaggacggcaaggtgtacatcaacatgcctggcagaggc

SEQ ID NO: 144 - DNA, 609 bp, synthetic construct
  misc_feature 1..609: note = Description of sequence: Construt: WT BR2.ICOS; Polypeptide structure: WT BR2 ECD, ICOS TM, ICOS intracellular
  source 1..609: mol_type = other DNA
acaatccccccccacgtgcagaagtccgtgaacaatgacatgatcgtcaccgacaacaacggcgctgtgaagtttccacaactgtgcaagttctgcgacgtgcggttcagcacatgcgataaccagaaaagctgtatgagcaattgctccattacaagcatctgtgaaaaacctcaggaggtgtgcgtggccgtgtggcggaagaacgacgagaacatcaccctggagaccgtgtgtcacgatcctaagctgccttaccacgacttcatcctggaagatgccgccagccctaagtgcatcatgaaggaaaagaaaaagcctggcgagaccttcttcatgtgctcttgttctagcgacgagtgcaacgataatatcatcttcagcgaggaatacaacaccagcaaccccgacctgctgctcgtgatctttcagttctggctgcctatcggctgcgccgcttttgtggtggtctgcatcctgggctgtatcctgatctgctggctgaccaagaagaagtacagctcttccgtgcacgaccccaacggcgagtacatgttcatgcgggccgtgaacaccgccaagaaaagcagactgacagatgtgaccctg

SEQ ID NO: 145 - DNA, 684 bp, synthetic construct
  misc_feature 1..684: note = Description of sequence: Construt: WT BR2.CD40; Polypeptide structure: WT BR2 ECD, CD40 TM, CD40 intracellular
  source 1..684: mol_type = other DNA
acaatccccccccacgtgcagaagtccgtgaacaatgacatgatcgtcaccgacaacaacggcgctgtgaagtttccacaactgtgcaagttctgcgacgtgcggttcagcacatgcgataaccagaaaagctgtatgagcaattgctccattacaagcatctgtgaaaaacctcaggaggtgtgcgtggccgtgtggcggaagaacgacgagaacatcaccctggagaccgtgtgtcacgatcctaagctgccttaccacgacttcatcctggaagatgccgccagccctaagtgcatcatgaaggaaaagaaaaagcctggcgagaccttcttcatgtgctcttgttctagcgacgagtgcaacgataatatcatcttcagcgaggaatacaacaccagcaaccccgacctgctgctcgtgatctttcaggccctggtggtgatccccatcatcttcggcatcctgttcgccattctgctggtgctggtctttatcaagaaggtggccaagaaacctacaaacaaggcccctcaccccaagcaggagcctcaggagatcaacttccccgacgacctgcctggaagcaataccgccgctccagtgcaagaaaccctgcacggctgccagcctgtgacccaggaagatggcaaagagtctagaatcagcgtgcaggagcggcag

SEQ ID NO: 146 - DNA, 621 bp, synthetic construct
  misc_feature 1..621: note = Description of sequence: Construt: WT BR2.OX40; Polypeptide structure: WT BR2 ECD, OX40 TM, OX40 intracellular
  source 1..621: mol_type = other DNA
acaatccccccccacgtgcagaagtccgtgaacaatgacatgatcgtcaccgacaacaacggcgctgtgaagtttccacaactgtgcaagttctgcgacgtgcggttcagcacatgcgataaccagaaaagctgtatgagcaattgctccattacaagcatctgtgaaaaacctcaggaggtgtgcgtggccgtgtggcggaagaacgacgagaacatcaccctggagaccgtgtgtcacgatcctaagctgccttaccacgacttcatcctggaagatgccgccagccctaagtgcatcatgaaggaaaagaaaaagcctggcgagaccttcttcatgtgctcttgttctagcgacgagtgcaacgataatatcatcttcagcgaggaatacaacaccagcaaccccgacctgctgctcgtgatctttcaggtggccgccatcctgggcctgggcctggtgctgggactgctgggccctctggctatcctgctggccctgtacctgctcagacgggaccagagactgccccccgacgcccacaagcctccaggcggcggatctttcagaacccctatccaggaggaacaggccgatgctcacagcacactggccaagatc

SEQ ID NO: 147 - DNA, 1059 bp, synthetic construct
  misc_feature 1..1059: note = Description of sequence: Construt: WT BR2.PD1 tm.MyD88; Polypeptide structure: WT BR2 ECD, PD1 TM, MyD88
  source 1..1059: mol_type = other DNA
acaatccccccccacgtgcagaagtccgtgaacaatgacatgatcgtcaccgacaacaacggcgctgtgaagtttccacaactgtgcaagttctgcgacgtgcggttcagcacatgcgataaccagaaaagctgtatgagcaattgctccattacaagcatctgtgaaaaacctcaggaggtgtgcgtggccgtgtggcggaagaacgacgagaacatcaccctggagaccgtgtgtcacgatcctaagctgccttaccacgacttcatcctggaagatgccgccagccctaagtgcatcatgaaggaaaagaaaaagcctggcgagaccttcttcatgtgctcttgttctagcgacgagtgcaacgataatatcatcttcagcgaggaatacaacaccagcaaccccgacctgctgctcgtgatctttcaggtcggcgtggtgggcggactgctgggctctctggtgctgctggtgtgggtgctggccgtgatctgcagcagagccgctagaggaacaatcggcgccagacggaccggccagatggccgccggaggccctggcgctggaagcgccgcacctgtgtcctctacatctagtctgcctctggccgctcttaatatgagagtgcggagaagactgagcctgttcctgaacgtgcgcacacaagtggccgctgattggactgccctggctgaagagatggacttcgagtacctggaaatcagacagctggaaacccaggccgaccccacaggccggctgctggacgcctggcagggcagacctggagccagcgtgggcagactgctggacctgctgaccaagctgggacgggacgacgtgctgctggaactgggcccctctattgaggaagattgccagaaatacatcctgaaacagcagcaggaggaggccgaaaagcctctgcaggtggccgccgtggacagcagcgtgcccagaaccgccgagctggctggcatcaccacactggatgatcctctgggccacatgcctgaaagattcgacgccttcatctgctactgtcctagcgacatc

SEQ ID NO: 148 - DNA, 594 bp, synthetic construct
  misc_feature 1..594: note = Description of sequence: Construt: dN25 BR2.CD28; Polypeptide structure: dN BR2 ECD, CD28 ECD (truncated), CD28 TM, CD28 intracellular
  source 1..594: mol_type = other DNA
cagctgtgcaagttctgcgacgtgcggttcagcacctgtgataaccagaaaagctgtatgagcaattgctctatcacctccatctgcgagaagcctcaggaggtgtgcgtggccgtgtggcggaagaacgacgagaacattacactggaaaccgtgtgtcacgatcctaagctgccttaccacgacttcatcctggaagatgccgccagccctaagtgcatcatgaaggaaaagaaaaagccaggcgagacatttttcatgtgctcctgtagcagcgacgagtgcaacgacaatatcatctttagcgaggaatacaacaccagcaaccccgacctgctcctggtcatcttccaatgtcctagccccctgttccccggtcctagcaaacctttctgggtgctggtggtggtgggcggcgtgctggcctgctacagcctgctggtcacagtggcctttatcatcttctgggtcagatctaagcggtccagactgctgcattctgattacatgaacatgacccctagaagacctggacctacaagaaagcactaccagccttacgcccctcctcgggacttcgccgcttatagaagc

SEQ ID NO: 149 - DNA, 594 bp, synthetic construct
  misc_feature 1..594: note = Description of sequence: Construt: dN25 BR2.CD28.YMFM; Polypeptide structure: dN25 BR2 ECD, CD28 ECD (truncated), CD28 TM, CD28.YMFM intracellular
  source 1..594: mol_type = other DNA
cagctgtgcaagttctgcgacgtgcggttcagcacctgtgataaccagaaaagctgtatgagcaattgctctatcacctccatctgcgagaagcctcaggaggtgtgcgtggccgtgtggcggaagaacgacgagaacattacactggaaaccgtgtgtcacgatcctaagctgccttaccacgacttcatcctggaagatgccgccagccctaagtgcatcatgaaggaaaagaaaaagccaggcgagacatttttcatgtgctcctgtagcagcgacgagtgcaacgacaatatcatctttagcgaggaatacaacaccagcaaccccgacctgctcctggtcatcttccaatgtcctagccccctgttccccggtcctagcaaacctttctgggtgctggtggtggtgggcggcgtgctggcctgctacagcctgctggtcacagtggcctttatcatcttctgggtcagatctaagcggtccagactgctgcattctgattacatgttcatgacccctagaagacctggacctacaagaaagcactaccagccttacgcccctcctcgggacttcgccgcttatagaagc

SEQ ID NO: 150 - DNA, 594 bp, synthetic construct
  misc_feature 1..594: note = Description of sequence: Construt: dN25 BR2.CD28.AYAA; Polypeptide structure: dN25 BR2 ECD, CD28 ECD (truncated), CD28 TM, CD28.AYAA intracellular
  source 1..594: mol_type = other DNA
cagctgtgcaagttctgcgacgtgcggttcagcacctgtgataaccagaaaagctgtatgagcaattgctctatcacctccatctgcgagaagcctcaggaggtgtgcgtggccgtgtggcggaagaacgacgagaacattacactggaaaccgtgtgtcacgatcctaagctgccttaccacgacttcatcctggaagatgccgccagccctaagtgcatcatgaaggaaaagaaaaagccaggcgagacatttttcatgtgctcctgtagcagcgacgagtgcaacgacaatatcatctttagcgaggaatacaacaccagcaaccccgacctgctcctggtcatcttccaatgtcctagccccctgttccccggtcctagcaaacctttctgggtgctggtggtggtgggcggcgtgctggcctgctacagcctgctggtcacagtggcctttatcatcttctgggtcagatctaagcggtccagactgctgcattctgattacatgaacatgacccctagaagacctggacctacaagaaagcactaccaggcctacgccgcccctcgggacttcgccgcttatagaagc

SEQ ID NO: 151 - DNA, 816 bp, synthetic construct
  misc_feature 1..816: note = Description of sequence: Construt: dN25 BR2.CD2(full); Polypeptide structure: dN25 BR2 ECD, CD2 ECD (truncated), CD2 TM, CD2 intracellular
  source 1..816: mol_type = other DNA
cagctgtgcaagttctgcgacgtgcggttcagcacctgtgataaccagaaaagctgtatgagcaattgctctatcacctccatctgcgagaagcctcaggaggtgtgcgtggccgtgtggcggaagaacgacgagaacattacactggaaaccgtgtgtcacgatcctaagctgccttaccacgacttcatcctggaagatgccgccagccctaagtgcatcatgaaggaaaagaaaaagccaggcgagacatttttcatgtgctcctgtagcagcgacgagtgcaacgacaatatcatctttagcgaggaatacaacaccagcaaccccgacctgctcctggtcatcttccaagtggagcctgtgtcctgtcctgagaagggactggacatctacctgatcatcggcatctgcggaggaggcagcctgctgatggtgttcgtggccctgctggtgttctacatcaccaagcggaagaagcagcggagcagacggaatgacgaggaactcgagacaagagcccatcgggtcgccacagaggaaagaggcagaaagccccaccagattcctgccagcacacctcagaaccctgctaccagccaacacccccccccccctcctggccacagatctcaggcccctagccaccggcccccgccacctggccaccgggtgcagcaccagcctcaaaaaagaccccctgctcctagcggcacacaggtgcaccagcagaaaggtcctccactgcctagacctcgggtgcagcctaagcctccacatggcgccgctgagaacagcttgtctcctagttctaat

SEQ ID NO: 152 - DNA, 723 bp, synthetic construct
  misc_feature 1..723: note = Description of sequence: Construt: dN25 BR2.CD2(short); Polypeptide structure: dN25 BR2 ECD, CD2 ECD (truncated), CD2 TM, CD2 intracellular (short)
  source 1..723: mol_type = other DNA
cagctgtgcaagttctgcgacgtgcggttcagcacctgtgataaccagaaaagctgtatgagcaattgctctatcacctccatctgcgagaagcctcaggaggtgtgcgtggccgtgtggcggaagaacgacgagaacattacactggaaaccgtgtgtcacgatcctaagctgccttaccacgacttcatcctggaagatgccgccagccctaagtgcatcatgaaggaaaagaaaaagccaggcgagacatttttcatgtgctcctgtagcagcgacgagtgcaacgacaatatcatctttagcgaggaatacaacaccagcaaccccgacctgctcctggtcatcttccaagtcgagcctgtgagctgccccgagaaggggctggacatctacctgatcatcggcatttgtggcggcggatctctgctgatggtgttcgtggccctgctggtgttctacatcaccaagagaaagaagcagacccctcagaaccccgccacaagccagcatcctccaccacctcccggccaccggagccaggccccaagtcacagacccccacctcctggccacagagtgcagcaccagccccagaagcggcctccagctcctagcggaacccaagtgcaccagcagaaaggccctcctctgcctcggcctagagtgcagcctaaacctccgcacggcgctgctgagaacagcttgtctccctccagcaat

SEQ ID NO: 153 - DNA, 579 bp, synthetic construct
  misc_feature 1..579: note = Description of sequence: Construt: dN25 BR2.DAP10.D57N; Polypeptide structure: dN25 BR2 ECD, DAP10 ECD, DAP10 TM D57N, DAP10 intracellular
  source 1..579: mol_type = other DNA
cagctgtgcaagttctgcgacgtgcggttcagcacctgtgataaccagaaaagctgtatgagcaattgctctatcacctccatctgcgagaagcctcaggaggtgtgcgtggccgtgtggcggaagaacgacgagaacattacactggaaaccgtgtgtcacgatcctaagctgccttaccacgacttcatcctggaagatgccgccagccctaagtgcatcatgaaggaaaagaaaaagccaggcgagacatttttcatgtgctcctgtagcagcgacgagtgcaacgacaatatcatctttagcgaggaatacaacaccagcaaccccgacctgctcctggtcatcttccaacagaccacacctggagaacggagcagcctccccgccttctaccccggcaccagcggcagctgcagcggatgtggcagcctgtctctgcctctgctggccggcctggtcgccgccaacgccgtggcttctctgctgatcgtgggcgccgtgttcctgtgcgccagacctagacggtccccagctcaggaggacggcaaggtgtacatcaacatgcctggcagaggc

SEQ ID NO: 154 - DNA, 531 bp, synthetic construct
  misc_feature 1..531: note = Description of sequence: Construt: dN25 BR2.ICOS; Polypeptide structure: dN25 BR2 ECD, ICOS TM, ICOS intracellular
  source 1..531: mol_type = other DNA
cagctgtgcaagttctgcgacgtgcggttcagcacctgtgataaccagaaaagctgtatgagcaattgctctatcacctccatctgcgagaagcctcaggaggtgtgcgtggccgtgtggcggaagaacgacgagaacattacactggaaaccgtgtgtcacgatcctaagctgccttaccacgacttcatcctggaagatgccgccagccctaagtgcatcatgaaggaaaagaaaaagccaggcgagacatttttcatgtgctcctgtagcagcgacgagtgcaacgacaatatcatctttagcgaggaatacaacaccagcaaccccgacctgctcctggtcatcttccaattctggctgcctatcggctgcgccgcttttgtggtggtctgcatcctgggctgtatcctgatctgctggctgaccaagaagaagtacagctcttccgtgcacgaccccaacggcgagtacatgttcatgcgggccgtgaacaccgccaagaaaagcagactgacagatgtgaccctg

SEQ ID NO: 155 - DNA, 606 bp, synthetic construct
  misc_feature 1..606: note = Description of sequence: Construt: dN25 BR2.CD40; Polypeptide structure: dN25 BR2 ECD, CD40 TM, CD40 intracellular
  source 1..606: mol_type = other DNA
cagctgtgcaagttctgcgacgtgcggttcagcacctgtgataaccagaaaagctgtatgagcaattgctctatcacctccatctgcgagaagcctcaggaggtgtgcgtggccgtgtggcggaagaacgacgagaacattacactggaaaccgtgtgtcacgatcctaagctgccttaccacgacttcatcctggaagatgccgccagccctaagtgcatcatgaaggaaaagaaaaagccaggcgagacatttttcatgtgctcctgtagcagcgacgagtgcaacgacaatatcatctttagcgaggaatacaacaccagcaaccccgacctgctcctggtcatcttccaagccctggtggtgatccccatcatcttcggcatcctgttcgccattctgctggtgctggtctttatcaagaaggtggccaagaaacctacaaacaaggcccctcaccccaagcaggagcctcaggagatcaacttccccgacgacctgcctggaagcaataccgccgctccagtgcaagaaaccctgcacggctgccagcctgtgacccaggaagatggcaaagagtctagaatcagcgtgcaggagcggcag

SEQ ID NO: 156 - DNA, 543 bp, synthetic construct
  misc_feature 1..543: note = Description of sequence: Construt: dN25 BR2.OX40; Polypeptide structure: dN25 BR2 ECD, OX40 TM, OX40 intracellular
  source 1..543: mol_type = other DNA
cagctgtgcaagttctgcgacgtgcggttcagcacctgtgataaccagaaaagctgtatgagcaattgctctatcacctccatctgcgagaagcctcaggaggtgtgcgtggccgtgtggcggaagaacgacgagaacattacactggaaaccgtgtgtcacgatcctaagctgccttaccacgacttcatcctggaagatgccgccagccctaagtgcatcatgaaggaaaagaaaaagccaggcgagacatttttcatgtgctcctgtagcagcgacgagtgcaacgacaatatcatctttagcgaggaatacaacaccagcaaccccgacctgctcctggtcatcttccaagtggccgccatcctgggcctgggcctggtgctgggactgctgggccctctggctatcctgctggccctgtacctgctcagacgggaccagagactgccccccgacgcccacaagcctccaggcggcggatctttcagaacccctatccaggaggaacaggccgatgctcacagcacactggccaagatc

SEQ ID NO: 157 - DNA, 672 bp, synthetic construct
  misc_feature 1..672: note = Description of sequence: Construt: dN25 BR2.BAFFR; Polypeptide structure: dN25 BR2 ECD, BAFFR TM, BAFFR intracellular
  source 1..672: mol_type = other DNA
cagctgtgcaagttctgcgacgtgcggttcagcacctgtgataaccagaaaagctgtatgagcaattgctctatcacctccatctgcgagaagcctcaggaggtgtgcgtggccgtgtggcggaagaacgacgagaacattacactggaaaccgtgtgtcacgatcctaagctgccttaccacgacttcatcctggaagatgccgccagccctaagtgcatcatgaaggaaaagaaaaagccaggcgagacatttttcatgtgctcctgtagcagcgacgagtgcaacgacaatatcatctttagcgaggaatacaacaccagcaaccccgacctgctcctggtcatcttccaattcggcgctcccgctctgctgggcctcgccctggtgctggccctggtcctggtgggcctggtgtcctggcggcggagacagagaagactgagaggcgccagcagcgccgaggcccctgatggcgataaggacgcccctgagcctctggacaaagtgatcatcctgagccccggcatcagcgacgctaccgcccctgcctggcctccaccaggcgaggaccccggaacaacccctcctggccacagcgtgcctgtgcccgccaccgagctgggatctacagaactggtgaccacaaagaccgccggccctgaacagcag

SEQ ID NO: 158 - DNA, 981 bp, synthetic construct
  misc_feature 1..981: note = Description of sequence: Construt: dN25 BR2.PD1 tm.MyD88; Polypeptide structure: dN25 BR2 ECD, PD1 TM, MyD88
  source 1..981: mol_type = other DNA
cagctgtgcaagttctgcgacgtgcggttcagcacctgtgataaccagaaaagctgtatgagcaattgctctatcacctccatctgcgagaagcctcaggaggtgtgcgtggccgtgtggcggaagaacgacgagaacattacactggaaaccgtgtgtcacgatcctaagctgccttaccacgacttcatcctggaagatgccgccagccctaagtgcatcatgaaggaaaagaaaaagccaggcgagacatttttcatgtgctcctgtagcagcgacgagtgcaacgacaatatcatctttagcgaggaatacaacaccagcaaccccgacctgctcctggtcatcttccaagtcggcgtggtgggcggactgctgggctctctggtgctgctggtgtgggtgctggccgtgatctgcagcagagccgctagaggaacaatcggcgccagacggaccggccagatggccgccggaggccctggcgctggaagcgccgcacctgtgtcctctacatctagtctgcctctggccgctcttaatatgagagtgcggagaagactgagcctgttcctgaacgtgcgcacacaagtggccgctgattggactgccctggctgaagagatggacttcgagtacctggaaatcagacagctggaaacccaggccgaccccacaggccggctgctggacgcctggcagggcagacctggagccagcgtgggcagactgctggacctgctgaccaagctgggacgggacgacgtgctgctggaactgggcccctctattgaggaagattgccagaaatacatcctgaaacagcagcaggaggaggccgaaaagcctctgcaggtggccgccgtggacagcagcgtgcccagaaccgccgagctggctggcatcaccacactggatgatcctctgggccacatgcctgaaagattcgacgccttcatctgctactgtcctagcgacatc

SEQ ID NO: 159 - protein, 20 aa, synthetic construct
  REGION 1..20: note = Description of sequence: GS linker
GGGGSGGGGSGGGGSGGGGS

SEQ ID NO: 160 - DNA, 546 bp, synthetic construct
  misc_feature 1..546: note = Description of sequence: Construt: CCR 15.1; Polypeptide structure: TPOR TM, TPOR JAK-binding domain, IL2RbYY signaling domain
  source 1..546: mol_type = other DNA
tcagaccctactagagtcgagaccgctaccgagaccgcttggatctctctggtgaccgccctgcacctggtgctgggcctgaacgccgtgctgggcctgctgctgctgaggaagcagttcccagcacactaccggagactgaggcacgcactgtggccaagcctgcccgacctgcacagggtgctgggacagtatctgagggatacagccgccctgagcccacctaaggcaaccgtgtccgacacatgcgaggaggtggaaccaagtctgctggaaatcctgccaaaatcctctgagcggacacccctgcccctgctcgaggacgagggagtggcaggagcaccaaccggcagctccccccagcctctgcagccactgtccggagaggacgatgcatactgcacattcccttctcgggacgatctgctgctgttctctccaagcggacagggagagtttcgggccctgaacgccagactgcccctgaataccgacgcctatctgagcctgcaggagctgcagggacaggaccccacacacctggtg

SEQ ID NO: 161 - DNA, 624 bp, synthetic construct
  misc_feature 1..624: note = Description of sequence: Construt: dnHACi PD1; Polypeptide structure: CD8 signal sequence (underlined), HA PD1 ECD, PD1 TM
  source 1..624: mol_type = other DNA
atggccctgccagtgaccgccctgctgctgccactggccctgctgctgcacgcagcaaggccacctggatggtttctggactcccctgataggccctggaatcccccaactttctcccctgccctgctggtggtcactgaaggcgacaacgccaccttcacatgcagcttttccaacacctctgagagcttccacgtgatctggcacagggagtccccatctggccagaccgacacactggcagcatttcctgaggaccgctcccagccaggacaggattgccggttcagagtgacccagctgcccaacggccgggactttcacatgtctgtggtgagagcccggagaaatgatagcggcacctacgtgtgcggcgtgatctccctggcccccaagatccagatcaaggagtctctgagggcagagctgagggtgaccgagaggagggcagaggtgcctacagcacacccaagcccttccccacggcccgcaggacagttccagacactggtggtgggagtggtgggaggcctgctgggcagcctggtgctgctggtgtgggtgctggctgtcatctgtagcagggccgcaagaggcaccattggggcacgaaggactgggcag

SEQ ID NO: 162 - protein, 182 aa, synthetic construct
  REGION 1..182: note = Description of sequence: Construct name: CCR 15.1; Polypeptide structure: C68
SDPTRVETATETAWISLVTALHLVLGLNAVLGLLLLRKQFPAHYRRLRHALWPSLPDLHRVLGQYLRDTAALSPPKATVSDTCEEVEPSLLEILPKSSERTPLPLLEDEGVAGAPTGSSPQPLQPLSGEDDAYCTFPSRDDLLLFSPSGQGEFRALNARLPLNTDAYLSLQELQGQDPTHLV

SEQ ID NO: 163 - protein, 208 aa, synthetic construct
  REGION 1..208: note = Description of sequence: Construct name: dnHACi PD1; Polypeptide structure: C68
MALPVTALLLPLALLLHAARPPGWFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLVVGVVGGLLGSLVLLVWVLAVICSRAARGTIGARRTGQ

SEQ ID NO: 164 - protein, 60 aa, synthetic construct
  REGION 1..60: note = Description of sequence: (GGGGS)4 linker
GGCGGCGGCGGCTCTGGAGGAGGAGGCAGCGGCGGAGGAGGCTCCGGAGGCGGCGGCTCT

SEQ ID NO: 165 - protein, 509 aa, synthetic construct
  REGION 1..509: note = Description of sequence: Construct name: 2G1 (DLL3 CAR); Polypeptide structure: rituximab mimotope RSR, 2G1 scFv, rituximab mimotope, CD8-Alpha hinge, CD8-Alpha transmembrane, CD8-Alpha cytoplasmic domain (truncated), 4-1BB (TNFRSF9, CD137) cytoplasmic domain, CD3? cytoplasmic domain
GGGGSCPYSNPSLCGGGGSQLQLQESGPGLVKPSETLSLTCTVSGGSISSSSYYWGWIRQPPGKGLEWIGSIYYSGNIYHNPSLKSRVSISVDTSKNQFSLRLSSVTAADTAVYYCAREIIVGATHFDYWGQGTLVTVSSGGGGSGGGGSGGGGSGGGGSAIQMTQSPSSLSASVGDRVTITCRASQGIRNDLGWYQQKPGKAPELLIYAASSLQSGVPSRFSGSGSGTDFTLTISSLQPEDFATYYCLQDYNYPLTFGPGTKVDIKGGGGSCPYSNPSLCGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 166 - protein, 223 aa, synthetic construct
  REGION 1..223: note = Description of sequence: Construct name: CCR 15.3; Polypeptide structure: TPOR TM, TPOR JAK-binding domain, IL2RbYYY signaling domain
SDPTRVETATETAWISLVTALLLVLGLNAVLGLLLLRKQFPAHYRRLRHALWPSLPDLHRVLGQYLRDTAALSPPKATVSDTCEEVEPSLLEILPKSSERTPLPLLEQQDKVPEPASLSSNHSLTSCFTNQGYFFFHLPDALEIEACQDEGVAGAPTGSSPQPLQPLSGEDDAYCTFPSRDDLLLFSPSGQGEFRALNARLPLNTDAYLSLQELQGQDPTHLV

SEQ ID NO: 167 - DNA, 669 bp, synthetic construct
  misc_feature 1..669: note = Description of sequence: Construt: CCR 15.3; Polypeptide structure: TPOR TM, TPOR JAK-binding domain, IL2RbYYY signaling domain
  source 1..669: mol_type = other DNA
tcagaccctactagagtcgagaccgctaccgagaccgcttggatctctctggtgaccgccctgctgctggtgctgggcctgaacgccgtgctgggcctgctgctgctgaggaagcagttcccagcacactaccggagactgaggcacgcactgtggccaagcctgcccgacctgcacagggtgctgggacagtatctgagggatacagccgccctgagcccacctaaggcaaccgtgtccgacacatgcgaggaggtggaaccaagtctgctggaaatcctgccaaaatcctctgagcggacacccctgcccctgctcgagcagcaggacaaggtgcccgagcctgcctccctgagctccaaccacagcctgacctcctgctttacaaatcagggctacttctttttccacctgcctgacgccctggagatcgaggcctgtcaggatgagggagtggcaggagcacctaccggctctagcccacagccactgcagccactgtctggagaggacgatgcctactgcacattccccagccgggacgatctgctgctgttttccccttctggacagggagagttccgggccctgaacgcaagactgccactgaataccgacgcctatctgtctctgcaggagctgcagggccaggaccccacacacctggtg

SEQ ID NO: 168-169 - skipped (2 entries)

SEQ ID NO: 170 - protein, 4 aa, synthetic construct
  REGION 1..4: note = Description of sequence: SH3 domain
PPPP

SEQ ID NO: 171 - protein, 5 aa, synthetic construct
  REGION 1..5: note = Description of sequence: SH3 domain
PPPPP

SEQ ID NO: 172 - protein, 4 aa, synthetic construct
  REGION 1..4: note = Description of sequence: SH3 domain
PPAP

SEQ ID NO: 173 - protein, 4 aa, synthetic construct
  REGION 1..4: note = Description of sequence: SH3 domain
PPLP

SEQ ID NO: 174 - protein, 5 aa, synthetic construct
  REGION 1..5: note = Description of sequence: SH3 domain
PLPRP

SEQ ID NO: 175 - protein, 4 aa, synthetic construct
  REGION 1..4: note = Description of sequence: SH3 domain
PKPP

SEQ ID NO: 176 - protein, 7 aa, synthetic construct
  REGION 1..7: note = Description of sequence: GYF binding domain
PPPPGHR

SEQ ID NO: 177 - protein, 109 aa, synthetic construct
  REGION 1..109: note = Description of sequence: TPOR transmembrane domain N∝
SDPTRVETATETAWILVLISLVTALHLVLGLSAVLGLLLLRWQFPAHYRRLRHALWPSLPDLHRVLGQYLRDTAALSPPKATVSDTCEEVEPSLLEILPKSSERTPLPL

SEQ ID NO: 178 - protein, 489 aa, synthetic construct
  REGION 1..489: note = Description of sequence: Exemplary CD70 CAR
MALPVTALLLPLALLLHAARPQVTLKESGPVLVKPTETLTLTCTVSGFSLSNARMGVTWIRQPPGKALEWLAHIFSNDEKSYSTSLKSRLTISKDTSKTQVVLTMTNMDPVDTATYYCARIRDYYDISSYYDYWGQGTLVSVSSGGGGSGGGGSGGGGSDIQMTQSPSAMSASVGDRVTITCRASQDISNYLAWFQQKPGKVPKRLIYAASSLQSGVPSRFSGSGSGTEFTLTISSLLPEDFATYYCLQLNSFPFTFGGGTKVEINTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 179 - protein, 569 aa, synthetic construct
  REGION 1..569: note = Description of sequence: Exemplary CD70 CAR
MALPVTALLLPLALLLHAARPGGGGSCPYSNPSLCSGGGGSGGGGSQVTLKESGPVLVKPTETLTLTCTVSGFSLSNARMGVTWIRQPPGKALEWLAHIFSNDEKSYSTSLKSRLTISKDTSKTQVVLTMTNMDPVDTATYYCARIRDYYDISSYYDYWGQGTLVSVSSGGGGSGGGGSGGGGSDIQMTQSPSAMSASVGDRVTITCRASQDISNYLAWFQQKPGKVPKRLIYAASSLQSGVPSRFSGSGSGTEFTLTISSLLPEDFATYYCLQLNSFPFTFGGGTKVEINGSGGGGSCPYSNPSLCSGGGGSELPTQGTFSNVSTNVSPAKPTTTACPYSNPSLCTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 180 - protein, 116 aa, synthetic construct
  REGION 1..116: note = Description of sequence: IL2Rb YYY
QQDKVPEPASLSSNHSLTSCFTNQGYFFFHLPDALEIEACQDEGVAGAPTGSSPQPLQPLSGEDDAYCTFPSRDDLLLFSPSGQGEFRALNARLPLNTDAYLSLQELQGQDPTHLV